(19) United States
(12) Patent Application Publication
(10) Pub. No.: US 2014/0227293 A1
(43) Pub. Date: Aug. 14, 2014

(54) METHOD FOR CONTROLLING TUMOR GROWTH, ANGIOGENESIS AND METASTASIS USING IMMUNOGLOBULIN CONTAINING AND PROLINE RICH RECEPTOR-1 (IGPR-1)

(71) Applicant: Nader Rahimi, Westwood, MA (US)

(72) Inventor: Nader Rahimi, Westwood, MA (US)

(73) Assignee: TRUSTEES OF BOSTON UNIVERSITY, Boston, MA (US)

(21) Appl. No.: 14/127,707

(22) PCT Filed: Jun. 29, 2012
(86) PCT No.: PCT/US2012/044877

§ 371 Date: Mar. 6, 2014

Related U.S. Application Data
(60) Provisional application No. 61/503,392, filed on Jun. 30, 2011.

(51) Int. Cl. A61K 38/17; C07K 16/28; A61K 31/7105

(52) U.S. Cl. CPC A61K 38/1774; A61K 31/7105; C07K 16/2803
USPC 424/172.1; 514/13.3; 514/44 A; 514/19.3; 530/350

(57) ABSTRACT

The present invention provides methods and compositions for the treatment and prevention of angiogenesis, or cancer, e.g., metastatic cancer by administering an effective amount of an inhibitor of immunoglobulin containing proline rich receptor-1 (IGPR-1) protein or expression. In particular, the present invention provides methods and compositions for the treatment and prevention of cancer by administering an effective amount of an inhibitor of immunoglobulin containing proline rich receptor-1 (IGPR-1) protein or expression which is a soluble extracellular domain IGPR-1. Another aspects relates to methods and compositions comprising a IGPR-1 polypeptide or functional fragment thereof to promote angiogenesis in a subject in need thereof, e.g., for treatment of infarcts, retinopathy, AMD and the like.

### CROSS REFERENCE TO RELATED APPLICATIONS

This application claims benefit under 35 U.S.C. §119(e) of U.S. Provisional Application No. 61/503,392 filed on Jun. 30, 2011, the contents of each of which are incorporated herein by reference in their entireties.

### GOVERNMENT SUPPORT

This invention was made with U.S. Government support under Grant Number: R01EY017955-01A2 awarded by the National Institute of Health (NIH). The Government has certain rights in the invention.

### SEQUENCE LISTING

The instant application contains a Sequence Listing which has been submitted in ASCII format via EFS-Web and is hereby incorporated by reference in its entirety. Said ASCII copy, created on Jun. 28, 2012, is named71586698.txt and is 50,820 bytes in size.

### FIELD OF THE INVENTION

The present invention relates generally to modulation of angiogenesis and inhibiting metastasis and migration using an inhibitor to the immunoglobulin containing proline rich receptor-1 (IGPR-1) polypeptide or fragments thereof. Other aspects relates to compositions, methods and kits for use in a method to inhibit metastasis of cancer cells in a subject.

### BACKGROUND OF THE INVENTION

The significance of cancer and its impact on society cannot be overstated. It has been estimated that residents in the United States carry a one in five lifetime risk of dying from cancer. The vast majority of the deaths in cancer can be attributed to metastasis. Metastasis has been described as “the most fearsome” aspect of cancer. Metastasis is the immediate marker of malignancy in a tumor and carries the greatest weight in terms of prognosis. Aside from preventing cancer, it has been suggested that “no achievement would confer greater benefit on patients than methods to block distant spread”.

Angiogenesis is a coordinated cascade of numerous complex cellular processes which includes endothelial cell's migration, proliferation, sprouting and lumen formation which ultimately leads to the formation of new vessels. These coordinated cellular events are regulated by the function of various cell surface receptors and soluble ligands (Carmeliet and Jain, 2011a; Rahimi, 2006). The ability of endothelial cells to form capillary tubes is prerequisite for the establishment of a continuous vessel lumen that routes the blood flow. Several key receptor tyrosine kinases such as VEGFR-1, VEGFR-2 and VEGFR-2, and cell adhesion molecules (CAMs) including cadherins, integrins, selectins, and immunoglobulin superfamily (IgSF) proteins all are involved in angiogenesis (Bach et al., 1998; Barreiro et al., 2002; Bazzoni, 2003). Role of pro-angiogenic molecules such as vascular endothelial growth factor (VEGF) and VEGF receptors (i.e., VEGFR-1, VEGFR-2 and VEGFR-3) are well-known in regulation of differentiation, survival, proliferation and migration of endothelial cells (Gory-Faure et al., 1999; Rahimi, 2006). Studies using knockouts or blocking antibodies also have demonstrated a key role for integrins in angiogenesis. Vascular endothelial cadherin (VE-cadherin), an endothelium-specific member of the cadherin family of adhesion proteins (Bach et al., 1998), and other CAM proteins such as PECAM-1, ICAM-1 and JAM-A are also linked to angiogenesis (Bach et al., 1998; Barclay, 2003; Bazzoni, 2003).

Transmembrane proteins play an essential role in normal development, tissue repair, immunity and normal tissue homeostasis and inappropriate or errors in their signaling contribute to development of many of human diseases such as cancer, autoimmunity, and metabolic disorders such as diabetes. Due to these significant properties, transmembrane proteins lend themselves to be explored as therapeutic agents or targets. It is thought that about 20%-30% of all encoded human proteins are extracellular or membrane associated (Venter et. al., 2001; Auerbach et al. 2002), as yet, most remain uncharacterized. The immunoglobulin superfamily (IgSF) cell surface proteins perhaps is the largest group of transmembrane proteins with approximately 765 member proteins (Lander et al., 2001). The immunoglobulin (Ig) domain is about 70-110 amino acids and forms a sandwich-like structure by two anti-parallel β-sheet strands (Barclay A., 2003). Based on the structure, they are classified as variable, constant, and intermediate Ig domains (Buljan and Bateman, 2009; Harpaz and Chothia, 1994).

The Ig containing adhesion molecules are known for their vital role in embryonic development and pathological conditions such as cancer and inflammation by modulating cell-cell adhesion, cell migration (DeLisser et al., 2010; Takai et al., 2008; Yamagata and Sanes, 2008). The Ig domains are engaged in protein-protein interaction such as homophilic (i.e., trans-dimerization) interaction of cell adhesion receptors and protein-ligand interactions such growth factor receptors and soluble growth factors (Barclay, 2003; Rahimi, 2006). The Ig containing cell adhesion molecules through homophilic and/or heterophilic interactions selectively contribute to the specificity of cell-cell recognition and cell adhesion (Barclay, 2003; Rahimi, 2006). At the intracellular compartment, they interact with various cytoplasmic signaling proteins, which are often linked to the cytoskeleton (Takai et al., 2008).

Several key receptor tyrosine kinases (e.g., VEGF receptors, PDGF receptors and Tie receptors) (Rahimi, 2006), adhesion molecules (e.g., PECAM-1, L1, NCAM and VCAM) (Jaap et al., 2007), and antigen presenting molecules (e.g., Class I and II MHC) are member of this superfamily (Jones et al., 2006). Ig containing adhesion molecules such as PECAM-1 (DeLisser et al., 2010), NCAM, VCAM, Nectins (Takai et al., 2008) and Sidekick proteins (Yamagata and Sanes, 2008) play vital role in cell-cell adhesion, cell migration and invasion which are key cellular processes during embryonic development and pathological conditions such as cancer and inflammation. The Ig domains are known to mediate protein-protein interaction such as homophilic (i.e., trans-dimerization) interaction of cell adhesion receptors and protein-ligand interactions such growth factor receptors and soluble growth factors (Barclay, 2003; Rahimi, 2006). The Ig containing cell adhesion molecules mediate cell-cell adhesion by homophilic and/or heterophilic interactions and determine the specificity of cell-cell recognition (Barclay, 2003; Brümmendorf and Lemmon, 2003).

### SUMMARY OF THE INVENTION

The present invention generally relates to inhibitors of the immunoglobulin containing and proline rich receptor 1 (herein referred to as “IGPR-1” or “IGRP1”) protein for the use in methods for inhibiting angiogenesis and/or for the treatment of cancer and prevention of cancer metastasis in a subject. Accordingly, the present invention relates to compositions comprising inhibitors of IGPR-1, and use as an anti-cancer therapy, and methods for the treatment of cancer by administering to a subject with cancer a composition comprising an inhibitor of IGPR-1. Another aspect of the present invention relates to methods and compositions comprising IGPR-1 inhibitors for inhibition of angiogenesis and treatment of angiogenesis-related diseases and disorders, for example, but not limited to age-related macular degeneration (AMD).

Herein, the inventors have demonstrated that an uncharacterized receptor, IGPR-1 functions as an adhesion molecule with a broad expression in epithelial and endothelial cells. Importantly, the inventors have demonstrated that IGPR-1 regulates cellular morphology, cell-cell interaction and cell migration. Furthermore, the inventors demonstrate that IGPR-1 associates with several SH3-containing proteins and regulates angiogenesis in vivo and in vitro.

Angiogenesis, the growth of new blood vessels from pre-existing vessels, is important physiological processes and is considered to play a key role in tumor growth and metastasis. Herein, the inventors have identified IGPR-1 gene as a novel cell adhesion receptor which is expressed in various human organs and tissues mainly in cells with epithelium and endothelium origins. The inventors discovered that IGPR-1 regulates cellular morphology, homophilic cell aggregation and cell-cell interaction, and that IGPR-1 activity also modulates actin stress fiber formation, focal adhesion and reduces cell migration. The inventors demonstrated that silencing expression of IGPR-1 by siRNA and by ectopic over-expression in endothelial cells showed that IGPR-1 regulates capillary tube formation in vitro and B16F melanoma cells engineered to express IGPR-1 displayed extensive angiogenesis in mouse matrigel angiogenesis model. Moreover, the inventors also discovered that IGPR-1, through its proline rich cytoplasmic domain associates with multiple SH3-containing signaling proteins including, SPIN90/WISH, BPAG1 and CANCB2. The inventors demonstrated that silencing expression of SPIN90/WISH by siRNA in endothelial cells showed that SPIN90/WISH is required for capillary tube formation. Accordingly, the inventors have demonstrated that IGPR-1 is a novel receptor that plays an important role in cell-cell interaction, cell migration and angiogenesis, and inhibition of IGPR-1 can be used to inhibit angiogenesis in vitro and in vivo.

Additionally, the inventors herein have demonstrated that in vitro, that expression of the IGPR-1 protein inhibits cell migration. However, in contrast, the inventors have surprisingly discovered that in vivo systems, expression of the IGPR-1 protein stimulated angiogenesis and tumor growth. The inventors discovered that this IGPR-1 mediated increase in angiogenesis and tumor growth in vivo could be prevented by inhibition of IGPR-1 protein function or its inhibition of IGPR-1 gene expression. Accordingly, in one embodiment, the invention relates to compositions comprising inhibitors of IGPR-1 protein function or its expression from the IGRP1 gene, and its use as an anti-cancer therapy, and methods for the treatment of cancer by administering to a subject with cancer a composition comprising an inhibitor of IGPR-1. Accordingly, another aspect of the present invention relates to methods and compositions comprising IGPR-1 inhibitors for inhibiting angiogenesis in a subject, and methods using IGPR-1 inhibitors for the treatment of angiogenesis-related diseases and disorders, for example, but not limited to age-related macular degeneration (AMD).

In some embodiments, an inhibitor of IGPR-1 expression can be any agent, e.g., siRNA gene silencing agents as disclosed herein. In some embodiments, an inhibitor of IGPR-1 protein is a non-functional fragment of the IGPR-1 polypeptide which is dominant negative inhibitor of IGPR-1 protein, e.g., a soluble recombinant extracellular domain of IGPR-1 (herein also referred to as “Ig-IGRP-1” as shown in FIG. 4H, such as a sequence of SEQ ID NO:5, SEQ ID NO: 6 or SEQ ID NO: 16) or alternatively, a dominant negative inhibitor of IGPR-1 which lacks the immunoglobulin (Ig) domain of IGPR-1 (referred to herein as “ΔN-IGPR-1” (see FIG. 4C), such as, for example SEQ ID NO: 4), or a fragment of SEQ ID NO: 4 or SEQ ID NO: 16 which inhibits the activity of the full length IGPR-1 protein of SEQ ID NO: 1 to inhibit angiogenesis. In some embodiments, such non-functional fragment of IGPR-1 which is Ig-IGPR1, corresponding to SEQ ID NO: 5, 6 or 16, or a fragment of SEQ ID NO: 5, 6 or 16 of Ig-IGRP-1 inhibits the cis-dimerization of the full length IGPR-1 polypeptide. In alternative embodiments, a non-functional fragment of IGRP-1 which is ΔN-IGPR-1 of SEQ ID NO: 4, or a fragment of SEQ ID NO:4 inhibits the trans-dimerization of the full length IGPR-1 polypeptide.

In some embodiments, an inhibitor of IGPR-1 is an antibody or a decoy ligand (including a soluble recombinant extracellular domain of IGPR-1), etc.

The inventors have used a unique strategy of bioinformatics coupled with cell culture-based analysis of human genome, a strategy referred to as “reverse proteomic” analysis, to probe the entire human genome and have discovered genes whose protein products suggest a potential function relevant to cancer metastasis. Accordingly, using such reverse proteomics, the inventors discovered IGPR-1 (immunoglobulin containing and proline rich receptor 1), which was discovered to be is expressed in various organs including vein, arteries and brain. Herein, it is demonstrated that forced expression of a dominant negative mutant of IGPR-1 in human kidney cells prevent the cells from growing or migrating. Accordingly, one aspect of the present invention relate to inhibiting IGPR-1 polypeptide to inhibition of metastasis in a subject. Another aspect of the present invention relate to a method of treating cancer in a subject by administering a subject a composition comprising an inhibitor of the IGPR-1 polypeptide to the subject.

Without wishing to be bound by theory, the immunoglobulin superfamily (IgSF) proteins are a conserved class of cell surface receptors that are known to modulate diverse biological roles in mammalian cells including, cell growth, motility, and adhesion. Herein, IGPR-1 (immunoglobulin containing and proline rich receptor-1) gene was identified as a novel cell adhesion receptor which is conserved across several species. IGPR-1 protein encompasses an extracellular domain containing a single immunoglobulin domain, two potential N-glycosylation sites, a transmembrane domain and a cytoplasmic domain with a proline rich motif. Immunohistochemical staining of human tissues demonstrate that IGPR-1 is widely expressed by epithelial and endothelial cells in various human organs including, breast, colon, bladder, bronchus, stomach, small intestine, and pancreas. IGPR-1 was also demonstrated to be expressed in certain human tumors yet its expression is undetectable in some others. IGPR-1 was discovered to regulate cellular morphology, focal adhesion and actin stress fiber formation.

Transfer of IGPR-1 into tumor cell lines including mouse B16F melanoma cells and human colorectal tumor cell lines, HT-29 and HCT-116 inhibited cell migration and invasion and it was discovered that the extracellular domain is required to modulate cellular migration. IGPR-1 interacts with several SH3 containing signaling proteins including, SPIN90/WISH, BPAG1 and CACNB2. Modulation of expression of SPIN90 in B16F cells either by over-expression or by siRNA showed that SPIN90 activity is critical for IGPR-1 dependent cell migration. Herein, it is demonstrated that IGPR-1 functions as an adhesion receptor which activates a distinct intracellular signaling pathway that regulates cell morphology, adhesion and motility. In particular, IGPR-1 was demonstrated to have anti-metastatic function and anti-invasive function of cancer cell in vitro.

However, in contrast to its in vitro anti-metastatic function and anti-invasive function, IGPR-1 was discovered to promote angiogenesis and tumor growth in vivo, and administration of an inhibitor, e.g., such as a soluble extracellular domain of IGPR-1 was demonstrated to inhibit angiogenesis and tumor growth in vivo.

Accordingly, one aspect of the present invention relates to a method of treating cancer in a subject at risk thereof, comprising administering to the subject an effective amount of a composition comprising an inhibitor of immunoglobulin containing proline rich receptor-1 (IGPR-1) protein or expression for the treatment and/or prevention of a malignancy or neoplasia disorder in the subject.

Another aspect of the present invention relates to a method of inhibiting endothelial cell migration, comprising contacting an endothelial cell with an inhibitor of immunoglobulin containing proline rich receptor-1 (IGPR-1) protein or expression.

Another aspect of the present invention relates to a method of treating cancer in a subject at risk thereof, comprising administering to the subject a composition comprising an inhibitor of SPIN90 gene expression or protein or expression for the treatment and/or prevention of a malignancy or neoplasia disorder in the subject.

One aspect of the present invention relates to a method of inhibiting angiogenesis in a subject, e.g., a human subject comprising administering to the subject a composition comprising an inhibitor of the IGPR-1 polypeptide.

Another aspect of the present invention relates to a method of treating cancer in a subject, e.g., a human subject, comprising administering to the subject an effective amount of a composition comprising an inhibitor of immunoglobulin containing proline rich receptor-1 (IGPR-1) protein or expression for the treatment and/or prevention of a malignancy or neoplasia disorder in the subject.

Another aspect of the present invention relates to a method of inhibiting endothelial cell migration comprising contacting an endothelial cell with an inhibitor of immunoglobulin containing proline rich receptor-1 (IGPR-1) protein or expression.

In all aspect of the invention as disclosed herein, the IGPR-1 protein corresponds to SEQ ID NO: 1 and is encoded by SEQ ID NO: 2, or a variant thereof.

In some embodiments of all aspects as disclosed herein, an inhibitor of IGPR-1 can be a mimetic of the full length IGPR-1 polypeptide which does not retain the full-length IGPR-1 signaling function, but can still bind to the natural ligand of IGPR-1 to inhibit angiogenesis. Accordingly, in some embodiments, an inhibitor of IGPR-1 can be a non-functional fragment of IGRP-1, for example a Ig-IGRP inhibitor of IGRP-1 which lacks the TM and extracellular domain of IGRP-1, or alternatively, a ΔN-IGPR-1 inhibitor of IGPR-1 which lacks the extracellular Ig domain of IGPR-1, as disclosed herein.

Accordingly, in some embodiments of all aspects as disclosed herein, an inhibitor of IGPR-1 is a non-functional fragment of IGPR-1 which is a soluble extracellular domain of IGPR-1, e.g., a fragment of IGPR-1 which comprises only the extracellular Ig-domain of the IGPR-1 protein, (e.g., lacks the transmembrane domain and intracellular/cytosolic domain), herein also referred to as “Ig-IGRP-1” as shown in FIG. 4H. In some embodiments, a Ig-IGRP-1 corresponds to sequence of SEQ ID NO: 6 or 16, or a is a fragment or variant thereof, where the Ig-IGPR-1 or a fragment or variant of Ig-IGRP1 inhibits the full length IGPR-1 polypeptide function and/or IGPR-1 cis-dimerization. In some embodiments, a fragment of Ig-IGPR-1 which inhibits the function of the IGPR-1 polypeptide and/or IGPR-1 cis-dimerization is at least about 60, or at least about 80, or at least about 100 N-terminal amino acids of SEQ ID NO: 5 or SEQ ID NO: 16 or a variant thereof. In some embodiments, a Ig-IGRP inhibitor comprising SEQ ID NO: 6 or SEQ ID NO: 16 or a fragment or variant thereof comprises a signal sequence at the N-terminus, e.g., SEQ ID NO: 7 or a variant or fragment thereof.

In alternative embodiments, an inhibitor of IGPR-1 is a non-functional fragment of IGPR-1 which serves as dominant negative inhibitor of IGPR-1 and comprises the transmembrane domain and the cytosolic/intracellular domain of IGRP-1 and where the extracellular Ig-domain of IGPR-1 is deleted, herein referred to herein as “ΔN-IGPR-1” (see FIG. 4C). In some embodiments, a ΔN-IGPR-1 inhibitor of IGPR-1 which lacks the extracellular Ig domain of IGPR-1 corresponds to SEQ ID NO: 4 or a fragment or variant thereof, where the ΔN-IGPR-1 or fragment or variant inhibits IGPR-1 polypeptide function and/or IGPR-1 trans-dimerization. In some embodiments, a fragment of ΔN-IGPR-1 which inhibits the function of the IGPR-1 polypeptide and/or IGPR-1 trans-dimerization is at least about 60, or at least about 80, or at least about 100 N-terminal amino acids or C-terminal amino acids of SEQ ID NO: 4 or a variant thereof.

In some embodiments, an inhibitor of IGPR-1 is an antibody or a decoy ligand (including a soluble recombinant extracellular domain of IGPR-1, e.g., Ig-IGRP), etc. In alternative embodiments, an inhibitor of IGPR-1 is selected from the group consisting of: RNAi agent, oligonucleotide, antibody inhibitor, peptide inhibitor, protein inhibitor, avidimir, and functional fragments or derivatives thereof. In some embodiments, a siRNA inhibitor of IGPR-1 corresponds to SEQ ID NO: 20 or 21 or variants or fragments thereof which inhibit the expression of IGRP-1 protein or mRNA.

In some embodiments for the prevention of angiogenesis or treatment of cancer in a subject, the subject can be further administered an inhibitor of SPIN90 polypeptide or SPIN90 gene expression.

In some embodiments, the methods and compositions comprising a IGPR-1 inhibitor as disclosed herein can be used to inhibit angiogenesis in a subject, e.g., where the subject has cancer, e.g., a cancer of the epithelium, for example but not limited to, a cancer selected from the group consisting of: bladder cancer, Breast cancer, Bronchus cancer, cancer of the Fallopian Tube, cancer of the gastrointestinal tract, cancer of esophagus, stomach cancer, colon cancer, cancer of the rectum, cancer of the small intestine, pancreatic cancer, cancer of the placenta, prostate cancer, skin cancer, testicular cancer, thyroid cancer, cancer of the thymus, endometrium cancer, cancer of the urethra. In some embodiments, the cancer is squamous Cell carcinoma (SCC), Infiltrating Duct carcinoma, adenocarcinoma, pillary carcinoma. In some embodiments, the cancer cell type is selected from the group consisting of: urothelim, tumor cells, glandular/Lobular Epithelium cells, bronchial Epithelium cells, fallopian tube lining Epithelium cells, squamous cell carcinoma cells, adenocarcinoma cells, stomach epithelium cells, intestinal epithelium cells, colonic epithelium cells, acniar cells, trophoblastic Epithelium cells, epidermal cells, karatinocytes, skin cells, testis semiferinstubulule cells, glandular epithelium cells of the thymus, thyroid cells, urothelium cells, endometrial Glandular cells.

In some embodiments of all aspects as disclosed herein, a subject administered an inhibitor of IGRP-1 is selected for treatment by identifying a subject with a cancer expressing IGPR-1.

In some embodiments of all aspects as disclosed herein, a subject administered an inhibitor of IGRP-1 has an angiogenesis-related disease characterized by increase in angiogenesis, for example, but not limited to a disease or disorder selected from the group consisting of cancer, macular degeneration; diabetic retinopathy; rheumatoid arthritis; Alzheimer's disease; obesity, psoriasis, atherosclerosis, vascular malformations, angiomata, and endometriosis. In some embodiments, a subject administered an inhibitor of IGRP-1 has neovascularization, for example, ocular neovascularization. In some embodiments, a subject administered an inhibitor of IGRP-1 has at least one of the disorders selected from the group comprising: age-related macular degeneration (AMD), diabetic retinopathy, retinopathy of prematurity (ROP), arthritis, rheumatoid arthritis (RA), osteoarthritis, cardiovascular disease.

In some embodiments of all aspects of the invention as disclosed herein, subject administered an inhibitor of IGRP-1 has cancer, for example, a metastatic cancer, a malignant cancer or a neoplasia disorder. In some embodiments, the cancer is of endothelial or epithelial origin.

In some embodiments of all aspects of the invention as disclosed herein, a subject to be treated is a mammal, e.g., a human. In some embodiments, a subject administered an inhibitor of IGRP-1 can be further administered an anti-angiogenic therapy in conjunction with the inhibitor of the IGPR-1 polypeptide, for example but not limited to an anti-angiogenic therapy is chemotherapy and/or radiation therapy.

Another aspect of the present invention relates to a method of treating cancer in a subject at risk thereof, comprising administering to the subject a composition comprising an inhibitor of SPIN90 gene expression or protein or expression for the treatment and/or prevention of a malignancy or neoplasia disorder in the subject.

Another aspect of the present invention relates to a method of inhibiting angiogenesis in a subject, comprising administering to the subject a composition comprising an inhibitor of SPIN90 gene expression or protein or expression.

In some embodiments of all aspects as disclosed herein, a subject administered an IGRP-1 inhibitor as disclosed herein is a subject in need thereof, where the subject in need thereof has at least one of the following: (i) increased IGRP-1 expression and/or (ii) has cancer expressing IGRP-1 and/or (iii) has cancer, e.g., metastatic cancer, and/or (iv) has an angiogenesis-related disease or disorder characterized by increased angiogenesis.

Another aspect of the present invention relates to a method to promote angiogenesis in a subject in need thereof, comprising administering to a subject a composition comprising an IGPR-1 polypeptide or a functional fragment thereof, where a functional fragment thereof is a IGPR-1 mimetic or has a substantially similar biological activity as full length IGRP-1 of SEQ ID NO: 1.

In some embodiments, a subject in need of administering a IGRP-1 polypeptide or a functional fragment thereof has an angiogenesis-related disorder characterized by a decrease in angiogenesis, for example, where the subject is a transplant recipient, or has undergone a transplant surgery. In some embodiments, the subject is, or will be a recipient of transplanted retinal pigment epithelium (RPE) cells.

In some embodiments, a subject administered a IGRP-1 polypeptide or a functional fragment of IGRP-1 in need of neovascularization of any one of the group selected from: a tissue engineering construct, an organ transplant, tissue repair, regenerative medicine, and a wound. In some embodiments, a subject administered a IGRP-1 polypeptide or a functional IGRP-1 fragment is in need of wound repair, or has had an infarct, cardiac infarct, stroke.

Another aspect of the present invention relates to an IGRP-1 inhibitor which is non-functional IGPR-1 fragment, which is soluble extracellular domain of IGPR-1 (Ig-IGRP-1), for inhibiting angiogenesis or endothelial cell migration in a subject in need thereof, wherein the soluble extracellular domain of IGPR-1 blocks the function of the IGPR-1 polypeptide and/or inhibits IGPR-1 cis-dimerization.

Another aspect of the present invention relates to use of a non-functional IGPR-1 fragment which is a soluble extracellular domain of IGPR-1 (Ig-IGRP-1) for the manufacture of a medicament for inhibiting angiogenesis or endothelial cell migration, in a subject in need thereof.

Another aspect of the present invention relates to an IGRP-1 inhibitor which is non-functional IGPR-1 fragment, which is a dominant negative inhibitor of IGPR-1 (ΔN-IGPR-1), for inhibiting angiogenesis or endothelial cell migration in a subject in need thereof, wherein the ΔN-IGPR-1 blocks the function of the full length IGPR-1 polypeptide, and/or inhibits IGPR-1 transdimerization.

Another aspect of the present invention relates to the use of a non-functional fragment of IGRP-1 which is a dominant negative inhibitor of IGPR-1 (ΔN-IGPR-1) for the manufacture of a medicament for inhibiting angiogenesis or endothelial cell migration, in a subject in need thereof.

Another aspect of the present invention relates to an siRNA directed specifically against the IGRP-1 gene, (e.g., but not limited to SEQ ID NO: 20 and 21 or a fragment or variant thereof), or the SPIN90 gene for inhibiting angiogenesis or endothelial cell migration in a subject in need thereof.

Another aspect of the present invention relates to the use of an siRNA directed specifically against the IGRP-1 gene (e.g., but not limited to SEQ ID NO: 20 and 21 or a fragment or variant thereof) or the SPIN90 gene for the manufacture of a medicament for inhibiting angiogenesis or endothelial cell migration, in a subject in need thereof.

All publications, patent applications, patents and other reference material mentioned are incorporated by reference in their entirety. In addition, the materials, methods and examples are only illustrative and are not intended to be limiting. The citation of references herein shall not be construed as an admission that such is prior art to the present invention

### DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-1H show the identification of IGPR-1 as a novel cell surface glycoprotein. FIG. 1A shows the deduced amino acid sequence of human IGPR-1 (SEQ ID NO: 1). Amino acids (1-22) (MGSPGMVLGL LVQIWALQEASS; SEQ ID NO: 16) are putative signal sequence (underlined). The immunoglobulin domain of IGPR-1 is shown in red bold (PN LLQVRQGSQA TLVCQVDQATAWERLRVKWT KDGAILCQPY ITNGSLSLGV CGPQGRLSWQ APSHLTLQLDPVSLNHSGAY VCWAAVEIPE LEEAEGNITR LFV; SEQ ID NO: 17), which can be used as a dominant negative inhibitor of IGPR-1 (Ig-IGPR-1) and proline rich cytoplasmic region is also shown (WGRRSCQQ RDSGNSPGNA FYSNVLYRPR GPPKKSEDCS GEGKDQRGQS IYSTSFPQPA PRQPHLASRP CPSPRPCPSPRPGHPVSMVR VSPRPSPTQQ PRPKGFPKVG EE; SEQ ID NO: 18). FIG. 1B shows a schematic of orthologs of IGPR-1 in various species. FIG. 1C shows the predicted immunoglobulin domain of IGPR-1 which is based on the Ig domain of myelin-associated glycoprotein. IGPR-1 was cloned into retroviral vector (pMSCV) and PAE cells were transduced with viruses of empty vector or IGPR-1. FIG. 1D shows whole cell lysates derived from PAE cells were subjected Western blot an analysis using anti-IGPR-1 antibody. FIG. 1E shows whole cell lysates derived from PAE cells expressing IGPR-1 were either untreated or treated with PNGase and immunoblotted with anti-IGPR-1 antibody. FIG. 1F shows immunofluorescence microscopy of PAE cells expressing IGPR-1. FIG. 1G shows PAE cells expressing IGPR-1 which were subjected to cell surface biotinylation and biotinylated IGPR-1 detected with Anti-SA-HRP antibody. FIG. 1H shows the same membrane was re-blotted for with anti-IGPR-1.

FIGS. 2A-2D show expression of IGPR-1 in human organs and tissues: FIG. 2A shows total RNA derived from various human organs which was subjected to qPCR using primers specifically designed to amplify exon 2 and 3 of IGPR-1 and the relative levels of IGPR-1. FIG. 2B shows cell lysates derived from various human tissues subjected to Western blot analysis using anti-IGPR-1 antibody. FIGS. 2C and 2D show a human tissue microarray of normal human tissues is stained for IGPR-1. The array was viewed under light microscope and pictures were taken (20×) (FIG. 2C) or (40×) (FIG. 2D). The arrows indicate blood vessels.

FIGS. 3A-3K show IGPR-1 activity regulates angiogenesis. FIGS. 3A and 3B show PAE cells expressing empty vector (pMSCV) (FIG. 3A) or IGPR-1 (FIG. 3B) which were subjected to matrigel assay and analyzed after 24 hours. FIG. 3C shows quantification of capillary tube formation. FIG. 3D shows the expression of IGPR-1 in PAE cells and protein loading control. FIGS. 3E and 3F shows HUVEC cells transfected with control (Ctr) siRNA (FIG. 3E) or IGPR-1 siRNA (FIG. 3F) and then assessed using the matrigel assay. FIGS. 3G and 3H show quantification of capillary tube formation of HUVEC cells and expression of IGPR-1. FIG. 3I shows B16F cells expressing empty vector or IGPR-1 were mixed with matrigel and injected under skin for 8 days and pictures were taken. FIG. 3J shows quantification of angiogenesis using Drabkin's Reagent which measures the hemoglobin content in tumor tissue. FIG. 3K shows the expression of IGPR-1 in B16F cells.

FIGS. 4A-4J show that IGPR-1 regulates cellular morphology and adhesion: Morphology of PAE cells expressing IGPR-1 and PAE cells expressing empty vector were assessed using light microscopy (top panel). FIG. 3A shows immunofluorescence microscopy of PAE cells expressing empty vector (pMSCV/PAE) or IGPR-1 (IGPR-1/PAE) which were stained with FITC-labeled Phalloidin. FIG. 4B shows light microscopy images of PAE cells expressing either empty vector or IGPR-1 which were trypsinized with 0.05% trypsin/EDTA for indicated times. FIG. 4C is a schematic of IGPR-1 and N-terminus deleted IGPR-1 ((ΔN-IGPR-1). FIG. 4D shows the expression of IGPR-1 and ΔN-IGPR-1 in PAE cells. FIGS. 4E, 4F, 4G show light microscopy images of PAE cells expressing empty vector (pMSCV), IGPR-1 or ΔN-IGPR-1 which were assessed using the aggregation assay as described in the method section. FIG. 4H is a schematic of the generation of recombinant GST-Ig-IGPR-1, which can be used as a dominant negative inhibitor of IGPR-1 as it comprises the Ig extracellular domain of IGPR-1 (Ig-IGPR-1). An Ig-IGPR-1 polypeptide can inhibit IGPR-1 when bound or unbound to GST, as it functions to prevent cis- or transdimerization of IGPR-1. FIG. 4I shows PAE cells expressing IGPR-1 which were incubated either with DMEM medium alone, DMEM plus GST, and DMEM plus GST-N terminus/extracellular domain of IGPR-1. After 15 minutes incubation cells were plated in 24-well plates and allowed to adhere. Pictures of cells were taken after 30 minutes incubation in 24-well plates. FIG. 4J shows results of adherent cells counted under microscope (three randomly selected fields were counted in each well) after removal of the non-adherent cells after 30 minutes post-plating.

FIGS. 5A-5L show IGPR-1 regulates focal adhesion, inhibits paxillin phosphorylation and cell migration. FIGS. 5A-5F show immunofluorescence of PAE cells expressing either empty vector (pMSCV/PAE) or IGPR-1 (IGPR-1/PAE) which were grown in 10% FBS/DMEM medium, and stained for vinculin (green) (FIGS. 5A, 5D), nuclei (purple) and F-actin (Phallodin; red) (FIGS. 5B, 5E), and merged images (FIG. 5C, 5F). FIG. 5G shows quantification of focal adhesion PAE cells expressing empty vector, pMSCV or IGPR-1. FIG. 5H shows anti-phospho-Y118 paxillin immunoblot analysis of cell lysates derived from PAE cells expressing empty vector and IGPR-1. FIG. 5I shows anti-IGPR-1 immunoblot analysis of cell lysates derived from PAE cells expressing empty vector and IGPR-1. FIG. 5J shows anti-IQGAP1 immunoblot analysis of cell lysates derived from PAE cells expressing empty vector and IGPR-1 for protein loading control. FIG. 5K shows PAE cells expressing empty vector (pMSCV/PAE) versus IGPR-1 (IGPR-1/PAE) which were assessed using the wounding assay and demonstrates migration of cells toward wound area, as described in the Materials and Methods section. FIG. 5L shows B16F cells expressing empty vector (pMSCV/B16F) versus IGPR-1 (IGPR-1/B16F) which were assessed using the wounding assay and migration of cells toward wound area.

FIGS. 6A-6G show that IGPR-1 associates with SH3 containing cytoplasmic signaling proteins. FIG. 6A is a schematic of detection of IGPR-1 associated SH3 containing proteins. A protein array containing SH3 domains of 34 different proteins was blotted with a recombinant cytoplasmic domain of IGPR-1 (rec-cIGPR-1) protein and was detected with anti-IGPR-1 antibody. FIG. 6B shows SH3 domains that interacted with rec-cIGPR-1 protein. FIG. 6C lists exemplary SH3 domains that were present in SH3 array but did not interact with IGPR-1. FIG. 6D shows a schematic of the SH3 domains in SPIN-90, BPAG1 and CACNB1 proteins which interact with IGPR-1. FIG. 6E shows an immunoblot with anti-IGPR-1 antibody after GST-SH3 pull down from cell lysates derived from PAE cells expressing IGPR-1, showing GST-SH3 domains of SPIN-90, CANCB2 and BPAG1. FIG. 6F shows PAE cells expressing empty vector and IGPR-1 which were subjected to immunoprecipitation with anti-IGPR-1 and immunoblotted for SPIN90. FIG. 6G shows the same membrane was re-blotted for IGPR-1.

FIGS. 7A-7G show SPIN90 activity regulates angiogenesis: FIG. 7A shows representative images of capillary tube formation of PAE cells with the empty vector (pMSCV/PAE) or induced to express IGPR-1 alone (IGPR-1) or co-express IGPR-1 with SPIN90 (IGPR-1/SPIN90) 48 hours after the matrigel-based angiogenesis assay. FIG. 7B shows quantification of capillary tube formation as determined using NIH image J software. FIG. 7C shows anti-IGPR-1 and FIG. 7D shows anti-SPIN90 immunoblot of whole cell lysates derived from PAE cells with the empty vector (pMSCV/PAE) or expressing IGPR-1 alone (IGPR-1) or co-expressing IGPR-1 with SPIN90 (IGPR-1/SPIN90). FIG. 7E shows HUVEC cells 46 hours after transfection with control siRNA (Ctr. siRNA) or SPIN90-siRNA (SPIN-siRNA) and assessed for matrigel-based capillary tube formation. FIG. 7F shows quantification of capillary tube formation. FIG. 7G shows the expression of SPIN90 and protein loading control, PLCγ1 in HUVEC cells.

FIGS. 8A-8H illustrate the inhibition of cell migration by IGPR-1. FIG. 8A shows that PAE cells and B16F melanoma cells expressing either empty vector or IGPR-1 were subjected to proliferation assay. Proliferation of PAE cells were measured over 24 and 48 hours in the presence of 10% FBS. FIG. 8B shows the proliferation of B16F cells measured in the absence (serum-starved) or in presence of 10% FBS for 24 hours. FIG. 8C shows the schematic presentation of IGPR-1 constructs and their expression in B16F cells. FIG. 8D show the migration of B16F cells expressing IGPR-1, N-terminus truncated IGPR-1 (ΔN-IGPR-1) and empty vector. Cells were grown to full confluent condition, then cells were wounded by a 5-ml pipette, cell were washed then were incubated with 10% FBS for 10 hours and viewed under microscope and pictures were taken. FIG. 8E shows the invasion of B16F cells expressing empty vector (pMSCV), IGPR-1 and ΔN-IGPR-1. The invasion assay was performed as described in the Materials and Methods section of the Examples and invasion of cells were measured after 48 hours. FIG. 8F shows the migration of HT-29 and HCT-116 cells over-expressing IGPR-1 and FIG. 8G shows the morphology of HT-29 cells expressing IGPR-1. FIG. 8H shows the expression levels of IGPR-1 in HT29 and HCT116.

FIGS. 9A-9C illustrate that IGPR-1 undergoes dimerization and regulates cell adhesion. FIG. 9A shows western blot analysis using anti-IGPR-1 antibody of PAE cells expressing IGPR-1 grown at normal (control), confluent or sparse culture condition for 12 hours cells with a cross-linker, BS3 (2 mM, 30 minutes at 4° C.). FIG. 9B shows anti-Hsp70 for protein loading control. FIG. 9C shows a schematic of proposed dimerization of IGPR-1.

FIGS. 10A-10I illustrate the regulation of cellular migration by SPIN90. FIGS. 10A and 10B show B16F cells expressing empty vector, GFP tagged SPIN90 were subjected to wounding assay and migration of cells assessed after 10 hours. FIGS. 10C and 10D show expression of SPIN90 and protein loading control. B16F cells expressing control siRNA and SPIN90 siRNA, B16F cells expression IGPR-1 treated with control siRNA or SPIN90 siRNA for 48 hours and cells were wounded as above for 10 hours and pictures were taken under microscope as shown in FIGS. 10E and 10F. FIG. 10G shows anti-SPIN90 immunoblot, FIG. 10H shows anti-IGPR-1 immunoblot, and FIG. 10I shows protein loading control using anti-IQGAP1.

FIG. 11 shows the immunohistochemical analysis of IGPR-1 expression in human tumor samples, where the human tumor tissue microarray is stained for IGPR-1, and the array was viewed under light microscope and pictures were taken (20×).

FIG. 12A-12B shows IGPR-1 promotes tumor angiogenesis in vivo and a dominant negative inhibitor to IGPR-1 can inhibit IGPR-1 mediated angiogeneis. FIG. 12A shows B16F cells (5×10⁶) expressing either IGPR-1 or empty vector (pMSCV) were mixed with Matrigel and injected under skin into C57 black mice (n=4). After 21 days, mice were sacrificed and tumor mass was photographed. FIG. 12B shows that a dominant negative inhibitor of IGPR-1, which soluble extracellular domain of IGPR-1, inhibits tumor growth and angiogenesis. B16F cells (5×10⁶) expressing either alone or mixed with soluble IGPR-1 corresponding to its extracellular domain (S-IGPR-1) and both groups then were mixed with Matrigel and injected under skin into nude mice (n=2). After 18 days, mice were sacrificed and tumor mass was photographed (left panel). The dissected tumor from the same mice are also shown ((right panel).

FIG. 13A-13C shows the expression of IGPR-1 in human eye. FIG. 13A shows a schematic of eye and the location of RPE (retinal pigment epithelium) cells. Postmortem human eye tissue was subjected to immunohistochemistry (IHC) using polyclonal anti-IGPR-1 antibody. FIG. 13B shows the eye section was subjected to IHC without anti-IGPR-1 (as a negative control). FIG. 13C shows the eye section was subjected to IHC with anti-IGPR-1 antibody. Arrow indicates expression of IGPR-1 in RPE and choroidal endothelial cells.

### DETAILED DESCRIPTION OF THE INVENTION

The present invention generally relates to inhibitors of the IGPR-1 (immunoglobulin containing and proline rich receptor 1) protein for the use in methods for the treatment of cancer and prevention of cancer metastasis in a subject. Accordingly, the present invention relates to compositions comprising inhibitors of IGPR-1, and use as an anti-cancer therapy, and methods for the treatment of cancer by administering to a subject with cancer a composition comprising an inhibitor of IGPR-1.

In some embodiments, an inhibitor can be any agent which inhibits the expression of IGPR-1 or inhibits the protein expressed from the IGPR-1 gene. In some embodiments, an agent which inhibits IGPR-1 is an inhibitor of an activator of IGPR-1, for example, an agent which inhibits the expression and/or function of SPIN90.

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

The term “modulator” refers to an agent which modules, e.g. increases or decreases the function of a gene or protein. Stated another way, the term “modulator” refers to an agent which increases or decreases the biological function of the molecule to which it is a modulator to. In some embodiments, a modulator of a IGPR-1 can be for example, refers to any agent or entity capable of inhibiting the expression or biological activity of the IGPR-1 gene. Thus, a modulator can operate to decrease the transcription, translation, post-transcriptional or post-translational processing of the IGPR-1 gene or otherwise inactivate the activity or function of the IGPR-1 protein, polypeptide or polynucleotide encoded by the IGPR-1 gene in any way. For example, in some embodiments, a modulator gene is an agent which inhibits IGPR-1 gene (e.g. antagonists such as small molecules or gene silencing RNAi molecules which target and inhibit IGPR-1).

In some embodiments, the modulation of a IGPR-1 gene includes decreasing the expression of the IGPR-1 gene, or level of mRNA molecule encoding a IGPR-1 protein by at least 5%, 10%, 20%, 30%, 40%, 50%, 60%, 70%, 80%, 90%, 95%, 2-fold, 3-fold, 4-fold, 5-fold or more different from that observed in the absence of the modulator. The % and/or fold difference can be calculated relative to the control or the non-control, for example, the % difference (% difference) can be calculated by: dividing the level of mRNA expression in the presence of a modulator (x)—level of mRNA expression in the absence of the modulator (y) by either: the level of mRNA expression in the absence of the modulator (y) or the level of mRNA expression in the presence of a modulator (x).

The term “antagonist” is well known in the art and can be used interchangeably herein with the term “inhibitor” and generally refers to an agent which inhibits or decreases the expression of a gene (e.g. inhibits the IGPR-1 gene) or the biological function of the IGPR-1 protein expressed by the IGPR-1 gene by a statistically significant amount relative to in the absence of an inhibitor. The term “inhibition” or “inhibit” or “reduce” when referring to the activity of an agent which inhibits the IGPR-1 gene as disclosed herein refers to prevention of cell migration or inhibit angiogenesis. However, for avoidance of doubt, “inhibit” means statistically significant decrease in IGPR-1 expression in the presence of an inhibitor of a IGPR-1 gene by at least about 10% as compared to in the absence of the inhibitor of the IGPR-1 gene, for example a decrease by at least about 20%, at least about 30%, at least about 40%, at least about 50%, or least about 60%, or least about 70%, or least about 80%, at least about 90% or more than 90% decrease or a 100% decrease in the expression of IGPR-1 transcript (e.g., mRNA) or IGPR-1 protein as compared to in the absence an inhibitor of a IGPR-1.

As used herein, an “inhibitor of IGPR-1” or “IGPR-1 inhibitor” are used interchangeably and refers to any molecule or agent which decreases or inhibits the expression of IGPR-1 or inhibits the consequences of activated IGPR-1, i.e. inhibits the downstream signaling of IGPR-1, or inhibits the binding of IGPR-1 to itself (e.g., inhibits IGPR-1 dimerization, either cis- or trans-dimerization) or binding other signaling proteins (e.g., SPIN90, BAG1, CACNB). For example, an IGPR-1 inhibitor can be an siRNA or dsRNA that inhibits the expression of IGPR-1, a IGPR-1 small molecule inhibitor, a IGPR-1 antagonist, a IGPR-1 inhibiting antibody, a dominant negative IGPR-1, or non-functional fragment of IGPR-1 (e.g., Ig-IGPR or ΔN-IGPR-1 as disclosed herein).

As used herein, the term “inhibiting IGPR-1 activity” refers to a decrease in the activity of IGPR-1 by at least 10%, at least 20%, at least 30%, at least 40%, at least 50%, at least 60%, at least 70%, at least 80%, at least 90%, at least 95%, at least 99%, or even 100% (i.e., no activity) in the presence of a IGPR-1 inhibitor as compared to the activity of IGPR-1 in the absence of such an inhibitor.

The terms “increased”, “increase” or “enhance” or “higher” are all used herein to generally mean an increase by a statically significant amount; for the avoidance of any doubt, the terms “increased”, “increase” or “enhance” or “higher” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “lower”, “reduced”, “reduction” or “decrease” or “inhibit” are all used herein generally to mean a decrease by a statistically significant amount. However, for avoidance of doubt, “lower”, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The term “agent” refers to any entity which is normally not present or not present at the levels being administered in the cell. Agent can be selected from a group comprising: chemicals; small molecules; nucleic acid sequences; nucleic acid analogues; proteins; peptides; aptamers; antibodies; or fragments thereof. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc. A protein and/or peptide or fragment thereof can be any protein of interest, for example, but are not limited to: mutated proteins; therapeutic proteins and truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. Alternatively, the agent can be intracellular within the cell as a result of introduction of a nucleic acid sequence into the cell and its transcription resulting in the production of the nucleic acid and/or protein modulator of a β2-AR regulator gene within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

As used herein, “gene silencing” or “gene silenced” in reference to an activity of an RNAi molecule, for example a siRNA or miRNA refers to a decrease in the mRNA level in a cell for a target gene (e.g. β2-AR regulator gene) by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene. By way of an example only, in some embodiments RNAi agents which serve to inhibit or gene silence are useful in the methods, kits and compositions disclosed herein to inhibit a IGPR-1 gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lin et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule.

The term “gene” used herein can be a genomic gene comprising transcriptional and/or translational regulatory sequences and/or a coding region and/or non-translated sequences (e.g., introns, 5′- and 3′-untranslated sequences and regulatory sequences). The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′- or 3′ untranslated sequences linked thereto. A gene can also be an amplified nucleic acid molecule produced in vitro comprising all or a part of the coding region and/or 5′- or 3′-untranslated sequences linked thereto.

The term “gene product(s)” as used herein refers to include RNA transcribed from a gene, or a polypeptide encoded by a gene or translated from RNA.

The term “nucleic acid” used herein refers to polynucleotides such as deoxyribonucleic acid (DNA), and, where appropriate, ribonucleic acid (RNA), polymers thereof in either single- or double-stranded form. Unless specifically limited, the term encompasses nucleic acids containing known analogs of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer, et al., Nucleic Acid Res. 19:5081 (1991); Ohtsuka, et al., J. Biol. Chem. 260:2605-2608 (1985), and Rossolini, et al., Mol. Cell. Probes 8:91-98 (1994)). The term nucleic acid is used interchangeably with gene, cDNA, and mRNA encoded by a gene. The term “nucleic acid” should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyedenosine, deoxycytidine, deoxyguanosine, and deoxythymidine. For purposes of clarity, when referring herein to a nucleotide of a nucleic acid, which can be DNA or RNA, the terms “adenosine”, “cytosine”, “guanosine”, and thymidine” are used. It is understood that if the nucleic acid is RNA, a nucleotide having a uracil base is uridine. The term “nucleotide” or nucleic acid as used herein is intended to refer to ribonucleotides, deoxyribonucleotides, acylic derivatives of nucleotides, and functional equivalents thereof, of any phosphorylation state. Functional equivalents of nucleotides are those that act as substrates for a polymerase as, for example, in an amplification method and artificial types of nucleic acids such as peptide nucleic acid (PNA) and locked nucleic acid (LNA) can be used. Functional equivalents of nucleotides are also those that can be formed into a polynucleotide that retains the ability to hybridize in a sequence specific manner to a target polynucleotide. As used herein, the term “polynucleotide” includes nucleotides of any number. A polynucleotide includes a nucleic acid molecule of any number of nucleotides including single-stranded RNA, DNA or complements thereof, double-stranded DNA or RNA, and the like.

The term “isolated” as used herein refers to the state of being substantially free of other material which is not the intended material. Stated another way, if the intended isolated product is a nucleic acid, the isolated nucleic acid is substantially free of other materials and/or contaminants such as proteins, lipids, carbohydrates, or other materials such as cellular debris or growth media. Typically, the term “isolated” is not intended to refer to a complete absence of these materials. Neither is the term “isolated” intended to refer the material is free from water, buffers, or salts, unless they are present in amounts that substantially interfere with the methods of the present invention. The term “isolated” as used herein when used with respect to nucleic acids, such as DNA or RNA, or proteins refers nucleic acids or peptides that are substantially free of cellular material, viral material, culture or suspension medium or chemical precursors or other chemical when isolated by the methods as disclosed herein. Moreover, an “isolated nucleic acid” is meant to include nucleic acid fragments which are not necessarily naturally occurring as fragments and can not typically be found in the natural state. Accordingly, an isolated nucleic acid encompass both an isolated heterologous and/or isolated recombinant nucleic acids. The term “isolated” as used herein can also refer to polypeptides which are isolated from other cellular materials and/or other proteins and is meant to encompass both purified and recombinant polypeptides.

The term “vector” used herein refers to a nucleic acid sequence containing an origin of replication. A vector can be a plasmid, bacteriophage, bacterial artificial chromosome or yeast artificial chromosome. A vector can be a DNA or RNA vector. A vector can be either a self replicating extrachromosomal vector or a vector which integrate into a host genome. The term “vectors” is used interchangeably with “plasmid” to refer to a nucleic acid molecule capable of transporting another nucleic acid to which it has been linked Vectors capable of directing the expression of genes and/or nucleic acid sequence to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. Other expression vectors can be used in different embodiments of the invention, for example, but are not limited to, plasmids, episomes, bacteriophages or viral vectors, and such vectors can integrate into the host's genome or replicate autonomously in the particular cell. Other forms of expression vectors known by those skilled in the art which serve the equivalent functions can also be used. Expression vectors comprise expression vectors for stable or transient expression encoding the DNA.

The terms “polypeptide” and “protein” are be used interchangeably herein. A “peptide” is a relatively short polypeptide, typically between 2 and 60 amino acids in length, e.g., between 5 and 50 amino acids in length. Polypeptides (typically over 60 amino acids in length) and peptides described herein may be composed of standard amino acids (i.e., the 20 L-alpha-amino acids that are specified by the genetic code, optionally further including selenocysteine and/or pyrrolysine). Polypeptides and peptides may comprise one or more non-standard amino acids. Non-standard amino acids can be amino acids that are found in naturally occurring polypeptides, e.g., as a result of post-translational modification, and/or amino acids that are not found in naturally occurring polypeptides. Polypeptides and peptides may comprise one or more amino acid analogs known in the art can be used. Beta-amino acids or D-amino acids may be used. One or more of the amino acids in a polypeptide or peptide may be modified, for example, by the addition of a chemical entity such as a carbohydrate group, a phosphate group, a fatty acid group, a linker for conjugation, functionalization, etc. A polypeptide that has a non-polypeptide moiety covalently or non-covalently associated may still be referred to as a “polypeptide”. Polypeptides may be purified from natural sources, produced in vitro or in vivo in suitable expression systems using recombinant DNA technology, synthesized through chemical means such as conventional solid phase peptide synthesis and/or using methods involving chemical ligation of synthesized peptides. The term “polypeptide sequence” or “peptide sequence” or “amino acid sequence” as used herein can refer to the polypeptide material itself or the peptide material itself and/or to the sequence information (i.e. the succession of letters or three letter codes used as abbreviations for amino acid names) that biochemically characterizes a polypeptide. Polypeptide sequences herein are presented in an N-terminal to C-terminal direction unless otherwise indicated.

The term “variant” as used herein refers to any polypeptide or peptide differing from a naturally occurring polypeptide by amino acid insertion(s), deletion(s), and/or substitution(s), created using, e.g., recombinant DNA techniques. In some embodiments, amino acid “substitutions” are the result of replacing one amino acid with another amino acid having similar structural and/or chemical properties, i.e., conservative amino acid replacements. “Conservative” amino acid substitutions may be made on the basis of similarity in any of a variety or properties such as side chain size, polarity, charge, solubility, hydrophobicity, hydrophilicity, and/or amphipathicity of the residues involved. For example, the non-polar (hydrophobic) amino acids include alanine, leucine, isoleucine, valine, glycine, proline, phenylalanine, tryptophan and methionine. The polar (hydrophilic), neutral amino acids include serine, threonine, tyrosine, asparagine, and glutamine. The positively charged (basic) amino acids include arginine, lysine and histidine. The negatively charged (acidic) amino acids include aspartic acid and glutamic acid. In some embodiments, cysteine is considered a non-polar amino acid. In some embodiments, insertions or deletions may range in size from about 1 to 20 amino acids, e.g., 1 to 10 amino acids. In some instances, larger domains may be removed without substantially affecting function. In certain embodiments, the sequence of a variant can be obtained by making no more than a total of 1, 2, 3, 5, 10, 15, or 20 amino acid additions, deletions, or substitutions to the sequence of a naturally occurring polypeptide. In some embodiments, not more than 1%, 5%, 10%, or 20% of the amino acids in a peptide, polypeptide or fragment thereof are insertions, deletions, or substitutions relative to the original polypeptide. In some embodiments, guidance in determining which amino acid residues may be replaced, added, or deleted without eliminating or substantially reducing activities of interest, may be obtained by comparing the sequence of the particular polypeptide with that of orthologous polypeptides from other organisms and avoiding sequence changes in regions of high conservation or by replacing amino acids with those found in orthologous sequences since amino acid residues that are conserved among various species may more likely be important for activity than amino acids that are not conserved.

The term “derivative” as used herein refers to peptides which have been chemically modified by techniques such as adding additional side chains, ubiquitination, labeling, pegylation (derivatization with polyethylene glycol), and insertion, deletion or substitution of amino acids, including insertion, deletion and substitution of amino acids and other molecules (such as amino acid mimetics or unnatural amino acids) that do not normally occur in the peptide sequence that is basis of the derivative, for example but not limited to insertion of ornithine which do not normally occur in human proteins. The term “derivative” is also intended to encompass all modified variants of the protein or peptides, variants, functional derivatives, analogues and fragments thereof, as well as peptides with substantial identity as compared to the reference peptide to which they refer to. The term derivative is also intended to encompass aptamers, peptidomimetics and retro-inverso peptides of the reference peptide to which they refer to. Amino acid substitutions include alterations in which an amino acid is replaced with a different naturally-occurring or a non-conventional amino acid residue. Such substitutions may be classified as “conservative”, in which case an amino acid residue contained in a polypeptide is replaced with another naturally occurring amino acid of similar character either in relation to polarity, side chain functionality or size.

Substitutions encompassed by the present invention may also be “non conservative”, in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments, amino acid substitutions are conservative.

As used herein, the terms “homologous” or “homologues” are used interchangeably, and when used to describe a polynucleotide or polypeptide, indicates that two polynucleotides or polypeptides, or designated sequences thereof, when optimally aligned and compared, for example using BLAST, version 2.2.14 with default parameters for an alignment (see herein) are identical, with appropriate nucleotide insertions or deletions or amino-acid insertions or deletions, in at least 70% of the nucleotides or amino acid residues, usually from about 75% to 99%, and more preferably at least about 98 to 99% of the nucleotides or amino acid residues. The term “homolog” or “homologous” as used herein also refers to homology with respect to structure and/or function. With respect to sequence homology, sequences are homologs if they are at least 50%, at least 60 at least 70%, at least 80%, at least 90%, at least 95% identical, at least 97% identical, or at least 99% identical. Determination of homologs of the genes or peptides of the present invention can be easily ascertained by the skilled artisan. Homologous sequences can be the same functional gene in different species.

The term “substantial identity” as used herein refers to two peptide sequences, when optimally aligned, such as by the programs GAP or BESTFIT using default gap weights, share at least about 65%, at least about . . . 70%, at least about . . . 80%, at least about . . . 90% sequence identity, at least about . . . 95% sequence identity or more (e.g., 99% sequence identity or higher). In some embodiments, residue positions which are not identical differ by conservative amino acid substitutions.

A “glycoprotein” as use herein is protein to which at least one carbohydrate chain (oligopolysaccharide) is covalently attached. A “proteoglycan” as used herein is a glycoprotein where at least one of the carbohydrate chains is a glycosaminoglycan, which is a long linear polymer of repeating disaccharides in which one member of the pair usually is a sugar acid (uronic acid) and the other is an amino sugar.

Substitutions encompassed by the present invention may also be “non conservative”, in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments, amino acid substitutions are conservative.

The term “biological sample” as used herein refers to a cell or population of cells or a quantity of tissue or fluid from a subject. Most often, the sample has been removed from a subject, but the term “biological sample” can also refer to cells or tissue analyzed in vivo, i.e. without removal from the subject. Often, a “biological sample” will contain cells from the animal, but the term can also refer to non-cellular biological material, such as non-cellular fractions of blood, saliva, or urine, that can be used to measure gene expression levels. Biological samples include, but are not limited to, tissue biopsies, scrapes (e.g. buccal scrapes), whole blood, plasma, serum, urine, saliva, cell culture, or cerebrospinal fluid. Biological samples also include tissue biopsies, cell culture. A biological sample or tissue sample can refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the sample is from a resection, bronchoscopic biopsy, or core needle biopsy of a primary or metastatic tumor, or a cellblock from pleural fluid. In addition, fine needle aspirate samples are used. Samples may be either paraffin-embedded or frozen tissue. The sample can be obtained by removing a sample of cells from a subject, but can also be accomplished by using previously isolated cells (e.g. isolated by another person), or by performing the methods of the invention in vivo. Biological sample also refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the biological samples can be prepared, for example biological samples may be fresh, fixed, frozen, or embedded in paraffin.

The term “tissue” is intended to include intact cells, blood, blood preparations such as plasma and serum, bones, joints, muscles, smooth muscles, and organs.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, interdisposition, affection. A disease and disorder, includes but is not limited to any condition manifested as one or more physical and/or psychological symptoms for which treatment is desirable, and includes previously and newly identified diseases and other disorders.

The term “basal cell” is a general term applied to any stratified or pseudostratified epithelium. It refers to cells which are juxtaposed to the basement membrane and under one or more additional epithelial layers. Many tissue can have both a two cell layer epithelium (basal and luminal cells) or a single layered epithelium. In the two cell layer, the cells adjacent to the basement membrane are termed “basal cells.”

The term ‘malignancy’ and ‘cancer’ are used interchangeably herein, and refers to any disease that is characterized by uncontrolled, abnormal growth of cells. Cancer cells can spread locally or through the bloodstream and lymphatic system to other parts of the body. The term is also intended to include any disease of an organ or tissue in mammals characterized by poorly-controlled or uncontrolled multiplication of normal or abnormal cells in that tissue and its effect on the body as a whole. Cancer diseases within the scope of the definition comprise benign neoplasms, dysplasias, hyperplasias as well as neoplasms showing metastatic growth or any other transformations like e.g. leukoplakias which often precede a breakout of cancer. The term “tumor” or “tumor cell” are used interchangeably herein, refers to the tissue mass or tissue type of cell that is undergoing abnormal proliferation.

As used herein, “metastasis” refers to the ability of cells of a cancer (e.g. a primary tumor, or a metastasis tumor) to be transmitted to other locations in the subject and to establish new tumors at such locations. An agent that “inhibits” cancer metastasis may function at any of a variety of steps in metastatic progression. For example, it may result in the delayed appearance of secondary tumors, slowed development of primary or secondary tumors, decreased occurrence of secondary tumors, slowed or decreased severity of secondary effects of disease, arrested tumor growth and regression of tumors, among others. In the extreme, complete inhibition is referred to herein as prevention (e.g., virtually complete inhibition, no metastasis if it had not occurred, no further metastasis if there had already been metastasis of a cancer, or virtually complete inhibition of the growth of a primary tumor caused by re-seeding of the tumor by a metastasized cell.

The term “hyperproliferative disease” or “hyperproliferative disorder” as used herein, refers to any condition in which a localized population of proliferating cells in an animal is not governed by the usual limitations of normal growth. Examples of hyperproliferative disorders include tumors, neoplasms, lymphomas and the like. A neoplasm is said to be benign if it does not undergo invasion or metastasis and malignant if it does either of these. A “metastatic” cell means that the cell can invade and destroy neighboring body structures. Hyperplasia is a form of cell proliferation involving an increase in cell number in a tissue or organ without significant alteration in structure or function. Metaplasia is a form of controlled cell growth in which one type of fully differentiated cell substitutes for another type of differentiated cell.

The term “neoplastic disease,” as used herein, refers to any abnormal growth of cells being either benign (non-cancerous) or malignant (cancerous).

A “metastatic” cell, as used herein, refers to a cell that has a potential for metastasis and is able to seed a tumor or a cell colony of interest. A “highly metastatic” cell, as used herein, refers to a cell that has a high potential for metastasis; e.g., cells from a cell line such as, but not limited to LM2, MDA-MB-231, PC-3, DU-145, Lewis Lung carcinoma.

A “tumorigenic cell,” as used herein, is a cell that, when introduced into a suitable site in a subject, can form a tumor. The cell may be non-metastatic or metastatic. A variety of types of tumorigenic and/or metastatic cells include cells from metastatic epithelial cancers, carcinomas, melanoma, leukemia, etc. The tumor cells may be, e.g., from cancers of breast, lung, colon, bladder, prostate, liver, gastrointestinal tract, endometrium, tracheal-bronchial tract, pancreas, liver, uterus, ovary, nasopharynges, prostate, bone or bone marrow, brain, skin or other suitable tissues or organs. In a preferred embodiment, the cancer cells are of human origin.

The term “tumor” or “tumor cell” are used interchangeably herein, and refers to the tissue mass or tissue type of cell that is undergoing abnormal proliferation.

A “cancer cell” refers to a cancerous, pre-cancerous or transformed cell, either in vivo, ex vivo, and in vitro tissue culture, that has spontaneous or induced phenotypic changes that do not necessarily involve the uptake of new genetic material. Although transformation can arise from infection with a transforming virus and incorporation of new genomic nucleic acid, or from the uptake of exogenous nucleic acid, it can also arise from spontaneous mutations within the genome that follow exposure to a carcinogen or other DNA-altering substance. Transformation/cancer is associated with, e.g., morphological changes, immortalization of cells aberrant growth control, foci formation, anchorage dependence, proliferation, malignancy, contact inhibition and density limitation of growth, growth factor or serum dependence, tumor specific markers levels, invasiveness, tumor growth or suppression in suitable animal hosts such as nude mice, and the like, in vitro, in vivo, and ex vivo (see also Freshney, Culture of Animal Cells: A Manual of Basic Technique (3rd ed. 1994)).

A “sarcoma” refers to a type of cancer cell that is derived from connective tissue, e.g., bone (osteosarcoma) cartilage (chondrosarcoma), muscle (rhabdomyosarcoma or rhabdosarcoma), fat cells (liposarcoma), lymphoid tissue (lymphosarcoma), collagen-producing fibroblasts (fibrosarcoma). Sarcomas may be induced by infection with certain viruses, e.g., Kaposi's sarcoma, Rous sarcoma virus, etc.

The term “angiogenesis” is broadly defined as the creation or spouting of new blood vessels from pre-existing blood vessels and is characterized by endothelial cell proliferation and migration triggered by pro-angiogenic factors. Angiogenesis can be a good and necessary process, for example, in wound healing, or it can be an aberrant and undesired process with detrimental consequences, such as the growth of solid tumors and metastasis, and hemangiomas. Aberrant angiogenesis can lead to certain pathological conditions such as death, blindness, and disfigurement. Angiogeneis and capillary elongation of about 1-2 mm requires both endothelial cell growth (including endothelial cell proliferation) and endothelial cell migration.

As used herein, the term “inhibiting angiogenesis” means the reduction or prevention of growth of new blood vessels. Inhibition includes slowing the rate of growth. The growth rate can be reduced by about 20%, about 30%, about 40%, about 50%, about 60%, about 70%, about 80%, about 90%, about 100%, about 125%, about 150% or more compared to a control, untreated condition. Inhibition also means no further growth of new blood vessels from the time of start of treatment administration. The term “inhibiting angiogenesis” also refers to a decrease in a measurable marker of angiogenesis by at least about 10%, at least about 20%, at least about 30%, at least about 40%, at least about 50%, at least about 60%, at least about 70%, at least about 80%, at least about 90%, at least about 95%, at least about 99%, or even 100% (i.e., absent) in the presence of an inhibitor of IGPR-1 as compared to the level of the measurable marker in the absence of an inhibitor of IGPR-1. Some non-limiting examples of measurable markers of angiogenesis include capillary density, endothelial cell proliferation, endothelial cell migration, and vessel ingrowth. Angiogenesis can be detected by methods known in the art.

As used herein, the term “inhibiting endothelial cell proliferation” refers to a decrease in the proliferation of endothelial cells of at least 10%, at least 20%, at least 30%, at least 40%, at least 50%, at least 60%, at least 70%, at least 80%, at least 90%, at least 95%, at least 99%, or even 100% (i.e., no growth) in the presence an inhibitor of IGPR-1 as compared to the level of proliferation in the absence of an inhibitor of IGPR-1.

As used herein, the term “migration” as used herein in reference to endothelial cell migration refers to all mechanisms and ways capillary blood vessels can grow or elongate or extend over increasing distances and surface areas.

As used herein, the term “inhibition of endothelial migration” refers to a decrease in the migration of endothelial cells through a porous membrane (e.g., using a commercially available migration assay kit such as BD BioCoat Angiogenesis System) of at least 10% in the presence of an inhibitor of IGPR-1, preferably the decrease is at least 20%, at least 30%, at least 40%, at least 50%, at least 60%, at least 70%, at least 80%, at least 90%, at least 95%, at least 99% decrease in the migration of endothelial cells through a porous membrane, or even 100% (i.e., no migration) in the presence of an IGPR-1 inhibitor as compared to in the absence of an IGPR-1 inhibitor agent.

As used herein, the term an “angiogenic disease or disorder” and an “angiogenesis-related disease” which are used in conjunction with the phrase “characterized by uncontrolled or increased angiogenesis”, refers to any pathological state or disease or disorder that is the direct result of aberrant blood vessel proliferation (e.g. diabetic retinopathy, AMD and hemangiomas) or undesired or pathological blood vessel proliferation (e.g. in the case cancer and tumor growth). The term also refers to diseases or disorders whose pathological progression is dependent on a good blood supply and thus blood vessel proliferation. Examples include but are not limited to abnormal vascular proliferation, ascites formation, psoriasis, age-related macular degeneration, thyroid hyperplasia, preeclampsia, rheumatoid arthritis (RA) and osteo-arthritis, Alzheimer's disease, obesity, pleural effusion, atherosclerosis, endometriosis, diabetic/other retinopathies, ocular neovascularization.

As used herein, the term an “angiogenesis-related disease” which used in conjunction with the phrase “characterized by a decrease in angiogenesis”, refers to any pathological state or disease or disorder that is the direct result of a loss of blood vessels or a reduction or inhibition of blood vessel proliferation. The term also refers to diseases or disorders whose pathological progression is due to a reduced blood supply or to disorders where it is desirable to increase the blood supply to a particular organ or tissue. Examples include, but are not limited to, ischemic diseases or ischemic injury, ischemia, transplantation therapy (such as, for example post-organ transplant therapy for transplantation of heart, lung, heart/lung, kidney, liver, and post-cell transplantation in cell based therapies such as stem cell therapies), stroke, retinopathy of prematurity (ROP) and the like.

As used herein, the term “ischemia” refers to inadequate or reduced blood supply (i.e. circulation) to a local area, for example due to a blockage of blood vessels to the area. Ischemic diseases include stroke, ischemic heart disease and the like.

As used herein, the term “ischemic injury” refers to conditions directly associated with reduced blood flow to tissue, for example due to a clot or obstruction of blood vessels which supply blood to the subject tissue and which result, inter alia, in lowered oxygen transport to such tissue, impaired tissue performance, tissue dysfunction and/or necrosis and can contribute to the pathogenesis of heart failure. Alternatively, where blood flow or organ perfusion can be quantitatively adequate, the oxygen carrying capacity of the blood or organ perfusion medium can be reduced, e.g., in hypoxic environment, such that oxygen supply to the tissue is lowered, and impaired tissue performance, tissue dysfunction, and/or tissue necrosis ensues. “Ischemia/reperfusion injury” refers to a subset of ischemic injury in which injury involves a period of reduced blood flow, followed by at least partial restoration of the blood flow. Ischemia/reperfusion injury involves an inflammatory response and oxidative damage accompanied by apoptosis that occur when blood flow has been restored to a tissue subjected to an interruption in blood flow. As used herein, the term “ischemic limb disease” refers to any disease resulting from lack of blood flow to a superficial limb or extremity (e.g., an arm, leg, hand, foot, toe, finger etc.). Ischemic limb disease results from complications due to diabetes or atherosclerosis, among others.

As used herein, the term “ischemic heart disease” refers to a condition in which the blood supply to the heart is decreased or reduced.

The terms “anticancer agent” and “anticancer drug,” as used herein, refer to any therapeutic agents (e.g., chemotherapeutic compounds and/or molecular therapeutic compounds), radiation therapies, or surgical interventions, used in the treatment of hyperproliferative diseases such as cancer (e.g., in mammals).

The term “anti-neoplastic agent,” as used herein, refers to any compound that retards the proliferation, growth, or spread of a targeted (e.g., malignant) neoplasm

As used herein, the term “therapeutically effective amount” or “effective amount” are used interchangeably and refer to the amount of an agent that is effective, at dosages and for periods of time necessary to achieve the desired therapeutic result, e.g., to decrease angiogenesis or to inhibit invasiveness of a metastatic cancer or to inhibit or reduce a cancer in a subject. By way of example only, an effective amount of an IGPR-1 inhibitor for treatment of an angiogenesis-related disease characterized by uncontrolled or increased angiogenesis will cause a reduction or even completely halt any new blood vessel formation. An effective amount for treating or ameliorating such an angiogenesis-related disease (i.e. one characterized by uncontrolled or increased angiogenesis) is an amount sufficient to result in a reduction or complete removal of the symptoms of the disorder, disease, or medical condition. The effective amount of a given therapeutic agent (i.e. IGPR-1 inhibitor) will vary with factors such as the nature of the agent, the route of administration, the size and species of the animal to receive the therapeutic agent, and the purpose of the administration. A therapeutically effective amount of the agents, factors, or inhibitors described herein, or functional derivatives thereof, can vary according to factors such as disease state, age, sex, and weight of the subject, and the ability of the therapeutic compound to elicit a desired response in the subject. A therapeutically effective amount is also one in which any toxic or detrimental effects of the therapeutic agent are outweighed by the therapeutically beneficial effects. The effective amount in each individual case can be determined empirically by a skilled artisan according to established methods in the art and without undue experimentation. In general, an IGPR-1 inhibitor functions as an effective anti-angiogenic agent is determined to be “therapeutically effective” in the methods described herein if (a) measurable symptom(s) of angiogenesis or an angiogenesis-related disease, (e.g., capillary density, tumor growth, rate of vessel formation) are decreased by at least 10% compared to the measurement prior to treatment onset, (b) the progression of the disease is halted (e.g., patients do not worsen, new vessels do not form, or the tumor does not continue to grow, or (c) symptoms are reduced or even ameliorated, for example, by measuring a reduction in tumor size or a reduction in vessel infiltration in the eye or elsewhere. Efficacy of treatment can be judged by an ordinarily skilled practitioner. Efficacy can be assessed in animal models of angiogenesis, cancer and tumor, for example treatment of a rodent with an experimental cancer, and any treatment or administration of an IGPR-1 inhibitor in a composition or formulation that leads to a decrease of at least one symptom of the cancer, for example a reduction in the size of the tumor or a cessation or slowing of the rate of growth of the tumor indicates effective treatment.

Stated another way, the phrases “therapeutically-effective” and “effective for the treatment, prevention, or inhibition”, are intended to qualify the amount of an IGPR-1 inhibitor as disclosed herein which will achieve the goal of reduction of the incidence or severity and/or frequency of incidence of a tumor growth, malignancy or neoplasia.

The term “prophylactic” or “therapeutic” treatment refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

A pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

The term “functional” when used in conjunction with “derivative” or “variant” or “fragment” refers to a polypeptide which possess a biological activity that is substantially similar to a biological activity of the entity or molecule of which it is a derivative or variant or fragment thereof. By “substantially similar” in this context is meant that at least 25%, at least 35%, at least 50% of the relevant or desired biological activity of a corresponding wild-type peptide is retained. In the instance of a fragment of soluble extracellular domain of IGPR-1 peptide (e.g., SEQ ID NO: 5), a functional fragment of SEQ ID NO: 5 or SEQ ID NO: 6 would be a protein or peptide comprising a portion of SEQ ID NO: 5 or SEQ ID NO: 6 which retained an activity for inhibiting IGPR-1 protein function, e.g., inhibiting angiogenesis; preferably the fragment of SEQ ID NO: 5 or SEQ ID NO: 5 retains at least 25%, at least 35%, at least 50% at least 60%, at least 70%, at least 80%, at least 90%, at least 95%, at least 100% or even higher (i.e., the variant or derivative has greater activity than the wild-type), e.g., at least 110%, at least 120%, or more activity compared to the full length SEQ ID NO: 5 or SEQ ID NO: 6 to inhibit the function of the full length IGPR-1 protein of SEQ ID NO: 1.

Accordingly, the term “non-functional” as used herein in conjunction with a “non-fragment of IGPR-1” refers to a polypeptide which comprises at least a portion of the IGPR-1 protein of SEQ ID NO:1 but does not retain the natural function of IGRP-1 of promoting angiogenesis. In some embodiments, a non-functional fragment of IGRP-1 still binds to the native ligands of IGRP-1 but does not allow IGPR-1 mediated intracellular signaling. Examples of non-functional fragments of IGRP-1 which can be used as IGPR-1 inhibitors for use in the methods and compositions of the invention include but are not limited to Ig-IGPR and ΔN-IGPR-1 as disclosed herein.

The term “antibody” is meant to be an immunoglobulin protein that is capable of binding an antigen. Antibody as used herein is meant to include antibody fragments, e.g. F(ab′)₂, Fab′, Fab, capable of binding the antigen or antigenic fragment of interest.

The term “humanized antibody” is used herein to describe complete antibody molecules, i.e. composed of two complete light chains and two complete heavy chains, as well as antibodies consisting only of antibody fragments, e.g. Fab, Fab′, F(ab′)₂, and Fv, wherein the CDRs are derived from a non-human source and the remaining portion of the Ig molecule or fragment thereof is derived from a human antibody, preferably produced from a nucleic acid sequence encoding a human antibody.

The terms “human antibody” and “humanized antibody” are used herein to describe an antibody of which all portions of the antibody molecule are derived from a nucleic acid sequence encoding a human antibody. Such human antibodies are most desirable for use in antibody therapies, as such antibodies would elicit little or no immune response in the human subject.

The term “chimeric antibody” is used herein to describe an antibody molecule as well as antibody fragments, as described above in the definition of the term “humanized antibody.” The term “chimeric antibody” encompasses humanized antibodies. Chimeric antibodies have at least one portion of a heavy or light chain amino acid sequence derived from a first mammalian species and another portion of the heavy or light chain amino acid sequence derived from a second, different mammalian species. In some embodiments, a variable region is derived from a non-human mammalian species and the constant region is derived from a human species. Specifically, the chimeric antibody is preferably produced from a 9 nucleotide sequence from a non-human mammal encoding a variable region and a nucleotide sequence from a human encoding a constant region of an antibody.

The term “label” refers to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

The term “epitope” as used herein refers to that portion of an antigen that makes contact with a particular antibody.

The term “treatment” refers to any treatment of a pathologic condition in a subject, particularly a human subject, and includes one or more of the following: (a) preventing a pathological condition from occurring in a subject which may be predisposed to the condition but has not yet been diagnosed with the condition and, accordingly, the treatment constitutes prophylactic treatment for the disease or condition; (b) inhibiting the pathological condition, i.e. arresting its development, (c) relieving the pathological condition, i.e. causing a regression of the pathological condition; or (d) relieving the conditions mediated by the pathologically condition. In some embodiments, the terms “treating” or “to treat” means to alleviate a symptom or a malignancy or neoplasm or eliminate the causation either on a temporary or permanent basis, or to prevent or slow the appearance of a symptom. The term “treatment” includes alleviation, elimination of causation of, or prevention of, undesirable symptoms associated with a malignancy, tumor growth or neoplasia disorder. Besides being useful for human treatment, the compositions and methods as disclosed herein are useful for any subject in need of treatment of a cancer, e.g., a cancer expressing IGPR-1, for example these combinations are also useful for treatment of mammals, including horses, dogs, cats, rats, mice, sheep, pigs, etc. The pathological condition which is modulated by the treatment as disclosed herein, e.g. by inhibitors of IGPR-1 covers all disease states which are generally acknowledged in the art to be associated with cancer.

The term “cells,” “host cells” or “recombinant host cells” are terms used interchangeably herein. It is understood that such terms refer not only to a particular cell type, but to the progeny or potential progeny of such a cell. Because certain modifications can occur in succeeding generations due to either mutation or environmental influences, such progeny can not, in fact, be identical to the parent cell, but are still included within the scope of the term as used herein.

The term “potency” refers to the minimum concentration at which an agent, e.g. inhibitor of IGPR-1 is able to achieve a desirable biological or therapeutic effect. The potency of an agent, e.g. inhibitor of IGPR-1 is typically proportional to its affinity for its ligand binding site on the IGPR-1 protein. In some cases, the potency may be non-linearly correlated with its affinity. In comparing the potency of a drug under different situations, (e.g., the potency of an inhibitor of IGPR-1), the dose-response curve can be determined under identical test conditions (e.g., in an in vitro or in vivo assay, in an appropriate animal model such a human patient).

The term “subject” for purposes of treatment refers to any living organism who is in need of treatment for cancer. A subject is typically a human subject or animal subject. The term includes, but is not limited to, humans, non-human primates such as chimpanzees and other apes and monkey species; farm animals such as cattle, sheep, pigs, goats and horses, domestic subjects such as dogs and cats, laboratory animals including rodents such as mice, rats and guinea pigs, and the like. The term does not denote a particular age or sex. Thus, adult and newborn subjects, as well as fetuses, whether male or female, are intended to be covered. The term “subject” is also intended to include living organisms susceptible to conditions or diseases caused or contributed bacteria, pathogens, disease states or conditions as generally disclosed, but not limited to, throughout this specification. Examples of subjects include humans, dogs, cats, cows, goats, and mice. The term subject is further intended to include transgenic species. In another embodiment, the subject is an experimental animal or animal substitute as a disease model.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N.J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of (3-Lactam antibiotics, Pharm. Biotech. 11,:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metah. Pharmacokinet, 15(2): 143-53; Balimane and Sinko (1999). Involvement of multiple transporters in the oral absorption of nucleoside analogues, Adv. Drug Delivery Rev., 39(1-3):183-209; Browne (1997). Fosphenyloin (Cerebyx), Clin. Neuropharmacol. 20(1): 1-12; Bundgaard (1979). Bioreversible derivatization of drugs-principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS PharmSci., 2(1): E6; Sadzuka Y. (2000). Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl 2:S15-27; Wang, W. et al. (1999) Prodrug approaches to the improved delivery of peptide drugs. Curr. Pharm. Des., 5(4):265-87.

As used herein, a “pharmaceutical carrier” is a pharmaceutically acceptable solvent, suspending agent or vehicle for delivering an agent (e.g. an inhibitor of IGPR-1) to the surface of a target cell, e.g. smooth muscle cell or basal cell or respiratory cell or to a subject. The carrier can be liquid or solid and is selected with the planned manner of administration in mind. Each carrier must be pharmaceutically “acceptable” in the sense of being compatible with other ingredients of the composition and non-injurious to the subject.

The term “substantially pure”, with respect to nucleic acid refers to a sample comprising nucleic acids that is at least about 75%, preferably at least about 85%, more preferably at least about 90%, and most preferably at least about 95% pure, with respect to nucleic acids in the sample. Recast, the terms “substantially pure” or “essentially purified”, with regard to a preparation of a nucleic acid sample refer to a nucleic acid preparation that contains fewer than about 20%, more preferably fewer than about 15%, 10%, 8%, 7%, most preferably fewer than about 5%, 4%, 3%, 2%, 1%, or less than 1%, of non-nucleic acid molecules, such a proteins and other biomolecules.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

The term “optional” or “optionally” means that the subsequent described event, circumstance or substituent may or may not occur, and that the description includes instances where the event or circumstance occurs and instances where it does not.

The articles “a” and “an” are used herein to refer to one or to more than one (i.e., at least one) of the grammatical object of the article. By way of example, “an element” means one element or more than one element. Thus, in this specification and the appended claims, the singular forms “a,” “an,” and “the” include plural references unless the context clearly dictates otherwise. Thus, for example, reference to a pharmaceutical composition comprising “an agent” includes reference to two or more agents.

As used herein, the term “comprising” means that other elements can also be present in addition to the defined elements presented. The use of “comprising” indicates inclusion rather than limitation. The term “consisting of” refers to compositions, methods, and respective components thereof as described herein, which are exclusive of any element not recited in that description of the embodiment. As used herein the term “consisting essentially of” refers to those elements required for a given embodiment. The term permits the presence of elements that do not materially affect the basic and novel or functional characteristic(s) of that embodiment of the invention.

Other than in the operating examples, or where otherwise indicated, all numbers expressing quantities of ingredients or reaction conditions used herein should be understood as modified in all instances by the term “about.” The term “about” when used in connection with percentages can mean±1%.

This invention is further illustrated by the examples which should not be construed as limiting. The contents of all references cited throughout this application, as well as the figures and tables are incorporated herein by reference. All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### General:

One aspect of the present invention generally relates to compositions comprising inhibitors of the IGPR-1 (immunoglobulin containing and proline rich receptor 1) protein, and their use in methods to treat a subject with an angiogenesis-related disease or disorder associated with uncontrolled or increased angiogenesis, e.g., for the treatment of cancer and prevention of cancer metastasis in a subject, and treatment of retinopathy. Accordingly, the present invention relates to compositions comprising inhibitors of IGPR-1, and use as an anti-cancer therapy, and methods for the treatment of cancer by administering to a subject with cancer a composition comprising an inhibitor of IGPR-1.

In another aspect of the present invention, the present invention relates to compositions comprising a IGPR-1 (immunoglobulin containing and proline rich receptor 1) protein or functional derivatives, mimetic or variants thereof, and their use in methods to treat a subject with an angiogenesis-related disease or disorder associated with a decrease in angiogenesis, e.g., for the treatment of AMD, ischemia, transplant therapy, cornea transplant and the like.

IGPR-1 (Immunoglobulin Containing and Proline Rich Receptor 1) Protein

The inventors have discovered that inhibition of IGPR-1 can be used to inhibit metastasis and for the treatment of cancer. In some embodiments, an inhibitor of IGPR-1 is a protein inhibitor, and in some embodiments, the inhibitor is any agent which inhibits the function of IGPR-1 or the expression of IGPR-1 from its gene.

As used herein, the IPGR-1 polypeptide sequence corresponds to Genbank Accession No. NP_{—}653216.1, and refers to SEQ ID NO: 1 as disclosed herein. IPGR1 is also known as IPGR-1, IGP1, or (immunoglobin containing proline rich receptor-1), or TMIGD2, or MGC23244. The human gene sequence encoding for IPGR1 protein corresponds to Genebank Accession no. NM_{—}144615.2 (SEQ ID NO: 2) or Gene ID: GI:281306837 (SEQ ID NO: 2)

Accordingly, the protein sequence of IGPR-1 is as follows: (the Signal sequence is shown in bold)
MGSPGMVLGL LVQIWALQEA SSLSVQQGPN LLQVRQGSQA TLVCQVDQAT AWERLRVKWT KDGAILCQPY ITNGSLSLGV CGPQGRLSWQ APSHLTLQLD PVSLNHSGAY VCWAAVEIPE LEEAEGNITR LFVDPDDPTQ NRNRIASFPG FLFVLLGVGS MGVAAIVWGA WFWGRRSCQQ RDSGNSPGNA FYSNVLYRPR GPPKKSEDCS GEGKDQRGQS IYSTSFPQPA PRQPHLASRP CPSPRPCPSP RPGHPVSMVR VSPRPSPTQQ PRPKGFPKVG EE (SEQ ID NO: 1)

Inhibitors of IGPR-1 (Immunoglobulin Containing and Proline Rich Receptor 1) Protein for Treatment of Angiogenesis-Related Disease and Disorders Associated with Increased Angiogenesis.

As discussed herein, the inventors have discovered that inhibition of IGPR-1 can be used to inhibit metastasis and for the treatment of cancer. In some embodiments, an inhibitor of IGPR-1 is a protein inhibitor, and in some embodiments, the inhibitor is any agent which inhibits the function of IGPR-1 or the expression of IGPR-1 from its gene.

RNAi inhibitors of IGPR-1.

Inhibition of the IGPR-1 gene can be by gene silencing RNAi molecules according to methods commonly known by a skilled artisan. For example, a gene silencing siRNA oligonucleotide duplexes targeted specifically to human IGPR-1 (GenBank No: NM_{—}144615.2) can readily be used to knockdown IGPR-1 expression. IGPR-1 mRNA can be successfully targeted using siRNAs; and other siRNA molecules may be readily prepared by those of skill in the art based on the known sequence of the target mRNA. To avoid doubt, the sequence of a human IGPR-1 is provided at, for example, GenBank Accession Nos. NM_{—}144615.2 (SEQ ID NO: 2)

As used herein, the term “IGPR-1 protein” refers to the nucleic acid of SEQ ID NO: 1 as disclosed herein, and homologues thereof, including conservative substitutions, additions, deletions therein not adversely affecting the structure of function. As used herein, the IGPR-1 protein is encoded by the nucleic acid sequence for human IGPR-1 transcript (SEQ ID NO: 2) is as follows:

| | |
|---|---|
| 1 | ggaagtctgt caactgggag ggggagaggg gggtgatggg ccaggaatgg ggtccccggg |
| 61 | catggtgctg ggcctcctgg tgcagatctg ggccctgcaa gaagcctcaa gcctgagcgt |
| 121 | gcagcagggg cccaacttgc tgcaggtgag gcagggcagt caggcgaccc tggtctgcca |
| 181 | ggtggaccag gccacagcct gggaacggct ccgtgttaag tggacaaagg atggggccat |
| 241 | cctgtgtcaa ccgtacatca ccaacggcag cctcagcctg ggggtctgcg ggccccaggg |
| 301 | acggctctcc tggcaggcac ccagccatct caccctgcag ctggaccctg tgagcctcaa |
| 361 | ccacagcggg gcgtacgtgt gctgggcggc cgtagagatt cctgagttgg aggaggctga |
| 421 | gggcaacata acaaggctct ttgtggaccc agatgacccc acacagaaca gaaaccggat |
| 481 | cgcaagcttc ccaggattcc tcttcgtgct gctgggggtg ggaagcatgg gtgtggctgc |
| 541 | gatcgtgtgg ggtgcctggt tctggggccg ccgcagctgc cagcaaaggg actcaggtaa |
| 601 | cagcccagga aatgcattct acagcaacgt cctataccgg ccccgggggg ccccaaagaa |
| 661 | gagtgaggac tgctctggag aggggaagga ccagaggggc cagagcattt attcaacctc |
| 721 | cttcccgcaa ccggcccccc gccagccgca cctggcgtca agaccctgcc ccagcccgag |
| 781 | accctgcccc agccccaggc ccggccaccc cgtctctatg gtcagggtct ctcctagacc |
| 841 | aagccccacc cagcagccga ggccaaaagg gttccccaaa gtgggagagg agtgagagat |
| 901 | cccaggagac ctcaacagga ccccacccat aggtacacac aaaaaagggg ggatcgaggc |
| 961 | cagacacggt ggctcacgcc tgtaatccca gcagtttggg aagccgaggc gggtggaaca |
| 1021 | cttgaggtca ggggtttgag accagcctgg cttgaacctg ggaggcggag gttgcagtga |
| 1081 | gccgagattg cgccactgca ctccagcctg ggcgacagag tgagactccg tctcaaaaaa |
| 1141 | aacaaaaagc aggaggattg ggagcctgtc agccccatcc tgagaccccg tcctcatttc |
| 1201 | tgtaatgatg gatctcgctc ccactttccc ccaagaacct aataaaggct tgtgaagaaa |
| 1261 | aagcaaaaaa aaaaaaaaaa aa |

An inhibitor of IGPR-1 can be any agent which inhibits the function of IGPR-1, such as antibodies, gene silencing RNAi molecules and the like. Commercial neutralizing antibodies of IGPR-1 are encompassed for use in the methods and compositions as disclosed herein. Additionally, small molecules agonists of IGPR-1 are known by one of ordinary skill in the art and are encompassed for use in the methods and compositions as disclosed herein as an inhibitor of the IGPR-1 protein function or its expression from the IGPR-1 gene.

In some embodiments a protein, or protein fragment or polypeptide of IGPR-1 can be used as an inhibitor of IGPR-1 in the methods, compositions and kits as disclosed herein. In some embodiments, a protein or protein fragment may be a protein, peptide or protein fragment of at least 10 amino acid sequence of IGPR-1 protein. In some embodiments, an inhibitor of IGPR-1 is a fragment or polypeptide of IGPR-1 which functions as a dominant negative or decoy molecule for ligands binding to endogenous IGPR-1, and therefore a fragment of the IGPR-1 polypeptide can inhibit the function of endogenous IGPR-1 expressed in cells.

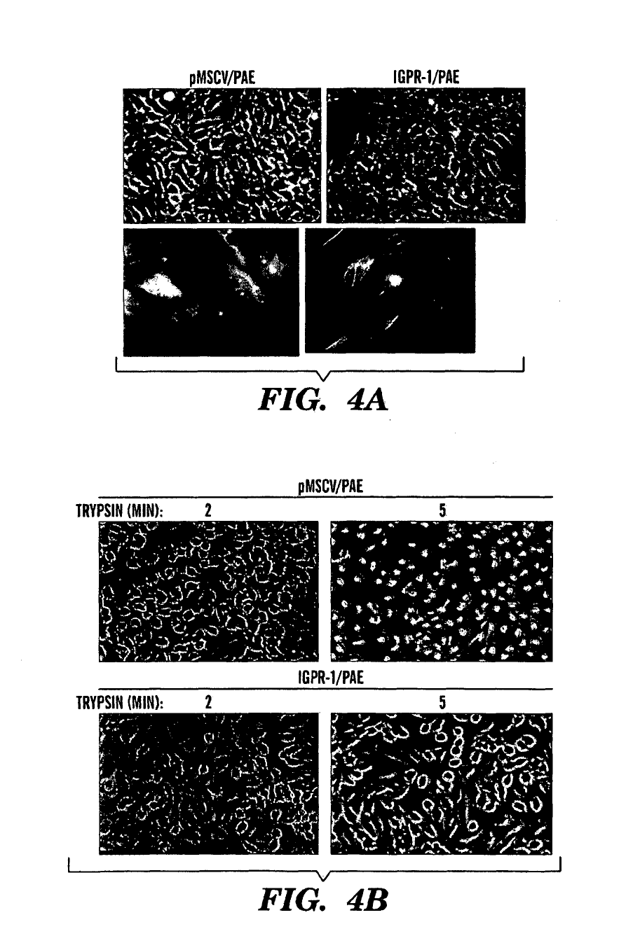

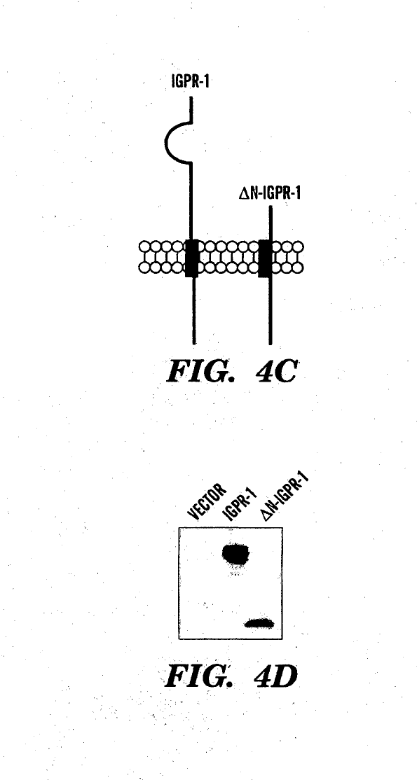

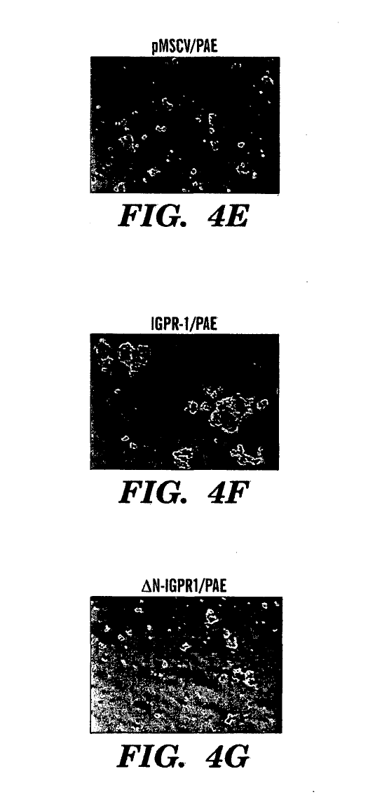

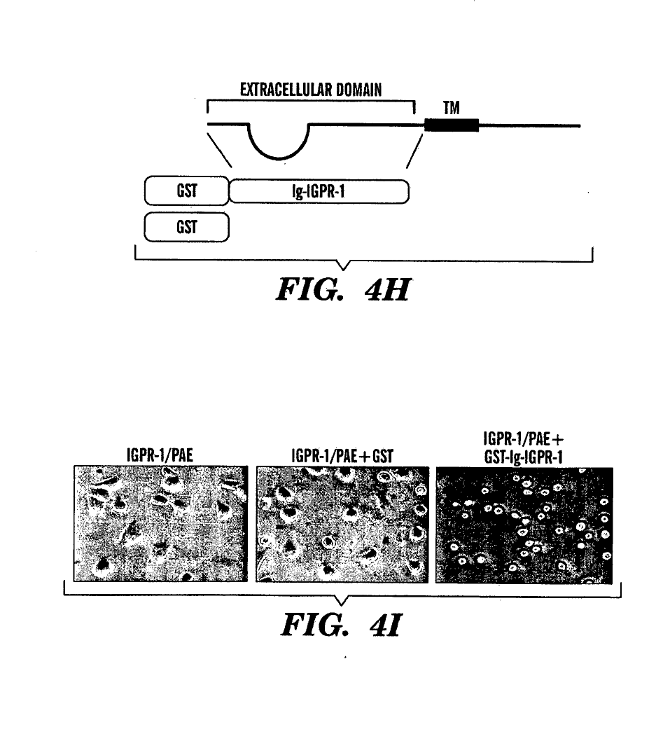

As disclosed in the Examples, a fragment of IGPR-1 protein can be used to function as a dominant negative protein inhibitor of IGPR-1. For example, one such dominant negative inhibitor can be the soluble recombinant domain of IGPR-1 (e.g., referred to herein as “Ig-IGPR-1”, as shown in FIG. 4H), which prevents cis- and/or trans-dimerization of the IGPR-1 which is required for IGPR-1 cell signaling. In alternative embodiments, a dominant negative inhibitor is ΔN-IGPR-1, as disclosed herein in the Examples, which has 133 amino acids deleted from the N-terminus (including deletion of the signal sequence) (See FIG. 4C), which includes the transmembrane region and intracellular/cytoplasmic domain of IGPR-1.

In some embodiments, a dominant negative inhibitor is at least about carboxyl most 60 amino acids of SEQ ID NO: 1, or at least about 70, or at least about 80 or at least about 90 or at least about 100 or more most-carboxyl amino acids of SEQ ID NO: 1. In some embodiments, a dominant negative inhibitor of IGPR-1 is at least about carboxyl most 60 amino acids of SEQ ID NO: 1, or at least about 70, or at least about 80 or at least about 90 or at least about 100 or more most-carboxylamino acids of the following amino acid sequence:

(SEQ ID NO: 4)
DPDDPTQ NRNRIASFPG FLFVLLGVGS MGVAAIVWGA
WFWGRRSCQQ RDSGNSPGNA FYSNVLYRPR GPPKKSEDCS
GEGKDQRGQS IYSTSFPQPA PRQPHLASRP CPSPRPCPSP
RPGHPVSMVR VSPRPSPTQQ PRPKGFPKVG EE

In some embodiments, as IGPR-1 functions as a dimer to promote tumor growth, in some embodiments a dominant negative inhibitor of IGPR-1 is a soluble recombinant extracellular domain of IGPR-1 (e.g., Ig-IGPR1, see FIG. 4H), as disclosed herein in the Examples. In some embodiments, a soluble recombinant extracellular domain of IGPR-1 (e.g., a Ig-IGRP-1) comprises at least 60 or at least about 70, or at least about 80, or at least about 90 or more than 90 most N-terminal amino acids of SEQ ID NO: 1. In some embodiments, a dominant negative inhibitor of IGPR-1 (e.g., a Ig-IGRP-1) comprises at least about 60, or at least about 70 or at least about 80 or at least about 90 or at least about 100 N-terminal amino acids of the following sequence:

LSVQQGPN LLQVRQGSQA TLVCQVDQAT AWERLRVKWT KDGAILCQPY ITNGSLSLGV CGPQGRLSWQ APSHLTLQLD PVSLNHSGAY VCWAAVEIPE LEEAEGNITR LFVDPDDPTQ NRNRIASFP (SEQ ID NO: 5). In some embodiments, an inhibitor of IGPR-1 protein which is a dominant negative inhibitor of IGPR-1, e.g., a soluble recombinant extracellular domain of IGPR-1 (e.g., a Ig-IGRP-1) comprises the following sequence or a biologically active or functional fragment thereof:

(SEQ ID NO: 6)
LSVQQGPN LLQVRQGSQA TLVCQVDQAT AWERLRVKWT
KDGAILCQPY ITNGSLSLGV CGPQGRLSWQ APSHLTLQLD
PVSLNHSGAY VCWAAVEIPE LEEAEGNITR LFV.

In some embodiments, an inhibitor of IGPR-1 protein which is a dominant negative inhibitor of IGPR-1, e.g., a soluble recombinant extracellular domain of IGPR-1 (Ig-IGPR-1) can further comprise an N-terminal signal peptide, for example, a signal peptide of MGSPGMVLGL LVQIVVALQEA SS (SEQ ID NO: 7) or a fragment thereof.

Agents in General which Function as Inhibitors of IGPR-1

In some embodiments, the present invention relates to agents which inhibit a IGPR-1 and/or SPIN 90 gene. In some embodiments, inhibition can be by inhibition of nucleic acid transcripts encoding a IGPR-1 gene and/or SPIN-90 gene, for example inhibition of messenger RNA (mRNA). In alternative embodiments, an agent which inhibits IGPR-1 inhibits the expression and/or the activity of the gene product of IGPR-1 gene, e.g., inhibits the IGPR-1 polypeptide or protein, or isoforms thereof. As used herein, the term “gene product” refers to RNA transcribed from a gene, or a polypeptide encoded by a gene or translated from RNA.

In some embodiments, the inventors discovered that SPIN-90 mediated the function of IGPR-1 inhibition of migration of cells in vitro, and based on the fact that in vivo, IGPR-1 has an opposite function and promotes tumor growth and angiogenesis, in some embodiments, inhibition of SPIN90 function or SPIN90 gene expression in vivo can also be used to inhibit IGPR-1-mediated tumor growth and angiogenesis according to the methods and compositions as disclosed herein. SPIN is also known as alias as NCK interacting protein with SH3 domain or “NCKIPSD”, AF3p21, ALL1-fused gene from chromosome 3p21, Dia interacting protein-1, Diaphanous protein interacting protein (DIP1), SH3 protein interacting with NCK, 90-KD, VacA-interacting protein, 54 kDa, VIP54. SPIN90 and is encoded by mRNA transcript RefSeq ID No: NM_{—}016453.2 (SEQ ID NO: 8) (isoform 1) or NM_{—}184231.1 (SEQ ID NO: 9) (isoform 2), which encode proteins NP_{—}057537.1 (SEQ ID NO: 10) and NP_{—}909119.1 (SEQ ID NO: 11) respectively. One can easily inhibit SPIN-90 according to any method commonly known in the art and as discussed herein, for example, using gene silencing RNAi agents as disclosed herein or dominant negative inhibitors of SPIN90 as disclosed herein. In some embodiments, one can use commercially available inhibitors of SPIN90, for example, commercially available inhibitors from Santa Cruz Biotechnology, Sigma, as well as inhibitor antibodies which are commercially available.

In some embodiments, one can also inhibit BAG1 and/or CACNB2. BAG is also known as BCL2-associated athanogene, or BPAG1 or HAP or RAP46 and is encoded by the mRNA transcript identified by RefSeq ID: NM_{—}001172415 (SEQ ID NO: 12) which encodes for the protein identified by RefSeq ID No: NP_{—}001165886.1 (SEQ ID NO: 13). CACNB2 is also known as calcium channel, voltage-dependent, beta 2 subunit or aliases CACNLB2; CAVB2; F1123743; MYSB, voltage-dependent L-type calcium channel subunit beta-2. CACNB2 is encoded by the mRNA transcript identified by the RefSeq ID No: NM_{—}000724.3 (SEQ ID NO: 14) which encodes for the protein identified by RefSeq ID No: NP_{—}000715.2 (SEQ ID NO: 15). In some embodiments, one can easily inhibit BPAG1 and/or CACNB2, according to any method commonly known in the art and as discussed herein, for example, using gene silencing RNAi agents as disclosed herein or dominant negative inhibitors of BPAG1 and/or CACNB2 as disclosed herein. In some embodiments, one can use commercially available inhibitors of BPAG1 and/or CACNB2, for example, commercially available inhibitors from Santa Cruz Biotechnology, Sigma, as well as inhibitor antibodies which are commercially available.

### Nucleic Acid Inhibitors of IGPR-1 Regulator Gene.

In some embodiments, inhibition of IGPR-1 is by an agent. One can use any agent, for example but are not limited to nucleic acids, nucleic acid analogues, peptides, phage, phagemids, polypeptides, peptidomimetics, ribosomes, aptamers, antibodies, small or large organic or inorganic molecules, or any combination thereof.

Agents useful in the methods as disclosed herein can also inhibit gene expression (i.e. suppress and/or repress the expression of the gene). Such agents are referred to in the art as “gene silencers” and are commonly known to those of ordinary skill in the art. Examples include, but are not limited to a nucleic acid sequence, for an RNA, DNA or nucleic acid analogue, and can be single or double stranded, and can be selected from a group comprising nucleic acid encoding a protein of interest, oligonucleotides, nucleic acids, nucleic acid analogues, for example but are not limited to peptide nucleic acid (PNA), pseudo-complementary PNA (pc-PNA), locked nucleic acids (LNA) and derivatives thereof etc. Nucleic acid agents also include, for example, but are not limited to nucleic acid sequences encoding proteins that act as transcriptional repressors, antisense molecules, ribozymes, small inhibitory nucleic acid sequences, for example but are not limited to RNAi, shRNAi, siRNA, micro RNAi (miRNA), antisense oligonucleotides, etc.

In some embodiments, an inhibitor of IGPR-1 is a RNAi agent. In some embodiments, a RNAi agent inhibits the expression of the IGPR-1 protein of SEQ ID NO:1. One of ordinary skill can select a RNAi agent to be used which inhibits the expression of SEQ ID NO:1 or inhibits the mRNA expression of SEQ ID NO:2 as disclosed herein. In some embodiments, an inhibitor of IGPR-1 is a siRNA agent, for example, but not limited to: CAGCAAAGGGACUCAGGUAUU (SEQ ID NO: 20) or AGGUAACAGCCCAGGAAAUUU (SEQ ID NO: 21) and variants and derivatives thereof. In some embodiments, a siRNA agent is a Locked nucleic acid (LNA) which targets the same region on the IGPR-1 mRNA of SEQ ID NO:2 as the siRNAs CAGCAAAGGGACUCAGGUAUU (SEQ ID NO: 20) or AGGUAACAGCCCAGGAAAUUU (SEQ ID NO: 21).

As used herein, agents useful in the method as inhibitors of IGPR-1 gene expression and/or inhibition of IGPR-1 protein function can be any type of entity, for example but are not limited to chemicals, nucleic acid sequences, nucleic acid analogues, proteins, peptides or fragments thereof. In some embodiments, the agent is any chemical, entity or moiety, including without limitation, synthetic and naturally-occurring non-proteinaceous entities. In certain embodiments the agent is a small molecule having a chemical moiety.

In alternative embodiments, agents useful in the methods as disclosed herein are proteins and/or peptides or fragment thereof, which inhibit the gene expression of IGPR-1 or the function of the IGPR-1 protein. Such agents include, for example but are not limited to protein variants, mutated proteins, therapeutic proteins, truncated proteins and protein fragments. Protein agents can also be selected from a group comprising mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. As disclosed herein, a protein which inhibit the function of IGPR-1 is a soluble extracellular dominant negative IGPR-1 protein, e.g., a protein of SEQ ID NO:5 or a functional fragment or variant thereof which inhibits wild-type full length IGPR-1 function.

Alternatively, agents useful in the methods as disclosed herein as inhibitors of IGPR-1 can be a chemicals, small molecule, large molecule or entity or moiety, including without limitation synthetic and naturally-occurring non-proteinaceous entities. In certain embodiments the agent is a small molecule having the chemical moieties as disclosed herein.

In some embodiments, agents that inhibit IGPR-1 is a nucleic acid. Nucleic acid inhibitors of IGPR-1 include, for example, but not are limited to, RNA interference-inducing (RNAi) molecules, for example but are not limited to siRNA, dsRNA, stRNA, shRNA and modified versions thereof, where the RNA interference (RNAi) molecule silences the gene expression from the IGPR-1 gene.

Accordingly, in some embodiments, inhibitors of IGPR-1 can inhibit IGPR-1 by any “gene silencing” methods commonly known by persons of ordinary skill in the art. In some embodiments, the nucleic acid inhibitor of IGPR-1 is an anti-sense oligonucleic acid, or a nucleic acid analogue, for example but are not limited to DNA, RNA, peptide-nucleic acid (PNA), pseudo-complementary PNA (pc-PNA), or locked nucleic acid (LNA) and the like. In alternative embodiments, the nucleic acid is DNA or RNA, and nucleic acid analogues, for example PNA, pcPNA and LNA. A nucleic acid can be single or double stranded, and can be selected from a group comprising nucleic acid encoding a protein of interest, oligonucleotides, PNA, etc. Such nucleic acid sequences include, for example, but are not limited to, nucleic acid sequence encoding proteins that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc.

In some embodiments single-stranded RNA (ssRNA), a form of RNA endogenously found in eukaryotic cells can be used to form an RNAi molecule. Cellular ssRNA molecules include messenger RNAs (and the progenitor pre-messenger RNAs), small nuclear RNAs, small nucleolar RNAs, transfer RNAs and ribosomal RNAs. Double-stranded RNA (dsRNA) induces a size-dependent immune response such that dsRNA larger than 30 bp activates the interferon response, while shorter dsRNAs feed into the cell's endogenous RNA interference machinery downstream of the Dicer enzyme.

RNA interference (RNAi) provides a powerful approach for inhibiting the expression of selected target polypeptides. RNAi uses small interfering RNA (siRNA) duplexes that target the messenger RNA encoding the target polypeptide for selective degradation. siRNA-dependent post-transcriptional silencing of gene expression involves cutting the target messenger RNA molecule at a site guided by the siRNA.

RNA interference (RNAi) is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease can be at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA can be chemically synthesized, can be produced by in vitro transcription, or can be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and can contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand can precede the nucleotide loop structure and the antisense strand can follow. These shRNAs can be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety).

The target gene or sequence of the RNA interfering agent can be a cellular gene or genomic sequence, e.g. the IGPR-1 gene sequence. A siRNA can be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. In addition to native RNA molecules, RNA suitable for inhibiting or interfering with the expression of a target sequence include RNA derivatives and analogs. Preferably, the siRNA is identical to its target sequence.

The siRNA preferably targets only one sequence. Each of the RNA interfering agents, such as siRNAs, can be screened for potential off-target effects by, for example, expression profiling. Such methods are known to one skilled in the art and are described, for example, in Jackson et al, Nature Biotechnology 6:635-637, 2003. In addition to expression profiling, one can also screen the potential target sequences for similar sequences in the sequence databases to identify potential sequences which can have off-target effects. For example, according to Jackson et al. (Id.) 15, or perhaps as few as 11 contiguous nucleotides of sequence identity are sufficient to direct silencing of non-targeted transcripts. Therefore, one can initially screen the proposed siRNAs to avoid potential off-target silencing using the sequence identity analysis by any known sequence comparison methods, such as BLAST.

siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. For example, siRNA containing D-arabinofuranosyl structures in place of the naturally-occurring D-ribonucleosides found in RNA can be used in RNAi molecules according to the present invention (U.S. Pat. No. 5,177,196). Other examples include RNA molecules containing the o-linkage between the sugar and the heterocyclic base of the nucleoside, which confers nuclease resistance and tight complementary strand binding to the oligonucleotides molecules similar to the oligonucleotides containing 2′-O-methyl ribose, arabinose and particularly D-arabinose (U.S. Pat. No. 5,177,196).

The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases can also be modified. Any modified base useful for inhibiting or interfering with the expression of a target sequence can be used. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can also be incorporated.

The most preferred siRNA modifications include 2′-deoxy-2′-fluorouridine or locked nucleic acid (LNA) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology. Preferably, the modifications involve minimal 2′-O-methyl modification, preferably excluding such modification. Modifications also preferably exclude modifications of the free 5′-hydroxyl groups of the siRNA.

siRNA and miRNA molecules having various “tails” covalently attached to either their 3′- or to their 5′-ends, or to both, are also known in the art and can be used to stabilize the siRNA and miRNA molecules delivered using the methods of the present invention. Generally speaking, intercalating groups, various kinds of reporter groups and lipophilic groups attached to the 3′ or 5′ ends of the RNA molecules are well known to one skilled in the art and are useful according to the methods of the present invention. Descriptions of syntheses of 3′-cholesterol or 3′-acridine modified oligonucleotides applicable to preparation of modified RNA molecules useful according to the present invention can be found, for example, in the articles: Gamper, H. B., Reed, M. W., Cox, T., Virosco, J. S., Adams, A. D., Gall, A., Scholler, J. K., and Meyer, R. B. (1993) Facile Preparation and Exonuclease Stability of 3′-Modified Oligodeoxynucleotides. Nucleic Acids Res. 21 145-150; and Reed, M. W., Adams, A. D., Nelson, J. S., and Meyer, R. B., Jr. (1991) Acridine and Cholesterol-Derivatized Solid Supports for Improved Synthesis of 3′-Modified Oligonucleotides. Bioconjugate Chem. 2 217-225 (1993).

Other siRNAs useful for targeting the IGPR-1 gene can be readily designed and tested. Accordingly, siRNAs useful for the methods described herein include siRNA molecules of about 15 to about 40 or about 15 to about 28 nucleotides in length, which are homologous to the IGPR-1 gene. In some embodiments, a IGPR-1 targeting siRNA molecules have a length of about 25 to about 29 nucleotides. In some embodiments, a IGPR-1 targeting siRNA molecules have a length of about 27, 28, 29, or 30 nucleotides. In some embodiments, a IGPR-1 targeting siRNA molecules can also comprise a 3′ hydroxyl group. In some embodiments, a IGPR-1 targeting siRNA molecules can be single-stranded or double stranded; such molecules can be blunt ended or comprise overhanging ends (e.g., 5′, 3′). In specific embodiments, the RNA molecule can be a double stranded and either blunt ended or comprises overhanging ends.

In one embodiment, at least one strand of the IGPR-1 targeting RNA molecule has a 3′ overhang from about 0 to about 6 nucleotides (e.g., pyrimidine nucleotides, purine nucleotides) in length. In other embodiments, the 3′ overhang is from about 1 to about 5 nucleotides, from about 1 to about 3 nucleotides and from about 2 to about 4 nucleotides in length. In one embodiment the IGPR-1 targeting RNA molecule is double stranded—one strand has a 3′ overhang and the other strand can be blunt-ended or have an overhang. In the embodiment in which the IGPR-1 targeting RNA molecule is double stranded and both strands comprise an overhang, the length of the overhangs can be the same or different for each strand. In a particular embodiment, the RNA of the present invention comprises about 19, 20, 21, or 22 nucleotides which are paired and which have overhangs of from about 1 to about 3, particularly about 2, nucleotides on both 3′ ends of the RNA. In one embodiment, the 3′ overhangs can be stabilized against degradation. In a preferred embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

IGPR-1 as disclosed herein in the Examples, siRNAs to IGPR-1 have been successfully used to target and inhibit IGPR-1 function. In some embodiments, where gene silencing RNAi of IGPR-1 are not commercially available, gene silencing RNAi agents targeting inhibition of IGPR-1 can be produced by one of ordinary skill in the art and according to the methods as disclosed herein. In some embodiments, the assessment of the expression and/or knock down of a IGPR-1 mRNA and/or protein can be determined using commercially available kits known by persons of ordinary skill in the art. Others can be readily prepared by those of skill in the art based on the known sequence of the target mRNA.

To avoid doubt, the sequence of a human IGPR-1 cDNA is provided at, for example, GenBank Accession Nos.: NM_{—}144615.2 (SEQ ID NO: 2) and can be used to design a gene silencing RNAi modulator which inhibits IGPR-1 mRNA expression. In some embodiments, an inhibitor of IGPR-1 is a siRNA agent, for example, but not limited to: CAGCAAAGGGACUCAGGUAUU (SEQ ID NO: 20) or AGGUAACAGCCCAGGAAAUUU (SEQ ID NO: 21) and variants and derivatives thereof.

In some embodiments, an inhibitor of IGPR-1 is a gene silencing RNAi agent which downregulates or decreases IGPR-1 mRNA levels is a 25-nt hairpin sequence. In some embodiments, an inhibitor of IGPR-1 is a gene silencing RNAi, such as, for example, a shRNA sequence.

In one embodiment, the RNA interfering agents used in the methods described herein are taken up actively by cells in vivo following intravenous injection, e.g., hydrodynamic injection, without the use of a vector, illustrating efficient in vivo delivery of the RNA interfering agents, e.g., the siRNAs used in the methods of the invention.

Other strategies for delivery of the RNA interfering agents, e.g., the siRNAs or shRNAs used in the methods of the invention, can also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the RNA interfering agents, e.g., the siRNAs or shRNAs of the invention, using a basic peptide by conjugating or mixing the RNA interfering agent with a basic peptide, e.g., a fragment of a TAT peptide, mixing with cationic lipids or formulating into particles.

As noted, the dsRNA, such as siRNA or shRNA can be delivered using an inducible vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used. In some embodiments, a vector can be a plasmid vector, a viral vector, or any other suitable vehicle adapted for the insertion and foreign sequence and for the introduction into eukaryotic cells. The vector can be an expression vector capable of directing the transcription of the DNA sequence of the agonist or antagonist nucleic acid molecules into RNA. Viral expression vectors can be selected from a group comprising, for example, reteroviruses, lentiviruses, Epstein Barr virus-, bovine papilloma virus, adenovirus- and adeno-associated-based vectors or hybrid virus of any of the above. In one embodiment, the vector is episomal. The use of a suitable episomal vector provides a means of maintaining the antagonist nucleic acid molecule in the subject in high copy number extra chromosomal DNA thereby eliminating potential effects of chromosomal integration.

RNA interference molecules and nucleic acid inhibitors useful in the methods as disclosed herein can be produced using any known techniques such as direct chemical synthesis, through processing of longer double stranded RNAs by exposure to recombinant Dicer protein or Drosophila embryo lysates, through an in vitro system derived from S2 cells, using phage RNA polymerase, RNA-dependant RNA polymerase, and DNA based vectors. Use of cell lysates or in vitro processing can further involve the subsequent isolation of the short, for example, about 21-23 nucleotide, siRNAs from the lysate, etc. Chemical synthesis usually proceeds by making two single stranded RNA-oligomers followed by the annealing of the two single stranded oligomers into a double stranded RNA. Other examples include methods disclosed in WO 99/32619 and WO 01/68836 that teach chemical and enzymatic synthesis of siRNA. Moreover, numerous commercial services are available for designing and manufacturing specific siRNAs (see, e.g., QIAGEN Inc., Valencia, Calif. and AMBION Inc., Austin, Tex.).

The terms “antimir” “microRNA inhibitor” or “miR inhibitor” are synonymous and refer to oligonucleotides that interfere with the activity of specific miRNAs. Inhibitors can adopt a variety of configurations including single stranded, double stranded (RNA/RNA or RNA/DNA duplexes), and hairpin designs, in general, microRNA inhibitors comprise one or more sequences or portions of sequences that are complementary or partially complementary with the mature strand (or strands) of the miRNA to be targeted, in addition, the miRNA inhibitor can also comprise additional sequences located 5′ and 3′ to the sequence that is the reverse complement of the mature miRNA. The additional sequences can be the reverse complements of the sequences that are adjacent to the mature miRNA in the pri-miRNA from which the mature miRNA is derived, or the additional sequences can be arbitrary sequences (having a mixture of A, G, C, U, or dT). In some embodiments, one or both of the additional sequences are arbitrary sequences capable of forming hairpins. Thus, in some embodiments, the sequence that is the reverse complement of the miRNA is flanked on the 5′ side and on the 3′ side by hairpin structures. MicroRNA inhibitors, when double stranded, can include mismatches between nucleotides on opposite strands.

In some embodiments, an agent is protein or polypeptide or RNAi agent which inhibits the expression of the IGPR-1 gene. In such embodiments cells can be modified (e.g., by homologous recombination) to provide increased expression of such an agent, for example by replacing, in whole or in part, the naturally occurring promoter with all or part of a heterologous promoter so that the cells express an inhibitor of IGPR-1, for example a protein or RNAi agent (e.g. gene silencing-RNAi agent). Typically, a heterologous promoter is inserted in such a manner that it is operatively linked to the desired nucleic acid encoding the agent. See, for example, PCT International Publication No. WO 94/12650 by Transkaryotic Therapies, Inc., PCT International Publication No. WO 92/20808 by Cell Genesys, Inc., and PCT International Publication No. WO 91/09955 by Applied Research Systems. Cells also can be engineered to express an endogenous gene comprising the inhibitor agent under the control of inducible regulatory elements, in which case the regulatory sequences of the endogenous gene can be replaced by homologous recombination. Gene activation techniques are described in U.S. Pat. No. 5,272,071 to Chappel; U.S. Pat. No. 5,578,461 to Sherwin et al.; PCT/US92/09627 (W093/09222) by Selden et al.; and PCT/US90/06436 (WO91/06667) by Skoultchi et al. The agent can be prepared by culturing transformed host cells under culture conditions suitable to express the miRNA. The resulting expressed agent can then be purified from such culture (i.e., from culture medium or cell extracts) using known purification processes, such as gel filtration and ion exchange chromatography. The purification of the peptide or nucleic acid agent inhibitor of IGPR-1 can also include an affinity column containing agents which will bind to the protein; one or more column steps over such affinity resins as concanavalin A-agarose, Heparin-toyopearl™ or Cibacrom blue 3GA Sepharose; one or more steps involving hydrophobic interaction chromatography using such resins as phenyl ether, butyl ether, or propyl ether; immunoaffinity chromatography, or complementary cDNA affinity chromatography.

In one embodiment, a nucleic acid inhibitor of IGPR-1, e.g. (gene silencing RNAi agent) can be obtained synthetically, for example, by chemically synthesizing a nucleic acid by any method of synthesis known to the skilled artisan. A synthesized nucleic acid inhibitor of IGPR-1 can then be purified by any method known in the art. Methods for chemical synthesis of nucleic acids include, but are not limited to, in vitro chemical synthesis using phosphotriester, phosphate or phosphoramidite chemistry and solid phase techniques, or via deoxynucleoside H-phosphonate intermediates (see U.S. Pat. No. 5,705,629 to Bhongle).

In some circumstances, for example, where increased nuclease stability of a nucleic acid inhibitor is desired, nucleic acids having nucleic acid analogs and/or modified internucleoside linkages can be used. Nucleic acids containing modified internucleoside linkages can also be synthesized using reagents and methods that are well known in the art. For example, methods of synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH₂—S—CH₂), dimethylene-sulfoxide (—CH₂—SO—CH₂), dimethylene-sulfone (—CH₂—SO₂—CH₂), 2′-O-alkyl, and 2′-deoxy-2′-fluoro′phosphorothioate internucleoside linkages are well known in the art (see Uhlmann et al., 1990, Chem. Rev. 90:543-584; Schneider et al., 1990, Tetrahedron Lett. 31:335 and references cited therein). U.S. Pat. Nos. 5,614,617 and 5,223,618 to Cook, et al., U.S. Pat. No. 5,714,606 to Acevedo, et al, U.S. Pat. No. 5,378,825 to Cook, et al., U.S. Pat. No. 5,672,697 and U.S. Pat. No. 5,466,786 to Buhr, et al., U.S. Pat. No. 5,777,092 to Cook, et al., U.S. Pat. No. 5,602,240 to De Mesmacker, et al., U.S. Pat. No. 5,610,289 to Cook, et al. and U.S. Pat. No. 5,858,988 to Wang, also describe nucleic acid analogs for enhanced nuclease stability and cellular uptake.

Synthetic siRNA molecules, including shRNA molecules, can also easily be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but are not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell. 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structures. Within cells, Dicer processes the short hairpin RNA (shRNA) into effective siRNA.

In some embodiments, an inhibitor of IGPR-1 is a gene silencing siRNA molecule which targets a IGPR-1 gene and targets the coding mRNA sequence of IGPR-1, beginning from about 25 to 50 nucleotides, from about 50 to 75 nucleotides, or from about 75 to 100 nucleotides downstream of the start codon. One method of designing a siRNA molecule of the present invention involves identifying the 29 nucleotide sequence motif AA(N29)TT (where N can be any nucleotide) (SEQ ID NO: 24), and selecting hits with at least 25%, 30%, 35%, 40%, 45%, 50%, 55%, 60%, 65%, 70% or 75% G/C content. The “TT” portion of the sequence is optional. Alternatively, if no such sequence is found, the search can be extended using the motif NA(N21), where N can be any nucleotide. In this situation, the 3′ end of the sense siRNA can be converted to TT to allow for the generation of a symmetric duplex with respect to the sequence composition of the sense and antisense 3′ overhangs. The antisense siRNA molecule can then be synthesized as the complement to nucleotide positions 1 to 21 of the 23 nucleotide sequence motif. The use of symmetric 3′ TT overhangs can be advantageous to ensure that the small interfering ribonucleoprotein particles (siRNPs) are formed with approximately equal ratios of sense and antisense target RNA-cleaving siRNPs (Elbashir et al. (2001) supra and Elbashir et al. 2001 supra). Analysis of sequence databases, including but not limited to the NCBI, BLAST, Derwent and GenSeq as well as commercially available oligosynthesis software such as Oligoengine®, can also be used to select siRNA sequences against EST libraries to ensure that only one gene is targeted.

siRNAs useful for the methods described herein include siRNA molecules of about 15 to about 40 or about 15 to about 28 nucleotides in length, which are homologous to the IGPR-1. Preferably, a targeting siRNA molecule to the IGPR-1 gene has a length of about 19 to about 25 nucleotides. More preferably, the targeting siRNA molecules have a length of about 19, 20, 21, or 22 nucleotides. The targeting siRNA molecules can also comprise a 3′ hydroxyl group. The targeting siRNA molecules can be single-stranded or double stranded; such molecules can be blunt ended or comprise overhanging ends (e.g., 5′, 3′). In specific embodiments, the RNA molecule is double stranded and either blunt ended or comprises overhanging ends.

In one embodiment, at least one strand of a IGPR-1 RNAi targeting RNA molecule has a 3′ overhang from about 0 to about 6 nucleotides (e.g., pyrimidine nucleotides, purine nucleotides) in length. In other embodiments, the 3′ overhang is from about 1 to about 5 nucleotides, from about 1 to about 3 nucleotides and from about 2 to about 4 nucleotides in length. In one embodiment the targeting RNA molecule is double stranded—one strand has a 3′ overhang and the other strand can be blunt-ended or have an overhang. In the embodiment in which the targeting RNA molecule is double stranded and both strands comprise an overhang, the length of the overhangs can be the same or different for each strand. In a particular embodiment, the RNA of the present invention comprises about 19, 20, 21, or 22 nucleotides which are paired and which have overhangs of from about 1 to about 3, particularly about 2, nucleotides on both 3′ ends of the RNA. In one embodiment, the 3′ overhangs can be stabilized against degradation. In a preferred embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

Oligonucleotide Modifications

Unmodified oligonucleotides can be less than optimal in some applications, e.g., unmodified oligonucleotides can be prone to degradation by e.g., cellular nucleases. Nucleases can hydrolyze nucleic acid phosphodiester bonds. However, chemical modifications to one or more of the subunits of oligonucleotide can confer improved properties, and, e.g., can render oligonucleotides more stable to nucleases.

Modified nucleic acids and nucleotide surrogates can include one or more of: (i) alteration, e.g., replacement, of one or both of the non-linking phosphate oxygens and/or of one or more of the linking phosphate oxygens in the phosphodiester backbone linakge. (ii) alteration, e.g., replacement, of a constituent of the ribose sugar, e.g., of the 2′ hydroxyl on the ribose sugar; (iii) wholesale replacement of the phosphate moiety with “dephospho” linkers; (iv) modification or replacement of a naturally occurring base with a non-natural base; (v) replacement or modification of the ribose-phosphate backbone; (vi) modification of the 3′ end or 5′ end of the oligonucleotide, e.g., removal, modification or replacement of a terminal phosphate group or conjugation of a moiety, e.g., a fluorescently labeled moiety, to either the 3′ or 5′ end of oligonucleotide; and (vii) modification of the sugar (e.g., six membered rings).

The terms replacement, modification, alteration, and the like, as used in this context, do not imply any process limitation, e.g., modification does not mean that one must start with a reference or naturally occurring ribonucleic acid and modify it to produce a modified ribonucleic acid bur rather modified simply indicates a difference from a naturally occurring molecule.

As oligonucleotides are polymers of subunits or monomers, many of the modifications described herein can occur at a position which is repeated within an oligonucleotide, e.g., a modification of a nucleobase, a sugar, a phosphate moiety, or the non-bridging oxygen of a phosphate moiety. It is not necessary for all positions in a given oligonucleotide to be uniformly modified, and in fact more than one of the aforementioned modifications can be incorporated in a single oligonucleotide or even at a single nucleoside within an oligonucleotide.

In some cases the modification will occur at all of the subject positions in the oligonucleotide but in many, and in fact in most cases it will not. By way of example, a modification can only occur at a 3′ or 5′ terminal position, can only occur in the internal region, can only occur in a terminal regions, e.g. at a position on a terminal nucleotide or in the last 2, 3, 4, 5, or 10 nucleotides of an oligonucleotide. A modification can occur in a double strand region, a single strand region, or in both. A modification can occur only in the double strand region of an oligonucleotide or can only occur in a single strand region of an oligonucleotide. E.g., a phosphorothioate modification at a non-bridging oxygen position can only occur at one or both termini, can only occur in a terminal regions, e.g., at a position on a terminal nucleotide or in the last 2, 3, 4, 5, or 10 nucleotides of a strand, or can occur in double strand and single strand regions, particularly at termini. The 5′ end or ends can be phosphorylated.

A modification described herein can be the sole modification, or the sole type of modification included on multiple nucleotides, or a modification can be combined with one or more other modifications described herein. The modifications described herein can also be combined onto an oligonucleotide, e.g. different nucleotides of an oligonucleotide have different modifications described herein.

In some embodiments it is particularly preferred, e.g., to enhance stability, to include particular nucleobases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang will be modified, e.g., with a modification described herein. Modifications can include, e.g., the use of modifications at the 2′ OH group of the ribose sugar, e.g., the use of deoxyribonucleotides, e.g., deoxythymidine, instead of ribonucleotides, and modifications in the phosphate group, e.g., phosphothioate modifications. Overhangs need not be homologous with the target sequence.

Specific Modifications to Oligonucleotide

the Phosphate Group

The phosphate group is a negatively charged species. The charge is distributed equally over the two non-bridging oxygen atoms. However, the phosphate group can be modified by replacing one of the oxygens with a different substituent. One result of this modification to RNA phosphate backbones can be increased resistance of the oligoribonucleotide to nucleolytic breakdown. Thus while not wishing to be bound by theory, it can be desirable in some embodiments to introduce alterations which result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. In certain embodiments, one of the non-bridging phosphate oxygen atoms in the phosphate backbone moiety can be replaced by any of the following: S, Se, BR₃ (R is hydrogen, alkyl, aryl), C (i.e. an alkyl group, an aryl group, etc. . . . ), H, NR₂ (R is hydrogen, alkyl, aryl), or OR (R is alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligoribonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of S, Se, B, C, H, N, or OR (R is alkyl or aryl).

The phosphate linker can also be modified by replacement of bridging oxygen, (i.e. oxygen that links the phosphate to the nucleoside), with nitrogen (bridged phosphoroamidates), sulfur (bridged phosphorothioates) and carbon (bridged methylenephosphonates). The replacement can occur at the either linking oxygen or at both the linking oxygens. When the bridging oxygen is the 3′-oxygen of a nucleoside, replacement with carbon is preferred. When the bridging oxygen is the 5′-oxygen of a nucleoside, replacement with nitrogen is preferred.

Replacement of the Phosphate Group

The phosphate group can be replaced by non-phosphorus containing connectors. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. Preferred replacements include the methylenecarbonylamino and methylenemethylimino groups.

Modified phosphate linkages where at least one of the oxygens linked to the phosphate has been replaced or the phosphate group has been replaced by a non-phosphorous group, are also referred to as “non-phosphodiester backbone linkage.”

Replacement of Ribophosphate Backbone

Oligonucleotide-mimicking scaffolds can also be constructed wherein the phosphate linker and ribose sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the morpholino, cyclobutyl, pyrrolidine and peptide nucleic acid (PNA) nucleoside surrogates. A preferred surrogate is a PNA surrogate.

Sugar Modifications

An oligonucleotide can include modification of all or some of the sugar groups of the nucleic acid. E.g., the 2′ hydroxyl group (OH) can be modified or replaced with a number of different “oxy” or “deoxy” substituents. While not being bound by theory, enhanced stability is expected since the hydroxyl can no longer be deprotonated to form a 2′-alkoxide ion. The 2′-alkoxide can catalyze degradation by intramolecular nucleophilic attack on the linker phosphorus atom. Again, while not wishing to be bound by theory, it can be desirable to some embodiments to introduce alterations in which alkoxide formation at the 2′ position is not possible.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; O-AMINE (AMINE=NH₂; alkylamino, dialkylamino, heterocyclyl, arylamino, diaryl amino, heteroaryl amino, or diheteroaryl amino, ethylene diamine, polyamino) and aminoalkoxy, O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e. deoxyribose sugars, which are of particular relevance to the overhang portions of partially ds RNA); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino), —NHC(O)R (R=alkyl, cycloalkyl, aryl, aralkyl, heteroaryl or sugar), cyano; mercapto; alkyl-thio-alkyl; thioalkoxy; thioalkyl; and alkyl, cycloalkyl, aryl, alkenyl and alkynyl, which can be optionally substituted with e.g., an amino functionality.

The sugar group can also contain one or more carbons that possess the opposite stereochemical configuration than that of the corresponding carbon in ribose. Thus, an oligonucleotide can include nucleotides containing e.g., arabinose, as the sugar. The monomer can have an alpha linkage at the 1′ position on the sugar, e.g., alpha-nucleosides. Oligonucleotides can also include “abasic” sugars, which lack a nucleobase at C-1′. These abasic sugars can also be further containing modifications at one or more of the constituent sugar atoms. Oligonucleotides can also contain one or more sugars that are in the L form, e.g. L-nucleosides.

Preferred substitutents are 2′-O-Me (2′-O-methyl), 2′-O-MOE (2′-O-methoxyethyl), 2′-F, 2′-O-[2-(methylamino)-2-oxoethyl](2′-O-NMA), 2′-S-methyl, 2′-O—CH2-(4′-C) (LNA), 2′-O—CH₂CH₂-(4′-C) (ENA), 2′-O-aminopropyl (2′-O-AP), 2′-O-dimethylaminoethyl (2′-β-DMAOE), 2′-O-dimethylaminopropyl (2′-O-DMAP) and 2′-O-dimethylaminoethyloxyethyl (2′-O-DMAEOE).

Terminal Modifications

The 3-prime (3′) and 5-prime (5′) ends of an oligonucleotide can be modified. Such modifications can be at the 3′ end, 5′ end or both ends of the molecule. They can include modification or replacement of an entire terminal phosphate or of one or more of the atoms of the phosphate group. E.g., the 3′ and 5′ ends of an oligonucleotide can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

When a linker/phosphate-functional molecular entity-linker/phosphate array is interposed between two strands of a dsRNA, this array can substitute for a hairpin RNA loop in a hairpin-type RNA agent.

Terminal modifications useful for modulating activity include modification of the 5′ end with phosphate or phosphate analogs. E.g., in preferred embodiments antisense strands of dsRNAs, are 5′ phosphorylated or include a phosphoryl analog at the 5′ prime terminus. 5′-phosphate modifications include those which are compatible with RISC mediated gene silencing. Modifications at the 5′-terminal end can also be useful in stimulating or inhibiting the immune system of a subject. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-beta-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g. RP(OH)(O)—O-5′-, (OH)₂(O)P-5′-CH₂—), 5′-alkyletherphosphonates (R=alkylether=methoxymethyl (MeOCH₂—), ethoxymethyl, etc., e.g. RP(OH)(O)—O-5′-). Other embodiments include replacement of oxygen/sulfur with BH₃, BH₃— and/or Se.

Terminal modifications can also be useful for monitoring distribution, and in such cases the preferred groups to be added include fluorophores, e.g., fluorscein or an ALEXA® dye, e.g., ALEXA® 488. Terminal modifications can also be useful for enhancing uptake, useful modifications for this include cholesterol. Terminal modifications can also be useful for cross-linking an RNA agent to another moiety; modifications useful for this include mitomycin C.

Nucleobases

Adenine, guanine, cytosine and uracil are the most common bases found in RNA. These bases can be modified or replaced to provide RNA's having improved properties. For example, nuclease resistant oligoribonucleotides can be prepared with these bases or with synthetic and natural nucleobases (e.g., inosine, thymine, xanthine, hypoxanthine, nubularine, isoguanisine, or tubercidine) and any one of the above modifications. Alternatively, substituted or modified analogs of any of the above bases and “universal bases” can be employed. Examples include 2-(halo)adenine, 2-(alkyl)adenine, 2-(propyl)adenine, 2 (amino)adenine, 2-(aminoalkyl)adenine, 2 (aminopropyl)adenine, 2 (methylthio) N⁶ (isopentenyl)adenine, 6 (alkyl)adenine, 6 (methyl)adenine, 7 (deaza)adenine, 8 (alkenyl)adenine, 8-(alkyl)adenine, 8 (alkynyl)adenine, 8 (amino)adenine, 8-(halo)adenine, 8-(hydroxyl)adenine, 8 (thioalkyl)adenine, 8-(thiol)adenine, N⁶-(isopentyl)adenine, N⁶ (methyl)adenine, N⁶, N⁶ (dimethyl)adenine, 2-(alkyl)guanine, 2 (propyl)guanine, 6-(alkyl)guanine, 6 (methyl)guanine, 7 (alkyl)guanine, 7 (methyl)guanine, 7 (deaza)guanine, 8 (alkyl)guanine, 8-(alkenyl)guanine, 8 (alkynyl)guanine, 8-(amino)guanine, 8 (halo)guanine, 8-(hydroxyl)guanine, 8 (thioalkyl)guanine, 8-(thiol)guanine, N (methyl)guanine, 2-(thio)cytosine, 3 (deaza) 5 (aza)cytosine, 3-(alkyl)cytosine, 3 (methyl)cytosine, 5-(alkyl)cytosine, 5-(alkynyl)cytosine, 5 (halo)cytosine, 5 (methyl)cytosine, 5 (propynyl)cytosine, 5 (propynyl)cytosine, 5 (trifluoromethyl)cytosine, 6-(azo)cytosine, N4 (acetyl)cytosine, 3 (3 amino-3 carboxypropyl)uracil, 2-(thio)uracil, 5 (methyl) 2 (thio)uracil, 5 (methylaminomethyl)-2 (thio)uracil, 4-(thio)uracil, 5 (methyl) 4 (thio)uracil, 5 (methylaminomethyl)-4 (thio)uracil, 5 (methyl) 2,4 (dithio)uracil, 5 (methylaminomethyl)-2,4 (dithio)uracil, 5 (2-aminopropyl)uracil, 5-(alkyl)uracil, 5-(alkynyl)uracil, 5-(allylamino)uracil, 5 (aminoallyl)uracil, 5 (aminoalkyl)uracil, 5 (guanidiniumalkyl)uracil, 5 (1,3-diazole-1-alkyl)uracil, 5-(cyanoalkyl)uracil, 5-(dialkylaminoalkyl)uracil, 5 (dimethylaminoalkyl)uracil, 5-(halo)uracil, 5-(methoxy)uracil, uracil-5 oxyacetic acid, 5 (methoxycarbonylmethyl)-2-(thio)uracil, 5 (methoxycarbonyl-methyl)uracil, 5 (propynyl)uracil, 5 (propynyl)uracil, 5 (trifluoromethyl)uracil, 6 (azo)uracil, dihydrouracil, N3 (methyl)uracil, 5-uracil (i.e., pseudouracil), 2 (thio)pseudouracil, 4 (thio)pseudouracil, 2,4-(dithio)pseudouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4 (thio)pseudouracil, 5-(methyl)-4 (thio)pseudouracil, 5-(alkyl)-2,4 (dithio)pseudouracil, 5-(methyl)-2,4 (dithio)pseudouracil, 1 substituted pseudouracil, 1 substituted 2(thio)-pseudouracil, 1 substituted 4 (thio)pseudouracil, 1 substituted 2,4-(dithio)pseudouracil, 1 (aminocarbonylethylenyl)-pseudouracil, 1 (aminocarbonylethylenyl)-2(thio)-pseudouracil, 1 (aminocarbonylethylenyl)-4 (thio)pseudouracil, 1 (aminocarbonylethylenyl)-2,4-(dithio)pseudouracil, 1 (aminoalkylaminocarbonylethylenyl)-pseudouracil, 1 (aminoalkylamino-carbonylethylenyl)-2(thio)-pseudouracil, 1 (aminoalkylaminocarbonylethylenyl)-4 (thio)pseudouracil, 1 (aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkylhydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(diaza)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenzyl, tetracenyl, pentacenyl, difluorotolyl, 4-(fluoro)-6-(methyl)benzimidazole, 4-(methyl)benzimidazole, 6-(azo)thymine, 2-pyridinone, 5 nitroindole, 3 nitropyrrole, 6-(aza)pyrimidine, 2 (amino)purine, 2,6-(diamino)purine, 5 substituted pyrimidines, N²-substituted purines, N⁶-substituted purines, O⁶-substituted purines, substituted 1,2,4-triazoles, or any O-alkylated or N-alkylated derivatives thereof;

Further purines and pyrimidines include those disclosed in U.S. Pat. No. 3,687,808, hereby incorporated by reference, those disclosed in the Concise Encyclopedia Of Polymer Science And Engineering, pages 858-859, Kroschwitz, J. I., ed. John Wiley & Sons, 1990, and those disclosed by Englisch et al., Angewandte Chemie, International Edition, 1991, 30, 613.

Cationic Groups

Modifications to oligonucleotides can also include attachment of one or more cationic groups to the sugar, base, and/or the phosphorus atom of a phosphate or modified phosphate backbone moiety. A cationic group can be attached to any atom capable of substitution on a natural, unusual or universal base. A preferred position is one that does not interfere with hybridization, i.e., does not interfere with the hydrogen bonding interactions needed for base pairing. A cationic group can be attached e.g., through the C2′ position of a sugar or analogous position in a cyclic or acyclic sugar surrogate. Cationic groups can include e.g., protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); or NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

Placement within an Oligonucleotide

Some modifications can preferably be included on an oligonucleotide at a particular location, e.g., at an internal position of a strand or on the 5′ or 3′ end of an oligonucleotide. A preferred location of a modification on an oligonucleotide, can confer preferred properties on the agent. For example, preferred locations of particular modifications can confer optimum gene silencing properties, or increased resistance to endonuclease or exonuclease activity.

One or more nucleotides of an oligonucleotide can have a 2′-5′ linkage. One or more nucleotides of an oligonucleotide can have inverted linkages, e.g. 3′-3′, 5′-5′,2′-2′ or 2′-3′ linkages.

An oligonucleotide can comprise at least one 5′-pyrimidine-purine-3′ (5′-PyPu-3′) dinucleotide wherein the pyrimidine is modified with a modification chosen independently from a group consisting of 2′-O-Me (2′-O-methyl), 2′-O-MOE (2′-O-methoxyethyl), 2′-F, 2′-β-[2-(methylamino)-2-oxoethyl] (2′-O-NMA), 2′-S-methyl, 2′-O—CH₂-(4′-C) (LNA) and 2′-O—CH₂CH₂-(4′-C) (ENA).

In one embodiment, the 5′-most pyrimidines in all occurrences of sequence motif 5′-pyrimidine-purine-3′ (5′-PyPu-3′) dinucleotide in the oligonucleotide are modified with a modification chosen from a group consisting of 2″-O-Me (2′-O-methyl), 2′-O-MOE (2′-β-methoxyethyl), 2′-F, 2′-O—[2-(methylamino)-2-oxoethyl] (2′-O-NMA), 2′-S-methyl, 2′-O—CH₂-(4′-C) (LNA) and 2′-O—CH₂CH₂-(4′-C) (ENA).

A double-stranded oligonucleotide can include at least one 5′-uridine-adenine-3′ (5′-UA-3′) dinucleotide wherein the uridine is a 2′-modified nucleotide, or a 5′-uridine-guanine-3′ (5′-UG-3′) dinucleotide, wherein the 5′-uridine is a 2′-modified nucleotide, or a terminal 5′-cytidine-adenine-3′ (5′-CA-3′) dinucleotide, wherein the 5′-cytidine is a 2′-modified nucleotide, or a terminal 5′-uridine-uridine-3′ (5′-UU-3′) dinucleotide, wherein the 5′-uridine is a 2′-modified nucleotide, or a terminal 5′-cytidine-cytidine-3′ (5′-CC-3′) dinucleotide,

wherein the 5′-cytidine is a 2′-modified nucleotide, or a terminal 5′-cytidine-uridine-3′ (5′-CU-3′) dinucleotide, wherein the 5′-cytidine is a 2′-modified nucleotide, or a terminal 5′-uridine-cytidine-3′ (5′-UC-3′) dinucleotide, wherein the 5′-uridine is a 2′-modified nucleotide. Double-stranded oligonucleotides including these modifications are particularly stabilized against endonuclease activity.

General References

The oligoribonucleotides and oligoribonucleosides used in accordance with this invention can be synthesized with solid phase synthesis, see for example “Oligonucleotide synthesis, a practical approach”, Ed. M. J. Gait, IRL Press, 1984; “Oligonucleotides and Analogues, A Practical Approach”, Ed. F. Eckstein, IRL Press, 1991 (especially Chapter 1, Modern machine-aided methods of oligodeoxyribonucleotide synthesis, Chapter 2, Oligoribonucleotide synthesis, Chapter 3,2′-O-Methyloligoribonucleotide-s: synthesis and applications, Chapter 4, Phosphorothioate oligonucleotides, Chapter 5, Synthesis of oligonucleotide phosphorodithioates, Chapter 6, Synthesis of oligo-2′-deoxyribonucleoside methylphosphonates, and. Chapter 7, Oligodeoxynucleotides containing modified bases. Other particularly useful synthetic procedures, reagents, blocking groups and reaction conditions are described in Martin, P., Hely. Chim. Acta, 1995, 78, 486-504; Beaucage, S. L. and Iyer, R. P., Tetrahedron, 1992, 48, 2223-2311 and Beaucage, S. L. and Iyer, R. P., Tetrahedron, 1993, 49, 6123-6194, or references referred to therein. Modification described in WO 00/44895, WO01/75164, or WO02/44321 can be used herein. The disclosure of all publications, patents, and published patent applications listed herein are hereby incorporated by reference.

Phosphate Group References

The preparation of phosphinate oligoribonucleotides is described in U.S. Pat. No. 5,508,270. The preparation of alkyl phosphonate oligoribonucleotides is described in U.S. Pat. No. 4,469,863. The preparation of phosphoramidite oligoribonucleotides is described in U.S. Pat. No. 5,256,775 or U.S. Pat. No. 5,366,878. The preparation of phosphotriester oligoribonucleotides is described in U.S. Pat. No. 5,023,243. The preparation of borano phosphate oligoribonucleotide is described in U.S. Pat. Nos. 5,130,302 and 5,177,198. The preparation of 3′-Deoxy-3′-amino phosphoramidate oligoribonucleotides is described in U.S. Pat. No. 5,476,925. 3′-Deoxy-3′-methylenephosphonate oligoribonucleotides is described in An, H, et al. J. Org. Chem. 2001, 66, 2789-2801. Preparation of sulfur bridged nucleotides is described in Sproat et al. Nucleosides Nucleotides 1988, 7,651 and Crosstick et al. Tetrahedron Lett. 1989, 30, 4693.

Sugar Group References

Modifications to the 2′ modifications can be found in Verma, S. et al. Annu. Rev. Biochem. 1998, 67, 99-134 and all references therein. Specific modifications to the ribose can be found in the following references: 2′-fluoro (Kawasaki et. al., J. Med. Chem., 1993, 36, 831-841), 2′-MOE (Martin, P. Hely. Chim. Acta 1996, 79, 1930-1938), “LNA” (Wengel, J. Acc. Chem. Res. 1999, 32, 301-310).

Replacement of the Phosphate Group References

Methylenemethylimino linked oligoribonucleosides, also identified herein as MMI linked oligoribonucleosides, methylenedimethylhydrazo linked oligoribonucleosides, also identified herein as MDH linked oligoribonucleo sides, and methylenecarbonylamino linked oligonucleosides, also identified herein as amide-3 linked oligoribonucleosides, and methyleneaminocarbonyl linked oligonucleosides, also identified herein as amide-4 linked oligoribonucleosides as well as mixed backbone compounds having, as for instance, alternating MMI and PO or PS linkages can be prepared as is described in U.S. Pat. Nos. 5,378,825, 5,386,023, 5,489,677 and in published PCT applications PCT/US92/04294 and PCT/US92/04305 (published as WO 92/20822 WO and 92/20823, respectively). Formacetal and thioformacetal linked oligoribonucleosides can be prepared as is described in U.S. Pat. Nos. 5,264,562 and 5,264,564. Ethylene oxide linked oligoribonucleosides can be prepared as is described in U.S. Pat. No. 5,223,618. Siloxane replacements are described in Cormier, J. F. et al. Nucleic Acids Res. 1988, 16, 4583. Carbonate replacements are described in Tittensor, J. R. J. Chem. Soc. C 1971, 1933. Carboxymethyl replacements are described in Edge, M. D. et al. J. Chem. Soc. Perkin Trans. 11972, 1991. Carbamate replacements are described in Stirchak, E. P. Nucleic Acids Res. 1989, 17, 6129.

Replacement of the Phosphate-Ribose Backbone References

Cyclobutyl sugar surrogate compounds can be prepared as is described in U.S. Pat. No. 5,359,044. Pyrrolidine sugar surrogate can be prepared as is described in U.S. Pat. No. 5,519,134. Morpholino sugar surrogates can be prepared as is described in U.S. Pat. Nos. 5,142,047 and 5,235,033, and other related patent disclosures. Peptide Nucleic Acids (PNAs) are known per se and can be prepared in accordance with any of the various procedures referred to in Peptide Nucleic Acids (PNA): Synthesis, Properties and Potential Applications, Bioorganic & Medicinal Chemistry, 1996, 4, 5-23. They can also be prepared in accordance with U.S. Pat. No. 5,539,083 which is incorporated herein in its entirety by reference.

Terminal Modification References.

Terminal modifications are described in Manoharan, M. et al. Antisense and Nucleic Acid Drug Development 12, 103-128 (2002) and references therein.

Nucleobases References

N-2 substituted purine nucleoside amidites can be prepared as is described in U.S. Pat. No. 5,459,255. 3-Deaza purine nucleoside amidites can be prepared as is described in U.S. Pat. No. 5,457,191. 5,6-Substituted pyrimidine nucleoside amidites can be prepared as is described in U.S. Pat. No. 5,614,617. 5-Propynyl pyrimidine nucleoside amidites can be prepared as is described in U.S. Pat. No. 5,484,908. Additional references are disclosed in the above section on base modifications

Oligonucleotide Production

The oligonucleotide compounds of the invention can be prepared using solution-phase or solid-phase organic synthesis. Organic synthesis offers the advantage that the oligonucleotide strands comprising non-natural or modified nucleotides can be easily prepared. Any other means for such synthesis known in the art can additionally or alternatively be employed. It is also known to use similar techniques to prepare other oligonucleotides, such as the phosphorothioates, phosphorodithioates and alkylated derivatives. The double-stranded oligonucleotide compounds of the invention can be prepared using a two-step procedure. First, the individual strands of the double-stranded molecule are prepared separately. Then, the component strands are annealed.

Regardless of the method of synthesis, the oligonucleotide can be prepared in a solution (e.g., an aqueous and/or organic solution) that is appropriate for formulation. For example, the oligonucleotide preparation can be precipitated and redissolved in pure double-distilled water, and lyophilized. The dried oligonucleotide can then be resuspended in a solution appropriate for the intended formulation process.

Teachings regarding the synthesis of particular modified oligonucleotides can be found in the following U.S. patents or pending patent applications: U.S. Pat. Nos. 5,138,045 and 5,218,105, drawn to polyamine conjugated oligonucleotides; U.S. Pat. No. 5,212,295, drawn to monomers for the preparation of oligonucleotides having chiral phosphorus linkages; U.S. Pat. Nos. 5,378,825 and 5,541,307, drawn to oligonucleotides having modified backbones; U.S. Pat. No. 5,386,023, drawn to backbone-modified oligonucleotides and the preparation thereof through reductive coupling; U.S. Pat. No. 5,457,191, drawn to modified nucleobases based on the 3-deazapurine ring system and methods of synthesis thereof; U.S. Pat. No. 5,459,255, drawn to modified nucleobases based on N-2 substituted purines; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having β-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups can be used as linkers to other moieties attached at any of a variety of positions of the nucleoside; U.S. Pat. Nos. 5,587,361 and 5,599,797, drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. No. 5,223,168, and U.S. Pat. No. 5,608,046, both drawn to conjugated 4′-desmethyl nucleoside analogs; U.S. Pat. Nos. 5,602,240, and 5,610,289, drawn to backbone-modified oligonucleotide analogs; and U.S. Pat. Nos. 6,262,241, and 5,459,255, drawn to, inter alia, methods of synthesizing 2′-fluoro-oligonucleotides.

Delivery of RNA Interfering Agents:

Methods of delivering RNAi agents, e.g., an siRNA, or vectors containing an RNAi agent, to the target cells (e.g., basal cells or cells of the lung ad/or respiratory system or other desired target cells) are well known to persons of ordinary skill in the art. In some embodiments, a RNAi agent (e.g. gene silencing—RNAi agent) which is an inhibitor of IGPR-1 can be administered to a subject via aerosol means, for example using a nebulizer and the like. In alternative embodiments, administration of a RNAi agent (e.g. gene silencing—RNAi agent) which is an inhibitor of IGPR-1 can include, for example (i) injection of a composition containing the RNA interfering agent, e.g., an siRNA, or (ii) directly contacting the cell, e.g., a cell of the respiratory system, with a composition comprising an RNAi agent, e.g., an siRNA. In another embodiment, RNAi agents, e.g., an siRNA can be injected directly into any blood vessel, such as vein, artery, venule or arteriole, via, e.g., hydrodynamic injection or catheterization. In some embodiments an RNAi inhibitor of IGPR-1 can delivered to specific organs, for example the liver, bone marrow or systemic administration.

Administration can be by a single injection or by two or more injections. In some embodiments, a RNAi agent is delivered in a pharmaceutically acceptable carrier. One or more RNAi agents can be used simultaneously, e.g. one or more gene silencing RNAi agent inhibitors of IGPR-1 can be together. The RNA interfering agents, e.g., the siRNA inhibitors of IGPR-1, can be delivered singly, or in combination with other RNA interfering agents, e.g., siRNAs, such as, for example siRNAs directed to other cellular genes. A gene silencing—RNAi agent inhibitor of IGPR-1 can also be administered in combination with other pharmaceutical agents which are used to treat or prevent neurodegenerative diseases or disorders.

In one embodiment, specific cells are targeted with RNA interference, limiting potential side effects of RNA interference caused by non-specific targeting of RNA interference. The method can use, for example, a complex or a fusion molecule comprising a cell targeting moiety and an RNA interference binding moiety that is used to deliver RNAi effectively into cells. For example, an antibody-protamine fusion protein when mixed with an siRNA, binds siRNA and selectively delivers the siRNA into cells expressing an antigen recognized by the antibody, resulting in silencing of gene expression only in those cells that express the antigen which is identified by the antibody. In some embodiments, the antibody can be any antibody which identifies an antigen expressed on cells expressing IGPR-1. In some embodiments, the antibody is an antibody which binds to the IGPR-1 antigen, but where the antibody can or does not inhibit IGPR-1 function. In some embodiments, the siRNA can be conjugated to a IGPR-1 antagonist, for example where the IGPR-1 antagonist is a polypeptide, and where the conjugation with the RNAi does not interrupt the function of the IGPR-1 antagonist.

In some embodiments, a siRNA or RNAi binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein.

In some embodiments, a viral-mediated delivery mechanism can also be employed to deliver siRNAs, e.g. siRNAs (e.g. gene silencing RNAi agents) inhibitors of IGPR-1, to cells in vitro and in vivo as described in Xia, H. et al. (2002) Nat Biotechnol 20(10):1006). Plasmid- or viral-mediated delivery mechanisms of shRNA can also be employed to deliver shRNAs to cells in vitro and in vivo as described in Rubinson, D. A., et al. ((2003) Nat. Genet. 33:401-406) and Stewart, S. A., et al. ((2003) RNA 9:493-501). Alternatively, in other embodiments, a RNAi agent, e.g., a gene silencing—RNAi agent inhibitor of IGPR-1 can also be introduced into cells via the vascular or extravascular circulation, the blood or lymph system, and the cerebrospinal fluid.

In general, any method of delivering a nucleic acid molecule can be adapted for use with an RNAi interference molecule (see e.g., Akhtar S, and Julian R L. (1992) Trends Cell. Biol. 2(5):139-144; WO94/02595, which are incorporated herein by reference in their entirety). However, there are three factors that are important to consider in order to successfully deliver an RNAi molecule in vivo: (a) biological stability of the RNAi molecule, (2) preventing non-specific effects, and (3) accumulation of the RNAi molecule in the target tissue. The non-specific effects of an RNAi molecule can be minimized by local administration by e.g., direct injection into a tissue including, for example, a tumor or topically administering the molecule.

Local administration of an RNAi molecule to a treatment site limits the exposure of the e.g., siRNA to systemic tissues and permits a lower dose of the RNAi molecule to be administered. Several studies have shown successful knockdown of gene products when an RNAi molecule is administered locally. For example, intraocular delivery of a VEGF siRNA by intravitreal injection in cynomolgus monkeys (Tolentino, M J., et al (2004) Retina 24:132-138) and subretinal injections in mice (Reich, S J., et al (2003) Mol. Vis. 9:210-216) were both shown to prevent neovascularization in an experimental model of age-related macular degeneration. In addition, direct intratumoral injection of an siRNA in mice reduces tumor volume (Pille, J., et al (2005) Mol. Ther. 11:267-274) and can prolong survival of tumor-bearing mice (Kim, W J., et al (2006) Mol. Ther. 14:343-350; Li, S., et al (2007) Mol. Ther. 15:515-523). RNA interference has also shown success with local delivery to the CNS by direct injection (Dorn, G., et al (2004) Nucleic Acids 32:e49; Tan, P H., et al (2005) Gene Ther. 12:59-66; Makimura, H., et al (2002) BMC Neurosci. 3:18; Shishkina, G T., et al (2004) Neuroscience 129:521-528; Thakker, E R., et al (2004) Proc. Natl. Acad. Sci. U.S.A. 101:17270-17275; Akaneya, Y., et al (2005) J. Neurophysiol. 93:594-602) and to the lungs by intranasal administration (Howard, K A., et al (2006) Mol. Ther. 14:476-484; Zhang, X., et al (2004) J. Biol. Chem. 279:10677-10684; Bitko, V., et al (2005) Nat. Med. 11:50-55).

For administering an RNAi molecule systemically for the treatment of a disease, the RNAi molecule can be either be modified or alternatively delivered using a drug delivery system; both methods act to prevent the rapid degradation of the RNAi molecule by endo- and exo-nucleases in vivo. Modification of the RNAi molecule or the pharmaceutical carrier can also permit targeting of the RNAi molecule to the target tissue and avoid undesirable off-target effects.

RNA interference molecules can be modified by chemical conjugation to lipophilic groups such as cholesterol to enhance cellular uptake and prevent degradation. For example, an siRNA directed against ApoB conjugated to a lipophilic cholesterol moiety was injected systemically into mice and resulted in knockdown of apoB mRNA in both the liver and jejunum (Soutschek, J., et al (2004) Nature 432:173-178). Conjugation of an RNAi molecule to an aptamer has been shown to inhibit tumor growth and mediate tumor regression in a mouse model of prostate cancer (McNamara, JO., et al (2006) Nat. Biotechnol. 24:1005-1015).

In an alternative embodiment, the RNAi molecules can be delivered using drug delivery systems such as e.g., a nanoparticle, a dendrimer, a polymer, liposomes, or a cationic delivery system. Positively charged cationic delivery systems facilitate binding of an RNA interference molecule (negatively charged) and also enhance interactions at the negatively charged cell membrane to permit efficient uptake of an siRNA by the cell. Cationic lipids, dendrimers, or polymers can either be bound to an RNA interference molecule, or induced to form a vesicle or micelle (see e.g., Kim S H., et al (2008) Journal of Controlled Release 129(2):107-116) that encases an RNAi molecule. The formation of vesicles or micelles further prevents degradation of the RNAi molecule when administered systemically. Methods for making and administering cationic-RNAi complexes are well within the abilities of one skilled in the art (see e.g., Sorensen, D R., et al (2003) J. Mol. Biol. 327:761-766; Verma, U N., et al (2003) Clin. Cancer Res. 9:1291-1300; Arnold, A S et al (2007) J. Hypertens. 25:197-205, which are incorporated herein by reference in their entirety).

Some non-limiting examples of drug delivery systems useful for systemic administration of RNAi include DOTAP (Sorensen, D R., et al (2003), supra; Verma, UN., et al (2003), supra), Oligofectamine, “solid nucleic acid lipid particles” (Zimmermann, T S., et al (2006) Nature 441:111-114), cardiolipin (Chien, P Y., et al (2005) Cancer Gene Ther. 12:321-328; Pal, A., et al (2005) Int J. Oncol. 26:1087-1091), polyethyleneimine (Bonnet M E., et al (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A., et al (2007) Biochem. Soc. Trans. 35:61-67; Yoo, H., et al (1999) Pharm. Res. 16:1799-1804). In some embodiments, an RNAi molecule forms a complex with cyclodextrin for systemic administration. Methods for administration and pharmaceutical compositions of RNAi molecules and cyclodextrins can be found in U.S. Pat. No. 7,427,605, which is herein incorporated by reference in its entirety. Specific methods for administering an RNAi molecule for the inhibition of angiogenesis can be found in e.g., U.S. Patent Application No. 20080152654, which is herein incorporated by reference in its entirety.

In some embodiments, the siRNA, dsRNA, or shRNA vector can be administered systemically, such as intravenously, e.g. via central venous catheter (CVC or central venous line or central venous access catheter) placed into a large vein in the neck (internal jugular vein), chest (subclavian vein) or groin (femoral vein). Methods of systemic delivery of siRNA, dsRNA, or shRNA vector are well known in the art, e.g. as described herein and in Gao and Huang, 2008, (Mol. Pharmaceutics, Web publication December 30) and review by Rossi, 2006, Gene Therapy, 13:583-584. The siRNA, dsRNA, or shRNA vector can be formulated in various ways, e.g. conjugation of a cholesterol moiety to one of the strands of the siRNA duplex for systemic delivery to the liver and jejunum (Soutschek J. et. al. 2004, Nature, 432:173-178), complexing of siRNAs to protamine fused with an antibody fragment for receptor-mediated targeting of siRNAs (Song E, et al. 2005, Nat. Biotechnol., 23: 709-717) and the use of a lipid bilayer system by Morrissey et al. 2005 (Nat Biotechnol., 23: 1002-1007). The lipid bilayer system produces biopolymers that are in the 120 nanometer diameter size range, and are labeled as SNALPs, for Stable-Nucleic-Acid-Lipid-Particles. The lipid combination protects the siRNAs from serum nucleases and allows cellular endosomal uptake and subsequent cytoplasmic release of the siRNAs (see WO/2006/007712). These references are incorporated by reference in their entirety.

The dose of the particular RNAi agent will be in an amount necessary to effect RNA interference, e.g., gene silencing of the IGPR-1 gene, thereby leading to decrease in IGPR-1 gene expression level and subsequent decrease in the IGPR-1 protein level.

It is also known that RNAi molecules do not have to match perfectly to their target sequence. Preferably, however, the 5′ and middle part of the antisense (guide) strand of the siRNA is perfectly complementary to the target nucleic acid sequence of a the IGPR-1 gene, e.g., SEQ ID NO: 2.

Accordingly, the RNAi molecules functioning as gene silencing—RNAi agents inhibitors of IGPR-1 as disclosed herein are for example, but are not limited to, unmodified and modified double stranded (ds) RNA molecules including short-temporal RNA (stRNA), small interfering RNA (siRNA), short-hairpin RNA (shRNA), microRNA (miRNA), double-stranded RNA (dsRNA), (see, e.g. Baulcombe, Science 297:2002-2003, 2002). The dsRNA molecules, e.g. siRNA, also can contain 3′ overhangs, preferably 3′UU or 3′TT overhangs. In one embodiment, the siRNA molecules of the present invention do not include RNA molecules that comprise ssRNA greater than about 30-40 bases, about 40-50 bases, about 50 bases or more. In one embodiment, the siRNA molecules of the present invention are double stranded for more than about 25%, more than about 50%, more than about 60%, more than about 70%, more than about 80%, more than about 90% of their length.

In some embodiments, a gene silencing RNAi nucleic acid inhibitors of IGPR-1 is any agent which binds to and inhibits the expression of IGPR-1 gene, where the expression of the IGPR-1 gene is inhibited.

In another embodiment of the invention, agents which are inhibitors of IGPR-1 are catalytic nucleic acid constructs, such as, for example ribozymes, which are capable of cleaving RNA transcripts and thereby preventing the production of wildtype protein. Ribozymes are targeted to and anneal with a particular sequence by virtue of two regions of sequence complementary to the target flanking the ribozyme catalytic site. After binding, the ribozyme cleaves the target in a site specific manner. The design and testing of ribozymes which specifically recognize and cleave sequences of the gene products described herein, for example for cleavage of a the IGPR-1 protein or IGPR-1 gene can be achieved by techniques well known to those skilled in the art (for example Lleber and Strauss, (1995) Mol Cell Biol 15:540.551, the disclosure of which is incorporated herein by reference).

### Proteins and Peptide Inhibitors of IGPR-1.

In some embodiments, an inhibitor of IGPR-1 is a protein and/or peptide inhibitor of the IGPR-1 protein of SEQ ID NO: 1 for example, but are not limited to mutated proteins; therapeutic proteins and recombinant proteins of SEQ ID NO: 1 as well as dominant negative inhibitors (e.g., C-terminal fragments of SEQ ID NO:1) as disclosed herein. Proteins and peptides inhibitors can also include for example mutated proteins, genetically modified proteins, peptides, synthetic peptides, recombinant proteins, chimeric proteins, antibodies, humanized proteins, humanized antibodies, chimeric antibodies, modified proteins and fragments thereof.

In some embodiments, an inhibitor of IGPR-1 is a dominant negative variants of the IGPR-1 protein, for example a non-functional variant of the IGPR-1 protein, e.g., a protein such SEQ ID NO: 5 or a fragment of at least about 50, or at least about 60, or at least about 70, or at least about 80 or at least about 90 or more than 90 amino acids of SEQ ID NO: 5. In some embodiments, a dominant negative inhibitor of the IGPR-1 protein is a soluble extracellular domain if the IGPR-1 protein, and can be any functional fragment of SEQ ID NO: 5 or SEQ ID NO: 6 which inhibits the biological activity of IGPR-1 protein to inhibit IGPR-1 mediated tumor growth and angiogenesis.

Antibodies

In some embodiments, an inhibitor of IGPR-1 protein or expression useful in the methods of the present invention include, for example, antibodies, including monoclonal, chimeric humanized, and recombinant antibodies and antigen-binding fragments thereof. In some embodiments, neutralizing antibodies can be used as inhibitors of the IGPR-1 protein. Antibodies are readily raised in animals such as rabbits or mice by immunization with the antigen. Immunized mice are particularly useful for providing sources of B cells for the manufacture of hybridomas, which in turn are cultured to produce large quantities of monoclonal antibodies.

In one embodiment of this invention, the inhibitor to the gene products identified herein can be an antibody molecule or the epitope-binding moiety of an antibody molecule and the like. Antibodies provide high binding avidity and unique specificity to a wide range of target antigens and haptens. Monoclonal antibodies useful in the practice of the present invention include whole antibody and fragments thereof and are generated in accordance with conventional techniques, such as hybridoma synthesis, recombinant DNA techniques and protein synthesis.

Useful monoclonal antibodies and fragments can be derived from any species (including humans) or can be formed as chimeric proteins which employ sequences from more than one species. Human monoclonal antibodies or “humanized” murine antibody are also used in accordance with the present invention. For example, murine monoclonal antibody can be “humanized” by genetically recombining the nucleotide sequence encoding the murine Fv region (i.e., containing the antigen binding sites) or the complementarily determining regions thereof with the nucleotide sequence encoding a human constant domain region and an Fc region. Humanized targeting moieties are recognized to decrease the immunoreactivity of the antibody or polypeptide in the host recipient, permitting an increase in the half-life and a reduction the possibly of adverse immune reactions in a manner similar to that disclosed in European Patent Application No. 0,411,893 A2. The murine monoclonal antibodies should preferably be employed in humanized form. Antigen binding activity is determined by the sequences and conformation of the amino acids of the six complementarily determining regions (CDRs) that are located (three each) on the light and heavy chains of the variable portion (Fv) of the antibody. The 25-kDa single-chain Fv (scFv) molecule, composed of a variable region (VL) of the light chain and a variable region (VH) of the heavy chain joined via a short peptide spacer sequence, is the smallest antibody fragment developed to date. Techniques have been developed to display scFv molecules on the surface of filamentous phage that contain the gene for the scFv. scFv molecules with a broad range of antigenic-specificities can be present in a single large pool of scFv-phage library. Some examples of high affinity monoclonal antibodies and chimeric derivatives thereof, useful in the methods of the present invention, are described in the European Patent Application EP 186,833; PCT Patent Application WO 92/16553; and U.S. Pat. No. 6,090,923.

Chimeric antibodies are immunoglobin molecules characterized by two or more segments or portions derived from different animal species. Generally, the variable region of the chimeric antibody is derived from a non-human mammalian antibody, such as murine monoclonal antibody, and the immunoglobin constant region is derived from a human immunoglobin molecule. Preferably, both regions and the combination have low immunogenicity as routinely determined.

One limitation of scFv molecules is their monovalent interaction with target antigen. One of the easiest methods of improving the binding of a scFv to its target antigen is to increase its functional affinity through the creation of a multimer. Association of identical scFv molecules to form diabodies, triabodies and tetrabodies can comprise a number of identical Fv modules. These reagents are therefore multivalent, but monospecific. The association of two different scFv molecules, each comprising a VH and VL domain derived from different parent Ig will form a fully functional bispecific diabody. A unique application of bispecific scFvs is to bind two sites simultaneously on the same target molecule via two (adjacent) surface epitopes. These reagents gain a significant avidity advantage over a single scFv or Fab fragments. A number of multivalent scFv-based structures has been engineered, including for example, miniantibodies, dimeric miniantibodies, minibodies, (scFv)₂, diabodies and triabodies. These molecules span a range of valence (two to four binding sites), size (50 to 120 kDa), flexibility and ease of production. Single chain Fv antibody fragments (scFvs) are predominantly monomeric when the VH and VL domains are joined by, polypeptide linkers of at least 12 residues. The monomer scFv is thermodynamically stable with linkers of 12 and 25 amino acids length under all conditions. The noncovalent diabody and triabody molecules are easy to engineer and are produced by shortening the peptide linker that connects the variable heavy and variable light chains of a single scFv molecule. The scFv dimers are joined by amphipathic helices that offer a high degree of flexibility and the miniantibody structure can be modified to create a dimeric bispecific (DiBi) miniantibody that contains two miniantibodies (four scFv molecules) connected via a double helix. Gene-fused or disulfide bonded scFv dimers provide an intermediate degree of flexibility and are generated by straightforward cloning techniques adding a C-terminal Gly4Cys (SEQ ID NO: 19) sequence. scFv-CH₃ minibodies are comprised of two scFv molecules joined to an IgG CH3 domain either directly (LD minibody) or via a very flexible hinge region (Flex minibody). With a molecular weight of approximately 80 kDa, these divalent constructs are capable of significant binding to antigens. The Flex minibody exhibits impressive tumor localization in mice. Bi- and tri-specific multimers can be formed by association of different scFv molecules. Increase in functional affinity can be reached when Fab or single chain Fv antibody fragments (scFv) fragments are complexed into dimers, trimers or larger aggregates. The most important advantage of multivalent scFvs over monovalent scFv and Fab fragments is the gain in functional binding affinity (avidity) to target antigens. High avidity requires that scFv multimers are capable of binding simultaneously to separate target antigens. The gain in functional affinity for scFv diabodies compared to scFv monomers is significant and is seen primarily in reduced off-rates, which result from multiple binding to two or more target antigens and to rebinding when one Fv dissociates. When such scFv molecules associate into multimers, they can be designed with either high avidity to a single target antigen or with multiple specificities to different target antigens. Multiple binding to antigens is dependent on correct alignment and orientation in the Fv modules. For full avidity in multivalent scFvs target, the antigen binding sites must point towards the same direction. If multiple binding is not sterically possible then apparent gains in functional affinity are likely to be due the effect of increased rebinding, which is dependent on diffusion rates and antigen concentration. Antibodies conjugated with moieties that improve their properties are also contemplated for the instant invention. For example, antibody conjugates with PEG that increases their half-life in vivo can be used for the present invention. Immune libraries are prepared by subjecting the genes encoding variable antibody fragments from the B lymphocytes of naive or immunized animals or patients to PCR amplification. Combinations of oligonucleotides which are specific for immunoglobulin genes or for the immunoglobulin gene families are used. Immunoglobulin germ line genes can be used to prepare semisynthetic antibody repertoires, with the complementarity-determining region of the variable fragments being amplified by PCR using degenerate primers. These single-pot libraries have the advantage that antibody fragments against a large number of antigens can be isolated from one single library. The phage-display technique can be used to increase the affinity of antibody fragments, with new libraries being prepared from already existing antibody fragments by random, codon-based or site-directed mutagenesis, by shuffling the chains of individual domains with those of fragments from naive repertoires or by using bacterial mutator strains.

Alternatively, a SCID-hu mouse, for example the model developed by Genpharm, can be used to produce antibodies, or fragments thereof. In one embodiment, a new type of high avidity binding molecule, termed peptabody, created by harnessing the effect of multivalent interaction is contemplated. A short peptide ligand was fused via a semirigid hinge region with the coiled-coil assembly domain of the cartilage oligomeric matrix protein, resulting in a pentameric multivalent binding molecule. In preferred embodiment of this invention, ligands and/or chimeric inhibitors can be targeted to tissue- or tumor-specific targets by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. Alternatively, two or more active agents and or inhibitors attached to targeting moieties can be administered, wherein each conjugate includes a targeting moiety, for example, a different antibody. Each antibody is reactive with a different target site epitope (associated with the same or a different target site antigen). The different antibodies with the agents attached accumulate additively at the desired target site. Antibody-based or non-antibody-based targeting moieties can be employed to deliver a ligand or the inhibitor to a target site. Preferably, a natural binding agent for an unregulated or disease associated antigen is used for this purpose.

Small Molecules

All of the applications set out in the above paragraphs are incorporated herein by reference. It is believed that any or all of the compounds disclosed in these documents are useful for treatment of cancers and/or diseases and disorders associated with angiogenesis, including, for example, but are not limited to, inhibiting angiogenesis in macular degeneration (AMD), and the like. In some embodiments, one of ordinary skill in the art can use other agents as inhibitors of IGPR-1, for example antibodies, decoy antibodies, or RNAi are effective for the treatment or prevention of cancers, inhibition of metastasis and/or treatment of diseases and disorders associated with angiogenesis as disclosed herein. In some embodiments, an inhibitor agent of IGPR-1 can be assessed in models to determine its ability to inhibit cancer metastases or invasion or to inhibit angiogenesis.

Angiogenesis:

Angiogenesis is a process of tissue vascularization that involves the growth of new developing blood vessels into a tissue, and is also referred to as neo-vascularization. Blood vessels are the means by which oxygen and nutrients are supplied to living tissues and waste products are removed from living tissue. When appropriate, angiogenesis is a critical biological process. It is essential in reproduction, development and wound repair. Conversely, inappropriate angiogenesis also referred to as pathological angiogenesis, can have severe negative consequences. For example, it is only after many solid tumors are vascularized as a result of angiogenesis that the tumors have a sufficient supply of oxygen and nutrients that permit it to grow rapidly and metastasize.

Maintaining the rate of angiogenesis in its proper equilibrium is so critical to a range of functions, it must be carefully regulated in order to maintain health. Abnormal angiogenesis or pathological angiogenesis occurs when the body loses at least some control of angiogenesis, resulting in either excessive or insufficient blood vessel growth.

Disease or disorders associated with pathological angiogenesis are those diseases and disorders which require or conversely, induce vascular growth are amenable to treatment with the compositions as disclosed herein. Such diseases represent a significant portion of all diseases for which medical treatment is sought, and include cancers, diabetic retinopathy, macular degeneration and inflammatory arthritis

For instance, in some embodiments, an inhibitor of IGPR-1 can be used to treat a subject with excess or induced angiogenesis. Alternatively, in some embodiments, conditions where decreased angiogenesis occurs which is normally required for natural healing include, without limitation, ulcers, strokes, and heart attacks can be treated with an IGPR-1 protein or a functional fragment or mimetic or variant thereof.

Treatment of Angiogenesis-Related Disorders Characterized by Increased Angiogenesis with Inhibitors of IGPR-1

Excessive blood vessel proliferation can result in tumor growth, tumor spread, blindness, psoriasis and rheumatoid arthritis. In these diseases with excess angiogenesis (e.g., referred to herein as “angiogenesis-related diseases with increased angiogenesis”), e.g., cancer, inhibition of angiogenesis is desirable. For example, in arthritis, new capillary blood vessels invade the joint and destroy cartilage. In diabetes, new capillaries invade the vitreous, bleed, and cause blindness. Tumor growth and metastasis are angiogenesis dependent. A tumor must continuously stimulate the growth of new capillary blood vessels for the tumor itself to grow.

In some embodiments, a subject amenable to the methods and compositions as disclosed herein are subjects with cancers which express IGPR-1.

In some embodiments, a cancer in which IGPR-1 protein function and/or its expression from the IGPR-1 gene is inhibited by the methods and compositions as disclosed herein is a cancer which are of epithelial or endothelia origin, e.g., including but not limited to, cancers of bronchial epithelial cells of lung, cancers of urothelium of the bladder, breast glandular and lobular epithelia cells, cancers of skin epidermis, cancers of epithelium of gastrointestinal and rectum, cancers of endometrial glands of the uterus, cancers of the ureter, cancers of fallopian tube epithelium, cancers of colonic epithelium, cancers of small bowl epithelium, cancers of stomach epithelium including both chief and parietal cells, cancers of trophoblastic epithelium of placenta, and pancreatic acinar cells.

In some embodiments, a subject amenable to the methods and compositions as disclosed herein and administering an inhibitor of IGPR-1 are subjects with cancers which express IGPR-1 including, but not limited to a subject with any one of the following cancers, squamous cell carcinoma of the esophagus, adenocarcinoma of the stomach, adenocarcinoma of the rectum, squamous cell carcinoma of the bladder, adenocarcinoma of the pancreases, squamous cell carcinoma of the uterus, and finally adenocarcinoma of the prostate, or papillary carcinoma of the thyroid.

In some embodiments, a subject amenable to the methods and compositions comprising an inhibitor of IGPR-1 as disclosed herein are subjects with expresses cancer genes selected from the group of HER2/Her-2, BRAC1 and BRAC2, Rb, p53, and variants thereof.

In some embodiments, examples of cancers that can be treated with inhibitors of IGPR-1 include, for example but are not limited to, the cancers listed in Table 1 and Table 2 as disclosed herein, which include but are not limited to: bladder cancer, Squamous Cell carcinoma (SCC), Breast cancer, Infiltrating Duct carcinoma, Bronchus cancer, cancer of the Fallopian Tube, cancer of the GI tract, including cancer of esophagus, stomach cancer, Adenocarcinoma, colon cancer, cancer of the rectum, cancer of the small intestine; pancreatic cancer, cancer of the placenta, prostate cancer, skin cancer, testicular cancer, thyroid cancer, cancer of the thymus, endometrium cancer, cancer of the urethra.

In some embodiments of all aspects of the invention, the method are applicable to the treatment of any cancer in a subject, preferably a mammalian subject or human subject, where the metastatic cancer is for example, but not limited to mescenchymal in origin (sarcomas); fibrosarcomas; myxosarcomas; liposarcomas; chondrosarcomas; osteogenic sarcomas; angiosarcomas; endotheliosarcomas; lymphangiosarcomas; synoviosarcomas; mesotheliosarcomas; Ewing's tumors; myelogenous leukemias; monocytic leukemias; malignant leukemias; lymphocytic leukemias; plasmacytomas; leiomyosarcomas; and rhabdomyosarcoma; cancers epithelial in origin (carcinomas); squamous cell or epidermal carcinomas; basal cell carcinomas; sweat gland carcinomas; sebaceous gland carcinomas; adenocarcinomas; papillary carcinomas; papillary adenocarcinomas; cystadenocarcinomas; medullary carcinomas; undifferentiated carcinomas (simplex carcinomas); bronchogenic carcinomas; bronchial carcinomas; melanocarcinomas; renal cell carcinomas; hepatocellular carcinomas; bile duct carcinomas; transitional cell carcinomas; squamous cell carcinomas; choriocarcinomas; seminomas; embryonal carcinomas; malignant teratomas; and terato carcinomas; leukemia; acute lymphocytic leukemia and acute myelocytic leukemia (myeloblastic, promyeloblastic, myelomonocytic; monocytic, and erythroleukemia); chronic leukemia; chronic myelocytic (granulocytic) leukemia; chronic lymphocytic leukemia; polycythemia vera; lymphoma; Hodgkin's disease; non-Hodgekin's disease; multiple mycloma; Waldenström's macroglobulinemia; heavy chain disease. In some embodiments, the cancer is lymphia; leukemia; sarcoma; adenomas. In some embodiments, the cancer is acute lympoblastic leukemia (ALL).

In some embodiments, the metastatic cancer is breast cancer. In some embodiments, the cancer is metastatic breast cancer. In some embodiments, the breast cancer is primary breast cancer. In some embodiments, the cancer is lung cancer, and in some embodiments, the lung cancer is metastatic lung cancer. In some embodiments, the cancer is prostate cancer, or colon cancer, or hepatocellular carcinoma.

In some embodiments, examples of cancers that can be treated with inhibitors of IGPR-1 include, for example but are not limited to, small or non-small cell lung, oat cell, papillary, bronchiolar, squamous cell, transitional cell, Walker), leukemia (e.g., B-cell, T-cell, HTLV, acute or chronic lymphocytic, mast cell, myeloid), histiocytoma, histiocytosis, Hodgkin disease, non-Hodgkin lymphoma, plasmacytoma, reticuloendotheliosis, adenoma, adenocarcinoma, adeno-fibroma, adenolymphoma, ameloblastoma, angiokeratoma, angiolymphoid hyperplasia with eosinophilia, sclerosing angioma, angiomatosis, apudoma, branchioma, malignant carcinoid syndrome, carcinoid heart disease, carcinosarcoma, colon cancer, prostate cancer, cementoma, cholan-gioma, cholesteatoma, chondrosarcoma, chondroblastoma, chondrosarcoma, chordoma, choristoma, craniopharyngioma, chrondroma, cylindroma, cystadenocar-cinoma, cystadenoma, cystosarcoma phyllodes, dysgerminoma, ependymoma, Ewing sarcoma, fibroma, fibrosarcoma, giant cell tumor, ganglioneuroma, glioblastoma, glomangioma, granulosa cell tumor, gynandroblastoma, hamartoma, hemangioendo-thelioma, hemangioma, hemangiopericytoma, hemangiosarcoma, hepatoma, hepatocellular cancer, islet cell tumor, Kaposi sarcoma, leiomyoma, leiomyosarcomas, leukosarcoma, Leydig cell tumor, lipoma, liposarcoma, lymphangioma, lymphangiomyoma, lymphangiosarcoma, medulloblastoma, meningioma, mesenchymoma, mesonephroma, mesothelioma, myoblastoma, myoma, myosarcoma, myxoma, myxosarcoma, neurilemmoma, neuroma, neuro-blastoma, neuroepithelioma, neurofibroma, neurofibromatosis, odontoma, osteoma, osteosarcoma, papilloma, paraganglioma, paraganglioma nonchromaffin, pinealoma, rhabdomyoma, rhabdomyosarcoma, Sertoli cell tumor, teratoma, theca cell tumor, and other diseases in which cells have become dysplastic, immortalized, or transformed.

In some embodiments, a subject amenable to the methods and compositions comprising an inhibitor of IGPR-1 as disclosed herein are mammalian subjects, for example, human subjects. In some embodiments, a subject amenable to the methods and compositions comprising an inhibitor of IGPR-1 as disclosed herein are subjects which have been selected to have expression of IGPR-1 in a biological sample obtained from the subject, e.g., a tumor biopsy sample and the like. Such subjects which have detectable levels and/or high levels of IGPR-1 expression in a biological sample obtained from the subject, e.g., a tumor biopsy sample, can be treated according to the methods as disclosed herein, e.g., administering a composition comprising a IGPR-1 inhibitor to the subject as disclosed herein.

In some embodiments, a subject amenable to administration of an inhibitor of IGPR-1 according to the methods as disclosed herein has excess or uncontrolled angiogenesis. In some embodiments, a subject amenable to treatment has neovascularization, such as neovascularization of the eye. Ocular neovascularization is the most common cause of blindness. Vessel loss and subsequent destructive neovascularization are also the two critical phases of many sight-threatening diseases. Ocular neovascularization is the most common cause of blindness in all age groups, including retinopathy of prematurity (ROP), diabetic retinopathy in working age-adults and age-related macular degeneration (AMD) in the elderly, and are all conditions related to pathological angiogenesis associated with excess angiogenesis in the eye.

Accordingly, in some embodiments, a subject with an angiogenesis-related disorder associated with increased angiogenesis of the eye is amenable to treatment with a composition comprising an IGPR-1 inhibitor as disclosed herein. In some embodiments, a subject with any one of retinopathy of prematurity (ROP), diabetic retinopathy, age-related macular degeneration (AMD) can be administered an inhibitor of IGPR-1 as disclosed herein.

Diabetic retinopathy is the most common diabetic eye disease and a leading cause of blindness in American adults. It is caused by changes in the blood vessels of the retina. In some cases of diabetic retinopathy, fragile, abnormal blood vessels develop and leak blood into the center of the eye, blurring vision. In others, abnormal new blood vessels grow on the surface of the retina.

Age-related macular degeneration is a degenerative condition of the macula (the central retina). It is the most common cause of vision loss in the United States in those 50 or older, and its prevalence increases with age of an individual. Age-related macular degeneration is caused by hardening of the arteries that nourish the retina. This deprives the sensitive retinal tissue of oxygen and nutrients that it needs to function and thrive. As a result, the central vision deteriorates. Ten percent of age related macular degeneration is caused by neovascularization, where new blood vessels form to improve the blood supply to oxygen-deprived retinal tissue.

Retinopathy of prematurity (ROP) is a potentially blinding disease, initiated by lack of retinal vascular growth after premature birth. The greatest risk factor for development of ROP is low birth weight and gestational age. Retinopathy of prematurity (ROP) is a potentially blinding eye disorder that primarily affects premature and underweight infants. The smaller a baby is at birth, the more likely that baby is to develop ROP. This disorder usually develops in both eyes, and is one of the most common causes of visual loss in childhood and can lead to lifelong vision impairment and blindness. About 1,100-1,500 infants annually develop ROP that is severe enough to require medical treatment. About 400-600 infants each year in the U.S. becomes legally blind from ROP. ROP occurs in two phases. (Simons, B. D. & Flynn, J. T. (1999) International Ophthalmology Clinics 39, 29-48). When infants are born prematurely the retina is incompletely vascularized. In infants who develop ROP, growth of vessels slows or ceases at birth leaving maturing but avascular and therefore hypoxic peripheral retina. (Ashton, N. (1966) Am J Ophthalmol 62, 412-35; Flynn, J. T., O'Grady, G. E., Herrera, J., Kushner, B. J., Cantolino, S. & Milam, W. (1977) Arch Ophthalmol 95, 217-23). This is the first phase of ROP. The extent of non-perfusion of the retina in the initial phase of ROP appears to determine the subsequent degree of neovascularization, the late destructive stage of ROP, with the attendant risk of retinal detachment and blindness. (Penn, J. S., Tolman, B. L. & Henry, M. M. (1994) Invest Ophthalmol V is Sci 35, 3429-35). If it were possible to allow blood vessels to grow normally in all premature infants, as they do in utero, the second damaging neovascular phase of ROP would not occur. When ROP was first described in 1942, the etiology was unknown. However, the liberal use of high supplemental oxygen in premature infants was soon associated with the disease and hyperoxia was shown to induce ROP-like retinopathy in neonatal animals with incompletely vascularized retinas. This suggested that an oxygen-regulated factor was involved. Expression of vascular endothelial growth factor (VEGF), which is necessary for normal vascular development, is oxygen-regulated and was found to be important for both phases of ROP. (Okamoto, N., Hofmann, F., Wood, J. M. & Campochiaro, P. A. (2000) American Journal of Pathology 156, 697-707). High supplemental oxygen affects the first phase of vascular growth in ROP animal models through suppression of VEGF expression. However, with current careful use of moderate oxygen supplementation, the oxygen level in patients is not a significant risk factor for ROP, yet the disease persists, suggesting that other factors are also involved. (Kinsey, V. E., Arnold, H. J., Kalina, R. E., Stern, L., Stahlman, M., Odell, G., Driscoll, J. M., Jr., Elliott, J. H., Payne, J. & Patz, A. (1977) Pediatrics 60, 655-68; Lucey, J. F. & Dangman, B. (1984) Pediatrics 73, 82-96).

In embodiments where the neovascularization is ocular neovascularization, for example diabetic retinopathy, age-related macular degeneration (AMD) and retinopathy of prematurity (ROP), a pharmaceutical composition comprising an inhibitor of IGPR-1 as disclosed herein can be administered to the subject prophylatically, for instance, if the subject has been identified to be at risk of developing retinopathies, and/or ocular neovascularization, for example diabetic retinopathies, AMD and ROP.

Thus, in one related embodiment, a tissue to be treated with an inhibitor of IGPR-1 is an inflamed tissue and the angiogenesis to be inhibited is inflamed tissue angiogenesis where there is neovascularization of inflamed tissue. Accordingly, in one embodiment, the method contemplates using inhibitor of IGPR-1 to inhibit of angiogenesis in arthritic tissues, such as in a patient with chronic articular rheumatism, in immune or non-immune inflamed tissues, in psoriatic tissue and the like.

In a related embodiment, a tissue to be treated with an inhibitor of IGPR-1 is a tumor tissue of a subject with a solid tumor, a metastases, a skin cancer, a breast cancer, a hemangioma or angiofibroma and the like cancer, and the angiogenesis to be inhibited is tumor tissue angiogenesis where there is neovascularization of a tumor tissue. Typical solid tumor tissues treatable by the pharmaceutical composition of the invention, includes for example, but not limited to tumors of the lung, pancreas, breast, colon, laryngeal, ovarian, and the like tissues. In some embodiment, the solid tumor tissue treatable by the present methods include thyroid, and the cancer type is medullary thyroid cancer. Inhibition of tumor tissue pathological angiogenesis is important due to the role neovascularization plays in tumor growth. In the absence of neovascularization of tumor tissue, the tumor tissue does not obtain the required nutrients, slows in growth, ceases additional growth, regresses and ultimately becomes necrotic resulting in killing of the tumor.

The methods are also effective against the formation of metastases because (1) their formation requires vascularization of a primary tumor so that the metastatic cancer cells can exit the primary tumor and (2) their establishment in a secondary site requires neovascularization to support growth of the metastases.

In a related embodiment, the invention contemplates the administration of a composition comprising an inhibitor of IGPR-1 as disclosed herein in conjunction with other therapies such as conventional chemotherapy directed against solid tumors and for control of establishment of metastases. The administration of a composition comprising an inhibitor of IGPR-1 is typically conducted during or after chemotherapy, although it is also encompassed within the present invention to inhibit angiogenesis after a regimen of chemotherapy at times where the tumor tissue will be responding to the toxic assault by inducing angiogenesis to recover by the provision of a blood supply and nutrients to the tumor tissue. In addition, a composition comprising an inhibitor of IGPR-1 as disclosed herein for the treatment of tumor-associated angiogenesis can be administrated prophylatically and/or before the development of a tumor, if the subject has been identified as to have a risk of developing cancer, for example to subjects that are positive for biomarkers of cancer cells or tumors. Insofar as the present methods apply to inhibition of tumor neovascularization, the methods can also apply to inhibition of tumor tissue growth, to inhibition of tumor metastases formation, and to regression of established tumors.

The anti-angiogenesis activity of an IGPR-1 inhibitor as disclosed herein can also be assessed in vivo according to the methods as disclosed in the Examples herein, or by a decrease in capillary density or neovascular infiltration using a Matrigel plug assay as described by e.g., Kragh M, et al., (2003) (Kragh M, Hjarnaa P J, Bramm E, Kristjansen P E, Rygaard J, and Binderup L. Int J Oncol. (2003) 22(2):305-11, which is herein incorporated by reference in its entirety) in a mammal treated with an IGPR-1 inhibitor, compared to capillary density or neovascular infiltration observed in the absence of an IGPR-1 inhibitor. A “decrease in capillary density” means a decrease of at least 5% in the presence of anti-angiogenic agent as compared to untreated subjects; preferably a decrease in capillary density is at least 10%, at least 20%, at least 30%, at least 40%, at least 50%, at least 60%, at least 70%, at least 80%, at least 90%, at least 95%, at least 99% lower, or even 100% (i.e., absent) in the presence of an IGPR-1 inhibitor as compared to that measured in the absence of administration of an IGPR-1 inhibitor.

Treatment of Angiogenesis-Related Disorders Characterized by Decreased Angiogenesis with IGPR-1 or Functional Fragments or Variants Thereof

In some embodiments, the present invention encompasses a composition comprising a IGPR-1 polypeptide or a functional fragment or variant thereof for the treatment of an angiogenesis-related disease or disorder associated with a decrease in angiogenesis. In some embodiments, such a disorder is where natural wound healing has failed due to decreased angiogenesis, such as diseases with insufficient blood vessel growth including but not limited to ulcers, strokes, and heart attacks.

Other disease and disorders where a composition comprising a IGPR-1 polypeptide or a functional fragment thereof can be used can be selected from any one or a combination of angiogenesis-related diseases or disorders associated with a decrease in angiogenesis; ischemic injury, ischemic heart disease, transplantation therapy, stroke. In some embodiments, a composition comprising a IGPR-1 polypeptide or a functional fragment thereof can be used to treat a subject after eye surgery, or in some embodiments, eye transplantation, e.g., cornea transplant, or a subject having, or have had a RPE (retinal pigment epithelium) cell transplant, to facilitate the survival and adhesion of the transplanted RPE cells. In some embodiments, a composition comprising a IGPR-1 polypeptide or a functional fragment thereof can be used to treat a subject after a skin transplant, e.g., a whole, or partial face transplant or other skin transplant procedure.

As used herein, the term “IGPR-1 polypeptide” encompasses a protein of SEQ ID NO: 1, either with or without the signal sequence, or a protein of SEQ ID NO: 1 that has a conservative substitution, or is a variant or functional fragment of SEQ ID NO:1 that retains IGPR-1 activity as that term is defined herein. By “retaining IGPR-1 activity” is meant that a polypeptide retains at least 50% of the IGPR-1 activity of a polypeptide of SEQ ID NO. 1. Also encompassed by the term “IGPR-1 polypeptide” are mammalian homologs of human IGPR-1 and conservative substitution variants or fragments of SEQ ID NO:1 that retain IGPR-1 activity.

In some embodiments, a IGPR-1 activator agent is administered to a subject with an angiogenesis-related disorder associated with a decreased angiogenesis. In one embodiment, an IGPR-1 activator agent is selected from, but is not limited to an antibody, a small molecule, a peptide, a polypeptide, nucleic acid, such as RNA or DNA which enhances the function of IGPR-1 gene or protein function.

Enhancing, stimulating or re-activating a gene's or protein's function can be achieved in a variety of ways. In one aspect of the invention an isolated nucleic acid molecule of SEQ ID NO:2 or a fragment thereof which encodes a functional IGPR-1 polypeptide, as described above, is administered to a subject in need thereof. Typically, a IGPR-1 nucleic acid molecule can be administered as pro-angiogenic agent to a subject to treat or prevent an angiogenesis-related disorder characterized by a decrease or loss in angiogenesis. In a further aspect, there is provided the use of a pro-angiogenic isolated nucleic acid molecule (i.e. IGPR-1 nucleic acid of SEQ ID NO:2), as described above, in the preparation of a medicament for the treatment of an angiogenesis-related disorder characterized by a decrease or loss in angiogenesis. In a further aspect, there is provided the use of an IGPR-1 isolated nucleic acid agent in the preparation of a medicament for the treatment of an angiogenesis-related disorder characterized by a decrease in angiogenesis.

Typically, a vector capable of expressing an IGPR-1 polypeptide of the invention, or a fragment or derivative thereof, can be administered to a subject to treat or prevent a disorder including, but not limited to, those described above. Transducing retroviral vectors are often used for somatic cell gene therapy because of their high efficiency of infection and stable integration and expression. A nucleic acid encoding an IGPR-1 polypeptide, or portions thereof, can be cloned into a retroviral vector and expression can be driven from its endogenous promoter or from the retroviral long terminal repeat or from a promoter specific for the target cell type of interest. Other viral vectors can be used and include, as is known in the art, adenoviruses, adeno-associated viruses, vaccinia viruses, papovaviruses, lentiviruses and retroviruses of avian, murine and human origin.

Gene therapy can be carried out according to established methods (See for example Friedman, 1991; Culver, 1996). A vector containing a nucleic acid molecule of the invention linked to expression control elements and capable of replicating inside the cells is prepared. Alternatively the vector can be replication deficient and can require helper cells for replication and use in gene therapy.

Gene transfer using non-viral methods of infection in vitro can also be used. These methods include direct injection of DNA, uptake of naked DNA in the presence of calcium phosphate, electroporation, protoplast fusion or liposome delivery. Gene transfer can also be achieved by delivery as a part of a human artificial chromosome or receptor-mediated gene transfer. This involves linking the DNA to a targeting molecule that will bind to specific cell-surface receptors to induce endocytosis and transfer of the DNA into mammalian cells. One such technique uses poly-L-lysine to link asialoglycoprotein to DNA. An adenovirus is also added to the complex to disrupt the lysosomes and thus allow the DNA to avoid degradation and move to the nucleus. Infusion of these particles intravenously has resulted in gene transfer into hepatocytes.

In a further aspect, a suitable IGPR-1 agent can also include peptides, phosphopeptides or small organic or inorganic compounds that can mimic the function of an IGPR-1 polypeptide, or can include an antibody specific for an IGPR-1 polypeptide that is able to enhance or increase the function or activity of a IGPR-1 polypeptide.

In a further aspect, a suitable an IGPR-1 activating agent can also include peptides, phosphopeptides or small organic or inorganic compounds that can mimic the function of a IGPR-1 polypeptide of the invention, or can include an antibody specific for a IGPR-1 that is able to enhance or increase the function or activity of a IGPR-1 polypeptide.

Peptides, phosphopeptides or small organic or inorganic compounds suitable for therapeutic applications can be identified using nucleic acids and polypeptides of IGPR-1 using drug screening applications, which are commonly known by one of ordinary skill in the art, and can be assessed for their pro-angiogenic or anti-angiogenic activity using the angiogenesis assays as described herein.

Selection of the appropriate agents and treatment methods can be made by those skilled in the art, according to conventional pharmaceutical principles. The combination of therapeutic agents and treatment methods can act synergistically to effect the treatment or prevention of the various disorders described above. Using this approach, therapeutic efficacy with lower dosages of each agent can be possible, thus reducing the potential for adverse side effects. Any of the therapeutic methods described herein can be applied to any subject, including, for example, mammals such as dogs, cats, cows, horses, rabbits, monkeys, and most preferably, humans.

Polypeptides and Peptides

In some embodiments, a pro-angiogenic agent is a IGPR-1 polypeptide or a functional portion thereof to promote angiogenesis, and can be administered to an individual in need thereof. In one approach, a soluble IGPR-1 polypeptide, produced, for example, in cultured cells bearing a recombinant IGPR-1 expression vector can be administered to the individual. In one embodiment, IGPR-1 can be overexpressed in an individual by gene therapy methodologies commonly known by one of ordinary skill in the art.

In a further aspect of the present invention, an pro-angiogenic agent is an isolated polypeptide of IGPR-1 comprising the sequence set forth in SEQ ID NO: 1. In some embodiments, a pro-angiogenic agent is an isolated polypeptide of IGPR-1 having at least 70%, preferably 85%, and more preferably 95%, identity to SEQ ID NO:1, or at least 70%, preferably 85%, and more preferably 95% or more of the same activity as compared to the wild-type protein of SEQ ID NO:1. Sequence identity is typically calculated using the BLAST algorithm, described in Altschul et al (1997) with the BLOSUM62 default matrix.

An pro-angiogenic polypeptide agent (i.e. IGPR-1 polypeptide or a functional portion thereof) will generally be administered intravenously. This approach rapidly delivers the protein throughout the system and maximizes the chance that the protein is intact when delivered. Alternatively, other routes of therapeutic protein administration are contemplated, such as by inhalation. Technologies for the administration of agents, including protein agents, as aerosols are well known and continue to advance. Alternatively, the polypeptide agent can be formulated for topical delivery, including, for example, preparation in liposomes. Further contemplated are, for example, transdermal administration, and rectal or vaginal administration.

### Generation of Recombinant IGPR-1 Protein as a Pro-Angiogenic Agent

Vectors for transduction of an IGPR-1-encoding sequence are well known in the art. While overexpression using a strong non-specific promoter, such as a CMV promoter, can be used, it can be helpful to include a tissue- or cell-type-specific promoter on the expression construct—for example, the use of a skeletal muscle-specific promoter or other cell-type-specific promoter can be advantageous, depending upon what cell type is used as a host. Further, treatment can include the administration of viral vectors that drive the expression of IGPR-1 polypeptide or a functional fragment thereof in infected host cells. Viral vectors are well known to those skilled in the art and discussed in more detail herein.

These vectors are readily adapted for use in the methods of the present invention. By the appropriate manipulation using recombinant DNA/molecular biology techniques to insert an operatively linked nucleic acid sequence encoding the gene to be expressed (e.g. IGPR-1 nucleic acid sequence of SEQ ID NO: 2) into the selected expression/delivery vector, many equivalent vectors for the practice of the methods described herein can be generated. It will be appreciated by those of skill in the art that cloned genes readily can be manipulated to alter the amino acid sequence of a protein.

Examples of expression vectors and host cells are the pET vectors (NOVAGEN®), pGEX vectors (GE Life Sciences), and pMAL vectors (New England labs. Inc.) for protein expression in E. coli host cell such as BL21, BL21(DE3) and AD494(DE3)pLysS, Rosetta (DE3), and Origami(DE3) ((NOVAGEN®); the strong CMV promoter-based pcDNA3.1 (INVITROGEN™ Inc.) and pCIneo vectors (Promega) for expression in mammalian cell lines such as CHO, COS, HEK-293, Jurkat, and MCF-7; replication incompetent adenoviral vector vectors pAdeno X, pAd5F35, pLP-Adeno-X-CMV (CLONTECH®), pAd/CMV/V5-DEST, pAd-DEST vector (INVITROGEN™ Inc.) for adenovirus-mediated gene transfer and expression in mammalian cells; pLNCX2, pLXSN, and pLAPSN retrovirus vectors for use with the RETRO-X™ system from Clontech for retroviral-mediated gene transfer and expression in mammalian cells; pLenti4/V5-DEST™, pLenti6/V5-DEST™, and pLenti6.2/V5-GW/lacZ (INVITROGEN™ Inc.) for lentivirus-mediated gene transfer and expression in mammalian cells; adenovirus-associated virus expression vectors such as pAAV-MCS, pAAV-IRES-hrGFP, and pAAV-RC vector (STRATAGENE®) for adeno-associated virus-mediated gene transfer and expression in mammalian cells; BACpak6 baculovirus (CLONTECH®) and pFastBac™ HT (INVITROGEN™ Inc.) for the expression in Spodopera frugiperda 9 (Sf9) and Sf11 insect cell lines; pMT/BiP/V5-His (INVITROGEN™ Inc.) for the expression in Drosophila Schneider S2 cells; Pichia expression vectors pPICZa, pPICZ, pFLDa and pFLD (INVITROGEN™ Inc.) for expression in Pichia pastoris and vectors pMETa and pMET for expression in P. methanolica; pYES2/GS and pYD1 (INVITROGEN™ Inc.) vectors for expression in yeast Saccharomyces cerevisiae. Recent advances in the large scale expression heterologous proteins in Chlamydomonas reinhardtii are described by Griesbeck C. et. al. 2006 Mol. Biotechnol. 34:213-33 and Fuhrmann M. 2004, Methods Mol. Med. 94:191-5. Foreign heterologous coding sequences are inserted into the genome of the nucleus, chloroplast and mitochodria by homologous recombination. The chloroplast expression vector p64 carrying the most versatile chloroplast selectable marker aminoglycoside adenyl transferase (aadA), which confer resistance to spectinomycin or streptomycin, can be used to express foreign protein in the chloroplast. Biolistic gene gun method is used to introduced the vector in the algae. Upon its entry into chloroplasts, the foreign DNA is released from the gene gun particles and integrates into the chloroplast genome through homologous recombination.

In one embodiment, the expression vector is a viral vector, such as a lentivirus, adenovirus, or adeno-associated virus. A simplified system for generating recombinant adenoviruses is presented by He T C. et. al. Proc. Natl. Acad. Sci. USA, 95:2509-2514, 1998. The gene of interest is first cloned into a shuttle vector, e.g. pAdTrack-CMV. The resultant plasmid is linearized by digesting with restriction endonuclease Pme I, and subsequently cotransformed into E. coli BJ5183 cells with an adenoviral backbone plasmid, e.g. pAdEasy-1 of STRATAGENE®'s ADEASY™ Adenoviral Vector System. Recombinant adenovirus vectors are selected for kanamycin resistance, and recombination confirmed by restriction endonuclease analyses. Finally, the linearized recombinant plasmid is transfected into adenovirus packaging cell lines, for example HEK 293 cells (E1-transformed human embryonic kidney cells) or 911 (E1-transformed human embryonic retinal cells) (Human Gene Therapy 7:215-222, 1996). Recombinant adenovirus are generated within the HEK 293 cells.

In one embodiment, the preferred viral vector is a lentiviral vector and there are many examples of use of lentiviral vectors for gene therapy for inherited disorders of haematopoietic cells and various types of cancer, and these references are hereby incorporated by reference (Klein, C. and Baum, C. (2004), Hematol. J., 5:103-111; Zufferey, R et. al. (1997), Nat. Biotechnol., 15:871-875; Morizono, K. et. al. (2005), Nat. Med., 11:346-352; Di Domenico, C. et. al. (2005). Hum. Gene Ther., 16:81-90). The HIV-1 based lentivirus can effectively transduce a broader host range than the Moloney Leukemia Virus (MoMLV)-base retroviral systems. Preparation of the recombinant lentivirus can be achieved using the pLenti4/V5-DEST™, pLenti6/V5-DEST™ or pLenti vectors together with VIRAPOWER™ Lentiviral Expression systems from INVITROGEN™ Inc.

In one embodiment, the expression viral vector can be a recombinant adeno-associated virus (rAAV) vector. Using rAAV vectors, genes can be delivered into a wide range of host cells including many different human and non-human cell lines or tissues. Because AAV is non-pathogenic and does not illicit an immune response, a multitude of pre-clinical studies have reported excellent safety profiles. rAAVs are capable of transducing a broad range of cell types and transduction is not dependent on active host cell division. High titers, >10⁸ viral particle/ml, are easily obtained in the supernatant and 10¹¹-10¹² viral particle/ml with further concentration. The transgene is integrated into the host genome so expression is long term and stable.

The use of alternative AAV serotypes other than AAV-2 (Davidson et al (2000), PNAS 97(7)3428-32; Passini et al (2003), J. Virol 77(12):7034-40) has demonstrated different cell tropisms and increased transduction capabilities. With respect to brain cancers, the development of novel injection techniques into the brain, specifically connection enhanced delivery (CED; Bobo et al (1994), PNAS 91(6):2076-80; Nguyen et al (2001), Neuroreport 12(9):1961-4), has significantly enhanced the ability to transduce large areas of the brain with an AAV vector.

Large scale preparation of AAV vectors is made by a three-plasmid cotransfection of a packaging cell line: AAV vector carrying the chimeric DNA coding sequence, AAV RC vector containing AAV rep and cap genes, and adenovirus helper plasmid pDF6, into 50×150 mm plates of subconfluent 293 cells. Cells are harvested three days after transfection, and viruses are released by three freeze-thaw cycles or by sonication.

AAV vectors are then purified by two different methods depending on the serotype of the vector. AAV2 vector is purified by the single-step gravity-flow column purification method based on its affinity for heparin (Auricchio, A., et. al., 2001, Human Gene therapy 12; 71-6; Summerford, C. and R. Samulski, 1998, J. Virol. 72:1438-45; Summerford, C. and R. Samulski, 1999, Nat. Med. 5: 587-88). AAV2/1 and AAV2/5 vectors are currently purified by three sequential CsCl gradients.

The cloned gene for an pro-angiogenic agent (i.e. the IGPR-1 gene) can be manipulated by a variety of well-known techniques for in vitro mutagenesis, among others, to produce variants of the naturally occurring human protein, herein referred to as muteins or variants or mutants of IGPR-1, which can be used in accordance with the methods and compositions described herein. The variation in primary structure of muteins of the IGPR-1 protein useful in the invention, for instance, can include deletions, additions and substitutions. The substitutions can be conservative or non-conservative. The differences between the natural protein and the mutein generally conserve desired properties, mitigate or eliminate undesired properties and add desired or new properties. For example, in some embodiments, a pro-angiogenic agent can be a IGPR-1 polypeptide of at least 50 amino acids of SEQ ID NO: 1, or a functional mutein or variant thereof.

In some embodiments, the expressed IGPR-1 polypeptide (as a pro-angiogenic agent) can also be a fusion polypeptide, fused, for example, to a polypeptide that targets the product to a desired location, or, for example, a tag that facilitates its purification, if so desired. Fusion to a polypeptide sequence that increases the stability of an expressed polypeptide, i.e. an expressed IGPR-1 polypeptide (as a pro-angiogenic agent) is also contemplated. For example, fusion to a serum protein, e.g., serum albumin, can increase the circulating half-life of a IGPR-1 polypeptide. Tags and fusion partners can be designed to be cleavable, if so desired. Another modification specifically contemplated is attachment, e.g., covalent attachment, to a polymer. In one aspect, polymers such as polyethylene glycol (PEG) or methoxypolyethylene glycol (mPEG) can increase the in vivo half-life of proteins to which they are conjugated. Methods of PEGylation of polypeptide agents are well known to those skilled in the art, as are considerations of, for example, how large a PEG polymer to use. In another aspect, biodegradable or absorbable polymers can provide extended, often localized, release of polypeptide agents. Such synthetic bioabsorbable, biocompatible polymers, which can release proteins over several weeks or months can include, for example, poly-α-hydroxy acids (e.g. polylactides, polyglycolides and their copolymers), polyanhydrides, polyorthoesters, segmented block copolymers of polyethylene glycol and polybutylene terephtalate (Polyactive™), tyrosine derivative polymers or poly(ester-amides). Suitable bioabsorbable polymers to be used in manufacturing of drug delivery materials and implants are discussed e.g. in U.S. Pat. Nos. 4,968,317 and 5,618,563, which are incorporated herein in their entirety by reference and among others, and in “Biomedical Polymers” edited by S. W. Shalaby, Carl Hanser Verlag, Munich, Vienna, N.Y., 1994 and in many references cited in the above publications. The particular bioabsorbable polymer that should be selected will depend upon the particular patient that is being treated.

### Pharmaceutical Formulations

In some embodiments, a pharmaceutical composition comprises an inhibitor IGPR-1 function or expression, and optionally a pharmaceutically acceptable carrier. The compositions encompassed by the invention may further comprise at least one pharmaceutically acceptable excipient. Excipients useful for preparing the dosages forms from the composition according to the invention and the instruments necessary to prepare them are described in U.S. Publication No.: 2003/0206954 and 2004/0052843, which are incorporated herein in their entirety by reference.

For administration to a subject, an inhibitor IGPR-1 function or expression can be provided in pharmaceutically acceptable compositions. A pharmaceutically acceptable composition can comprise a therapeutically-effective amount of an inhibitor IGPR-1 function or expression formulated together with one or more pharmaceutically acceptable carriers (additives) and/or diluents.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the inhibitor IGPR-1 function or expression.

As described in detail below, the pharmaceutical compositions of the present invention comprising an inhibitor IGPR-1 function or expression can be specially formulated for administration to a subject in solid, liquid or gel form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, a GHK tripeptide can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquids such as suspensions (e.g., aqueous or non-aqueous liquid suspensions, oil-in-water emulsions, or water-in-oil liquid emulsions), solutions, and elixirs; and sterile solids (e.g., crystalline or amorphous solids) that can be reconstituted to provide liquid dosage forms.

In methods of treatment described herein, the administration of an inhibitor IGPR-1 function or expression can be for either “prophylactic” or “therapeutic” purpose. When provided prophylactically, an inhibitor IGPR-1 function or expression can be administered to a subject in advance of any symptom of a cancer. The prophylactic administration of an inhibitor IGPR-1 function or expression serves to prevent a metastatic or invasive cancer from occurring, e.g., where a subject has an increased chance of having a cancer which expresses IGPR-1, or already has a cancer or epithelial or endothelial origin, or a cancer which expresses IGPR-1 and wishes to prevent its metastasis to different sites, as disclosed herein. When provided therapeutically, an inhibitor IGPR-1 function or expression is provided at (or after) the onset of a symptom or indication of cancer. Thus, an inhibitor IGPR-1 function or expression can be provided prior to the onset of a cancer or tumor in a subject.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions comprising an inhibitor IGPR-1 function or expression that exhibit large therapeutic indices are preferred.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC₅₀ (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay. The dosage may be determined by a physician and adjusted, as necessary, to suit observed effects of the treatment.

In certain embodiments, the effective dose of a composition comprising an inhibitor IGPR-1 function or expression is administered to a patient once or multiple times. In certain embodiments, the effective dose of a composition comprising an inhibitor IGPR-1 function or expression is administered to a patient repeatedly. Patients can be administered a therapeutic amount of a composition comprising an inhibitor IGPR-1 function or expression, such as 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg or 50 mg/kg. A composition comprising an inhibitor IGPR-1 function or expression can be administered over a period of time, such as over a 5 minute, 10 minute, 15 minute, 20 minute, or 25 minute period. The administration is repeated, for example, on a regular basis, such as hourly for 3 hours, 6 hours, 12 hours or longer or such as biweekly (i.e., every two weeks) for one month, two months, three months, four months or longer. After an initial treatment regimen, the treatments can be administered on a less frequent basis. For example, after administration biweekly for three months, administration can be repeated once per month, for six months or a year or longer. Administration of a composition comprising an inhibitor IGPR-1 function or expression can reduce levels of a marker or symptom of cancer by at least 10%, at least 15%, at least 20%, at least 25%, at least 30%, at least 40%, at least 50%, at least 60%, at least 70%, at least 80% or at least 90% or more.

The dosage of a an inhibitor IGPR-1 function or expression can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

Combination Therapies

As disclosed herein, an inhibitor IGPR-1 function or expression can be administrated to a subject alone, or optionally in combination (e.g. simultaneously with, sequentially or separately) with one or more pharmaceutically active agents, e.g. a second therapeutic agent known to be beneficial in treating cancer. For example, exemplary pharmaceutically active compound include, but are not limited to, those found in Harrison's principals of Internal Medicine, 13^{th} Edition, Eds. T. R. Harrison et al. McGraw-Hill N.Y., N.Y.; Physicians Desk Reference, 50^{th} Edition, 1997, Oradell N.J., Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete contents of all of which are incorporated herein by reference.

In some embodiments, a composition comprising an inhibitor IGPR-1 function or expression and a pharmaceutically active agent can be administrated to the subject in the same pharmaceutical composition or in different pharmaceutical compositions (at the same time or at different times). When administrated at different times, a composition comprising an inhibitor IGPR-1 function or expression and the additional pharmaceutically active agent can be administered within 5 minutes, 10 minutes, 20 minutes, 60 minutes, 2 hours, 3 hours, 4, hours, 8 hours, 12 hours, 24 hours of administration of the other. When a composition comprising an inhibitor IGPR-1 function or expression and the pharmaceutically active agent are administered in different pharmaceutical compositions, routes of administration can be different. For example, a composition comprising an inhibitor IGPR-1 function or expression can be administered by any appropriate route known in the art including, but not limited to oral or parenteral routes, including intravenous, intramuscular, subcutaneous, transdermal, airway (aerosol), pulmonary, nasal, rectal, and topical (including buccal and sublingual) administration, and the pharmaceutically active agent is administered by a different route, e.g. a route commonly used in the art for administration of the pharmaceutically active agent.

In some embodiments, a composition comprising an inhibitor IGPR-1 function or expression can precede, can be concurrent with and/or follow the pharmaceutically active agent by intervals ranging from minutes to weeks. In embodiments where a composition comprising an inhibitor IGPR-1 function or expression and a pharmaceutically active agent are applied separately to a cell, tissue or organism, one would generally ensure that a significant period of time did not expire between the time of each delivery, such that the composition comprising an inhibitor IGPR-1 function or expression and a pharmaceutically active agent would still be able to exert an advantageously combined effect on the cell, tissue or organism.

In some embodiments, the invention contemplates the use of a composition comprising an inhibitor IGPR-1 function or expression and the practice of the methods described herein in conjunction with other therapies such as surgery, e.g., cancer resection.

Aerosol Formulations

In some embodiments, where the cancer is of epithelial origin or located in the lung, a composition comprising an inhibitor IGPR-1 function or expression can be administered directly to the airways of a subject in the form of an aerosol or by nebulization. For use as aerosols, an inhibitor IGPR-1 function or expression in solution or suspension may be packaged in a pressurized aerosol container together with suitable propellants, for example, hydrocarbon propellants like propane, butane, or isobutane with conventional adjuvants. An inhibitor IGPR-1 function or expression can also be administered in a non-pressurized form such as in a nebulizer or atomizer.

The term “nebulization” is well known in the art to include reducing liquid to a fine spray. Preferably, by such nebulization small liquid droplets of uniform size are produced from a larger body of liquid in a controlled manner. Nebulization can be achieved by any suitable means therefore, including by using many nebulizers known and marketed today. For example, an AEROMIST pneumatic nebulizer available from Inhalation Plastic, Inc. of Niles, Ill. When the active ingredients are adapted to be administered, either together or individually, via nebulizer(s) they can be in the form of a nebulized aqueous suspension or solution, with or without a suitable pH or tonicity adjustment, either as a unit dose or multidose device.

As is well known, any suitable gas can be used to apply pressure during the nebulization, with preferred gases to date being those which are chemically inert to an inhibitor IGPR-1 function or expression. Exemplary gases including, but are not limited to, nitrogen, argon or helium can be used to high advantage.

In some embodiments, an inhibitor IGPR-1 function or expression can also be administered directly to the airways in the form of a dry powder. For use as a dry powder, an inhibitor IGPR-1 function or expression can be administered by use of an inhaler. Exemplary inhalers include metered dose inhalers and dry powdered inhalers.

A metered dose inhaler or “MDI” is a pressure resistant canister or container filled with a product such as a pharmaceutical composition dissolved in a liquefied propellant or micronized particles suspended in a liquefied propellant. The propellants which can be used include chlorofluorocarbons, hydrocarbons or hydrofluoroalkanes. Especially preferred propellants are P134a (tetrafluoroethane) and P227 (heptafluoropropane) each of which may be used alone or in combination. They are optionally used in combination with one or more other propellants and/or one or more surfactants and/or one or more other excipients, for example ethanol, a lubricant, an anti-oxidant and/or a stabilizing agent. The correct dosage of the composition is delivered to the patient.

A dry powder inhaler (i.e. Turbuhaler (Astra AB)) is a system operable with a source of pressurized air to produce dry powder particles of a pharmaceutical composition that is compacted into a very small volume.

Dry powder aerosols for inhalation therapy are generally produced with mean diameters primarily in the range of <5 μm. As the diameter of particles exceeds 3 μm, there is increasingly less phagocytosis by macrophages. However, increasing the particle size also has been found to minimize the probability of particles (possessing standard mass density) entering the airways and acini due to excessive deposition in the oropharyngeal or nasal regions.

Suitable powder compositions include, by way of illustration, powdered preparations of an inhibitor IGPR-1 function or expression thoroughly intermixed with lactose, or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants, and co-solvents and may be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic an diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S, and R. H. Muller, J. Controlled Release, 22: 263-272 (1992); Tabata, Y, and Y. Wada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

Oral Dosage Formulations

Pharmaceutical compositions comprising an inhibitor IGPR-1 function or expression can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington's Pharmaceutical Sciences, 18th ed., Mack Publishing, Easton, Pa. (1990).

Typical oral dosage forms of the compositions of the disclosure are prepared by combining the pharmaceutically acceptable salt of disclosed compounds in an intimate admixture with at least one excipient according to conventional pharmaceutical compounding techniques. Excipients can take a wide variety of forms depending on the form of the composition desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, kaolin, diluents, granulating agents, lubricants, binders, and disintegrating agents. Due to their ease of administration, tablets and capsules represent the most advantageous solid oral dosage unit forms, in which case solid pharmaceutical excipients are used. If desired, tablets can be coated by standard aqueous or nonaqueous techniques. These dosage forms can be prepared by any of the methods of pharmacy. In general, pharmaceutical compositions and dosage forms are prepared by uniformly and intimately admixing the active ingredient(s) with liquid carriers, finely divided solid carriers, or both, and then shaping the product into the desired presentation if necessary.

For example, a tablet can be prepared by compression or molding. Compressed tablets can be prepared by compressing in a suitable machine the active ingredient(s) in a free-flowing form, such as a powder or granules, optionally mixed with one or more excipients. Molded tablets can be made by molding in a suitable machine a mixture of the powdered compound moistened with an inert liquid diluent. Examples of excipients that can be used in oral dosage forms of the disclosure include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, and AVICEL-PH-105 (available from FMC Corporation, American Viscose Division, Avicel Sales, Marcus Hook, Pa., U.S.A.), and mixtures thereof. An exemplary suitable binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the disclosure is typically present in from about 50 to about 99 weight percent of the pharmaceutical composition or dosage form.

Disintegrants are used in the compositions of the disclosure to provide tablets that disintegrate when exposed to an aqueous environment. Tablets that contain too much disintegrant may swell, crack, or disintegrate in storage, while those that contain too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) should be used to form solid oral dosage forms of the disclosure. The amount of disintegrant used varies based upon the type of formulation and mode of administration, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used to form pharmaceutical compositions and dosage forms of the disclosure include, but are not limited to, agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, and mixtures thereof.

Lubricants that can be used to form pharmaceutical compositions and dosage forms of the disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL® 200, manufactured by W. R. Grace Co. of Baltimore, Md.), a coagulated aerosol of synthetic silica (marketed by Degussa Co. of Piano, Tex.), CAB-O-SIL® (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

This disclosure further encompasses lactose-free pharmaceutical compositions and dosage forms, wherein such compositions preferably contain little, if any, lactose or other mono- or disaccharides. As used herein, the term “lactose-free” means that the amount of lactose present, if any, is insufficient to substantially increase the degradation rate of an active ingredient.

Lactose-free compositions of the disclosure can comprise excipients which are well known in the art and are listed in the USP(XXI)/NF (XVI), which is incorporated herein by reference. In general, lactose-free compositions comprise a pharmaceutically acceptable salt of an HIF inhibitor, a binder/filler, and a lubricant in pharmaceutically compatible and pharmaceutically acceptable amounts. Preferred lactose-free dosage forms comprise a pharmaceutically acceptable salt of the disclosed compounds, microcrystalline cellulose, pre-gelatinized starch, and magnesium stearate.

This disclosure further encompasses anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

For oral administration, the dosage should contain at least at least 0.1% of an inhibitor IGPR-1 function or expression. The percentage of an inhibitor IGPR-1 function or expression in these compositions may, of course, be varied and may conveniently be between about 2% to about 60% of the weight of the unit. The amount of an inhibitor IGPR-1 function or expression in such therapeutically useful compositions is such that a suitable dosage will be obtained.

Controlled Release Forms

In some embodiments, an inhibitor IGPR-1 function or expression can be administered by controlled- or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Chemg-ju, Controlled Release Dosage Form Design, 2 (Technomic Publishing, Lancaster, Pa.: 2000).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, Duolite® A568 and Duolite® AP143 (Rohm & Haas, Spring House, Pa. USA).

One embodiment of the disclosure encompasses a unit dosage form that includes a pharmaceutically acceptable salt of the disclosed compounds (e.g., a sodium, potassium, or lithium salt), or a polymorph, solvate, hydrate, dehydrate, co-crystal, anhydrous, or amorphous form thereof, and one or more pharmaceutically acceptable excipients or diluents, wherein the pharmaceutical composition or dosage form is formulated for controlled-release. Specific dosage forms utilize an osmotic drug delivery system.

A particular and well-known osmotic drug delivery system is referred to as OROS® (Alza Corporation, Mountain View, Calif. USA). This technology can readily be adapted for the delivery of compounds and compositions of the disclosure. Various aspects of the technology are disclosed in U.S. Pat. Nos. 6,375,978 B1; 6,368,626 B1; 6,342,249 B1; 6,333,050 B2; 6,287,295 B1; 6,283,953 B1; 6,270,787 B1; 6,245,357 B1; and 6,132,420; each of which is incorporated herein by reference. Specific adaptations of OROS® that can be used to administer compounds and compositions of the disclosure include, but are not limited to, the OROS® Push-Pull™, Delayed Push-Pull™, Multi-Layer Push-Pull™, and Push-Stick™ Systems, all of which are well known. See, e.g. worldwide website alza.com. Additional OROS® systems that can be used for the controlled oral delivery of compounds and compositions of the disclosure include OROS®-CT and L-OROS®; see, Delivery Times, vol. 11, issue II (Alza Corporation).

Conventional OROS® oral dosage forms are made by compressing a drug powder (e.g., a GHK tripeptide which is a salt) into a hard tablet, coating the tablet with cellulose derivatives to form a semi-permeable membrane, and then drilling an orifice in the coating (e.g., with a laser). Kim, Chemg-ju, Controlled Release Dosage Form Design, 231-238 (Technomic Publishing, Lancaster, Pa.: 2000). The advantage of such dosage forms is that the delivery rate of the drug is not influenced by physiological or experimental conditions. Even a drug with a pH-dependent solubility can be delivered at a constant rate regardless of the pH of the delivery medium. But because these advantages are provided by a build-up of osmotic pressure within the dosage form after administration, conventional OROS® drug delivery systems cannot be used to effectively delivery drugs with low water solubility.

In some embodiments, a specific dosage form of an inhibitor IGPR-1 function or expression compositions of the disclosure includes: a wall defining a cavity, the wall having an exit orifice formed or formable therein and at least a portion of the wall being semipermeable; an expandable layer located within the cavity remote from the exit orifice and in fluid communication with the semipermeable portion of the wall; a dry or substantially dry state drug layer located within the cavity adjacent the exit orifice and in direct or indirect contacting relationship with the expandable layer; and a flow-promoting layer interposed between the inner surface of the wall and at least the external surface of the drug layer located within the cavity, wherein the drug layer includes an inhibitor IGPR-1 function or expression, or a polymorph, solvate, hydrate, dehydrate, co-crystal, anhydrous, or amorphous form thereof. See U.S. Pat. No. 6,368,626, the entirety of which is incorporated herein by reference.

In another embodiment, a specific dosage form of an inhibitor IGPR-1 function or expression includes: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a liquid, active agent formulation absorbed in porous particles, the porous particles being adapted to resist compaction forces sufficient to form a compacted drug layer without significant exudation of the liquid, active agent formulation, the dosage form optionally having a placebo layer between the exit orifice and the drug layer, wherein the active agent formulation comprises a GHK tripeptide, or a polymorph, solvate, hydrate, dehydrate, co-crystal, anhydrous, or amorphous form thereof. See U.S. Pat. No. 6,342,249, the entirety of which is incorporated herein by reference.

In some embodiments, an inhibitor IGPR-1 function or expression is administered to a subject by sustained release or in pulses. Pulse therapy is not a form of discontinuous administration of the same amount of a composition over time, but comprises administration of the same dose of the composition at a reduced frequency or administration of reduced doses. Sustained release or pulse administration is particularly preferred when the respiratory disorder occurs continuously in the subject, for example where the subject has continuous symptoms of a respiratory disorder. Each pulse dose can be reduced and the total amount of drug administered over the course of treatment to the patient is minimized.

In some embodiments, individual pulses can be delivered to the patient continuously over a period of several hours, such as about 2, 4, 6, 8, 10, 12, 14 or 16 hours, or several days, such as 2, 3, 4, 5, 6, or 7 days, preferably from about 1 hour to about 24 hours and more preferably from about 3 hours to about 9 hours.

In some embodiments, an interval between pulses or an interval of no delivery is greater than 24 hours and preferably greater than 48 hours, and can be for even longer such as for 3, 4, 5, 6, 7, 8, 9 or 10 days, two, three or four weeks or even longer. As the results achieved may be surprising, the interval between pulses, when necessary, can be determined by one of ordinary skill in the art. Often, the interval between pulses can be calculated by administering another dose of the composition when the composition or the active component of the composition is no longer detectable in the patient prior to delivery of the next pulse. Intervals can also be calculated from the in vivo half-life of the composition. Intervals may be calculated as greater than the in vivo half-life, or 2, 3, 4, 5 and even 10 times greater the composition half-life.

In some embodiments, the number of pulses in a single therapeutic regimen may be as little as two, but is typically from about 5 to 10, 10 to 20, 15 to 30 or more. In fact, patients can receive drugs for life according to the methods of this invention without the problems and inconveniences associated with current therapies. Compositions can be administered by most any means, but are preferable delivered to the patient as an injection (e.g. intravenous, subcutaneous, and intraarterial), infusion or instillation. Various methods and apparatus for pulsing compositions by infusion or other forms of delivery to the patient are disclosed in U.S. Pat. Nos. 4,747,825; 4,723,958; 4,948,592; 4,965,251 and 5,403,590. Sustained release can also be accomplished by means of an osmotic pump. In some embodiments, an inhibitor IGPR-1 function or expression is administered over a period of several days, such as 2, 3, 4, 5, 6 or 7 days.

Parenteral Dosage Forms

In some embodiments, parenteral dosage forms of a modulator of an inhibitor IGPR-1 function or expression can also be administered to a subject with a respiratory disorder by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, administration DUROS®-type dosage forms, and dose-dumping.

Suitable vehicles that can be used to provide parenteral dosage forms of an inhibitor IGPR-1 function or expression as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Compounds that alter or modify the solubility of a pharmaceutically acceptable salt of a GHK tripeptide as disclosed herein can also be incorporated into the parenteral dosage forms of the disclosure, including conventional and controlled-release parenteral dosage forms.

Topical, Transdermal and Mucosal Dosage Forms

In some embodiments, an inhibitor IGPR-1 function or expression can be administered to a subject topically. In some embodiments, topical dosage forms of an inhibitor IGPR-1 function or expression include, but are not limited to, creams, lotions, ointments, gels, shampoos, sprays, aerosols, solutions, emulsions, and other forms known to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington's Pharmaceutical Sciences, 18.sup.th Ed., Mack Publishing, Easton, Pa. (1990).

Transdermal and mucosal dosage forms of the compositions comprising a modulator of an inhibitor IGPR-1 function or expression as disclosed herein include, but are not limited to, ophthalmic solutions, patches, sprays, aerosols, creams, lotions, suppositories, ointments, gels, solutions, emulsions, suspensions, or other forms known to one of skill in the art. See, e.g., Remington's Pharmaceutical Sciences, 18th Ed., Mack Publishing, Easton, Pa. (1990); and Introduction to Pharmaceutical Dosage Forms, 4th Ed., Lea & Febiger, Philadelphia, Pa. (1985). Dosage forms suitable for treating mucosal tissues within the oral cavity can be formulated as mouthwashes, as oral gels, or as buccal patches. Additional transdermal dosage forms include “reservoir type” or “matrix type” patches, which can be applied to the skin and worn for a specific period of time to permit the penetration of a desired amount of active ingredient.

Examples of transdermal dosage forms and methods of administration that can be used to administer the active ingredient(s) of the disclosure include, but are not limited to, those disclosed in U.S. Pat. Nos. 4,624,665; 4,655,767; 4,687,481; 4,797,284; 4,810,499; 4,834,978; 4,877,618; 4,880,633; 4,917,895; 4,927,687; 4,956,171; 5,035,894; 5,091,186; 5,163,899; 5,232,702; 5,234,690; 5,273,755; 5,273,756; 5,308,625; 5,356,632; 5,358,715; 5,372,579; 5,421,816; 5,466,465; 5,494,680; 5,505,958; 5,554,381; 5,560,922; 5,585,111; 5,656,285; 5,667,798; 5,698,217; 5,741,511; 5,747,783; 5,770,219; 5,814,599; 5,817,332; 5,833,647; 5,879,322; and 5,906,830, each of which are incorporated herein by reference in their entirety.

Suitable excipients (e.g., carriers and diluents) and other materials that can be used to provide transdermal and mucosal dosage forms encompassed by this disclosure are well known to those skilled in the pharmaceutical arts, and depend on the particular tissue or organ to which a given pharmaceutical composition or dosage form will be applied. With that fact in mind, typical excipients include, but are not limited to water, acetone, ethanol, ethylene glycol, propylene glycol, butane-1,3-diol, isopropyl myristate, isopropyl palmitate, mineral oil, and mixtures thereof, to form dosage forms that are non-toxic and pharmaceutically acceptable.

Depending on the specific tissue to be treated, additional components may be used prior to, in conjunction with, or subsequent to treatment with an inhibitor IGPR-1 function or expression. For example, penetration enhancers can be used to assist in delivering the active ingredients to or across the tissue. Suitable penetration enhancers include, but are not limited to: acetone; various alcohols such as ethanol, oleyl, an tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as TWEEN 80 (polysorbate 80) and SPAN 60 (sorbitan monostearate).

The pH of a pharmaceutical composition or dosage form, or of the tissue to which the pharmaceutical composition or dosage form is applied, may also be adjusted to improve delivery of the active ingredient(s). Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of the active ingredient(s) so as to improve delivery. In this regard, stearates can serve as a lipid vehicle for the formulation, as an emulsifying agent or surfactant, and as a delivery-enhancing or penetration-enhancing agent. Different hydrates, dehydrates, co-crystals, solvates, polymorphs, anhydrous, or amorphous forms of the pharmaceutically acceptable salt of an inhibitor IGPR-1 function or expression can be used to further adjust the properties of the resulting composition.

As used herein, an “effective amount” or “therapeutically effective amount” means the dose or effective amount to be administered to a subject and the frequency of administration to a subject which is sufficient to obtain a therapeutic effect as readily determined by one of ordinary skill in the art, by the use of known techniques and by observing results obtained under analogous circumstances. The dose or effective amount to be administered to a subject and the frequency of administration to a subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of a subject to be treated, and other relevant circumstances.

The phrase “therapeutically effective” indicates the capability of a combination of agents to prevent, or reduce the severity of, the disorder or its undesirable symptoms, while avoiding adverse side effects typically associated with alternative therapies. Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

The amounts of an inhibitor IGPR-1 function or expression that are used in the methods and compositions as disclosed herein can be amounts are sufficient to for treatment, prevention or inhibition of a malignancy or tumor growth. In some embodiments, the amount an inhibitor IGPR-1 function or expression that is used in the compositions and methods as disclosed herein ranges from about 0.001 to about 100 milligrams per day per kilogram of body weight of a subject (mg/day/kg), and in some embodiments from about 0.05 to about 50 mg/day/kg, even more preferably from about 1 to about 20 mg/day/kg.

In some embodiments, an inhibitor IGPR-1 function or expression is administered to a subject at a frequency and dose sufficient for the inhibitor IGPR-1 function or expression to be biologically active over an extended period of time, for example but not limited to at least one month, at least 2 months or at least 3 months etc. Preferably, an inhibitor IGPR-1 function or expression is biologically active of the entire period of time that the cancer is present. Accordingly, in some embodiments, an inhibitor IGPR-1 function or expression can be administered on a regular scheduled administration or it can be administered continually for example by a pump or catheter.

The frequency of dose of an inhibitor IGPR-1 function or expression will depend upon the half-life of the inhibitor IGPR-1 function or expression, for example dependent on its hydrolysis, presence of functional metabolite, or precursor thereof. If an inhibitor IGPR-1 function or expression or analog, hydrolysis product, metabolite, or precursor thereof has a short half-life (e.g. from about 2 to 10 hours) it can be necessary to give one or more doses per day. Alternatively, if an inhibitor IGPR-1 function or expression or analog, hydrolysis product, metabolite, or precursor thereof has a long half-life (e.g. from about 2 to about 15 days) it may only be necessary to give a dosage once per day, per week, or even once every 1 or 2 months. A preferred dosage rate is to administer the dosage amounts described above to a subject once per day. It will be apparent to those skilled in the art that it is possible, and perhaps desirable, to combine various times and methods of administration in the practice of the present methods.

In some embodiments, an inhibitor IGPR-1 function or expression can be administered to the subject on a regular schedule and for an extended period of time, for example for at least one month, or at least 2 months or at least 3 months, for at least 6 months, for at least 8 months or more. In some embodiments, an inhibitor IGPR-1 function or expression for use in the methods and compositions as disclosed herein can exceed 2500 milligrams per kilogram of body weight per day (mg/kg/day) up to, or above the maximum tolerated dose (MTD) of an inhibitor IGPR-1 function or expression in humans. A MTD as used herein is operationally defined in toxicology as the highest daily dose of an agent that does not cause overt toxicity in humans. MTD is typically assessed by administering the agent in a ninety-day study in laboratory mice or rats, and is a dose used for longer-term safety assessment in the same species, usually lasting two years or a lifetime.

The frequency of dose will depend upon the half-life of the inhibitor IGPR-1 function or expression or an analog, hydrolysis product, metabolite, or precursor thereof. If an inhibitor IGPR-1 function or expression such as cyclosporine or analog, hydrolysis product, metabolite, or precursor thereof has a short half-life (e.g. from about 2 to 10 hours) it can be necessary to give one or more doses per day. Alternatively, if an inhibitor IGPR-1 function or expression hydrolysis product, metabolite, or precursor thereof has a long half-life (e.g. from about 2 to about 15 days) it may only be necessary to give a dosage once per day, per week, or even once every 1 or 2 months. A preferred dosage rate is to administer the dosage amounts described above to a subject once per day. It will be apparent to those skilled in the art that it is possible, and perhaps desirable, to combine various times and methods of administration in the practice of the present methods.

In one embodiment, the compositions comprising an inhibitor IGPR-1 function or expression as disclosed herein can be administered initially by intravenous injection to bring blood levels to a suitable level. An oral dosage form then maintains the subject's composition levels. Additionally, other forms of administration, dependent upon the subject's condition and as indicated above, can be used. The quantity to be administered will vary for a subject being treated and will be dependent on the type of inhibitor IGPR-1 function or expression being delivered. In some embodiments the dosage of an inhibitor IGPR-1 function or expression can vary from about 100 ng/kg of body weight to 100 mg/kg of body weight per day and preferably will be from 1 mg/kg to 10 mg/kg per day.

It will be understood, however, that the specific dose level for any particular subject will depend upon a variety of factors including the activity of the specific compound employed, the half-life of the compound, the age, body weight, general health, sex, diet, time of administration, route of administration, rate of excretion, drug combination and the severity of the particular auto-immune disease or the severity of the need for immunosuppressive therapy. Dosage levels of immunosuppressant agents, for example cyclosporine can be on the order from about 0.05 mg to about 50 mg per kilogram of body weight per day are useful in the compositions and methods as disclosed herein (from about 2.5 mg to about 2.5 g per patient per day). In some embodiments, a immunosuppressant, such as cyclosporine for use in the methods and compositions as disclosed herein can exceed 50 mg per kilogram of body weight per day (mg/kg/day) up to, or above the maximum tolerated dose (MTD) for the immunosuppressant, such as cyclosporine in humans.

The amount of an inhibitor IGPR-1 function or expression can be combined with the carrier materials to produce a single dosage form will vary depending upon the subject host treated and the particular, mode of administration. For example, a formulation intended for the oral administration of humans may contain from 2.5 mg/kg to 2.5 g/kg of body weight of an inhibitor IGPR-1 function or expression with an appropriate and convenient amount of carrier material which may vary from about 5 to about 95 percent of the total composition. Dosage unit forms will generally contain between from about 5 mg to about 500 mg/kg per day body weight of an inhibitor IGPR-1 function or expression.

It is noted that humans are treated generally longer than the mice or other experimental animals exemplified herein which treatment has a length proportional to the length of the disease process and drug effectiveness. The doses can be single doses or multiple doses over a period of several days, but single doses are preferred.

In some embodiments of the invention, an inhibitor IGPR-1 function or expression can be administered substantially simultaneously with another agent, meaning that both agents can be provided in a single dosage, for example by mixing the agents and incorporating the mixture into a single capsule or within a short time of each other. In alternative embodiments, an inhibitor IGPR-1 function or expression can be administered substantially simultaneously with another agent by administration in separate dosages within a short time period, for example within one hour or less, 45 minutes or less, 30 minutes or less, 15 minutes or less, 10 minutes or less, 5 minutes or less and all time periods in between. Alternatively, an inhibitor IGPR-1 function or expression can be administered sequentially with another agent, meaning that separate dosages, and possibly even separate dosage forms an inhibitor IGPR-1 function or expression and another agent can be administered at separate times, for example on a staggered schedule but with equal frequency of administration of an inhibitor IGPR-1 function or expression and the other agent. Different agents have different half lives, thus one can stagger schedules and still maintain both agents being effective in an individual. In any case, it is preferable that, among successive time periods of a sufficient length, for example one day, the weight ratio an inhibitor IGPR-1 function or expression administered to the weight ratio of the other agent administered remains constant.

In alternative embodiments, a subject is administered an inhibitor IGPR-1 function or expression continuously or can be administered an inhibitor IGPR-1 function or expression in repeated doses. By way of an example but not as a limitation, a subject can be continuously administered an inhibitor IGPR-1 function or expression by any suitable means such as catheterization or by pump administration. In alternative embodiments, a subject can be administered an inhibitor IGPR-1 function or expression at regular intervals, for example but not limited to daily, twice a day, twice a week, monthly etc by any suitable means known by persons of ordinary skill in the art and disclosed herein to keep the agents active in an individual.

In alternative embodiments, a subject can be administered an inhibitor IGPR-1 function or expression by pulse chase schedules. For example, a subject is administered an inhibitor IGPR-1 function or expression for a brief period of time (the pulse) and then a subject is administered the other agent, such as for a longer period (the chase). In such embodiments, a subject can be administered varying amounts of each agent for each pulse-chase administration regimen, and can swap the pulse compound and the chase agent in any order.

The term “pharmacologically effective amount” shall mean that amount of an inhibitor IGPR-1 function or expression or pharmaceutical agent that will elicit the biological or medical response of a tissue, system, animal or human that is being sought by a researcher or clinician. This amount can be a therapeutically effective amount.

The term “biologically active” refers to a compound that is capable of eliciting a biological response in a tissue, system, animal or human.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included for use of the composition and method(s) as disclosed herein are the isomeric forms and tautomers and the pharmaceutically-acceptable salts of an inhibitor IGPR-1 function or expression. Isomers of an inhibitor IGPR-1 function or expression include their diastereomers, enantiomers, and racemates as well as their structural to isomers and are disclosed herein. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, α-hydroxybutyric, galactaric, and galacturonic acids.

Suitable pharmaceutically-acceptable base addition salts of compounds can be used in the compositions and methods as disclosed herein, such as for example, metallic ion salts and organic ion salts. More preferred metallic ion salts include, but are not limited to, appropriate alkali metal (group Ia) salts, alkaline earth metal (group Ia) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention. Pharmaceutically acceptable esters include, but are not limited to, the alkyl esters of the COX-2 inhibitors.

For methods of prevention of a malignancy or tumor growth in a subject in need thereof, for example a subject with a cancer expressing IGPR-1 can be any subject, for example any human or animal subject, and preferably is a subject that is in need of treatment of a cancer and/or metastatic cancer.

In connection with the inventive method, the compositions and methods as disclosed herein comprising an inhibitor IGPR-1 function or expression can be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition can be at or near body temperature.

The phrase “administration” in defining the use of an inhibitor IGPR-1 function or expression is intended to encompass administration of each agent in a manner and in a regimen that will provide beneficial effects of the inhibitor IGPR-1 function or expression.

The phrases “therapeutically-effective” and “effective for the treatment, prevention, or inhibition”, are intended to qualify the amount of each NSAID such as a COX-2 agent and immunosuppressive agents such as cyclosporine for use in the immunosuppressive therapy which will achieve the goal of reduction of the incidence or severity and/or frequency of incidence of a tumor growth, malignancy or neoplasia associated with immunosuppression.

In some embodiments, the compositions and methods as disclosed herein comprising an inhibitor IGPR-1 function or expression can be administered orally, for example, as tablets, coated tablets, dragees, troches, lozenges, gums, aqueous or oily suspensions, dispersible powders or granules, emulsions, hard or soft capsules, or syrups or elixirs. Compositions intended for oral use can be prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients can be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets can be uncoated or they can be coated by known techniques to delay disintegration and adsorption in the gastrointestinal tract and thereby provide a sustained action over a longer period. For example, a time delay material such as glyceryl monostearate or glyceryl distearate can be employed.

Formulations for oral use can also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents can be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions can also contain one or more preservatives, for example, ethyl or n-propyl p-hydroxybenzoate, one or more coloring agents, one or more flavoring agents, or one or more sweetening agents, such as sucrose or saccharin.

Oily suspensions can be formulated by suspending the active ingredients in an omega-3 fatty acid, a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents, such as those set forth above, and flavoring agents can be added to provide a palatable oral preparation. These compositions can be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, can also be present.

Syrups and elixirs containing the novel combination can be formulated with sweetening agents, for example glycerol, sorbitol or sucrose. Such formulations may also contain demulcent, preservative and flavoring and coloring agents.

A pharmaceutical composition comprising an inhibitor IGPR-1 function or expression as disclosed herein can also be administered parenterally, either subcutaneously, or intravenously, or intramuscularly, or intrasternally, or by infusion techniques, in the form of sterile injectable aqueous or olagenous suspensions. Such suspensions can be formulated according to the known art using those suitable dispersing of wetting agents and suspending agents which have been mentioned above, or other acceptable agents. The sterile injectable preparation can also be a sterile injectable solution or suspension in a non-toxic parenterally-acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, n-3 polyunsaturated fatty acids may find use in the preparation of injectables.

In some embodiments, composition as disclosed herein comprising an inhibitor IGPR-1 function or expression can be formulated for targeted delivery to the cancer, e.g., to the colon if it is a colon cancer. Formulation of the compositions comprising an inhibitor IGPR-1 function or expression as disclosed herein for targeted delivery to the colon can be performed according the methods as disclosed in European Patent Applications EP825854 and EP827389 which are incorporated herein in their entirety by reference. Other examples of dosages and formulations for targeted delivery to the colon are disclosed in U.S. Pat. No. 5,171,580, which is incorporated herein by reference, teaches a preparation for delivery in the large intestine and especially the colon, comprising an active containing core coated with three protection layers of coatings having different solubilities. The inner layer is Eudragit® S, with a coating thickness of about 40-120 microns, the intermediate coating layer is a swellable polymer with a coating thickness of about 40-120 microns, and the outer layer is cellulose acetate phthalate, hydroxypropyl methyl cellulose phthalate, polyvinyl acetate phthalate, hydroxyethyl cellulose phthalate, cellulose acetate tetrahydrophthalate, or Eudragit® L. U.S. Pat. No. 4,910,021 which is incorporated herein by reference, teaches a targeted delivery system wherein the composition comprises a hard or soft gelatin capsule containing an active ingredient such as insulin and an absorption promoter. The capsule is coated with a film forming composition being sufficiently soluble at a pH above 7 as to be capable of permitting the erosion or dissolution of said capsule. The film forming composition is preferably a mixture of Eudragit® L, Eudragit® RS, and Eudragit® S at specific ratios to provide solubility above a pH of 7. U.S. Pat. No. 4,432,966 which is incorporated herein by reference, teaches a compressed tablet with an active agent, coated with a first coating layer comprising a mixture of microcrystalline cellulose and lower alkyl ether of a cellulose film-forming organic polymer such as ethyl cellulose, and a second coating layer selected from cellulose acetylphthalate, hydroxypropyl methylcellulose phthalate, benzophenyl salicylate, cellulose acetosuccinate, copolymers of styrene and of maleic acid, formulated gelatin, salol, keratin, stearic acid, myristic acid, gluten, acrylic and methacrylic resins, and copolymers of maleic acid and phthalic acid derivatives. EP-A-572,942 and EP-A-621,032 which are incorporated herein by reference, also describe colon specific dosage units with multiple polymer coatings. Each uses a pH independent inner layer and an outer coating like that of the inner layer of the present invention.

A pharmaceutical composition comprising an inhibitor IGPR-1 function or expression as disclosed herein can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The compositions and methods as disclosed herein comprising an inhibitor IGPR-1 function or expression can also be administered topically, in the form of patches, creams, ointments, jellies, collyriums, solutions or suspensions. Of course, the compositions of the present invention can be administered by routes of administration other than topical administration. Also, as mentioned above, an inhibitor IGPR-1 function or expression can be administered separately, with each agent administered by any of the above mentioned administration routes. As a non-limiting example, the compositions and methods as disclosed herein can be administered orally in any or the above mentioned forms (e.g. in capsule form) while the immunosuppressive agents such as cyclosporine is administered topically (e.g. as a cream).

Daily dosages can vary within wide limits and will be adjusted to the subject requirements in each particular case. In general, for administration to adults, an appropriate daily dosage has been described above, although the limits that were identified as being preferred can be exceeded if necessary. The daily dosage can be administered as a single dosage or in divided dosages. Various delivery systems include capsules, tablets, and gelatin capsules, for example.

Any suitable route and any combination of routes of administration can be employed for providing a subject with an effective dosage of a combined therapy of the present invention. For example, oral, rectal, transdermal, parenteral (subcutaneous, intramuscular, intravenous), intrathecal, and like forms of administration can be employed. Dosage forms include tablets, troches, dispersions, suspensions, solutions, capsules, patches, and the like.

The composition of the present invention is administered and dosed in accordance with good medical practice, taking into account the clinical condition of the individual subject, the site and method of administration, scheduling of administration, subject age, sex, body weight and other factors known to medical practitioners.

In the method of the present invention, the composition of the present invention can be administered in various ways. It should be noted that it can be administered as the compound or as pharmaceutically acceptable salt thereof and can be administered alone or as an active ingredient in combination with pharmaceutically acceptable carriers, diluents, adjuvants and vehicles. The composition can be administered orally, subcutaneously or parenterally including intravenous, intraarterial, intramuscular, intraperitoneally, and intranasal administration as well as intrathecal and infusion techniques. Implants of the composition are also useful. A subject being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

When administering the composition of the present invention parenterally, it is generally formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, potyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions comprising an inhibitor IGPR-1 function or expression can be prepared by incorporating the compounds utilized in practicing the present invention in the required amount of the appropriate solvent with various other ingredients, as desired.

A pharmacological formulation comprising an inhibitor IGPR-1 function or expression as disclosed herein can be administered to a subject in an injectable formulation containing any compatible carrier, such as various vehicle, adjuvants, additives, and diluents; or the composition utilized in the present invention can be administered parenterally to a subject in the form of slow-release subcutaneous implants or targeted delivery systems such as monoclonal antibodies, vectored delivery, iontophoretic, polymer matrices, liposomes, microspheres and nanospheres. Examples of delivery systems useful in the present invention include those disclosed in U.S. Pat. Nos. 5,225,182; 5,169,383; 5,167,616; 4,959,217; 4,925,678; 4,487,603; 4,486,194; 4,447,233; 4,447,224; 4,439,196; and 4,475,196, which are incorporated herein by reference. Many other such implants, delivery systems, and modules are well known to those skilled in the art.

A pharmacological formulation comprising an inhibitor IGPR-1 function or expression as disclosed herein can be administered orally to the subject. Conventional methods such as administering the composition in tablets, suspensions, solutions, emulsions, capsules, powders, syrups and the like are usable. Known techniques that deliver the composition orally or intravenously and retain the biological activity are preferred.

In another embodiment, an inhibitor IGPR-1 function or expression can be administered alone or in conjunction with a standard tumor therapy, such as chemotherapy, immunotherapy, surgery, hormone therapy or radiation therapy or surgery.

While not wishing to be bound by any theory, a composition comprising an inhibitor IGPR-1 function or expression as disclosed herein can be used to inhibit a malignancy or neoplasia or prevent the occurrence or increase of tumor growth. In some embodiments, the pharmaceutical compositions and methods comprising an inhibitor IGPR-1 function or expression as disclosed herein are administered in conjunction with the standard antitumor therapy and, in addition, can be administered on a continuing basis after the standard antitumor therapy. Chemotherapy or radiation therapy can then be repeated along with the continuation of the administration of the compositions and methods as disclosed herein comprising immunosuppressive agents such as cyclosporine and a COX-2 selective inhibitor.

In some embodiments, the compositions comprising an inhibitor IGPR-1 function or expression and methods as disclosed herein can also be used in combination with existing therapeutic agents for the treatment of cancer. Suitable agents to be used in combination include, but are not limited: (i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas), antimetabolites (for example antifolates such as fluoropyrimidines like 5-fluorouracil and tegafur, raltitrexed, methotrexate, cytosine arabinoside, hydroxyurea, gemcitabine and paclitaxel (Taxol[R]), antitumour antibiotics (for example anthracyclines like adriamycin, bleomycin, doxorubicin, daunomycin, epirubicin, idarubicin, mitomycin-C, dactinomycin and mithramycin), antimitotic agents (for example vinca alkaloids like vincristine, vinblastine, vindesine and vinorelbine and taxoids like taxol and taxotere), and topoisomerase inhibitors (for example epipodophyllotoxins like etoposide and teniposide, amsacrine, topotecan and camptothecin), (ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5 α-reductase such as finasteride, (iii) Agents which inhibit cancer cell invasion (for example metalloproteinase inhibitors like marimastat and inhibitors of urokinase plasminogen activator receptor function), (iv) inhibitors of growth factor function, for example such inhibitors include growth factor antibodies, growth factor receptor antibodies (for example the anti-erbb2 antibody trastuzumab [Herceptin] and the anti-erbb 1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy) quinazolin-4-amine (erlotinib, OSI-774) and 6-acrylamido-N-(3-chloro-4-fluorophenyl)-7-(3-morpholinopropoxy) quinazolin-4-amine (CI 1033)), for example inhibitors of the platelet-derived growth factor family and for example inhibitors of the hepatocyte growth factor family, (v) antiangiogenic agents such as those which inhibit the effects of vascular endothelial growth factor, (for example the anti-vascular endothelial cell growth factor antibody bevacizumab [Avastin], compounds such as those disclosed in International Patent Applications WO 97/22596, WO 97/30035, WO 97/32856 and WO 98/13354, which are incorporated herein by reference) and compounds that work by other mechanisms (for example linomide, inhibitors of integrin v³ function and angiostatin), (vi) vascular damaging agents such as Combretastatin A4 and compounds disclosed in International Patent Applications WO 99/02166, WO00/40529, WO 00/41669, WO01/92224, WO02/04434 and WO02/08213, which are incorporated herein by reference, (vii) antisense therapies, for example those which are directed to the targets listed above, such as ISIS 2503, an anti-ras antisense, (viii) gene therapy approaches, including for example approaches to replace aberrant genes such as aberrant p53 or aberrant BRCA 1 or BRCA2, GDEPT (gene-directed enzyme pro-drug therapy) approaches such as those using cytosine deaminase, thymidine kinase or a bacterial nitroreductase enzyme and approaches to increase subject tolerance to chemotherapy or radiotherapy such as multi-drug resistance gene therapy, and (ix) immunotherapy approaches, including for example ex-vivo and in-vivo approaches to increase the immunogenicity of subject tumor cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumor cell lines and approaches using anti-idiotypic antibodies.

Some embodiments of the present invention may be defined in any of the following numbered paragraphs:
1. A method of inhibiting angiogenesis in a subject, comprising administering to the subject a composition comprising an inhibitor of the IGPR-1 polypeptide.
2. A method of treating cancer in a subject at risk thereof, comprising administering to the subject an effective amount of a composition comprising an inhibitor of immunoglobulin containing proline rich receptor-1 (IGPR-1) protein or expression for the treatment and/or prevention of a malignancy or neoplasia disorder in the subject.
3. The method of paragraphs 1 or 2, wherein the IGPR-1 is encoded by SEQ ID NO: 2.
4. The method of any of paragraphs 1 or 2, wherein the inhibitor of IGPR-1 is a soluble extracellular domain of IGPR-1.
5. The method of any of paragraphs 1 or 2, wherein the inhibitor of IGPR-1 is a dominant negative inhibitor of IGPR-1 of SEQ ID NO: 4 or a functional fragment thereof which inhibits IGPR-1 polypeptide trans-dimerization.
6. The method of any of paragraphs 1 to 5, wherein the soluble extracellular domain of IGPR-1 comprises SEQ ID NO: 6 or SEQ ID NO: 16 or a functional fragment thereof.
7. The method of any of paragraphs 1 to 6, wherein a functional fragment of a soluble extracellular domain of IGPR-1 is at least about 60 N-terminal amino acids of SEQ ID NO: 6 or SEQ ID NO: 16.
8. The method of any of paragraphs 1 to 7, wherein a functional fragment of a soluble extracellular domain of IGPR-1 is at least about 80 N-terminal amino acids of SEQ ID NO: 6 or SEQ ID NO: 16.
9. The method of any of paragraphs 1 to 8, wherein a functional fragment of a soluble extracellular domain of IGPR-1 is at least about 100 N-terminal amino acids of SEQ ID NO: 6 or SEQ ID NO: 16.
10. The method of any of paragraphs 1 to 9, wherein the inhibitor of IGPR-1 is selected from the group consisting of: RNAi agent, oligonucleotide, antibody inhibitor, peptide inhibitor, protein inhibitor, avidimir, and functional fragments or derivatives thereof.
11. The method of any of paragraphs 1 to 10, further comprising administering to the subject an inhibitor of SPIN90 polypeptide or SPIN90 gene expression.
12. The method of paragraph 1, wherein the subject has cancer.
13. The method of paragraph 12, wherein the cancer is a cancer of the epithelium.
14. The method of paragraph 12, wherein the cancer is selected from the group consisting of: bladder cancer, Breast cancer, Bronchus cancer, cancer of the Fallopian Tube, cancer of the gastrointestinal tract, cancer of esophagus, stomach cancer, colon cancer, cancer of the rectum, cancer of the small intestine, pancreatic cancer, cancer of the placenta, prostate cancer, skin cancer, testicular cancer, thyroid cancer, cancer of the thymus, endometrium cancer, cancer of the urethra.
15. The method of paragraph 12, wherein the cancer is a Squamous Cell carcinoma (SCC), Infiltrating Duct carcinoma, adenocarcinoma, pillary carcinoma,
16. The method of paragraph 12, wherein the cancer cell type is selected from the group consisting of: urothelim, tumor cells, glandular/Lobular Epithelium cells, bronchial Epithelium cells, fallopian tube lining Epithelium cells, squamous cell carcinoma cells, adenocarcinoma cells, stomach epithelium cells, intestinal epithelium cells, colonic epithelium cells, acniar cells, trophoblastic Epithelium cells, epidermal cells, karatinocytes, skin cells, testis semiferinstubulule cells, glandular epithelium cells of the thymus, thyroid cells, urothelium cells, endometrial Glandular cells.
17. The method of any of paragraphs 1 to 16, wherein the subject is selected for treatment by identifying a subject with a cancer expressing IGPR-1.
18. The method of paragraph 1, wherein the subject has an angiogenesis-related disease characterized by increase in angiogenesis.
19. The method of paragraph 18, wherein the angiogenesis-related disease characterized by an increase in angiogenesis is selected from the group consisting of cancer, macular degeneration; diabetic retinopathy; rheumatoid arthritis; Alzheimer's disease; obesity, psoriasis, atherosclerosis, vascular malformations, angiomata, and endometriosis.
20. The method of paragraph 1, wherein the subject as neovascularization.
21. The method of paragraph 17, wherein the subject has ocular neovascularization.
22. The method of paragraph 17, wherein the subject has at least one of the disorders selected from the group comprising: age-related macular degeneration (AMD), diabetic retinopathy, retinopathy of prematurity (ROP), arthritis, rheumatoid arthritis (RA), osteoarthritis, cardiovascular disease.
23. The method of paragraph 2, wherein the cancer is a metastatic cancer, a malignant cancer or a neoplasia disorder.
24. The method of paragraph 2 or 20, wherein the cancer is of endothelial or epithelial origin.
25. The method of any of paragraphs 1 to 24, wherein the subject is a mammal.
26. The method of paragraph 25, wherein the mammal is a human.
27. The method of any of paragraphs 1 to 26, further comprising administering an anti-angiogenic therapy in conjunction with the inhibitor of the IGPR-1 polypeptide.
28. The method of paragraph 27, wherein an anti-angiogenic therapy is chemotherapy and/or radiation therapy.
29. A method of inhibiting endothelial cell migration, comprising contacting an endothelial cell with an inhibitor of immunoglobulin containing proline rich receptor-1 (IGPR-1) protein or expression.
30. The method of paragraph 29, wherein the IGPR-1 is encoded by SEQ ID NO: 2.
31. The method of any of paragraph 29 or 30, wherein the inhibitor of IGPR-1 is a dominant negative inhibitor of IGPR-1 of SEQ ID NO: 4 or a functional fragment thereof which inhibits IGPR-1 polypeptide function or IGPR-1 trans-dimerization.
32. The method of paragraph 29 or 30, wherein the inhibitor of IGPR-1 is a soluble extracellular domain of IGPR-1.
33. The method of any of paragraphs 29 to 32, wherein the soluble extracellular domain of IGPR-1 comprises SEQ ID NO: 6 or SEQ ID NO: 16 or a functional fragment thereof.
34. The method of any of paragraphs 29 to 33, wherein a functional fragment of a soluble extracellular domain of IGPR-1 is at least about 60 N-terminal amino acids of SEQ ID NO: 6 or SEQ ID NO: 16.
35. The method of any of paragraphs 29 to 34, wherein a functional fragment of a soluble extracellular domain of IGPR-1 is at least about 80 N-terminal amino acids of SEQ ID NO: 6 or SEQ ID NO: 16.
36. The method of any of paragraphs 29 to 35, wherein a functional fragment of a soluble extracellular domain of IGPR-1 is at least about 100 N-terminal amino acids of SEQ ID NO: 6 or SEQ ID NO: 16.
37. The method of any of paragraphs 29 to 36, wherein the inhibitor of IGPR-1 is selected from the group consisting of: RNAi agent, oligonucleotide, antibody inhibitor, peptide inhibitor, protein inhibitor, avidimir, and functional fragments or derivatives thereof.
38. The method of any of paragraphs 29 to 37, further comprising administering an inhibitor of SPIN90 gene expression or protein.
39. The method of paragraph 29, wherein the endothelial cell is a human endothelial cell.
40. A method of treating cancer in a subject at risk thereof, comprising administering to the subject a composition comprising an inhibitor of SPIN90 gene expression or protein or expression for the treatment and/or prevention of a malignancy or neoplasia disorder in the subject.
41. A method of inhibiting angiogenesis in a subject, comprising administering to the subject a composition comprising an inhibitor of SPIN90 gene expression or protein or expression.
42. A method to promote angiogenesis in a subject in need thereof, comprising administering to a subject a composition comprising an IGPR-1 polypeptide or a functional fragment thereof.
43. The method of paragraph 42, wherein the subject in need thereof has an angiogenesis-related disorder characterized by a decrease in angiogenesis.
44. The method of paragraph 43, wherein the subject is a transplant recipient, or has undergone a transplant surgery.
45. The method of paragraph 44, wherein the subject is a recipient of transplanted retinal pigment epithelium (RPE) cells.
46. The method of paragraph 42, wherein the subject in need thereof is in need of neovascularization of any one of the group selected from: a tissue engineering construct, an organ transplant, tissue repair, regenerative medicine, and a wound.
47. The method of paragraph 43, wherein the subject is in need thereof is in need of wound repair.
48. The method of paragraph 43, wherein the subject has had an infarct, cardiac infarct, stroke.
49. The use of a soluble extracellular domain of IGPR-1 for inhibiting angiogenesis or endothelial cell migration in a subject in need thereof, wherein the soluble extracellular domain of IGPR-1 blocks the function of the IGPR-1 polypeptide.
50. The use of a soluble extracellular domain of IGPR-1 for the manufacture of a medicament for inhibiting angiogenesis or endothelial cell migration, in a subject in need thereof.
51. The use of paragraphs 49 or 50, wherein the soluble extracellular domain of IGPR-1 comprises SEQ ID NO: 6 or SEQ ID NO: 16, or a variant or functional fragment thereof.
52. The use of paragraphs 49 to 51, wherein the soluble extracellular domain of IGPR-1 comprises at least 60 N-terminal amino acids of SEQ ID NO: 6 or SEQ ID NO: 16, or a variant or functional fragment thereof.
53. The use of paragraphs 47 to 52, wherein the soluble extracellular domain of IGPR-1 comprises at least about 80 N-terminal amino acids of SEQ ID NO: 6 or SEQ ID NO: 16, or a variant or functional fragment thereof.
54. The use of paragraphs 47 to 53, wherein the soluble extracellular domain of IGPR-1 comprises least about 100 N-terminal amino acids of SEQ ID NO: 6 or SEQ ID NO: 16, or a variant or functional fragment thereof.
55. The use of paragraphs 47 to 53, wherein a variant or functional fragment of SEQ ID NO: 6 or SEQ ID NO: 16 inhibits IGPR-1 polypeptide or cis-dimerization of IGPR-1.
56. The use of a dominant negative inhibitor of IGPR-1 for inhibiting angiogenesis or endothelial cell migration in a subject in need thereof, wherein the dominant negative inhibitor polypeptide of IGRP-1 blocks the function of the IGPR-1 polypeptide.
57. The use of a dominant negative inhibitor of IGPR-1 for the manufacture of a medicament for inhibiting angiogenesis or endothelial cell migration, in a subject in need thereof.
58. The use of paragraphs 56 to 57, wherein the dominant negative inhibitor of IGPR-1 comprises least about 60 C-terminal amino acids of SEQ ID NO: 4 or a functional fragment thereof which inhibits IGPR-1 polypeptide function or IGPR-1 trans-dimerization.
59. The use of an siRNA directed specifically against the IGRP-1 gene or the SPIN90 gene for inhibiting angiogenesis or endothelial cell migration in a subject in need thereof.
60. The use of an siRNA directed specifically against the IGRP-1 gene or the SPIN90 gene for the manufacture of a medicament for inhibiting angiogenesis or endothelial cell migration, in a subject in need thereof.

In view of the above, it will be seen that the several advantages of the invention are achieved and other advantageous results obtained. As various changes could be made in the above methods and compositions without departing from the scope of the invention, it is intended that all matter contained in this application shall be interpreted as illustrative and not in a limiting sense.

Other embodiments within the scope of the embodiments herein will be apparent to one skilled in the art from consideration of the specification or practice of the invention as disclosed herein. It is intended that the specification be considered to be exemplary only, with the scope and spirit of the invention being indicated by the embodiments.

The discussion of the references herein is intended merely to summarize the assertions made by their authors and no admission is made that any reference constitutes prior art. Applicants reserve the right to challenge the accuracy and pertinency of the cited references.

### EXAMPLES

The examples presented herein relate to the methods and compositions comprising inhibits of IGPR-1 for the treatment of cancer and cancer metastasis. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Materials and Methods

General reagents: SH3 array was purchased from Panomics, BS3 was purchased from Peirce Biotechnology. BPAG1 and CACNB2 plasmids were purchased from ATCC. SPIN90 cDNA was kindly provided by Dr. Woo Keun Song (Department of Life Science and Molecular Disease Research Center, Gwangju Institute of Science and Technology, Gwangju, Korea). BPAG1, and CANCB2 plasmids were purchased from ATCC. Immunohistochemistry/Immunofluorescence microscopy reagents including, Primary Staining Kit, DAKO EnVision system-HRP (DAB) for Rabbit Primary ABs (Cat# K4010) DAKO—AB Diluent, with background reducer (Cat#53022 DAKO), Citrate buffer (Cat#51966) DAKO-Protein Block Serum Free (Cat# X0909). Hematoxylin (Cat#AMH100907) was purchased from Thermo Scientific. SPIN90 siRNA (cat #SC-76564) and anti-SPIN90 antibody (cat #SC-79100) were purchased from Santa Cruz Biotechnology. Proliferation kit was purchased from Promega. PNGase F was purchased from New England Biolabs. Total human RNA was purchased from Biochain Inc.

Plasmids and Antibodies:

The cDNA corresponding to IGPR-1 (MGC:23244, IMAGE:4811204) which was purchased from open biosystems, was PCR amplified and cloned into retroviral vector, pMSCV.puro (Invitrogen, Inc) via Xho I and EcoR I restriction sites. IGPR-1 was cloned in frame of pcDNA3.1.His.Myc vector (Invitrogen) and the identity of IGPR-1 was confirmed by sequencing. The N-terminus-truncated IGPR-1 (ΔN-IGPR-1) was PCR amplified using IGPR-1 as a template which results in the deletion of 133 N-terminal amino acids. SH3 domains of SPIN90, BPAG1, and CANCB2 were PCR amplified and cloned into pGEX4T2 vector (Pharmacia, Inc). Polyclonal rabbit anti-IGPR-1 antibody was developed by injecting a KHL conjugated peptide corresponding to the cytoplasmic domain of IGPR-1 into rabbits. Anti-IGPR-1 antibody purified by protein-A agrose column. The specificity of the antibody was further tested by pre-incubation with the corresponding peptide with antibody which eliminated its ability to detect IGPR-1. SPIN-90 siRNA (cat# sc-76564) was purchased from Santa Cruz Biotechnology. IGPR-1 siRNAs were custom made and synthesized by Thermo Scientific/Dharmacon; CAGCAAAGGGACUCAGGUAUU (SEQ ID NO: 20) and AGGUAACAGCCCAGGAAAUUU (SEQ ID NO: 21). The qPCR primers used for IGPR-1 are CTG AGT TGG AGG AGG CTG AG (SEQ ID NO: 22) and CGA TCC GGT TTC TGT TCT GT (SEQ ID NO: 23).

Invasion Assay:

24-well transwell plates were coated with growth factor-reduced Matrigel (BD Bioscience) 10 mg/ml (100 ul/transwell) and B16F cells (1×106) expressing IGPR-1 or N-terminus deleted IGPR-1 were platted for 48 hours. After 48 hours transwells were removed and stained with Diff-Quick solution. The non-invaded cells were scraped off with a cotton swab and invaded cells were counted under a light microscope (two transwell/group and three fields from each transwell were counted).

Aggregation Assay:

Single cell suspensions were obtained with detaching of cell from plate with 1 mM EDTA in PBS solution. The cells were then washed twice in 10% DMEM medium. The cells were re-suspended in DMEM. Approximately 5×10⁵ cells per 2.5 ml were incubated in 6-well plate (pre-coated with 1% bovine serum albumin at 37° C. for 2 hours) with gentle shaking at 37° C. for 1 hour, followed without shaking for 1 hour. Cells were viewed under light microscope and pictures were taken.

Cell Adhesion Assay:

For cell adhesion assay, cells expressing IGPR-1 were detached from plate incubated in suspension for 2 hours with GST or GST-Ig domain of IGPR-1, and re-plated onto tissue culture dishes and allowed to adhere for 2 h at 37° C. Non-adherent cells were removed by washing twice with PBS, after which the adherent cells were fixed for 5 min in methanol and counted under a microscope.

RT-qPCR Analysis:

RT-qPCR reactions were performed as described by the manufacturer, and 18S was used as an internal control.

Cell Proliferation:

Proliferation assay was analyzed by measuring the capacity of cells to reduce 3-(4,5-Dimethylthiazol-2-yl)-2,5-diphenyltetrazoliumbromide (MTT) to formazan and assay was performed as manufacturer's recommendation.

Mouse Matrigel Angiogenesis Assay:

Mice (6 animals for each experimental group) were injected with matrigel (10 mg/ml), plus B16 melanoma cells (1×10⁷) which were engineered to express IGPR-1 or empty vector. Before injection, the animals were sedated with Avertin (0.3 ml per 20 g mouse). Using 25-gauge needle 0.3 ml of matrigel mixture was injected sub-dermally into mice. After 8 days animals were scarified and tumor-induced angiogenesis and matrigel plugs were removed for further analysis as described (Meyer et al., 2011a). The tumor plugs were homogenized in 1 mL of deionized H₂O on ice and cleared by centrifugation at 10,000 rpm for 5 minutes at 4° C. The supernatant was collected and used in duplicate to measure hemoglobin content with Drabkin's reagent along with hemoglobin standards as suggested by the manufacturer (Sigma/Aldrich Chemical). The absorbance was read at 540 nm.

Endothelial Cell Capillary Tube Formation and Migration Assays:

Endothelial cells were seeded on matrigel with endothelial cell growth medium (Clonetics Co., San Diego, Calif., USA) and capillary tube formation was viewed under microscope and photographed after 24 hours as described ((Meyer et al., 2008). Quantification of capillary tube formation was established by NIH Image J program. Migration assay was performed basically by creating a “wound” in a cell monolayer by scratch using the tip of the tissue culture pipette. After 10 hours images were captured under microscope and documented.

Migration/Wounding Assay:

Wounding assay was performed by wounding the monolayer of cells expressing IGPR-1 or other plasmids as indicated in the figure legends of each cells by scraping with an 5 nil tissue culture pipette, washed, and incubated with 10% FBS in DMEM for 6 hours. Migration was assessed visually under light microscope and pictures were taken and presented. In some occasions cells were subjected to modified Boyden chamber migration assay. Briefly, to measure cell motility, Transwell culture inserts (8-mm pore size) (Costar, Inc.) were coated uniformly with gelatin (0.25% w/v, Sigma) on both sides for 2 min at room temperature. Membranes were washed twice with serum-free DMEM medium and inserted into a 24-well culture plate with DMEM plus 10% FBS. Cells (2×104 in 100 μl) were plated in the insert and incubated for 6-8 h at 37° C. Following the incubation, excess medium was removed, and cells were fixed and cells on the upper side of the membrane were removed by wiping with cotton. Cells on the underside of the membrane were counted under microscope. Cell motility is expressed as the number of migrating cells per well (three fields/well were randomly counted).

SH3 Array Analysis:

The SH3 array (purchased from panomics, Inc). The array contains 34 SH3 domains derived from 34 individual proteins. The array membrane was incubated with purified GST-IGPR-1 (cytoplasmic region) for one hour and after washing with Western rinse buffer, the membrane was incubated with anti-IGPR-1 antibody. After one hour incubation, the membrane washed and incubated with HRP-anti-rabbit antibody and detected with chemiluminescent reagent.

GST-Pull Down Assay:

In vitro GST fusion protein binding experiments were performed as described (Meyer et al., 2008). Briefly, equal numbers of cells were grown to 90% confluency. Cells were lysed in ice-cold lysis buffer supplemented with 2 mM Na₃VO₄ and a protease inhibitor cocktail. Equal amounts of the appropriate immobilized GST fusion proteins were incubated with normalized whole cell lysates by rocking for 3 h at 4° C. The beads were washed four times in the presence of protease inhibitors and Na₃VO₄, and proteins were eluted and analyzed in Western blot analysis using IGPR-1 antibody.

Site-Directed Mutagenesis:

All the site-directed mutagenesis was performed using PCR-based site directed mutagenesis strategy (Meyer et al., 2004; Meyer et al., 2009). The identities of deletions were confirmed by sequencing the plasmids. All the cDNAs were either cloned into pcDNA3.1His.Myc vector or into retroviral vector, pMSCV.puro (clontech, Inc). In some cases the PCR products were cloned into pGEX2T vector and used to make GST-fusion protein in E. Coli.

Virus Production and Transient Transfection:

For virus production pMSCV.puro vector containing IGPR-1 or other cDNA of interest were transfected into 293-GPG cells and viral supernatants were collected for 7 days, concentrated by centrifugation and used as previously described (Rahimi et al., 2000; Meyer et al., 2009).

Immunohistochemical Analysis:

Normal human tissue microarray (cat# MC0961) and human tumor microarray were purchased from Us BioMax Inc (Rockville, Md.). Tumor microarray (cat#MC613) is a multiple organ, tumor microarray, 3 cases of each type of 19 types of cancer (bladder, breast, cervix, colon, esophagus, heart, kidney, liver, lung, ovary, pancreas, prostate, rectum, skin, stomach, testis, thyroid and uterus) and 1 prostate hyperplasia plus 3 normal tissues, single core per case. Immunohistochemical staining was performed using rabbit anti-IGPR-1 antibody. After standard formalin fixation and paraffin embedding (FFPE) all FFPE slides were subjected to heat induced epitope retrieval through the BioCare Medical Decloaking chamber, with citrate buffer at pH 6.0. Antibody detection was preformed with anti-Rabbit-HRP Envision+ kit (Dako) with extra-blocking with serum free protein block (Dako) for 1 hour.

Immunoprecipitation and Western Blotting:

Cells were prepared and lysed as described (Meyer et al., 2005). Briefly, cells were washed twice with H/S buffer (25 mM HEPES, pH 7.4, 150 mM NaCl, and 2 mM Na₃VO4) and lysed in lysis (EB) buffer (10 mM Tris-HCl, 10% glycerol, pH 7.4, 5 mM EDTA, 50 mM NaCl, 50 mM NaF, 1% Triton X-100, 1 mM phenylmethylsulfonyl fluoride [PMSF], 2 mM Na₃VO4, and 20 m/ml aprotinin). Where indicated, equal amounts of protein representing equal numbers of cells from total cell lysates were either directly resolved by SDS-PAGE or were immunoprecipitated by using appropriate antibodies before SDS-PAGE and immunoblotting. Normalized whole cell lysates were subjected to Western blot analysis using IGPR-1 antibody or with appropriate antibody as indicated in the figure legends. In some instances, the membranes were stripped by incubating them in a buffer containing 6.25 mM Tris-HCl, pH 6.8, 2% SDS, and 100 mM (3-mercaptoethanol in 50° C. for 30 min and re-probed with the desired antibody. Cell surface biotinylation was performed as following. Equal numbers of PAE cells expressing IGPR-1 were washed three times with ice-cold PBS. Surface proteins were biotinylated by incubating cells with 0.5 mg/ml of the membrane-impermeable biotin analog EZ-Link™ Sulfo-NHS-LC-Biotin (sulfosuccinimidyl-6-(biotinamido) Hexanoate) (Pierce) in ice-cold PBS (pH 7.4) for 30 min at 4° C. Unreacted biotin was quenched and removed by three washes with ice-cold H/S buffer (25 mm HEPES, pH 7.4, 150 mm NaCl, and 2 mm Na₃VO4) at 4° C. Biotinylated cells were lysed and equal amounts of protein from each lysate were immunoprecipitated using a rabbit polyclonal anti-IGPR-1 antibody and immunoprecipitated proteins subjected to Western blot analysis using streptavidin-horseradish peroxidase conjugate and were detected by ECL system (Amersham Biosciences).

Immunofluorescence Microscopy:

PAE cells expressing IPR-1 were fixed with 4% Paraformaldehyde for 30 minutes and after washing with PBS (3×) permablized with 0.01% Triton X-100 for 1 min. Cells were incubated with 5% bovine serum albumin (BSA) for 45 minutes with rocking and then incubated with anti-IPR-1 antibody and after extensive washing and incubation with secondary antibody were mounted with anti-fade/4′,6-diamidino-2-phenylindone (DAPI) (vectastain) (Vector laboratories, Burlingame, Calif.)

### Example 1

Herein, the inventors have demonstrated that IGPR-1 functions as a cell adhesion receptor with a broad expression in normal human organs and tissues mainly by epithelial and endothelial cells. IGPR-1 undergoes cis- and trans-dimerization and through its unique proline-rich cytoplasmic motif recruits several key signaling proteins involved in cell adhesion and cytoskeleton rearrangement. IGPR-1 inhibits migration and invasion of human colorectal tumor cell lines, HT-29 and HCT-116. The inventors have therefore demonstrated that IGPR-1 modulates cellular migration and adhesion, and in particular, overexpression or agonist activation of IGPR-1 or SPIN90 has an anti-metastatic and anti-invasive function and can inhibit cellular migration.

Identification of IGPR-1 as a Novel Cell Surface Receptor

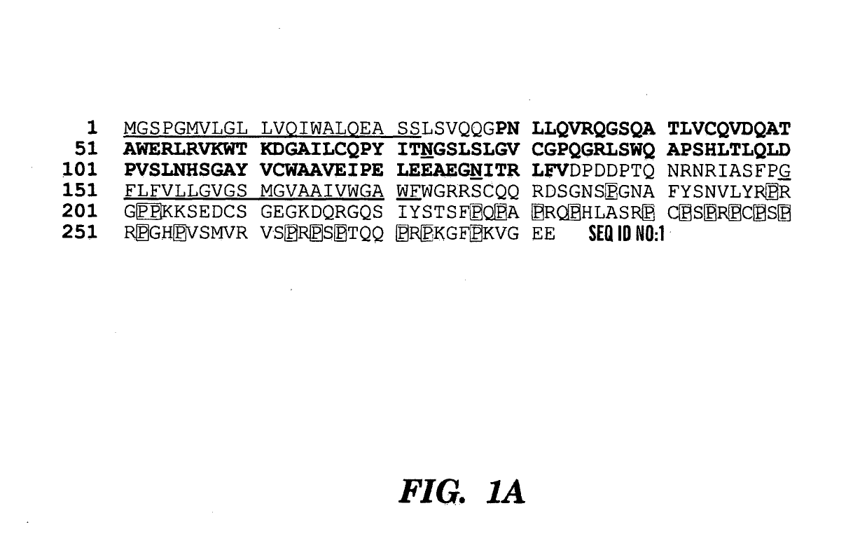

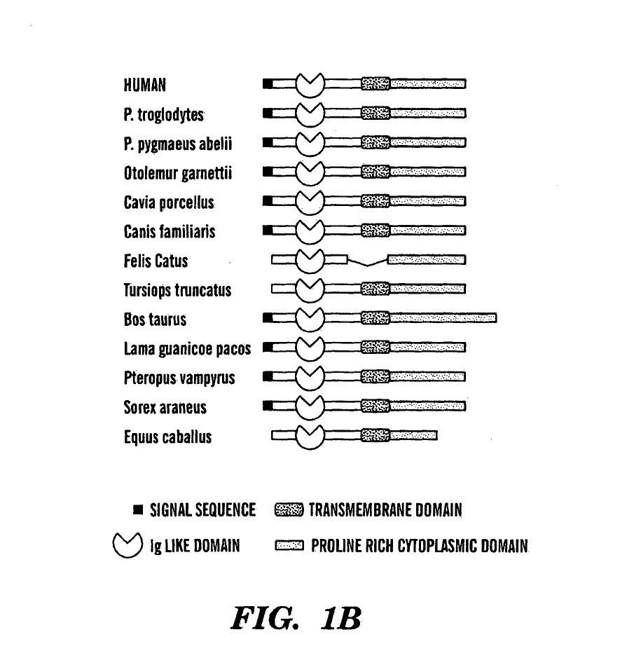

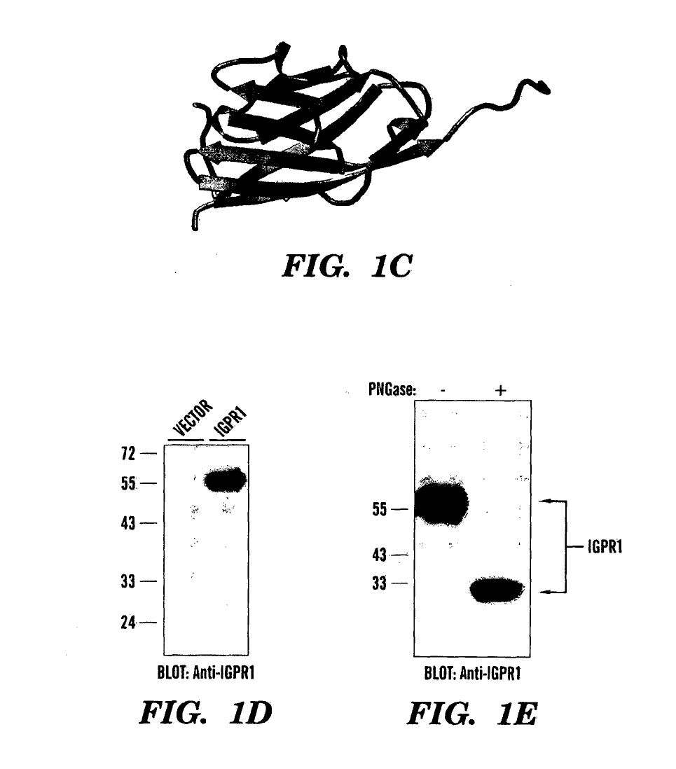

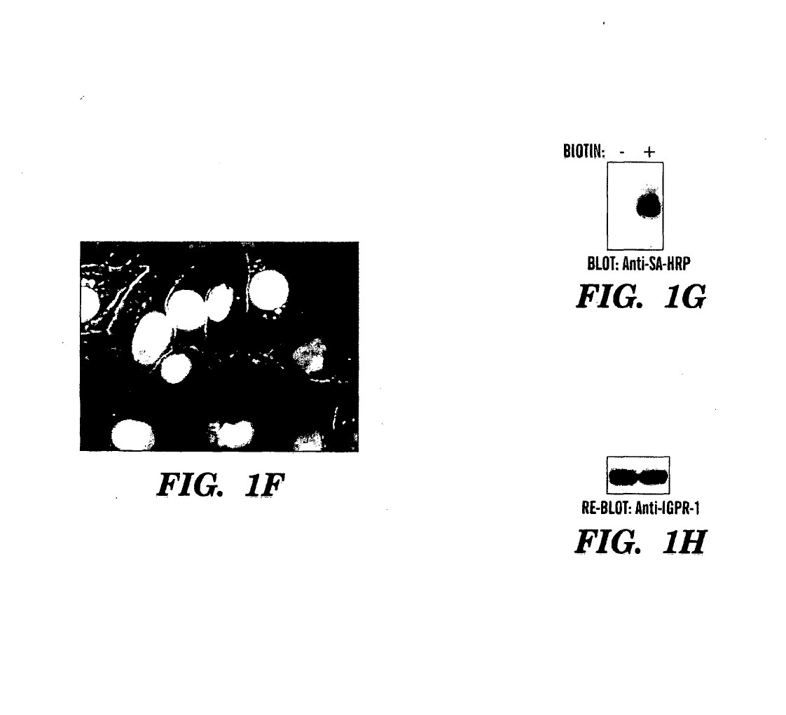

Searching the human genome sequence database for Ig containing proteins inventors identified an uncharacterized protein, transmembrane and immunoglobulin domain containing protein 2 (TMIGD2) that has a single immunoglobulin domain (Ig domain), a single transmembrane domain and a stretch of 110 amino acid cytoplasmic region highly rich in proline residues (FIG. 1A). The extracellular region of this protein also contains two possible glycosylation sites (FIG. 1A). Because of the presence of an immunoglobulin domain in its extracellular region, and presence of proline-rich motif in its cytoplasmic region, inventors named this protein immunoglobulin and proline-rich receptor-1 (IGPR-1). Ortholog of human IGPR-1 is found only in eukaryotes including, primates, guinea pig, canines, feline, dolphin, bovine, llama, bat, common Shrew, and horse (FIG. 1B). Interestingly, IGPR-1 gene is absent in mouse and rat genomes. The immunoglobulin domain of IGPR-1 was predicted to be Ig V (variable) fold and found highly similar to the Ig domain of myelin-associated glycoprotein (MAG) (14). Using IPKO as a template we constructed a structural model of IGPR-1. IGPR-1 seems to adapt a typical IgV-like fold consisting of a sandwich of two anti-parallel β-sheets (FIG. 1C).

In order to examine its cellular and biochemical properties, IGPR-1 was cloned into a retroviral expression vector and expressed in PAE (porcine aortic endothelial) cells. Moreover, to detect expression of IGPR-1, the inventors developed a polyclonal anti-IGPR-1 antibody against its cytoplasmic domain which specifically recognizes IGPR-1 (FIG. 1D). The predicted molecular weight of IGPR-1 protein is 31 kDa, however, the apparent molecular weight of IGPR-1 ectopically expressed in PAE cells as detected by Western blot analysis was approximately 55 Kda (FIG. 1D). The inventors assessed if a higher molecular weight of IGPR-1 was due to glycosylation at its extracellular region. Treatment of cell lysates derived from PAE cells with PNGase (N-Glycosidase F), which is known to hydrolyze nearly all types of N-glycan chains from glycoproteins, generated a 31 Kda protein (FIG. 1E), demonstrating that IGPR-1 is highly glycosylated in vivo and that glycosylation is responsible for its apparent high molecular weight. Interestingly, treatment of cells with tunicamycin, a commonly used inhibitor of N-linked glycosylation in vivo which is known to cause ER stress also caused a rapid degradation of IGPR-1, demonstrating that glycosylation of IGPR-1 is important for its stability and agents that induce ER stress can promote the degradation of IGPR-1 (data not shown). Since IGPR-1 is predicted to be a plasma membrane protein, the inventors also analyzed its membrane localization in PAE cells. The immunofluorescence microscopy assessment of cells expressing IGPR-1 showed that IGPR-1 is localized in the plasma membrane (FIG. 1F). Additional analysis including cell surface biotinylation further demonstrated cell surface localization of IGPR-1 (FIG. 1G). Taken together, the inventors have established that IGPR-1 is a novel immunoglobulin containing membranous glycoprotein.

### Example 2

### IGPR-1 is Expressed in Various Human Organs and Cells

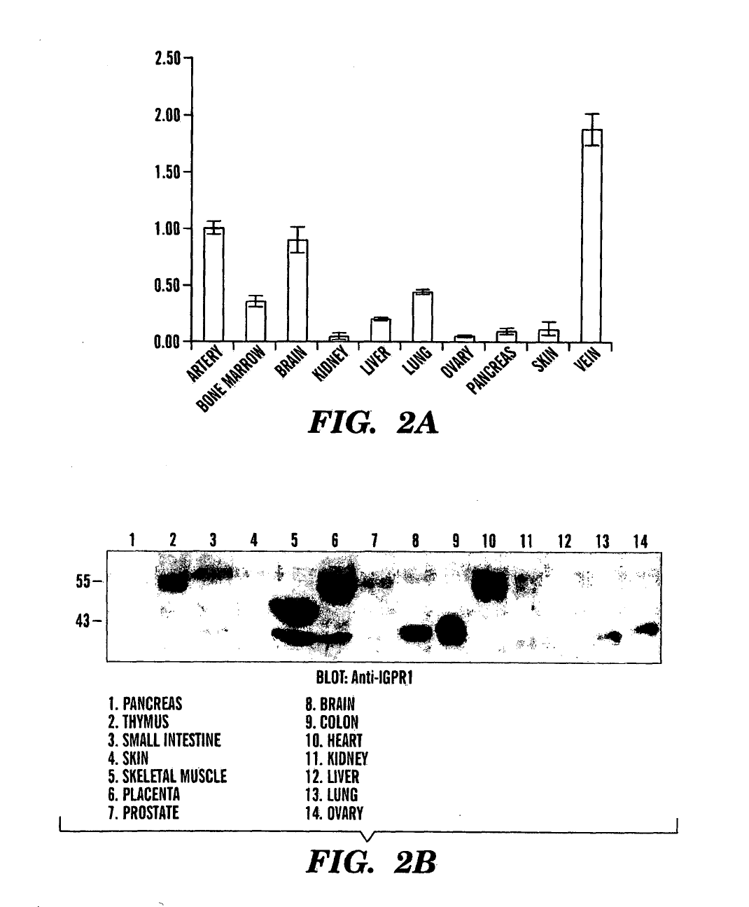

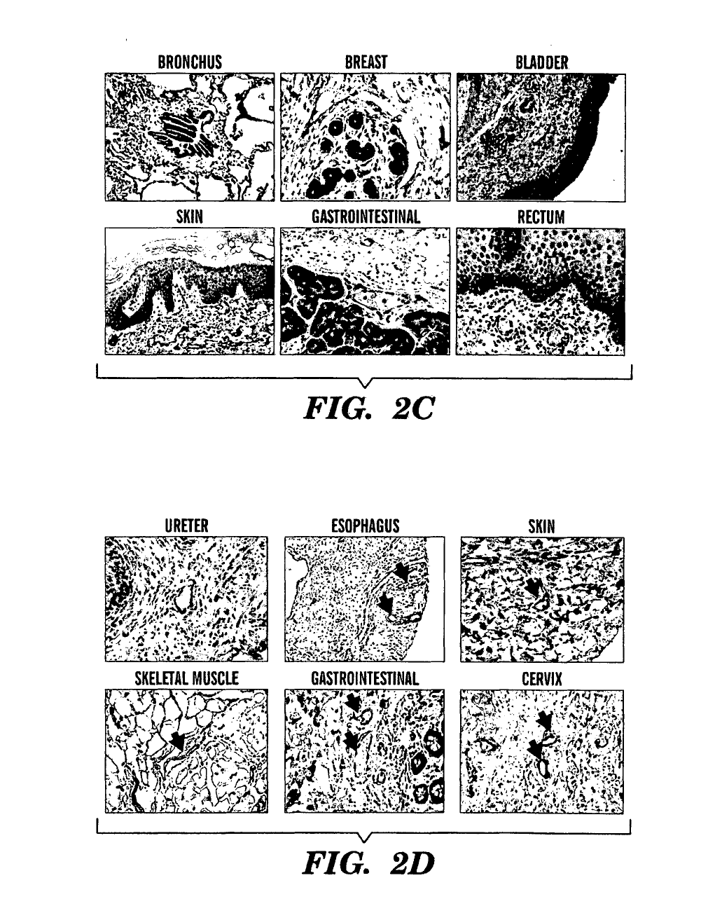

To gain insight into the tissue distribution of IGPR-1, the inventors examined its expression in normal human tissues, by analyzing the transcript of IGPR-1 by qPCR. The qPCR analysis using primers designed for exon 2 and 3 of IGPR-1 showed that IGPR-1 transcript was highly present in artery, vein and brain (FIG. 2A). IGPR-1 transcript was also moderately detected in bone marrow, liver and lung. IGPR-1 transcript, however, was relatively low in kidney, ovary, pancreas and skin (FIG. 2A). Additional analysis using qPCR primers designed for exon 3 and 4 yielded similar results (data not shown), confirming that IGPR-1 transcript is broadly present in these tissues. To examine expression of IGPR-1 in human tissues at protein level, cell lysates derived from various human organs/tissues were analyzed for expression of IGPR-1. As shown IGPR-1 protein was detected in thymus, placenta, heart, small intestine, skin and kidney with an apparent molecular weight of 55 kDa (FIG. 2B). Interestingly, cell lysates derived from skeletal muscle, brain, colon, lung and ovary IGPR-1 was detected with an apparent molecular weight of 35 kDa (FIG. 2B), suggesting that perhaps IGPR-1 in these cell lysates is not fully glycosylated, albeit one could not discount possible proteolytic degradation due to sample preparation and handling

To further determine cellular distribution of IGPR-1, the inventors used a normal human tissue microarray consisting of major human organs/tissues and subjected to immunohistochemical analysis using anti-IGPR-1 antibody. Immunohistochemical staining showed that IGPR-1 is mainly expressed by cells with epithelial origin, including bronchial epithelial cells of lung, breast glandular and lobular epithelia cells, urothelium of the bladder, skin epidermis, epithelium of gastrointestinal and rectum (FIG. 2C). Moreover, endometrial glands of the uterus, the ureter, fallopian tube epithelium, colonic epithelium, small bowl epithelium, stomach epithelium including both chief and parietal cells, trophoblastic epithelium of placenta, and pancreatic acinar cells were all positive for IGPR-1 (data not shown). Off note, thyroid, cerebellum, cerebral cortex, and thymus were negative for IGPR-1 (data not shown). Moreover, endometrial glands of the uterus (N=3), the ureter (N=2), fallopian tube epithelium (N=3), colonic epithelium (N=3), small bowl epithelium (N=3), stomach epithelium including both chief and parietal cells (N=3), trophoblastic epithelium of placenta, and pancreatic acinar cells (N=3) were all positive for IGPR-1 (Table 1). The tissue distribution of IGPR-1 in other organs is summarized in table 1. Beyond epithelial cells that were positive for IGPR-1 across the tissue microarray staining, endothelial cells present in vein and arteries also consistently were positive for IGPR-1, as shown in tissue sections derived from ureter, esophagus, skin, skeletal muscle, gastrointestinal, and cervix (FIG. 2D). As noted, IGPR-1 transcript was also highest in vein and artery (FIG. 2A). Altogether, the data demonstrate that IGPR-1 is expressed in various organs however its expression is mainly present in cells with epithelial and endothelial cell types.

TABLE 1
Cancer from tissue Types expressing IGPR-1.

| | | Positive Staining | |
|---|---|---|---|
| Tissue Type | Pathology Diagnosis | detected | Positive/total |
| Adrenal Gland | Normal | — | 0 of 3 |
| Bladder | Normal | Urothelim | 3 of 3 |
| Bladder | Squamous Cell carcinoma | Tumor cells | 2 of 3 |
| Bone Marrow | Normal | — | 0 of 1 |
| Brain | Astrocytoma | — | 0 of 3 |
| Breast | Normal | Glandular/Lobular | 4 of 4 |
| | | Epithelium | |
| Breast | Infiltrating Duct carcinoma | Tumor cells | 2 of 3 |
| bronchus | Normal | Bronchial Epithelium | 1 of 3 |
| eye | Normal | — | 0 of 3 |
| cerebellum | Normal | — | 0 of 3 |
| Cerebral Cortex | Normal | — | 0 of 3 |
| Fallopian Tube | Normal | Lining Epithelium | 3 of 3 |
| GI - Esophagus | Normal | — | 0 of 3 |
| GI - Esophagus | Squamous Cell Carcinoma | Tumor cells | 2 of 3 |
| GI - Stomach | Normal | Stomach Epithelium | 3 of 3 |
| GI - stomach | Adenocarcinoma | Tumor cells | 2 of 3 |
| GI - Small intestine | Normal | Intestinal Epithelium | 3 of 3 |
| GI - Colon | Normal | Colonic Epithelium | 3 of 3 |
| GI - Colon | Adenocarcinoma | Tumor cells - | 3 of 3 |
| | | Punctate | |
| GI-Rectum | Normal | — | 0 of 3 |
| GI-Rectum | Adenocarcinoma | Tumor cells | 3 of 3 |
| Heart | Normal | — | 0 of 3 |
| Kidney | Normal | — | 0 of 3 |
| Kidney | Paraganglioma | — | 0 of 3 |
| liver | Normal | — | 0 of 3 |
| Liver | Hepatocellular carcinoma | — | 0 of 3 |
| Lung | Normal | — | 0 of 3 |
| Lung | Squamous Cell Carcinoma | — | 0 of 3 |
| Ovary | Normal | — | 0 of 3 |
| ovary | Celar Cell Carcinoma | — | 0 of 3 |
| Pancreas | Normal | Acniar cells | 3 of 3 |
| Pancreas | Adrenocarcinoma | Tumor Cells | 3 of 3 |
| Parathyroid | Normal | — | 1 of 1 |
| Pituitary Gland | Normal | — | 2 of 2 |
| Placenta | Normal | Trophoblastic | 1 of 3 |
| | | Epithelium | |
| Prostate | Normal | Epidermis - | 3 of 3 |
| | | Karatinocytes | |
| Prostate | Adrenocarcinima | Tumor Cells | 3 of 3 |
| Skin | Normal | Epidermis | 3 of 3 |
| Skin | Squamous Cell Carcinoma | Tumor cells - light | 2 of 3 |
| Spinal Cord | Normal | — | 0 of 3 |
| Spleen | Normal | — | 0 of 3 |
| Striated Muscle | Normal | — | 0 of 3 |
| Testis | Normal | Semiferinstubules | 3 of 3 |
| Testis | Lymphoma | — | 0 of 3 |
| Thymus | Normal | Glandular Epithelium | 3 of 3 |
| Thyroid | Normal | — | 0 of 3 |
| Thyriod | Pipillary Carcinoma | Tumor Cells | 3 of 3 |
| Tonsil | Normal | — | 0 of 3 |
| Ureter | Normal | Urothelium | 3 of 3 |
| Uterus - cervix | Normal | — | 0 of 3 |
| Uterus - | Normal | Endometrial | 3 of 3 |
| endometrium | | Glandular Epithelium | |
| Uterus - | Squamous Cell Carcinoma | Tumor Cells | 2 of 3 |
| endometrium | | | |

Expression of IGFR-1 in Human Tumors.

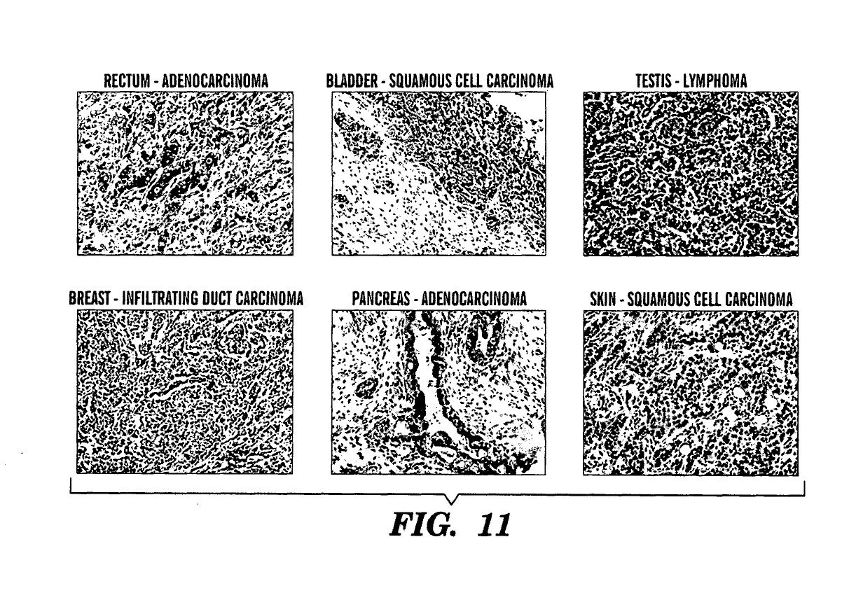

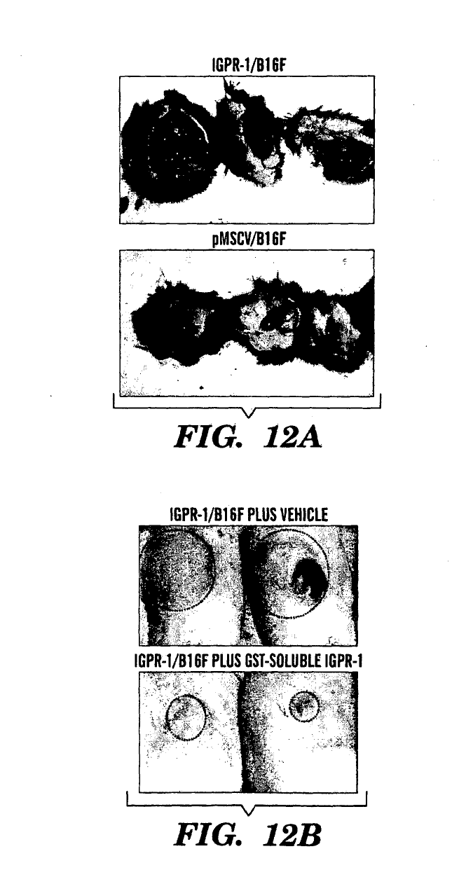

To further examine possible biological importance of IGPR-1 in human cancers, inventors analyzed IGPR-1 expression in human cancers. Analysis of a human tumor tissue microarray consisting of 17 different common cancer types showed that overall IGPR-1 is expressed in various human tumor tissues is shown in Table 2. IGPR-1 staining was positive for the squamous cell carcinoma (grade III) of the esophagus (N=2 of 3), adenocarcinoma (grade II) of the stomach (N=1 of 3), adenocarcinoma (grade II) of the rectum (N=3), squamous cell carcinoma (grade I) of the bladder (N=2 of 3), adenocarcinoma (grade III) of the pancreases (N=3), squamous cell carcinoma (grade II) of the uterus (N=2 or 3), and finally adenocarcinoma (grade III) of the prostate (N=3) (FIG. 11). Of note, a strong staining of IGPR-1 was observed only in the papillary carcinoma (grade II) of the thyroid (FIG. 11).

Interestingly, IGPR-1 staining in some tumors including, infiltrating ductal carcinomas of the breast (N=3), adenocarcinoma (grade III) of the pancreases (N=3), papillary carcinoma (grade I) of the thyroid, squamous cell carcinoma (grade II) of the skin (N=3), hepatocellular carcinoma (grade III) of the liver (N=3), squamous cell carcinoma (grade III) of the lung (n=3), paraganglioma of the kidney (N=3), lymphoma of the testis (N=3) and clear cell carcinoma of the ovary (N=3) was below levels of detection (FIG. 11). The data demonstrate that expression of IGPR-1 is differentially regulated in tumor cells depending to their stage/development and other parameters associated with pathobiology of tumor cells.

TABLE 2
17 different human common cancer types that express IGPR-1.

| | | | Number of |
|---|---|---|---|
| | | IGPR-1 | Positive/ |
| | | staining | total |
| Tissue Type | Pathology Diagnosis | observed | analyzed |
| GI-stomach | Adenocarcinoma | + | 2 of 3 |
| GI-Colon | Adenocarcinoma | + | 3 of 3 |
| GI-Rectum | Adenocarcinoma | + | 3 of 3 |
| Pancreas | Adenocarcinoma | + | 3 of 3 |
| Prostate | Adenocarcinoma | + | 3 of 3 |
| Brain | Astrocytosma | − | 0 of 3 |
| Ovary | Celar cell carcinoma | − | 0 of 3 |
| liver | Hepatocellular carcinoma | − | 0 of 3 |
| Breast | Infiltrating Duct Carcinoma | + | 3 of 3 |
| Testis | Lymphoma | − | 0 of 3 |
| Thyroid | Papillary carcinoma | ++ | 3 of 3 |
| Kidney | Paraganglioma | − | 0 of 3 |
| Bladder | Squamous Cell Carcinoma | + | 2 of 3 |
| GI - Esphagus | Squamous Cell Carcinoma | + | 2 of 3 |
| Lung | Squamous Cell Carcinoma | − | 0 of 3 |
| Skin | Squamous Cell Carcinoma | −/+ | 2 of 3 |
| Uterus - | Squamous Cell Carcinoma | + | 2 of 3 |
| endometrium | | | |

### Example 2

### IGPR-1 Activity Regulates Angiogenesis In Vivo and In Vitro

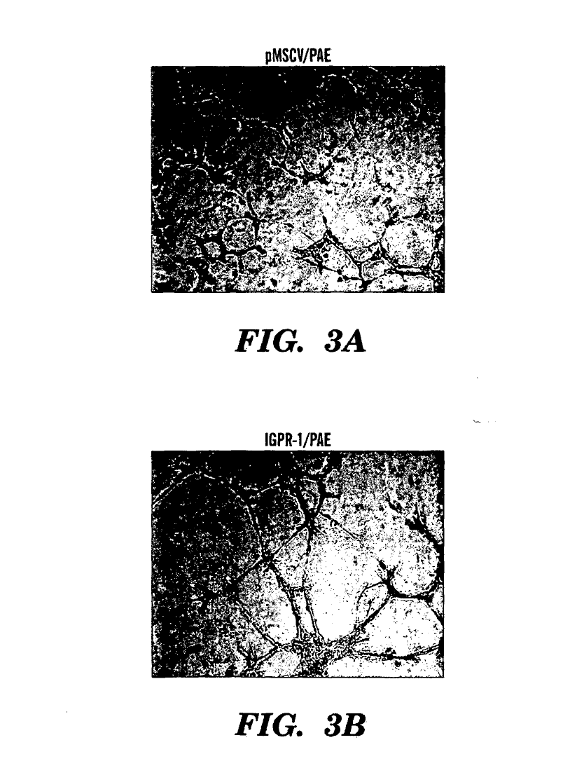

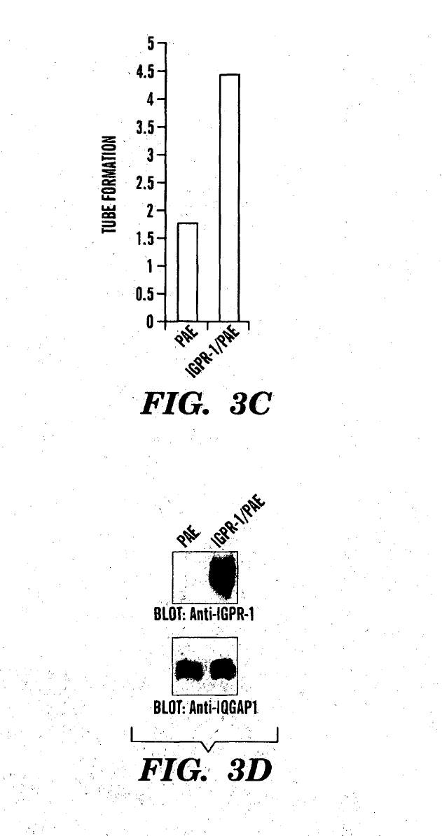

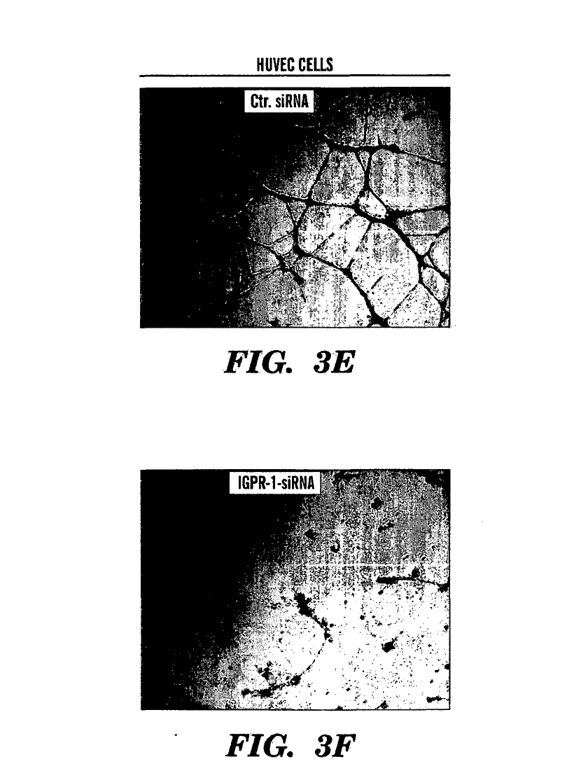

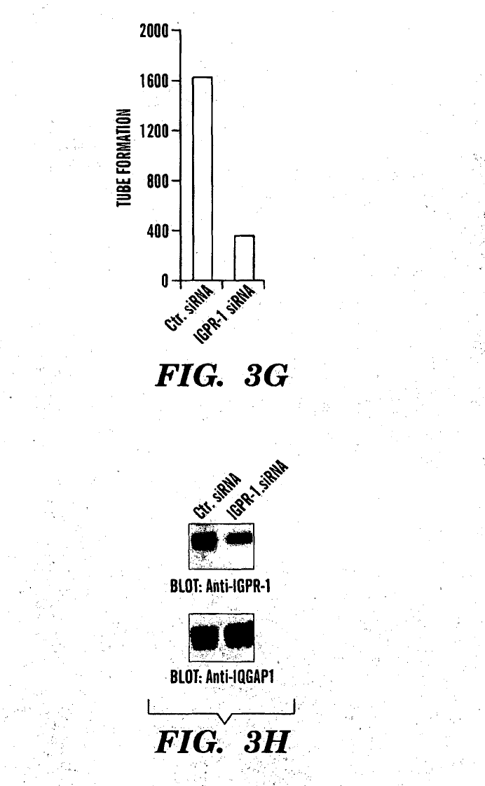

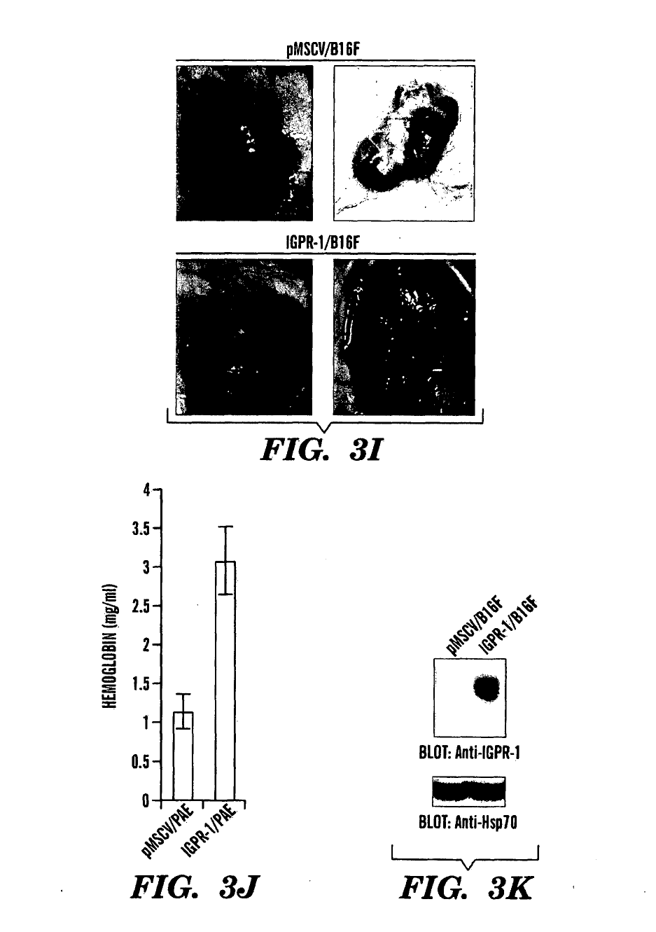

Adhesion molecules are known to regulate capillary tube formation of endothelial cells and angiogenesis (6,14). IGPR-1 is expressed by endothelial cells (FIG. 2D), indicating a role of IGPR-1 in angiogenesis. To examine role of IGPR-1 in angiogenesis, the inventors subjected PAE (porcine aortic endothelial) cells over-expressing IGPR-1 to an in vitro matrigel-based angiogenesis assay. The inventors demonstrated that PAE cells engineered to express IGPR-1 undergo increased capillary tube formation (FIG. 3A as compared to 3B). Quantification of capillary tube formation of PAE cells and expression of IGPR-1 in PAE cells also are shown (FIGS. 3C and 3D).

To examine role of IGPR-1 in angiogenesis, in particular role of endogenous IGPR-1 in capillary tube formation of endothelial cells, the inventors silenced expression of IGPR-1 in HUVEC cells by siRNA. IGPR-1 siRNA significantly reduced expression of IGPR-1 in HUVEC cells (FIG. 3H). Knockdown of IGPR-1 by siRNA also resulted in a significant reduction in the capillary tube formation of HUVEC cells (FIG. 3F). To address in vivo potential of IGPR-1 in angiogenesis, B16F melanoma cells were engineered to express IGPR-land subjected to in vivo matrigel plug angiogenesis assay. Expression of IGPR-1 in B16F cells significantly increased B16F cells potential to stimulate angiogenesis as blood vessels surrounding tumor mass are visibly notable in B16F cells expressing IGPR-1 (FIG. 3I). Angiogenesis associated with tumor cells was further quantified by measuring the hemoglobin amount in the tumor cells (FIG. 3J). Expression of IGPR-1 in B16F cells is shown (FIG. 3K). Taken together, the data demonstrates that expression of IGPR-1 in endothelial cells regulates angiogenesis.

### Example 3

### IGPR-1 Promotes a Morphological Change and Regulates Focal Adhesion

The inventors noted that PAE cells ectopically expressing IGPR-1 display a distinct morphological change. PAE cells expressing IGPR-1 displayed rectangular shape morphology and were appeared to be in tight contact, where the typical morphology of PAE cells are more elongated (FIG. 4A). To demonstrate morphological changes associated with IGPR-1 in PAE cells we stained these cells for actin filament formation using FITC-labeled phalloidin. Expression of IGPR-1 in PAE cells significantly increased actin filament formation ((FIG. 4A) indicating that IGPR-1 activity induces morphological changes in PAE cells. In general, adhesive properties of a given cell type determines trypsinization time in cell culture. PAE cells expressing IGPR-1 were also resistant to trypsinization. PAE cells within 2-5 minutes treatment with tissue culture trypsin/EDTA medium become fully dislodged from tissue culture plate as they all showed a round up morphology. However, most of PAE cells expressing IGPR-1 after 5 minutes treatment with trypsin/EDTA medium were still attached to plate and mostly retained their adherent morphology (FIG. 4B). After 8-10 minutes eventually all were detached from plate. The observation demonstrates that IGPR-1 is involved in regulation of cell morphology and adhesion.

Since over-expression of IGPR-1 increases adhesive phenotype of PAE cells, the inventors next examined the role of IGPR-1 in cell-cell interaction. To this end, PAE cell lines expressing IGPR-1 and ΔN-IGPR-1 (where the extracellular domain of IGPR-1 was deleted) were generated and subjected to aggregation assay. Expression of IGPR-1 and ΔN-IGPR-1 in PAE cells is shown (FIGS. 4C and 4D). PAE cells expressing wild type IGPR-1 but not ΔN-IGPR-1 or empty vector formed large aggregates of cells (FIG. 4E-4G), demonstrating that IGPR-1 mediates homophilic cell-cell interaction and extracellular domain is necessary for its function to mediate cell-cell interaction. To examine role of IGPR-1 in cell adhesion and the role of its immunoglobulin containing extracellular domain in this process we generated a recombinant GST-immunoglobulin (Ig) containing extracellular domain of IGPR-1 (FIG. 4H). PAE cells expressing IGPR-1 and PAE cells expressing empty vector were pre-incubated with GST protein both adhered to plate in as similar manner. However, incubation of PAE cells expressing IGPR-1 with soluble extracellular domain of IGPR-1 totally inhibited adhesion/spreading of these cells to plate (FIG. 4I). As noted pre-incubation of cells with soluble IGPR-1 only were loosely adhered with no spreading (FIG. 4I) and they come off the tissue plate (data not shown). Quantification of adherent cells also is shown (FIG. 4J). Altogether, the data demonstrate that IGPR-1 activity regulates cell-cell interaction and adhesion and its extracellular domain is critically important for its ability to mediate these cellular events.

### Example 4

### IGPR-1 Activity Regulates Focal Adhesion and Cell Migration

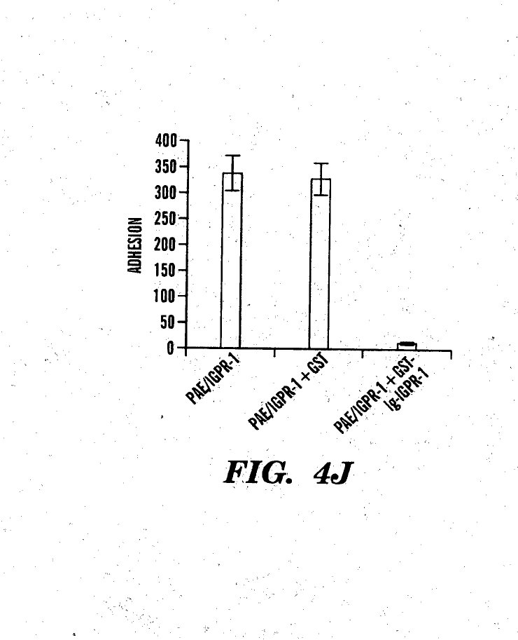

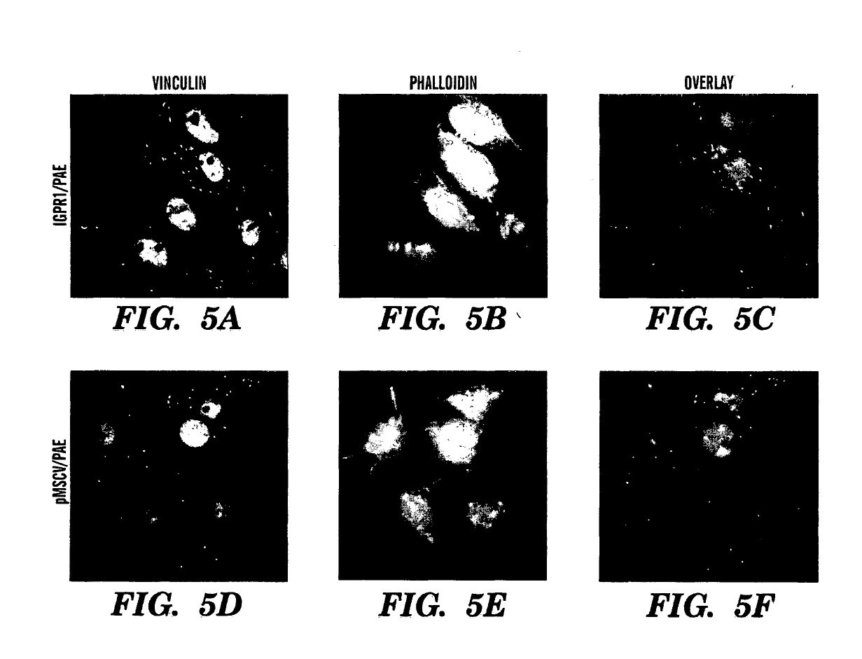

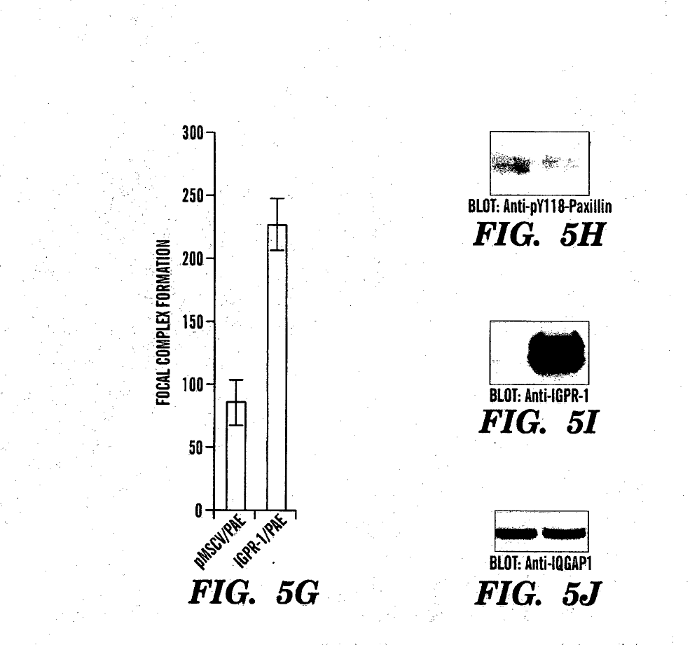

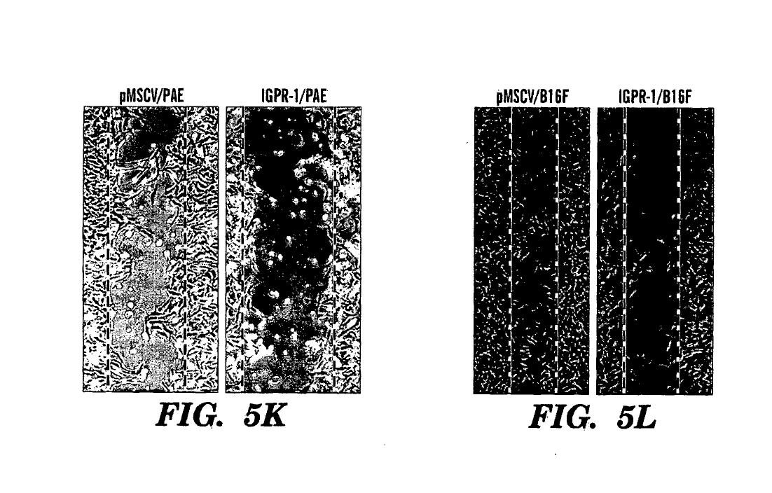

To further investigate biological responses associated with IGPR-1 activity in PAE cells the inventors analyzed focal adhesion formation in PAE cells. Focal adhesion regulates key cellular functions including, cell morphology, motility, survival, and ability to adhere to a substrate. Immunofluorescence staining of PAE cells with vinculin demonstrated that expression of IGPR-1 in PAE cells markedly increases focal adhesion as measured by vinculin localization with respect to focal contact in these cells (FIG. 5A). Of note, IGPR-1 expression in PAE cells increased both number and size of focal adhesions (FIG. 5A). Interestingly, adhesions in PAE cells expressing IGPR-1 were higher at the cells' periphery and consistent with their observed morphology, they were aligned along each other with a tight contact (Compare FIG. 5C to 5F). Quantitative analysis of number of focal adhesions (i.e., based on the number of focal adhesions) showed that IGPR-1 expression in PAE cells resulted in an increased number focal adhesion by 62% (FIG. 5G). Phalloidin staining of cells for actin with FITC-labeled phalloidin also demonstrated that expression of IGPR-1 in PAE cells alters actin stress fibers and cytoskeleton remodeling. PAE cells expressing empty vector forms stress fibers primarily at periphery of cells with membrane ruffling which is reflective of actin filament assembly (FIG. 5F). PAE cells expressing IGPR-1, however, displayed a distinct actin stress fiber formation mainly in central regions of the cells with a significant reduced level of membrane ruffles (FIG. 5B compared to 5E). Taken together, the data demonstrate that IGPR-1 regulates focal adhesion and actin stress fiber remodeling.

Because IGPR-1 activity regulates focal adhesion and reduced membrane ruffles, which are linked to cellular migration, the inventors next analyzed the function of IGPR-1 in cell migration. As the activity of various proteins including Paxillin, an adaptor protein that localizes to focal adhesion which have been implicated in the regulation of different steps of cell migration (Abou-Zeid et al., 2006; Abou Zeid et al., 2006), the inventors measured phosphorylation of paxillin. The expression of IGPR-1 in PAE cells reduces phosphorylation of paxillin (FIG. 5H). Phosphorylation of paxillin is known to inhibit cell migration (Abou-Zeid et al., 2006; Abou Zeid et al., 2006; Zaidel-Bar et al., 2007) demonstrate that IGPR-1 activity inhibits cell migration. To address direct role of IGPR-1 in cell migration the inventors subjected PAE cells and B16F cells expressing IGPR-1 to a wounding assay. The expression of IGPR-1 in two different cell types (PAE and B16F cells) significantly inhibited cell migration (FIGS. 5K and 5L). Taken together, the inventors have demonstrated that IGPR-1 stimulates dephosphorylation of paxillin and inhibits cellular migration.

### Example 5

### IGPR-1 Inhibits Cell Motility and Cell Invasion

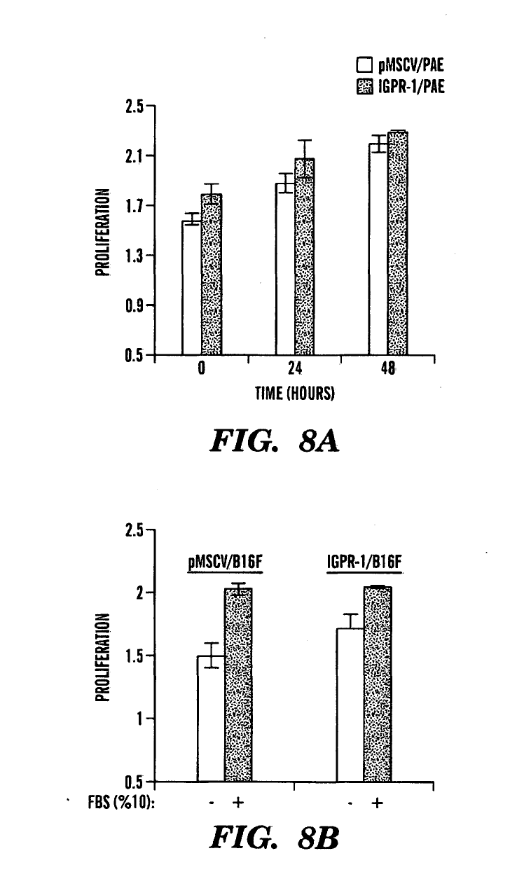

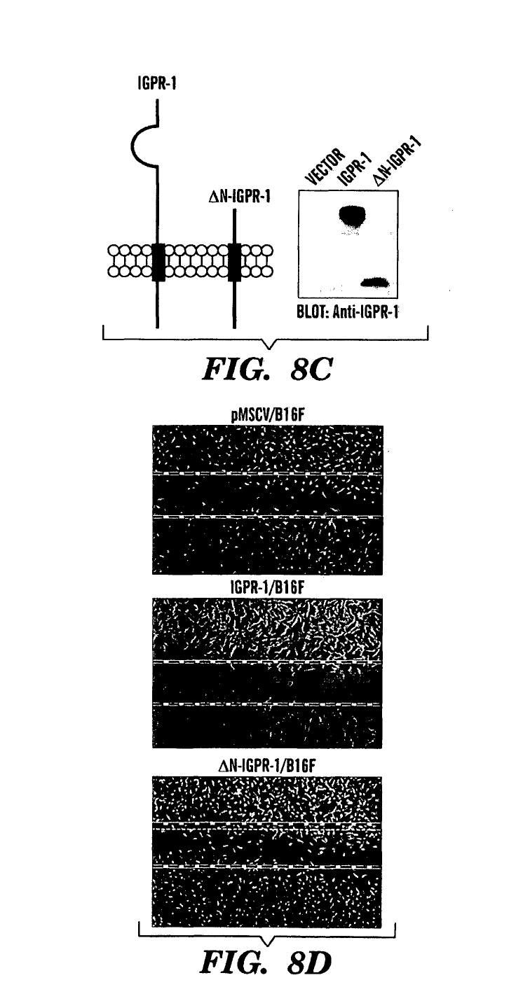

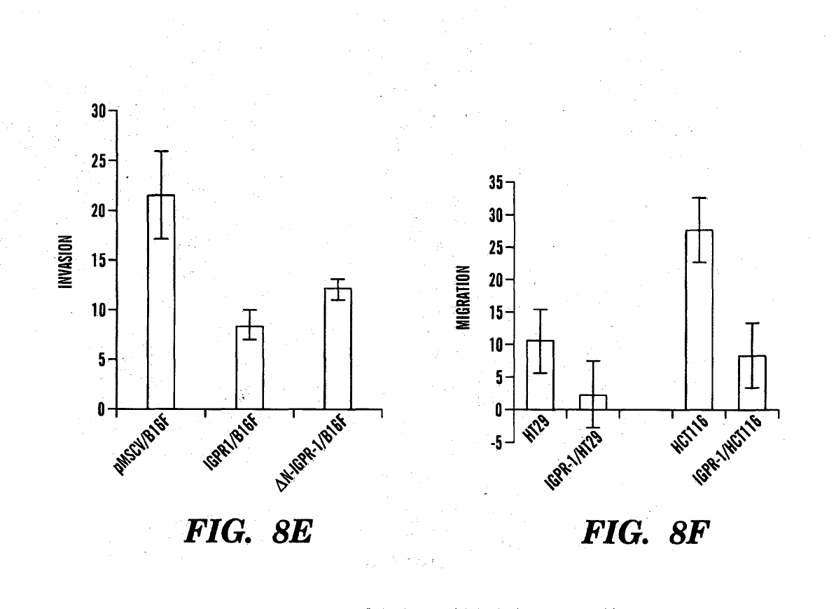

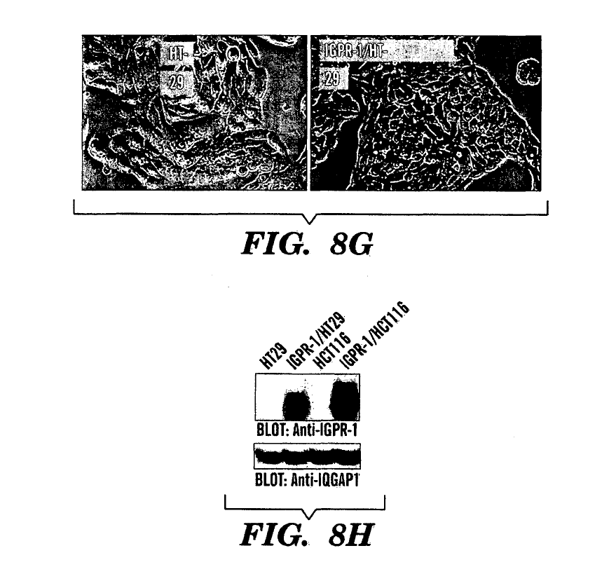

Focal adhesion and actin remodeling play a central role in various critical cellular functions such as cell growth and motility. To establish possible biological function of IGPR-1, inventors initially analyzed proliferation of PAE expressing IGPR-1. The growth rate of PAE cells expressing IGPR-1 versus PAE cells expressing empty vector were measured for 24 and 48 hours. Although proliferation of PAE cells expressing IGPR-1 were slightly higher than PAE cells expressing empty vector, the increased proliferation however, was not significant (FIG. 8A). To further investigate possible role of IGPR-1 in cell proliferation inventors expressed IGPR-1 in B16F (mouse melanoma) cells and analyzed their proliferation. The growth of B16F cells expressing IGPR-1 compared to B16F cells expressing empty vector in serum-free growth medium or in the %10 FBS virtually were the same (FIG. 8B), suggesting that IGFR-1 activity is not associated with proliferative events in PAE or B16F cells.

To test possible role of IGPR-1 in cell migration inventors initially measured migration of PAE cells expressing IGPR-1. The initial data showed that expression of IGPR-1 in PAE cells inhibits cellular migration both in a Boyden chamber modified migration assay and in a wounding assay. To further analyze possible role of IGPR-1 in cell migration in context of tumor cells inventors created B16F cells expressing either full length IGPR-1 or extracellular domain deleted IGPR-1 (ΔN-IGPR-1) where the N-terminal 133 amino acids encoding most of the extracellular domain including its immunoglobulin domain is deleted (FIG. 8C). B16F cells expressing wild type IGPR-1 and ΔN-IGPR-1 were subjected to the wounding/migration assay. The data showed that expression of wild type IGPR-1 in B16F cells significantly inhibits migration of B16F cells (FIG. 8D). B16F cells expressing ΔN-IGPR-1 migrated similar to control B16F cells expressing empty vector (FIG. 8D). B16F cells expressing ΔN-IGPR-1 or empty vector after 15 hours fully populated the wounding area where only a few B16F cells expressing IGPR-1 were migrated (data not shown). The data demonstrates that IGPR-1 inhibits cell migration and its extracellular domain is required for its ability to modulate cellular migration.

Since IGPR-1 inhibits cell motility inventors decided to further investigate its function further by examining its role in invasion of B16F cells in a Matrigel-based invasion assay. The result showed that B16F cells expressing IGPR-1 were significantly less invasive compared to B16F cells expressing empty vector (FIG. 8D). Moreover, deletion of immunoglobulin containing extracellular region also abolished the anti-invasive characteristic of IGPR-1 (FIG. 8D), suggesting that the presence of immunoglobulin domain is required for IGPR-1 to convey its anti-motility and anti-invasive function in these cells. To address role of IGPR-1 in migration of human tumor cells inventors over-expressed IGPR-1 in HT-29 and HCT116 (colorectal adenocarcinoma cells) cells and analyzed their migration in Boyden chamber migration assay. Expression of IGPR-1 in these tumor cell lines significantly inhibited their migration (FIG. 8F). Expression of IGPR-1 in HT-29 cells also considerably changed the morphology of these cells (FIG. 8G). Taken together the data demonstrate that IGPR-1 activity inhibits tumor cell migration.

### Example 6

### IGPR-1 Associates with SH3 Containing Cytoplasmic Signaling Proteins Involved with Cell Adhesion

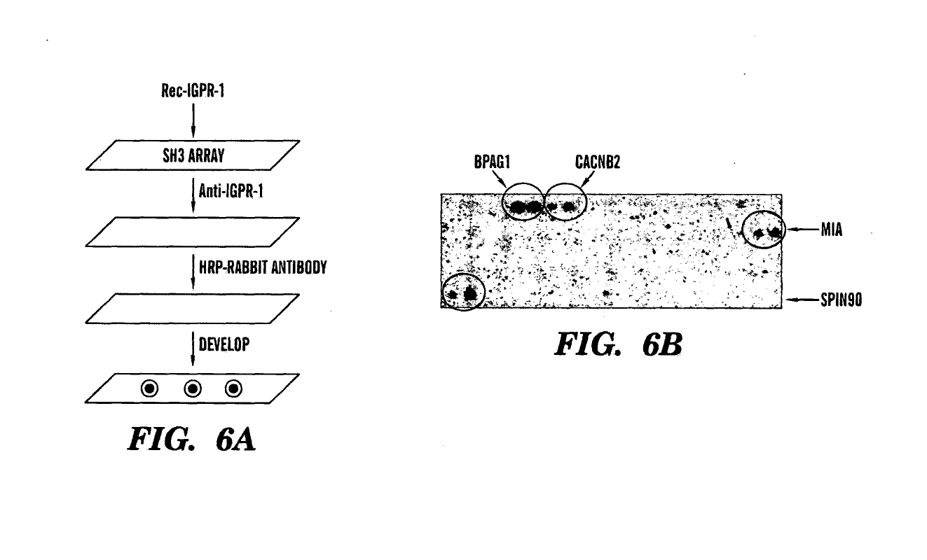

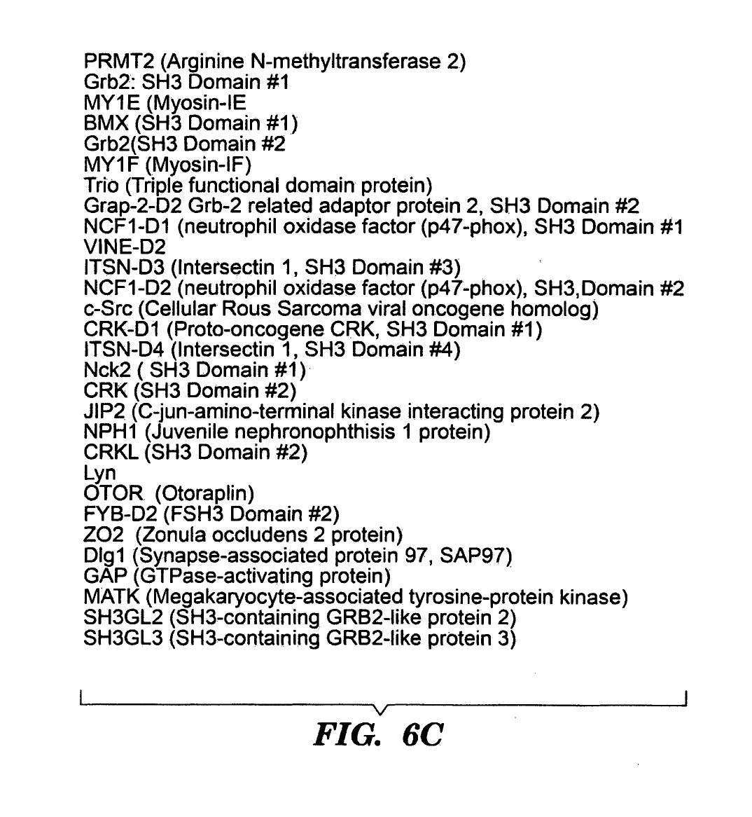

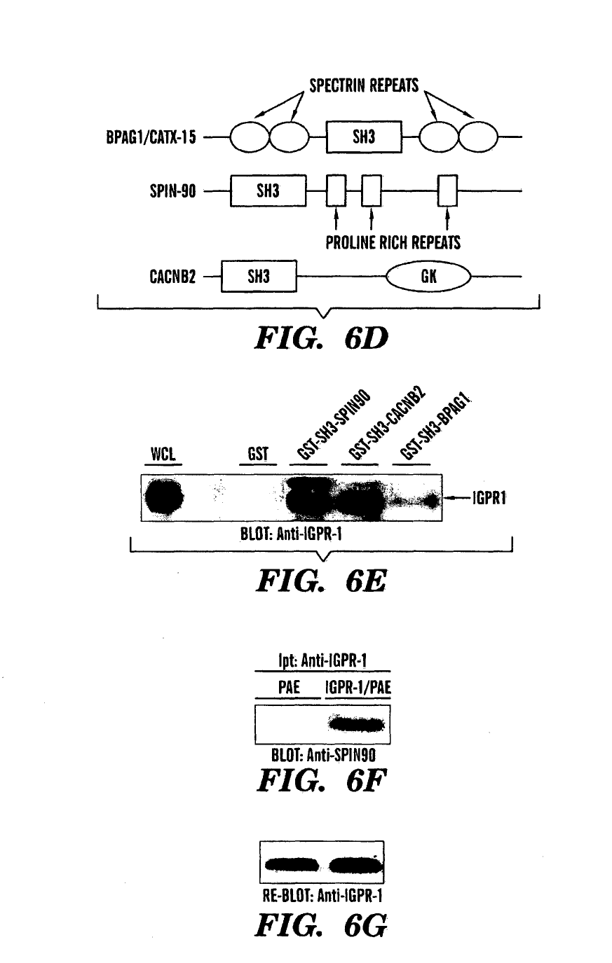

The cytoplasmic region of IGPR-1 is an emblematic of proline rich motif (FIG. 1A). Proline-rich motif is known to interact with SH3 containing signaling proteins (Kaneko et al., 2008). To identify signaling proteins that interact with IGPR-1 and potentially mediating its cellular function, we used a SH3 protein array consisting of 34 SH3 domains derived from 34 individual proteins. The blotting of the SH3 array membrane with purified recombinant GST-proline rich motif of IGPR-1 identified four distinct SH3 domains including SPIN90/WISH(SH3 Protein Interacting with Nck, 90 kDa), CACNB2 (calcium channel 132), BPAG1(Bullous pemphigoid antigen-1) and MIA (Melanoma inhibitory activity) (FIG. 6B).

SPIN90 is known to play a key role in cell adhesion and in the actin cytoskeleton reorganization (Lim et al., 2001; Takenawa and Suetsugu, 2007). The calcium channel β2 (CACNB2) is one of the subunits of L-type voltage-pendent calcium channels. Voltage-dependent calcium channels are oligomeric proteins composed of α1, α2δ, β (1-4) and γ subunit in which they control calcium entry into cells (Yamagata and Sanes, 2008). The β subunit is member of MAGUK-like protein (membrane-associated guanylate kinase) proteins with a guanylate kinase (GK) domain and an SH3 (Src homology 3) domain. BPAG1 is a member of plakins family proteins, comprises cytoskeleton binding proteins (Fuchs and Karakesisoglou, 2001; Fuchs and Yang, 1999; Fuchs et al., 2004)). BPAG1 is involved in anchoring keratin intermediate filaments to the cytoplasmic side of hemidesmosomes (Matsumura et al., 1997). Melanoma inhibitory activity (MIA) is a small secreted protein that interacts with extracellular matrix proteins (Bosserhoff et al., 1999).

To further validate the array data, the inventors created GST recombinant SH3 domain of SPIN90, BPAG1 and CANCB2 and tested their binding to IGPR-1 in a GST-pull down assay. The MIA binding to IGPR-1 was not considered for further analysis due the fact that it is a secreted protein (Bosserhoff, et al., 1999). The data demonstrated that IGPR-1 strongly binds to SH3 domains of SPIN90, BPAG1 and CANB2 (FIG. 6E). The binding of SPIN90 and BPAG1 was stronger than the CANCB2, demonstrating that SH3 containing proteins interacts with IGPR-1 more readily than the SH3 domain of CANCB2. The inventors further validated binding in vivo binding of SPIN90 with IGPR-1 by co-immunoprecipitation approach in PAE cells expressing IGPR-1. Cell lysates derived from PAE cells expressing IGPR-1 immunoprecipitated with anti-IGPR-1 selectively co-precipitated with endogenous SPIN90 as detected with anti-SPIN90 antibody (FIG. 6F). Taken together, the data demonstrate that SPIN90, BPAG1, and CACNB2 through their SH3 domains are recruited to IGPR-1 and play a role in relaying IGPR-1 mediated cellular responses such as cellular migration and invasion.

### Example 7

### SPIN90 Activity is Required for Angiogenesis

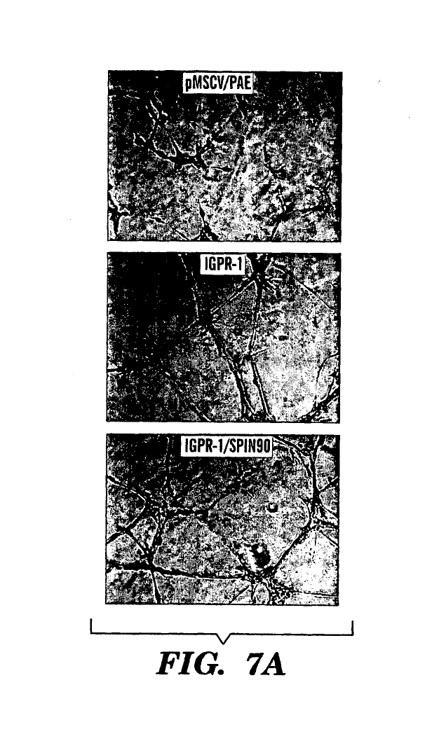

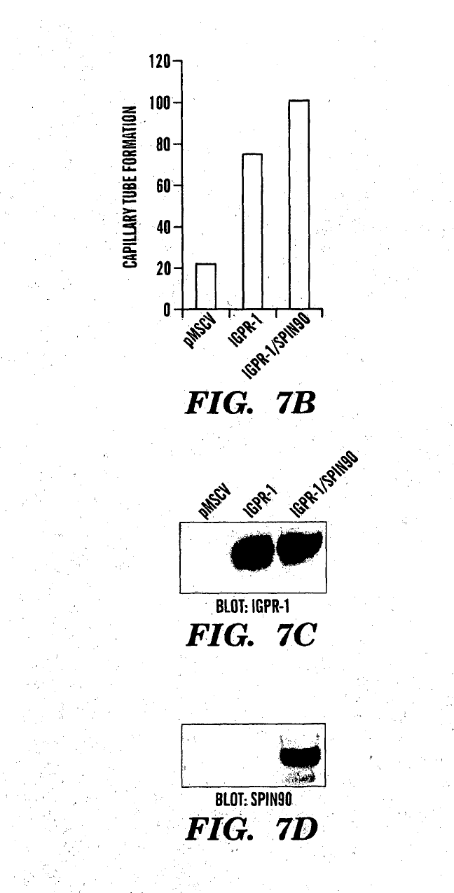

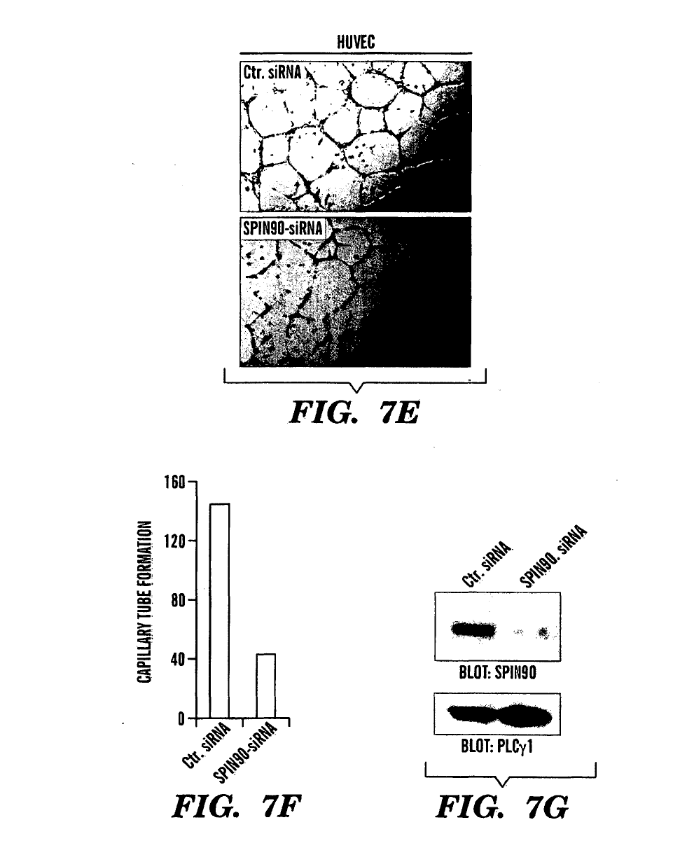

Since SPIN90 activity is linked to cellular adhesion and morphology (Lim et al., 2003; Takenawa and Suetsugu, 2007), and the inventors discovery of SPIN90 as an IGPR-1 interacting protein, indicates that SPIN90 may play a role in IGPR-1 mediated angiogenesis. To further determine the biological importance of SPIN90 in angiogenesis the inventors over-expressed SPIN90 in PAE cells and examined its potential to stimulate capillary tube formation of PAE cells. Expression of SPIN90 in PAE cells increased capillary tube formation (FIG. 7A), indicating that SPIN90 activity is linked to angiogenic events in endothelial cells. Quantification of capillary tube formation and expression of IGPR-1 and SPIN90 in PAE also are shown (FIG. 7B, 7C). To address the biological importance of endogenous SPIN90 in angiogenesis, the inventors silenced expression of SPIN90 by siRNA (using SPIN90 siRNAs commercially available from Santa Cruz Biotechnology, cat no: sc-76564) and examined the biological consequence of depletion of SPIN90 in HUVEC cell. Depletion of SPIN90 significantly reduced capillary tube formation of HUVEC cells (FIG. 7E). Quantification of capillary tube formation of HUVEC cells transfected with control siRNA or SPIN90 siRNA is shown (FIG. 7F). The effect of SPIN90 siRNA in silencing expression of SPIN90 in HUVEC cells is shown (FIG. 7G). Taken together, the inventors have demonstrated that SPIN90 associates with IGPR-1 through its SH3 domain and its activity regulates angiogenesis.

### Example 8

### SPIN90 Inhibits Cellular Migration

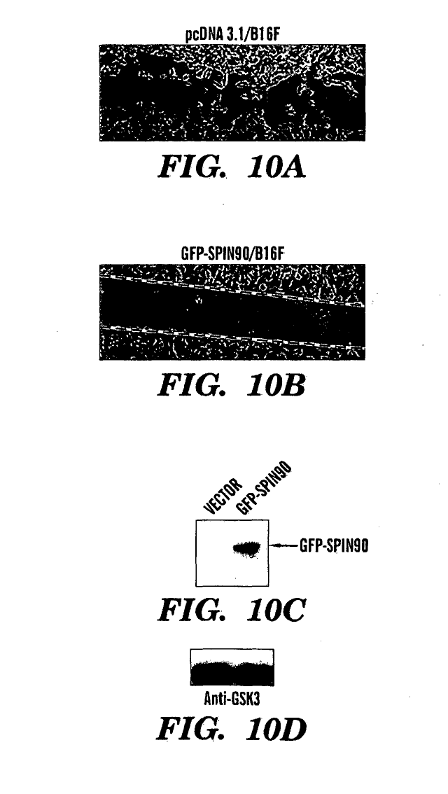

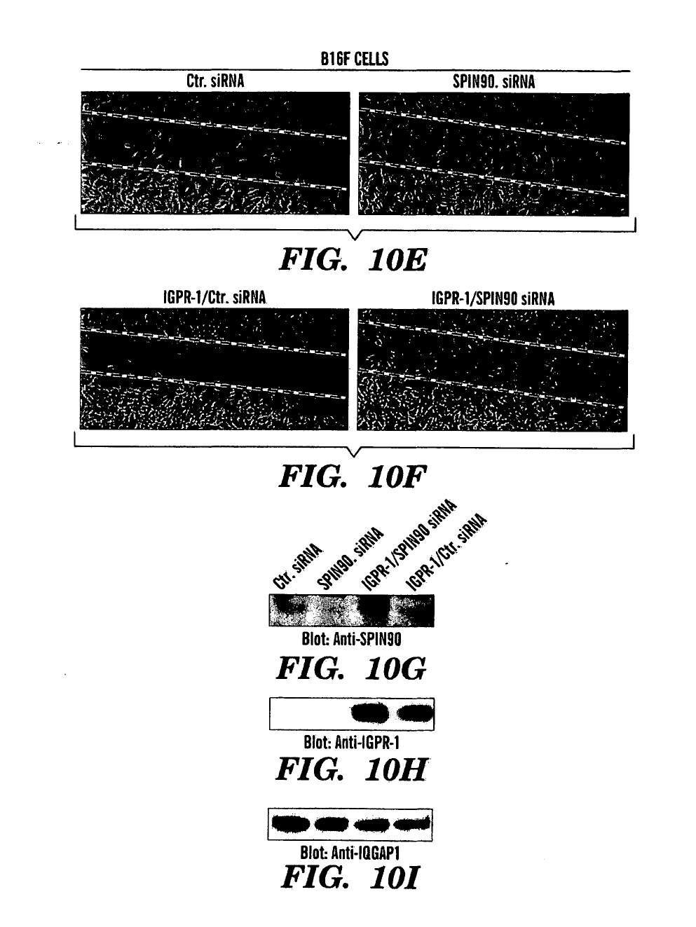

To demonstrate the biological importance of association of SPIN90 with IGPR-1 in particular, its possible role in relaying inhibitory function of IGPR-1 in cell migration, inventors initially over-expressed SPIN90 in B16F melanoma cells and analyzed its effect in migration of B 16F cells in wounding assay. The data showed that over-expression of SPIN90 significantly inhibits migration of B16F cells, suggesting that SPIN90 is involved in modulation of B16F cells migration (FIG. 10A). To further establish role of SPIN90 in migration of B16F cells, in particular role of endogenous SPIN90 in cell migration, inventors knockdown expression of SPIN90 in B16F cells with siRNA strategy and analyzed their migration. Silencing of SPIN90 expression resulted in a marked increase in the migration of B16F cells (FIG. 10D), demonstrating that SPIN90 activity negatively regulates cellular migration. To address role of SPIN90 in IGPR-1-dependent inhibition of cell migration, inventors knockdown expression of SPIN90 in B16F cells in the background of IGPR-1. As shown, silencing expression of SPIN90 fully reversed the IGPR-1-dependent inhibition of migration of B16F cells (FIG. 10E). Taken together, the data demonstrate that IGPR-1 employs SPIN90 to modulate cellular migration.

### Example 9

### IGPR-1 Regulates Cell-Cell Interaction and Undergoes Cis- and Trans-Dimerization

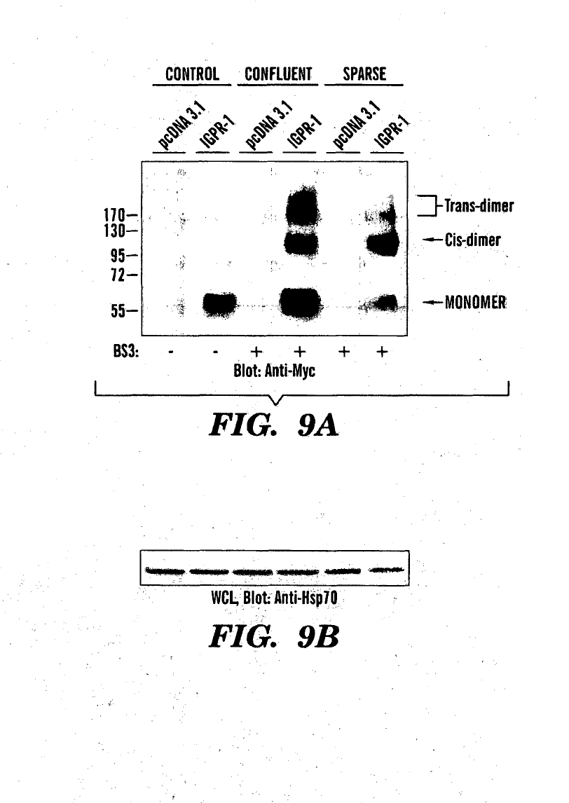

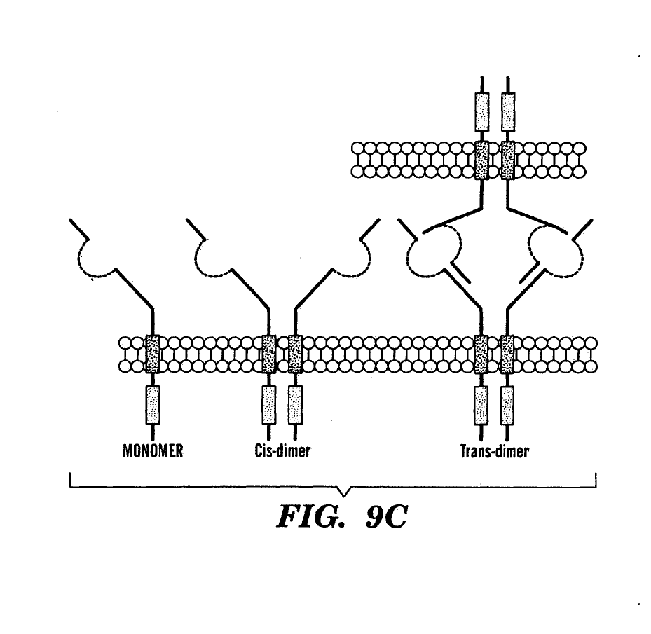

To determine whether IGPR-1 is involved in cell-cell interaction inventors subjected PAE cells expressing IGPR-1 and ΔN-IGPR-1 to aggregation/cell-cell adhesion assay. PAE cells expressing wild type IGPR-1 but not ΔN-IGPR-1 or empty vector formed a large aggregates of cells (FIG. 4E), demonstrating that IGPR-1 mediates cell-cell adhesion and the extracellular domain is required for its function. To further establish role of IGPR-1 in cell-cell interaction, the inventors investigated dimerization of IGPR-1 particularly its possible trans-dimerization. Inventors reasoned that if cells are plated in confluent condition, IGPR-1 could form trans-dimerization and possibly cis-dimerization (see FIG. 9C). However, plating the cells in a sparse condition, IGPR-1 will be less able to form trans-dimerization. The result, as detected by western blot analysis showed that in confluent condition, three major protein bands corresponding to IGPR-1 were detected (FIG. 9A); a low molecular weight band corresponds to monomeric form of IGPR-1, a medium size (˜110 KDa) and high molecular weight (˜200 KDa) band (FIG. 9A). The appearance of high molecular weight IGPR-1 in sparse condition was significantly less than the cells platted in confluent condition, the appearance of medium size ((˜110 KDa) was remained the same (FIG. 4F). The medium size (˜110 KDa) IGPR-1 probably represents a cis-dimerization of IGPR-1, where the higher molecular weight (˜200 KDa) may correspond to trans-dimerization of IGPR-1 (FIG. 9C). Altogether, the data indicate that IGPR-1 undergoes cis- and trans-dimerization and mediates cell-cell interaction.

### Example 8

Age related macular degeneration (AMD) is the leading cause of vision loss in the USA (Friedman et al., 2004). Although the cause of AMD is not known, retinal pigment epithelium (RPE) cells dysfunction and angiogenesis are hallmarks of pathology of AMD and hence targeting RPE cells and angiogenesis represent the most important area of research for the development of therapeutic agents for treatment of AMD (Green W R, 1999; Bressler et al., 1994; Kozlowski, 2012). RPE cells support healthy photoreceptors function by acting as “nursemaids” and maintain the outer blood retina barrier. As the photoreceptor and RPE cells slowly die, blood vessels (angiogenesis, proliferation of endothelial cells) start to grow from their normal location in the choroid into beneath of the retina. These newly formed blood vessels often leak and bleed, resulting in vision loss or impairment (Ding et al., 2009). RPE cell transplant currently being investigated as a therapeutic strategy for AMD treatment, however, this strategy is not very successful because the transplanted cells often do not adhere and die (Huang et al., 2011). The formation of adhesive contacts between RPE cells is essential for their ability to support photoreceptors function and maintain the outer blood retina barrier. To date, the molecular mechanisms of RPE adhesion is largely unknown and the putative gene products that regulate RPE cell-cell interaction yet to be identified.

The inventors herein have identified a novel cell adhesion molecule called IGPR-1 (immunoglobulin containing proline rich receptor-1) IGPR-1 which regulates epithelial and endothelial cell-cell interaction. Manipulation of expression of IGPR-1 in endothelial cells demonstrated that IGPR-1 plays an important role in angiogenesis, underscoring its therapeutic potential in angiogenesis-associated diseases ranging from cancer to AMD. Given the fact that IGPR-1 is expressed both in endothelial and epithelial cells and the aberrant function of these cells types (i.e., RPE cells and endothelial cells) are the major source of pathology and treatment strategies in AMD, the inventors next examined expression of IGPR-1 in human eye tissue.

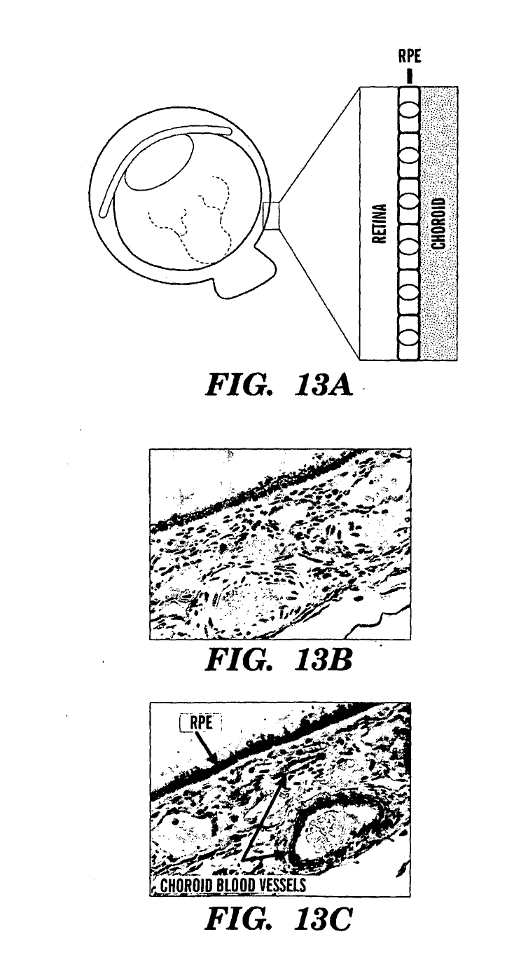

The inventors demonstrate that in normal human eye IGPR-1 is exclusively expressed in RPE cells and in choroidal endothelial cells (FIG. 13). The demonstrate that targeting angiogenesis based on the known role of IGPR-1 in angiogenesis and its expression in RPE cells could provide a novel strategy for treatment of AMD.

### Example 9

In this study inventors have identified IGPR-1 as a novel cell surface protein expressed in various human organs and tissues prominently in cells with epithelial origin. IGPR-1 mediates cell adhesion and inhibits cellular migration and its extracellular immunoglobulin containing domain is required for its function. Based on the observed characteristics of IGPR-1 such as promoting cellular aggregation, increasing resistance of cells to trypsin treatment, morphological change of cells, and increase in focal adhesion inventors propose IGPR-1 as a novel cell adhesion molecule (CAM). To date various class of CAMs have been described including, cadherins, mucins, integrins, selectins, and the immunoglobulin (Ig) superfamily (Takai et al., 2008). CAMs are known to mediate homophilic (like-binds-like) adhesion between cells of a single type and heterophilic adhesion between cells of different types. CAMs are uniformly distributed along the regions of plasma membranes that contact other cells, and the cytoplasmic regions of these proteins are usually connected to cytoskeleton (Takai et al., 2008).

CAMs are known to interact with a distinct signaling proteins linked to cytoskeleton. For example, Cadherins are known to interact with β-catenin (Harris and Tepass, 2010), Nectins interact with the filamentous (F)-actin-binding protein afadin and the cell polarity protein partitioning defective-3 (PAR3) (Takai et al., 2003). ICAM-1 associates with α-actinin, ERM proteins, including moesin and ezrin (Barreiro et al., 2002) and JAMs bind to ZO1 (Bazzoni, 2003). Among all the known CAMs, IGPR-1 appears to be unique in term of its ability to interact with a unique set of SH3 containing proteins including, SPIN90, CANCB2, and BPAG1. Activity of SPIN90 activity is linked to cell adhesion and actin cytoskeleton reorganization (Lim et al., 2003; Takenawa and Suetsugu, 2007). Although direct role SPIN90 in cell migration has not been established, recent studies, however, have linked SPIN90 activity to lamellipodia-like protrusion formation in COST cells treated with PDGF, a process that is associated with cell motility (Kim et al., 2006; Teodorofa et al., 2009). Our observation indicates that over-expression of SPIN90 in B16F cells inhibits cell migration and silencing its expression significantly increases cell migration. Furthermore, silencing SPIN90 expression in B16F cells reverses IGPR-1 mediated inhibition of cell migration, demonstrating that SPIN90 activity negatively regulates cell migration. SPIN90 has been shown to interact with actin and this interaction is thought is required for its involvement lamellipodia formation (Kim et al., 2007). SPIN90 is required for IGPR-1 dependent inhibition of cell migration, suggesting that perhaps IGPR-1 alters association of SPIN90 with actin complexes or its localization therefore prevents it from participation in lamellipodia formation. SPIN90 also has been shown to be phosphorylated be by MAP (ERK1/ERK2) kinases (Lim et al., 2003), raising a possibility that IGPR-1 may inhibit SPIN90 by interfering with its phosphorylation. Moreover, lamellipodia is considered to be F-actin based cell migration and recent studies have questioned whether F-actin based cell migration is the sole regulator of cell migration (Ponti, et al., 2004; Gupton et al., 2005; Bach et al., 2009). Alternatively, cells can migrate without lamellipodia through myosin II and tropomyosin (Ponti, et al., 2004; Gupton et al., 2005).

Consistent with its observed effect on cell adhesion and migration, transfer of IGPR-1 into PAE cells significantly increases expression of vinculin. Vinculin is a major constitutive component of focal adhesion and adherens junctions where it binds to several cytoskeletal proteins linking microfilaments to CAMs such as cadherins and integrins (Pawlak and Helfman, 2001). Increased expression of vinculin have been shown to inhibit cell migration, tumorigenicity (Fernadez, et al., 1992a; Fernadez, et al., 1992b; Fernadez, et al., 1993), and to modulate filopodia and lamellipodia (Varnum-Finney and Reichart, 1994). Thus, increased expression of vinculin could be one possible mechanism through which IGPR-1 could regulate cell adhesion and motility.

CAMs are known to interact with a distinct signaling proteins linked to cytoskeleton. For example, Cadherins are known to interact with β-catenin (Harris and Tepass, 2010), Nectins interact with the filamentous (F)-actin-binding protein afadin and the cell polarity protein partitioning defective-3 (PAR3) (Takai et al., 2003). ICAM-1 associates with α-actinin, ERM proteins, including moesin and ezrin (Barreiro et al., 2002) and JAMs bind to ZO1 (Bazzoni, 2003). Among all the known CAMs, IGPR-1 appears to be unique in term of its ability to interact with a unique set of SH3 containing proteins including, SPIN90, CANCB2, and BPAG1. Activity of SPIN90 activity is linked to cell adhesion and actin cytoskeleton reorganization (Lim et al., 2003; Takenawa and Suetsugu, 2007). Although direct role SPIN90 in cell migration has not been established, recent studies, however, have linked SPIN90 activity to lamellipodia-like protrusion formation in COST cells treated with PDGF, a process that is associated with cell motility (Kim et al., 2006; Teodorofa et al., 2009). Our observation indicates that over-expression of SPIN90 in B16F cells inhibits cell migration and silencing its expression significantly increases cell migration. Furthermore, silencing SPIN90 expression in B16F cells reverses IGPR-1 mediated inhibition of cell migration, demonstrating that SPIN90 activity negatively regulates cell migration. SPIN90 has been shown to interact with actin and this interaction is thought is required for its involvement lamellipodia formation (Kim et al., 2007). SPIN90 is required for IGPR-1 dependent inhibition of cell migration, suggesting that perhaps IGPR-1 alters association of SPIN90 with actin complexes or its localization therefore prevents it from participation in lamellipodia formation. SPIN90 also has been shown to be phosphorylated be by MAP (ERK1/ERK2) kinases (Lim et al., 2003), raising a possibility that IGPR-1 may inhibit SPIN90 by interfering with its phosphorylation. Moreover, lamellipodia is considered to be F-actin based cell migration and recent studies have questioned whether F-actin based cell migration is the sole regulator of cell migration (Ponti, et al., 2004; Gupton et al., 2005; Bach et al., 2009). Alternatively, cells can migrate without lamellipodia through myosin II and tropomyosin (Ponti, et al., 2004; Gupton et al., 2005).

The calcium channel β2 (CACNB2) is one of the subunits of L-type voltage-pendent calcium channels involved in the calcium entry into cells (Yamakage and Namiki, 2002) but its role in cell adhesion is not known. BPAG1 is a member of plakins suprfamily, binds to cytoskeleton (Fuchs and Yang, 1999; Fuchs and Karakesisoglou, 2001) and is involved in anchoring keratin intermediate filaments to the cytoplasmic side of hemidesmosomes (Jones et al., 1994). It is highly likely that both BPAG1 and CANCB2 contribute to GPR-1 mediated cellular events, albeit their function yet to be established in IGPR-1 signaling.

Consistent with the CAM-mediated phenotypic characteristics such as focal adhesion and cellular morphology, introducing IGPR-1 to PAE cells resulted in the acquisition of cubical morphology and elevated focal adhesion. These cells also were resistant to trypsin/EDTA-mediated detachment from tissue culture plate and have acquired increased cell adhesion properties. CAMs play essential role in the regulation of cell movement and cellular invasion, the data presented in this work demonstrate that IGPR-1 also modulates cell migration and invasiveness and the Ig containing extracellular domain of IGPR-1 is required for its ability to inhibit cell migration and invasion. The function of IGPR-1 is highly correlated with those of other well-known CAMs such as cadherins. Homophilic engagements of cadherins, is known to stabilize cell-cell contacts to form adherent-junctions in epithelial cells, fibroblasts and endothelial cells (Harris and Tepass, 2010). Our data demonstrate that IGPR-1 may also mediate cell-cell adhesion through homo- and trans-dimerization.

Another interesting and important aspect of this work is that IGPR-1 is mainly expressed in cells with epithelial origin as epithelium of various organs/tissues was positive for IGPR-1, indicating that IGPR-1 is a novel epithelial cell marker with a significant role in normal pathobiology of epithelial cells. IGPR-1 is expressed in various human tumors although at variable levels. In some tumors IGPR-1 expression appears to be either undetected or reduced, indicating that transcriptional regulation of IGPR-1 in tumor cells may be relevant for development/acquisition of tumor phenotypes such as migration and invasion. In conclusion, the identification of IGPR-1 is a novel CAM and its signaling components regulate key cellular events such as cell movement and invasion, and IGPR-1 is relevant as a target for human diseases such as cancer.

### Example 10

### IGPR-1 Regulates Tumor Growth and Angiogenesis In Vivo

The inventors have used immunohistochemical analysis of human tissues demonstrated that IGPR-1 is mainly expressed by epithelial and endothelial cells. Surprisingly, ectopic expression of IGPR-1 in certain tumor cell lines (such as B16F, mouse melanoma) also inhibits cell migration in cell culture system (Rahimi et al., submitted for publication, 2011).

In contrast to what inventors observed in cell culture system sub-dermal injection of tumor cells, B16F cells ectopically expressing IGPR-1 into mice stimulated tumor growth and angiogenesis, as noted formation of blood vessels surrounding tumor mass (FIG. 10). The size of tumor were also larger than B16F cells expressing control empty vector (FIG. 10). This demonstrates that IGPR-1 contributes to tumor-angiogenesis by interacting with endothelial cells.

### Example 11

### Soluble Extracellular Domain of IGPR-1 Inhibits Tumor Growth and Angiogenesis

Based on the above observation in Example 10, the inventors assessed the role of IGPR-1 in tumor growth and angiogenesis, in particular, assessed the role of its extracellular domain in mediating tumor growth and angiogenesis. To this end, the inventors generated an inhibitor of IGPR-1 which is a soluble recombinant extracellular domain of IGPR-1, (e.g., a dominant negative IGPR-1 protein comprising the immunoglobuling domain only, e.g., Ig-IGPR-1) (FIG. 4H)), and mixed this soluble recombinant protein with B16F cells expressing IGPR-1 before injecting them into mice. The result demonstrated that inhibition of IGPR-1 using a soluble Ig-domain of IGPR-1 (e.g., Ig-IGPR-1) inhibits IGPR-1-dependent tumor growth and angiogenesis as both tumor size and angiogenic phenotype of B16F cells expressing IGPR-1 were significantly reduced (FIG. 11).

### REFERENCES

All references are incorporated herein in their entirety by reference.
Abou Zeid, N., et al. (2006). Serine phosphorylation regulates paxillin turnover during cell migration. Cell communication and signaling: CCS 4, 8.
Aiello LP, Pierce EA, Foley E D, et al. Suppression of retinal neovascularization in vivo by inhibition of vascular endothelial growth factor (VEGF) using soluble VEGF-receptor chimeric proteins. Pro Natl Acad Sci USA. 1995; 92:10457-10461.
Auerbach D, et al. Genetic approaches to the identification of interactions between membrane proteins in yeast. J Recept Signal Transduct Res. 2002 February-November; 22(1-4):471-81.
Bach, T. L., et al. (1998). VE-Cadherin mediates endothelial cell capillary tube formation in fibrin and collagen gels. Experimental cell research 238, 324-334.
Barclay, A. N. (2003). Membrane proteins with immunoglobulin-like domains—a master superfamily of interaction molecules. Seminars in immunology 15, 215-223.
Barreiro, O., et al. (2002). Dynamic interaction of VCAM-1 and ICAM-1 with moesin and ezrin in a novel endothelial docking structure for adherent leukocytes. The Journal of cell biology 157, 1233-1245.
Bazzoni, G. (2003). The JAM family of junctional adhesion molecules. Current opinion in cell biology 15, 525-530.
Bressler N M, Silva J C, Bressler S B, Fine S L, Green W R (1994) Clinicopathologic correlation of drusen and retinal pigment epithelial abnormalities in age-related macular degeneration. Retina 14: 130-142.
Bosserhoff, A. K., et al. (1999). MIA, a novel serum marker for progression of malignant melanoma. Anticancer research 19, 2691-2693.
Brümmendorf, T. and Lemmon, V. Immunoglobulin superfamily receptors: cis-interactions intracellular adapters and alternative splicing regulate adhesion. Current Opinion in Cell Biology 2001, 13:611-618.
Bulj an M, Bateman A. The evolution of protein domain families. Biochem Soc Trans. 2009 August; 37(Pt 4):751-5.
Carmeliet, P., and Jain, R. K. (2011a). Molecular mechanisms and clinical applications of angiogenesis. Nature 473, 298-307.
Carmeliet, P., and Jain, R. K. (2011b). Principles and mechanisms of vessel normalization for cancer and other angiogenic diseases. Nature reviews. Drug discovery 10, 417-427.
DeLisser, H., et al. (2010). Vascular endothelial platelet endothelial cell adhesion molecule 1 (PECAM-1) regulates advanced metastatic progression. Proceedings of the National Academy of Sciences of the United States of America 107, 18616-18621.
Ding X, Patel M, Chan CC. Molecular pathology of age-related macular degeneration. Prog Retin Eye Res. 2009 January; 28(1):1-18. Epub 2008 Nov. 6.
Friedman DS, O'Colmain B J, Munoz B, et al. (2004) Prevalence of age-related macular degeneration in the United States. Arch Ophthalmol 122:564-572
Fuchs, E., and Karakesisoglou, I. (2001). Bridging cytoskeletal intersections. Genes & development 15, 1-14.
Fuchs, E., and Yang, Y. (1999). Crossroads on cytoskeletal highways. Cell 98, 547-550.
Fuchs, M., et al. (2004). Dynamics of cell adhesion and motility in living cells is altered by a single amino acid change in E-cadherin fused to enhanced green fluorescent protein. Cell motility and the cytoskeleton 59, 50-61.
Gory-Faure, S et al. (1999). Role of vascular endothelial-cadherin in vascular morphogenesis. Development 126, 2093-2102.
Green W R (1999) Histopathology of age-related macular degeneration. Mol V is 5: 27.
Harpaz Y, Chothia C. Many of the immunoglobulin superfamily domains in cell adhesion molecules and surface receptors belong to a new structural set which is close to that containing variable domains. J Mol. Biol. 1994 May 13; 238(4):528-39.
Harris, T. J., and Tepass, U. (2010). Adherens junctions: from molecules to morphogenesis. Nature reviews. Molecular cell biology 11, 502-514.
Huang Y, Enzmann V, Ildstad S T. Stem cell-based therapeutic applications in retinal degenerative diseases. Stem Cell Rev. 2011 June; 7(2):434-45.
Jaap D. van Buul, et al. Endothelial Signaling by Ig-Like Cell Adhesion Molecules. Arterioscler Thromb Vasc Biol 2007; 27;1870-1876.
Jones E Y, et al. Nat Rev Immunol. 2006 April; 6(4):271-82.
Kaneko, T., Li, L., and Li, S. S. (2008). The SH3 domain—a family of versatile peptide- and protein-recognition module. Frontiers in bioscience: a journal and virtual library 13, 4938-4952.
Kim, D. J., et al. Interaction of SPIN90 with Arp2/3 complex mediates lamellipodia and actin comet tail formation, J. Biol. Chem. 281 (1) (2006), pp. 617-625.
Kozlowski MR. RPE cell senescence: a key contributor to age-related macular degeneration. Med. Hypotheses. 2012 April; 78(4):505-10
Lander E S, et al., Initial sequencing and analysis of the human genome. Nature. 2001 Feb. 15; 409(6822):860-921.
Lim C S, et al. Regulation of SPIN90 phosphorylation and interaction with Nck by ERK and cell adhesion. J Biol. Chem. 2003 Dec. 26; 278(52):52116-23.
Lim, C. S., et al. (2001). SPIN90 (SH3 protein interacting with Nck, 90 kDa), an adaptor protein that is developmentally regulated during cardiac myocyte differentiation. The Journal of biological chemistry 276, 12871-12878.
Matsumura, T., Wolff, K., and Petzelbauer, P. (1997). Endothelial cell tube formation depends on cadherin 5 and CD31 interactions with filamentous actin. J Immunol 158, 3408-3416.
Meyer R D, Singh A J, Rahimi N. The carboxyl terminus controls ligand-dependent activation of VEGFR-2 and its signaling. J Biol. Chem. 2004 Jan. 2; 279(1):735-42
Meyer, R. D., Husain, D., and Rahimi, N. (2011a). c-Cbl inhibits angiogenesis and tumor growth by suppressing activation of PLCgamma1. Oncogene 30, 2198-2206.
Meyer, R. D., et al. (2006). Leucine motif-dependent tyrosine autophosphorylation of type III receptor tyrosine kinases. The Journal of biological chemistry 281, 8620-8627.
Meyer, R. D., Sacks, D. B., and Rahimi, N. (2008). IQGAP1-dependent signaling pathway regulates endothelial cell proliferation and angiogenesis. PloS one 3, e3848.
Meyer, R. D., et al. (2011b). PEST motif serine and tyrosine phosphorylation controls vascular endothelial growth factor receptor 2 stability and downregulation. Molecular and cellular biology 31, 2010-2025.
Pawlak, G., and Helfman, D. M. (2001). Cytoskeletal changes in cell transformation and tumorigenesis. Current opinion in genetics & development 11, 41-47.
Rahimi, N. (2006). VEGFR-1 and VEGFR-2: two non-identical twins with a unique physiognomy. Frontiers in bioscience: a journal and virtual library 11, 818-829.
Rahimi, N., Dayanir, V., and Lashkari, K. (2000). Receptor chimeras indicate that the vascular endothelial growth factor receptor-1 (VEGFR-1) modulates mitogenic activity of VEGFR-2 in endothelial cells. The Journal of biological chemistry 275, 16986-16992.
Rahimi N, Dayanir V, Lashkari K. Receptor chimeras indicate that the vascular endothelial growth factor receptor-1 (VEGFR-1) modulates mitogenic activity of VEGFR-2 in endothelial cells. J Biol. Chem. 2000 Jun. 2; 275(22):16986-92
Rahimi N, et al. Identification of ligand-induced proteolytic cleavage and ectodomain shedding of VEGFR-1/FLT1 in leukemic cancer cells. Cancer Res. 2009 Mar. 15; 69(6):2607-14.
Rahimi N, Rezazadeh K, Mahoney J E, Hartsough E, Meyer RD. Identification of IGPR-1 as a novel adhesion molecule involved in angiogenesis. Mol Biol Cell. 2012 May; 23(9):1646-56. Epub 2012 Mar. 14.
Sonnenberg A, Liem R K. Plakins in development and disease. Exp Cell Res. 2007 Jun. 10; 313(10):2189-203.
Takai, Y., et al. (2008). Nectins and nectin-like molecules: roles in contact inhibition of cell movement and proliferation. Nature reviews. Molecular cell biology 9, 603-615.
Takenawa, T., and Mild, H. (2001). WASP and WAVE family proteins: key molecules for rapid rearrangement of cortical actin filaments and cell movement. Journal of cell science 114, 1801-1809.
Takenawa, T., and Suetsugu, S. (2007). The WASP-WAVE protein network: connecting the membrane to the cytoskeleton. Nature reviews. Molecular cell biology 8, 37-48.
Teodorof C, et al. SPIN90-IRSp53 complex participates in Rac-induced membrane ruffling. Exp Cell Res. 2009, 315(14):2410-9.
Venter J C, et al., e sequence of the human genome. Science. 2001 Feb. 16; 291(5507):1304-51.
Yamagata, M., and Sanes, J. R. (2008). Dscam and Sidekick proteins direct lamina-specific synaptic connections in vertebrate retina. Nature 451, 465-469.
Yamakage M, Namiki A. Calcium channels—basic aspects of their structure, function and gene encoding; anesthetic action on the channels—a review. Can J Anaesth. 2002 February; 49(2):151-64.
Zaidel-Bar, R., et al. (2007). A paxillin tyrosine phosphorylation switch regulates the assembly and form of cell-matrix adhesions. Journal of cell science 120, 137-148.

1. A method of inhibiting angiogenesis in a subject, comprising administering to the subject a composition comprising an inhibitor of the immunoglobulin containing proline rich receptor-1 (IGPR-1) polypeptide.

2. A method of treating cancer in a subject at risk thereof, comprising administering to the subject an effective amount of a composition comprising an inhibitor of immunoglobulin containing proline rich receptor-1 (IGPR-1) protein or expression for the treatment and/or prevention of a malignancy or neoplasia disorder in the subject.

3.-60. (canceled)

61. The method of claim 1, wherein the inhibitor of IGPR-1 is a dominant negative inhibitor of IGPR-1 of SEQ ID NO: 4 or a fragment thereof which inhibits IGPR-1 polypeptide function and/or trans-dimerization.

62. The method of claim 1, wherein the inhibitor of IGPR-1 is a soluble extracellular domain of IGPR-1 which comprises SEQ ID NO: 6 or SEQ ID NO: 16 or a fragment of at least about 60 N-terminal amino acids of SEQ ID NO: 6 or SEQ ID NO: 16.

63. The method of claim 1, wherein the inhibitor of IGPR-1 is selected from the group consisting of: RNAi agent, oligonucleotide, antibody inhibitor, peptide inhibitor, protein inhibitor, avidimir, and functional fragments or derivatives thereof.

64. The method of claim 1, further comprising administering to the subject an inhibitor of SPIN90 polypeptide or SPIN90 gene expression.

65. The method of claim 1, wherein the subject has a disease characterized by an increase in angiogenesis selected from the group consisting of cancer, macular degeneration; diabetic retinopathy; rheumatoid arthritis; Alzheimer's disease; obesity, psoriasis, atherosclerosis, vascular malformations, angiomata, neovascularization, ocular neovascularization, and endometriosis.

66. The method of claim 1, wherein the subject has at least one of the disorders selected from the group comprising: age-related macular degeneration (AMD), diabetic retinopathy, retinopathy of prematurity (ROP), arthritis, rheumatoid arthritis (RA), osteoarthritis, cardiovascular disease.

67. The method of claim 2, wherein the inhibitor of IGPR-1 is a dominant negative inhibitor of IGPR-1 of SEQ ID NO: 4 or a fragment thereof which inhibits IGPR-1 polypeptide function and/or trans-dimerization.

68. The method of claim 2, wherein the inhibitor of IGPR-1 is a soluble extracellular domain of IGPR-1 which comprises SEQ ID NO: 6 or SEQ ID NO: 16 or a fragment of at least about 60 N-terminal amino acids of SEQ ID NO: 6 or SEQ ID NO: 16.

69. The method of claim 2, wherein the inhibitor of IGPR-1 is selected from the group consisting of: RNAi agent, oligonucleotide, antibody inhibitor, peptide inhibitor, protein inhibitor, avidimir, and functional fragments or derivatives thereof.

70. The method of claim 2, further comprising administering to the subject an inhibitor of SPIN90 polypeptide or SPIN90 gene expression.

71. The method of claim 2, wherein the cancer is a metastatic cancer, a malignant cancer or a neoplasia disorder.

72. The method of claim 2, wherein the cancer is of endothelial or epithelial origin or cancer of the epithelium.

73. The method of claim 2, wherein the cancer is selected from the group consisting of: bladder cancer, Breast cancer, Bronchus cancer, cancer of the Fallopian Tube, cancer of the gastrointestinal tract, cancer of esophagus, stomach cancer, colon cancer, cancer of the rectum, cancer of the small intestine, pancreatic cancer, cancer of the placenta, prostate cancer, skin cancer, testicular cancer, thyroid cancer, cancer of the thymus, endometrium cancer, Squamous Cell carcinoma (SCC), Infiltrating Duct carcinoma, adenocarcinoma, pillary carcinoma, cancer of the urethra.

74. The method of claim 2, wherein the cancer is a cancer of a cell type selected from the group consisting of: urothelim, tumor cells, glandular/Lobular Epithelium cells, bronchial Epithelium cells, fallopian tube lining Epithelium cells, squamous cell carcinoma cells, adenocarcinoma cells, stomach epithelium cells, intestinal epithelium cells, colonic epithelium cells, acniar cells, trophoblastic Epithelium cells, epidermal cells, karatinocytes, skin cells, testis semiferinstubulule cells, glandular epithelium cells of the thymus, thyroid cells, urothelium cells, endometrial Glandular cells.

75. The method of claim 2, wherein the subject is selected for treatment by identifying a subject with a cancer expressing IGPR-1.

76. The method of claim 2, wherein the inhibitor IGPR-1 inhibits endothelial cell migration.

77. The method of claim 76, wherein the endothelial cell is a human endothelial cell.

78. A method to promote angiogenesis in a subject in need thereof, comprising administering to a subject a composition comprising an IGPR-1 polypeptide comprising amino acids of SEQ ID NO: 2 or a functional fragment thereof.

79. The method of claim 78, wherein the subject in need thereof is selected from the group consisting of: a subject with an angiogenesis-related disorder characterized by a decrease in angiogenesis, a transplant recipient, or a subject who has undergone a transplant surgery, or a subject in need of neovascularization, a subject in need of tissue repair, regenerative medicine, and repair of a wound, or a subject who has had an infarct, cardiac infarct or stroke.

80. The method of claim 79, wherein the transplant recipient is a recipient of transplanted RPE cells.

81. A soluble extracellular domain of IGPR-1 for inhibiting angiogenesis or endothelial cell migration in a subject in need thereof, wherein the soluble extracellular domain of IGPR-1 inhibits IGPR-1 polypeptide function and/or cis-dimerization.

82. The soluble extracellular domain of IGPR-1 of claim 81, wherein the soluble extracellular domain of IGPR-1 comprises SEQ ID NO: 6 or SEQ ID NO: 16, or a variant or fragment of at least 60 N-terminal amino acids of SEQ ID NO: 6 or SEQ ID NO: 16 which inhibits IGPR-1 polypeptide function and/or cis-dimerization.

83. A dominant negative inhibitor of IGPR-1 comprising at least about 60 C-terminal amino acids of SEQ ID NO: 4 or a fragment thereof which inhibits IGPR-1 polypeptide function or IGPR-1 trans-dimerization.


SEQUENCE LISTING (24 sequences)

SEQ ID NO: 1 - protein, 282 aa, Homo sapiens
MGSPGMVLGLLVQIWALQEASSLSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPGFLFVLLGVGSMGVAAIVWGAWFWGRRSCQQRDSGNSPGNAFYSNVLYRPRGPPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 2 - DNA, 1282 bp, Homo sapiens
ggaagtctgtcaactgggagggggagaggggggtgatgggccaggaatggggtccccgggcatggtgctgggcctcctggtgcagatctgggccctgcaagaagcctcaagcctgagcgtgcagcaggggcccaacttgctgcaggtgaggcagggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaacataacaaggctctttgtggacccagatgaccccacacagaacagaaaccggatcgcaagcttcccaggattcctcttcgtgctgctgggggtgggaagcatgggtgtggctgcgatcgtgtggggtgcctggttctggggccgccgcagctgccagcaaagggactcaggtaacagcccaggaaatgcattctacagcaacgtcctataccggccccggggggccccaaagaagagtgaggactgctctggagaggggaaggaccagaggggccagagcatttattcaacctccttcccgcaaccggccccccgccagccgcacctggcgtcaagaccctgccccagcccgagaccctgccccagccccaggcccggccaccccgtctctatggtcagggtctctcctagaccaagccccacccagcagccgaggccaaaagggttccccaaagtgggagaggagtgagagatcccaggagacctcaacaggaccccacccataggtacacacaaaaaaggggggatcgaggccagacacggtggctcacgcctgtaatcccagcagtttgggaagccgaggcgggtggaacacttgaggtcaggggtttgagaccagcctggcttgaacctgggaggcggaggttgcagtgagccgagattgcgccactgcactccagcctgggcgacagagtgagactccgtctcaaaaaaaacaaaaagcaggaggattgggagcctgtcagccccatcctgagaccccgtcctcatttctgtaatgatggatctcgctcccactttcccccaagaacctaataaaggcttgtgaagaaaaagcaaaaaaaaaaaaaaaaaa

SEQ ID NO: 3 - skipped

SEQ ID NO: 4 - protein, 149 aa, Homo sapiens
DPDDPTQNRNRIASFPGFLFVLLGVGSMGVAAIVWGAWFWGRRSCQQRDSGNSPGNAFYSNVLYRPRGPPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 5 - protein, 127 aa, Homo sapiens
LSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFP

SEQ ID NO: 6 - protein, 111 aa, Homo sapiens
LSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFV

SEQ ID NO: 7 - protein, 22 aa, Homo sapiens
MGSPGMVLGLLVQIWALQEASS

SEQ ID NO: 8 - DNA, 3023 bp, Homo sapiens
ggcgggacaaccgcggcgcggctcggcgctcccggcctggcgttgtggcgccgcagcccttgccaccttccctgcacctgccctgcccggcctcatgtaccgcgcgctgtacgcgttccgctcggcggagcccaacgcgctggcgttcgccgcgggcgagaccttcctggtgctagagcgaagcagcgcgcactggtggctggccgcgcgggcgcgcagtggtgagacgggctacgtgccgccagcctacctgcgccgcctgcagggcctggagcaggatgtcctccaggccattgaccgggccatcgaggctgtacacaacacagccatgcgggatggtggcaagtacagcctggaacagcgtggagtcctccagaagctgatccaccaccggaaagagaccctgtcacgcagaggcccttcagcctccagtgttgcagttatgacctcatcaaccagtgaccaccacttggatgctgctgcagccaggcagcccaatggggtgtgtcgagctgggttcgagcggcagcacagcctacccagttctgagcatcttggggcagatggaggcctctaccagatcccacttccatcttcccagatcccaccacagcctcgccgagcagcacccaccacaccgcccccaccagtgaagcgccgagaccgcgaggccctgatggcctctgggagtggtggccacaacaccatgccctccgggggtaactctgtgtccagcggctcctcagtcagcagcacctccctggacacgctctataccagctccagcccatctgaaccaggctccagctgctcacccacacccccacctgtgccccgccgaggcacccacaccaccgtgtcccaagtccagccccctccctccaaggcatcagcacctgaaccccctgcagaagaagaagtggcaactggtacaacctcagcctctgatgacctggaagccctgggtacactgagcctggggaccacagaggagaaggcagcagctgaggcggctgtgcccaggaccattggggccgagctgatggagctggtgcggagaaacactggcctgagccacgaattatgccgggtggccatcggcatcatagtgggtcacatccaggcctcggtgccggccagctcaccagtcatggagcaggtcctcctctcactcgtagagggcaaggacctcagcatggccctgccctcagggcaggtctgccacgaccagcagaggctggaggtgatctttgcagacctggctcgccggaaggacgacgcccagcagcgcagttgggcactatatgaggatgagggtgtcatccgctgctacctagaggagctgctgcatattctgactgatgcagaccctgaagtttgcaagaaaatgtgcaagagaaacgagttcgagtctgtcctggccttggtggcctattaccaaatggaacaccgagcatcactgcggctgctgctcctcaagtgctttggcgccatgtgcagcctggatgcagccatcatctccacgcttgtgtcatccgtgctgcctgtagagctggcgagggacatgcagacagacacgcaggaccaccagaaactctgttactctgccctcatcctggccatggtcttctccatgggagaggcagtgccctatgcacactatgagcacctgggcacgcctttcgcccagttcctactgaacatcgtcgaggatgggctgcccttggacaccacagagcagctgccggacctctgcgtgaacctgcttctggctctcaacctgcacctgccagctgctgaccagaatgtcatcatggctgccctgagcaaacacgccaatgtcaagatcttctccgagaagctgttgttgctcctgaacagaggggatgaccctgtgcgcatcttcaaacatgagccacagccaccacactctgtcctcaagttcctgcaggacgtgtttggcagcccggccacagctgccatcttctaccacacagacatgatggctctcattgacatcactgtgcggcacatcgcagacctgtcaccaggagacaagctgcgcatggagtacctctccctgatgcatgctatagtccgcaccacaccctacctgcagcaccgccaccggctacccgacctgcaggccatactgcgacgcatcctgaatgaggaggagacctcaccccagtgccagatggaccgcatgattgtccgagagatgtgcaaggaattcctggtgctgggggaggctcccagctagcaccttgctgtcctcccttccctgcagctccagtcagtgtgcaggggactcaggggcttggccctaagaatgttttgcactgacagtgggaggggataaggtggcagaaggaacctgagcaggacccccacctgaagtagaacttaagagaggggccaagtgtaggattggggaccatggtctgggagctgttctggggcagggggaatatgcaagctaaagcccctctataccctggtaggcttcctcttcagcatgcccccctcttcaccccacacccatgtgtacactcagagtcctgctgccgctccaggctggaccagacccccatccccacccctcctggtctgtatcctgggcctcagcaagccatgcccctggggtgaggggaggcatctcccaccagctctattctttgccttagctttgcagtgagtgctgctcatgtcctcccctccacccgcacatgggggtcgctgcccttcactcctacccccagaggccttggccaagctgctgctttgaaagcgggatcttggagacaggccatccatcctggagcctcatggaacaggatgatggcactgagaaagccaatgaccgaatctcttttctctgtaaaaatgtagactgaaaagccatgtgtattttcctatgtgctgtagctctcctttggaaataaatcacaggcatctggaaaaaaaaaaaaaaaaaaaaaaaaaaaaaaaaaaaaaaaaaaaaaaaaaaaaaaaaaaaaaaa

SEQ ID NO: 9 - DNA, 2968 bp, Homo sapiens
ggcgggacaaccgcggcgcggctcggcgctcccggcctggcgttgtggcgccgcagcccttgccaccttccctgcacctgccctgcccggcctcatgtaccgcgcgctgtacgcgttccgctcggcggagcccaacgcgctggcgttcgccgcgggcgagaccttcctggtgctagagcgaagcagcgcgcactggtggctggccgcgcgggcgcgcagtggtgagacgggctacgtgccgccagcctacctgcgccgcctgcagggcctggagcaggatgtcctccaggccattgaccgggccatcgaggctgtacacaacacagccatgcgggatggtggcaagtacagcctggaacagcgtggagtcctccagaagctgatccaccaccggaaagagaccctgtcacgcagaggcccttcagcctccagtgttgcagttatgacctcatcaaccagtgaccaccacttggatgctgctgcagccaggcagcccaatggggtgtgtcgagctgggttcgagcggcagcacagcctacccagttctgagcatcttggggcagatggaggcctctaccagatcccaccacagcctcgccgagcagcacccaccacaccgcccccaccagtgaagcgccgagaccgcgaggccctgatggcctctgggagtggtggccacaacaccatgccctccgggggtaactctgtgtccagcggctcctcagtcagcagcacctccctggacacgctctataccagctccagcccatctgaaccaggctccagctgctcacccacacccccacctgtgccccgccgaggcacccacaccaccgtgtcccaagtccagccccctccctccaaggcatcagcacctgaaccccctgcagaagaagaagtggcaactggtacaacctcagcctctgatgacctggaagccctgggtacactgagcctggggaccacagaggagaaggcagcagctgaggcggctgtgcccaggaccattggggccgagctgatggagctggtgcggagaaacactggcctgagccacgaattatgccgggtggccatcggcatcatagtgggtcacatccaggcctcggtgccggccagctcaccagtcatggagcaggtcctcctctcactcgtagagggcaaggacctcagcatggccctgccctcagggcaggtctgccacgaccagcagaggctggaggtgatctttgcagacctggctcgccggaaggacgacgcccagcagcgcagttgggcactatatgaggatgagggtgtcatccgctgctacctagaggagctgctgcatattctgactgatgcagaccctgaagtttgcaagaaaatgtgcaagagaaacgagttcgagtctgtcctggccttggtggcctattaccaaatggaacaccgagcatcactgcggctgctgctcctcaagtgctttggcgccatgtgcagcctggatgcagccatcatctccacgcttgtgtcatccgtgctgcctgtagagctggcgagggacatgcagacagacacgcaggaccaccagaaactctgttactctgccctcatcctggccatggtcttctccatgggagaggcagtgccctatgcacactatgagcacctgggcacgcctttcgcccagttcctactgaacatcgtcgaggatgggctgcccttggacaccacagagcagctgccggacctctgcgtgaacctgcttctggctctcaacctgcacctgccagctgctgaccagaatgtcatcatggctgccctgagcaaacacgccaatgtcaagatcttctccgagaagctgttgttgctcctgaacagaggggatgaccctgtgcgcatcttcaaacatgagccacagccaccacactctgtcctcaagttcctgcaggacgtgtttggcagcccggccacagctgccatcttctaccacacagacatgatggctctcattgacatcactgtgcggcacatcgcagacctgtcaccaggagacaagctgcgcatggagtacctctccctgatgcatgctatagtccgcaccacaccctacctgcagcaccgccaccggctacccgacctgcaggccatactgcgacgcatcctgaatgaggaggagacctcaccccagtgccagatggaccgcatgattgtccgagagatgtgcaaggaattcctggtgctgggggaggctcccagctagcaccttgctgtcctcccttccctgcagctccagtcagtgtgcaggggactcaggggcttggccctaagaatgttttgcactgacagtgggaggggataaggtggcagaaggaacctgagcaggacccccacctgaagtagaacttaagagaggggccaagtgtaggattggggaccatggtctgggagctgttctggggcagggggaatatgcaagctaaagcccctctataccctggtaggcttcctcttcagcatgcccccctcttcaccccacacccatgtgtacactcagagtcctgctgccgctccaggctggaccagacccccatccccacccctcctggtctgtatcctgggcctcagcaagccatgcccctggggtgaggggaggcatctcccaccagctctattctttgccttagctttgcagtgagtgctgctcatgtcctcccctccacccgcacatgggggtcgctgcccttcactcctacccccagaggccttggccaagctgctgctttgaaagcgggatcttggagacaggccatccatcctggagcctcatggaacaggatgatggcactgagaaagccaatgaccgaatctcttttctctgtaaaaatgtagactgaaaagccatgtgtattttcctatgtgctgtagctctcctttggaaataaatcacaggcatctggaaaaaaaaaaaaaaaaaaaaaaaaaaaaa

SEQ ID NO: 10 - protein, 722 aa, Homo sapiens
MYRALYAFRSAEPNALAFAAGETFLVLERSSAHWWLAARARSGETGYVPPAYLRRLQGLEQDVLQAIDRAIEAVHNTAMRDGGKYSLEQRGVLQKLIHHRKETLSRRGPSASSVAVMTSSTSDHHLDAAAARQPNGVCRAGFERQHSLPSSEHLGADGGLYQIPLPSSQIPPQPRRAAPTTPPPPVKRRDREALMASGSGGHNTMPSGGNSVSSGSSVSSTSLDTLYTSSSPSEPGSSCSPTPPPVPRRGTHTTVSQVQPPPSKASAPEPPAEEEVATGTTSASDDLEALGTLSLGTTEEKAAAEAAVPRTIGAELMELVRRNTGLSHELCRVAIGIIVGHIQASVPASSPVMEQVLLSLVEGKDLSMALPSGQVCHDQQRLEVIFADLARRKDDAQQRSWALYEDEGVIRCYLEELLHILTDADPEVCKKMCKRNEFESVLALVAYYQMEHRASLRLLLLKCFGAMCSLDAAIISTLVSSVLPVELARDMQTDTQDHQKLCYSALILAMVFSMGEAVPYAHYEHLGTPFAQFLLNIVEDGLPLDTTEQLPDLCVNLLLALNLHLPAADQNVIMAALSKHANVKIFSEKLLLLLNRGDDPVRIFKHEPQPPHSVLKFLQDVFGSPATAAIFYHTDMMALIDITVRHIADLSPGDKLRMEYLSLMHAIVRTTPYLQHRHRLPDLQAILRRILNEEETSPQCQMDRMIVREMCKEFLVLGEAPS

SEQ ID NO: 11 - protein, 715 aa, Homo sapiens
MYRALYAFRSAEPNALAFAAGETFLVLERSSAHWWLAARARSGETGYVPPAYLRRLQGLEQDVLQAIDRAIEAVHNTAMRDGGKYSLEQRGVLQKLIHHRKETLSRRGPSASSVAVMTSSTSDHHLDAAAARQPNGVCRAGFERQHSLPSSEHLGADGGLYQIPPQPRRAAPTTPPPPVKRRDREALMASGSGGHNTMPSGGNSVSSGSSVSSTSLDTLYTSSSPSEPGSSCSPTPPPVPRRGTHTTVSQVQPPPSKASAPEPPAEEEVATGTTSASDDLEALGTLSLGTTEEKAAAEAAVPRTIGAELMELVRRNTGLSHELCRVAIGIIVGHIQASVPASSPVMEQVLLSLVEGKDLSMALPSGQVCHDQQRLEVIFADLARRKDDAQQRSWALYEDEGVIRCYLEELLHILTDADPEVCKKMCKRNEFESVLALVAYYQMEHRASLRLLLLKCFGAMCSLDAAIISTLVSSVLPVELARDMQTDTQDHQKLCYSALILAMVFSMGEAVPYAHYEHLGTPFAQFLLNIVEDGLPLDTTEQLPDLCVNLLLALNLHLPAADQNVIMAALSKHANVKIFSEKLLLLLNRGDDPVRIFKHEPQPPHSVLKFLQDVFGSPATAAIFYHTDMMALIDITVRHIADLSPGDKLRMEYLSLMHAIVRTTPYLQHRHRLPDLQAILRRILNEEETSPQCQMDRMIVREMCKEFLVLGEAPS

SEQ ID NO: 12 - DNA, 3885 bp, Homo sapiens
aggccggggcggggctgggaagtagtcgggcggggttgtgagacgccgcgctcagcttccatcgctgggcggtcaacaagtgcgggcctggctcagcgcgggggggcgcggagaccgcgaggcgaccgggagcggctgggttcccggctgcgcgcccttcggccaggccgggagccgcgccagtcggagcccccggcccagcgtggtccgcctccctctcggcgtccacctgcccggagtactgccagcgggcatgaccgacccaccaggggcgccgccgccggcgctcgcaggccgcggatgaagaagaaaacccggcgccgctcgacccggagcgaggagttgacccggagcgaggagttgaccctgagtgaggaagcgacctggagtgaagaggcgacccagagtgaggaggcgacccagggcgaagagatgaatcggagccaggaggtgacccgggacgaggagtcgacccggagcgaggaggtgaccagggaggaaatggcggcagctgggctcaccgtgactgtcacccacagcaatgagaagcacgaccttcatgttacctcccagcagggcagcagtgaaccagttgtccaagacctggcccaggttgttgaagaggtcataggggttccacagtcttttcagaaactcatatttaagggaaaatctctgaaggaaatggaaacaccgttgtcagcacttggaatacaagatggttgccgggtcatgttaattgggaaaaagaacagtccacaggaagaggttgaactaaagaagttgaaacatttggagaagtctgtggagaagatagctgaccagctggaagagttgaataaagagcttactggaatccagcagggttttctgcccaaggatttgcaagctgaagctctctgcaaacttgataggagagtaaaagccacaatagagcagtttatgaagatcttggaggagattgacacactgatcctgccagaaaatttcaaagacagtagattgaaaaggaaaggcttggtaaaaaaggttcaggcattcctagccgagtgtgacacagtggagcagaacatctgccaggagactgagcggctgcagtctacaaactttgccctggccgagtgaggtgtagcagaaaaaggctgtgctgccctgaagaatggcgccaccagctctgccgtctctggagcggaatttacctgatttcttcagggctgctgggggcaactggccatttgccaattttcctactctcacactggttctcaatgaaaaatagtgtctttgtgattttgagtaaagctcctatctgttttctccttctgtctctgtggttgtactgtccagcaatccaccttttctggagagggccacctctgcccaaattttcccagctgtttggacctctgggtgctttctttgggctggtgagagctctaatttgccttgggccagtttcaggtttataggccccctcagtcttcagatacatgagggcttctttgctcttgtgatcgtgtagtcccatagctgtaaaaccagaatcaccaggaggttgcacctagtcaggaatattgggaatggcctagaacaaggtgtttggcacataagtagaccacttatccctcattgtgacctaattccagagcatctggctgggttgttgggttctagactttgtcctcacctcccagtgaccctgactagccacaggccatgagataccagggggccgttccttggatggagcctgtggttgatgcaaggcttccttgtccccaagcaagtcttcagaaggttagaacccagtgttgactgagtctgtgcttgaaaccaggccagagccatggattaggaagggcaaagagaaggcaccagaatgagtaaagcaggcaggtggtgaagccaaccataaacttctcaggagtgacatgtgcttccttcaaaggcatttttgttaaccatatccttctgagttctatgtttccttcacagctgttctatccattttgtggactgtcccccacccccaccccatcattgtttttaaaaaattaaggcctggcgcagcagctcatgcctataatcccagcactttgggaggctgaggcgggcggatcacttgaggccaggagtttgagaccagcccaggcaacatagcaaaaccccattctgctttaaaaaaaaaaaaaaaaaaaattagcttggcgtagtggcatgtgcctataatcccagctactggggaggctgaggcacaagaatcatttgaacctgggaggtagaggttgctgtgagccgagattacgcccctgcactccagcctgggtcacagagtgagactccatctcagaaaaaaaaaaaattgagtcaggtgcagtagctccttcctgtagtcccagctacttgggaggctgaggctagaggatcacttgagcccaggagtttgagtctagtctgggcaacatagcaagaccccatctctaaaatttaagtaagtaaaagtagataaataaaaagaaaaaaaaactgtttatgtgctcatcataaagtagaagagtggtttgcttttttttttttttttggattaatgaggaaatcattctgtggctctagtcataatttatgcttaataacattgatagtagccctttgcgctataactctacctaaagactcacatcatttggcagagagagagtcgttgaagtcccaggaattcaggactgggcaggttaagacctcagacaaggtagtagaggtagacttgtggacaaggctcgggtcccagcccaccgcaccccaactttaatcagagtggttcactattgatctatttttgtgtgatagctgtgtggcgtgggccacaacatttaatgagaagttactgtgcaccaaactgccgaacaccattctaaactattcatatatattagtcatttaattcttacataacttgagaggtagacagatatccttattttagagatgaggaaaccaagagaacttaggtcattagcgcaaggttgtagagtaagcggcaaagccaagacacaaagctgggtggtttggtttcagagccagtgcttttcccctctactgtactgcctctcaaccaacacagggttgcacaggcccattctctgatttttttcctcttgtcctctgcctctccctctagctcccacttcctctctgctctagttcattttctttagagcagcccgagtgatcatgaagtgcaaatcttgccatgtcagtcccctgcttagaaccctccaatggctcactttctctttaggcaaaagtctttaccccatgccttctcccatctcatctcaaccccctcatttgttggctgtctgctgtcagccactcttctttcaggtcctcagatgcactgcaccctctcctgcctgggggtctttgctcctgctactacctctgcttgaacagctcctcaccttccttcctccaaccctacccttgtataggtgacttttgttcatccttcagaattcaactcacatgtctcttgcatggagaaccctcacctactgtgttgagaccctgtccagcccccaggtgggatcctctctcgacttcccatacatttctttcacagcatttacatagtccatgatagtttacttgtgggattatttggttaatctttgcctttaacaccagggttccttgggtgaaggagcttctttatcttggtaacagcattatttcaagcataacttgtaatatagttatattacatatataacatatatatatataacataacatatataacatatataacaagcataacttgttatatagtcttgtatatagtaagacctcaataaatatttggagaacaaaaaaaaaaaaaaa

SEQ ID NO: 13 - protein, 230 aa, Homo sapiens
MNRSQEVTRDEESTRSEEVTREEMAAAGLTVTVTHSNEKHDLHVTSQQGSSEPVVQDLAQVVEEVIGVPQSFQKLIFKGKSLKEMETPLSALGIQDGCRVMLIGKKNSPQEEVELKKLKHLEKSVEKIADQLEELNKELTGIQQGFLPKDLQAEALCKLDRRVKATIEQFMKILEEIDTLILPENFKDSRLKRKGLVKKVQAFLAECDTVEQNICQETERLQSTNFALAE

SEQ ID NO: 14 - DNA, 4468 bp, Homo sapiens
agcaaatcagatatgcccaaaacgttacagaggtaacagtgaatccaggatgctgctgtcagcctgtgcattgtgaagaaggcaatcataggcgagcagccgcgggagcaggaacagcagcgtgctaagaagcagtcacataaacagcagcaggagtaggcctcctgcttttcaaaagcagagtactgcagggtcgcgaaatgcaagacactcagatgtttgaaaatctcccgagttgagaatggctactgtaaaagcgtcaccaagaaactctgacgatctggacagtcctaactctgtgttagcaatacttacttccggaaaattaatgctacttcttgtagatttttgcaaataggaaacccccttgaagaagatctcaaattacgccccccacccccaaaaaaagacaaacaggggagaacaaagttttggcatgcctgcaggaacggtggcttttttagaaactacctaggaggcagaagctaagtgatttgctcatgcctcttacctgggagtagaaggtgggaagaaatggaccgaggctgtgacgagaagacaaggcacagtgcagcttggtgaagccacacgctgactgcgttctgccccctcttcatgcagtgctgcgggctggtgcatcgccggcgagtacgggtgtcctatggttcggcagactcctacactagccgtccatccgattccgatgtatctctggaggaggaccgggaggcagtgcgcagagaagcggagcggcaggcccaggcacagttggaaaaagcaaagacaaagcccgttgcatttgcggttcggacaaatgtcagctacagtgcggcccatgaagatgatgttccagtgcctggcatggccatctcattcgaagcaaaagattttctgcatgttaaggaaaaatttaacaatgactggtggatagggcgattggtaaaagaaggctgtgaaatcggattcattccaagcccagtcaaactagaaaacatgaggctgcagcatgaacagagagccaagcaagggaaattctactccagtaaatcaggaggaaattcatcatccagtttgggtgacatagtacctagttccagaaaatcaacacctccatcatctgctatagacatagatgctactggcttagatgcagaagaaaatgatattccagcaaaccaccgctcccctaaacccagtgcaaacagtgtaacgtcaccccactccaaagagaaaagaatgcccttctttaagaagacagagcacactcctccgtatgatgtggtaccttccatgcgaccagtggtcctagtgggcccttctctgaagggctacgaggtcacagatatgatgcaaaaagcgctgtttgattttttaaaacacagatttgaagggcggatatccatcacaagggtcaccgctgacatctcgcttgccaaacgctcggtattaaacaatcccagtaagcacgcaataatagaaagatccaacacaaggtcaagcttagcggaagttcagagtgaaatcgaaaggatttttgaacttgcaagaacattgcagttggtggtccttgacgcggatacaattaatcatccagctcaactcagtaaaacctccttggcccctattatagtatatgtaaagatttcttctcctaaggttttacaaaggttaataaaatctcgagggaaatctcaagctaaacacctcaacgtccagatggtagcagctgataaactggctcagtgtcctccagagctgttcgatgtgatcttggatgagaaccagcttgaggatgcctgtgagcaccttgccgactatctggaggcctactggaaggccacccatcctcccagcagtagcctccccaaccctctccttagccgtacattagccacttcaagtctgcctcttagccccaccctagcctctaattcacagggttctcaaggtgatcagaggactgatcgctccgctcctatccgttctgcttcccaagctgaagaagaacctagtgtggaaccagtcaagaaatcccagcaccgctcttcctcctcagccccacaccacaaccatcgcagtgggacaagtcgcggcctctccaggcaagagacatttgactcggaaacccaggagagtcgagactctgcctacgtagagccaaaggaagattattcccatgaccacgtggaccactatgcctcacaccgtgaccacaaccacagagacgagacccacgggagcagtgaccacagacacagggagtcccggcaccgttcccgggacgtggatcgagagcaggaccacaacgagtgcaacaagcagcgcagccgtcataaatccaaggatcgctactgtgaaaaggatggagaagtgatatcaaaaaaacggaatgaggctggggagtggaacagggatgtttacatccgccaatgagttttgcccgtttgtgtttttttttttttttttttgaagtcttgtataactaacagcatccccaaaacaaagtctttggggtctacactgcaatcatatgtgatctgtcttgtaatattttgtattattgctgttgcttgaatagcaatagcatggatagagtattgagatactttttcttttgtaagtgctacataaattggcctggtatggctgcagtcctccggttgcatactggactcttcaaaaactgttttgggtagctgccacttgaacaaaatctgttgccacccaggtgatgttagtgttttaagaaatgtagttgatgtatccaacaagccagaatcagcacagataaaaagtggaatttcttgtttctccagatttttaatacgttaatacgcaggcatctgatttgcatattcattcatggaccactgtttcttgcttgtacctctggctgactaaatttggggacagattcagtcttgccttacacaaaggggatcataaagttagaatctattttctatgtactagtactgtgtactgtatagacagtttgtaaatgttatttctgcaaacaaacaccttcttattatatatataatatatatatatatcagtttgatcacactattttagagtcttaatgccaagtcagcagatttgctttatgaattacagggactagaaatgcccacattcaggaaatttgtaataacattgtctagacacctatcctcattctagtagaaagtgtgtacatactgtaaatatgtgtgattgcttgacttgaaaaggtttgaattctgaatgttataccatccttgtaagtaagtttgtaatttccaccataaattatggtaaatataaaactccagaggttgttctactccatacagttcacactgattgtgacacattcttagtagctagtgtctgttctagtcactgcactggagtctacgagccggaactcgctatatgcacgtgtgtgtgtccgtatgtaagaaagtgtgcaccgagtgactgaatggttgagatgaattggaatgctgaagactaacgaagaaactagagactgatatcgagcattctgcccacctcgctctgtatttaattaattgtgctatatgttgctttaacaacccattgagcagtcagggaatgtgagtaagcttgctgccaaaggtaactaggaaagcattcatctgctgcctccttgtttttgctcctagagagtgaaaatacaggcaattttactgtgagtgtttcactggaaatgtacaatctttgtgtgttagagtatttgttttagtaagaaatgtttacacagcttgtggaattatttcgtgggaaaataaatttttataacttctcccacttcaatttctaaccttgcctattgttcctgttgtttgtttacctcccagttacctaccattcctccccaccaccgactccagcaggttcactgtctgtcagaacccagaagtgcttcttataaccaaagtttctgttcttcagaagaaatcagggcaaaatggggtgacttgaagtgaataaaatgttaagaatattatcctacataagacatgtacacagaaggggaaccttgaagacattatctccatgcctcaattacactgctgtaagaagcttacaatgtcatcatgtttaaggctaagaccttttccgtgtctcaagtgtatattttgtccagttataactggatggtaagacagtattaagagtgcaagtacctggcacttgaagtttgtcccaggaaaatgcctgtgtataattacctaacttcagatctgcacattaacttatttaacaaaataaatcagccgggcgcagtggctcatgcctgtaatcccagcaatttgggaggctgaggagggtggatcacctgaggtcaggagttcaagaccagcctggccaacgtggcgaaaccccgtctctactaaaaagataaaaaattagccaggtgtggtggtgcacacctgtaatcccagctacttgggag

SEQ ID NO: 15 - protein, 605 aa, Homo sapiens
MQCCGLVHRRRVRVSYGSADSYTSRPSDSDVSLEEDREAVRREAERQAQAQLEKAKTKPVAFAVRTNVSYSAAHEDDVPVPGMAISFEAKDFLHVKEKFNNDWWIGRLVKEGCEIGFIPSPVKLENMRLQHEQRAKQGKFYSSKSGGNSSSSLGDIVPSSRKSTPPSSAIDIDATGLDAEENDIPANHRSPKPSANSVTSPHSKEKRMPFFKKTEHTPPYDVVPSMRPVVLVGPSLKGYEVTDMMQKALFDFLKHRFEGRISITRVTADISLAKRSVLNNPSKHAIIERSNTRSSLAEVQSEIERIFELARTLQLVVLDADTINHPAQLSKTSLAPIIVYVKISSPKVLQRLIKSRGKSQAKHLNVQMVAADKLAQCPPELFDVILDENQLEDACEHLADYLEAYWKATHPPSSSLPNPLLSRTLATSSLPLSPTLASNSQGSQGDQRTDRSAPIRSASQAEEEPSVEPVKKSQHRSSSSAPHHNHRSGTSRGLSRQETFDSETQESRDSAYVEPKEDYSHDHVDHYASHRDHNHRDETHGSSDHRHRESRHRSRDVDREQDHNECNKQRSRHKSKDRYCEKDGEVISKKRNEAGEWNRDVYIRQ

SEQ ID NO: 16 - protein, 22 aa, Homo sapiens
MGSPGMVLGLLVQIWALQEASS

SEQ ID NO: 17 - protein, 105 aa, Homo sapiens
PNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFV

SEQ ID NO: 18 - protein, 110 aa, Homo sapiens
WGRRSCQQRDSGNSPGNAFYSNVLYRPRGPPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 19 - protein, 5 aa, Artificial Sequence
  source: /note=“Description of Artificial Sequence: Synthetic peptide”
GGGGC

SEQ ID NO: 20 - RNA, 21 nt, Artificial Sequence
  source: /note=“Description of Artificial Sequence: Synthetic oligonucleotide”
cagcaaagggacucagguauu

SEQ ID NO: 21 - RNA, 21 nt, Artificial Sequence
  source: /note=“Description of Artificial Sequence: Synthetic oligonucleotide”
agguaacagcccaggaaauuu

SEQ ID NO: 22 - DNA, 20 bp, Artificial Sequence
  source: /note=“Description of Artificial Sequence: Synthetic primer”
ctgagttggaggaggctgag

SEQ ID NO: 23 - DNA, 20 bp, Artificial Sequence
  source: /note=“Description of Artificial Sequence: Synthetic primer”
cgatccggtttctgttctgt

SEQ ID NO: 24 - DNA, 33 bp, Artificial Sequence
  source: /note=“Description of Artificial Sequence: Synthetic oligonucleotide”
  modified_base (3)..(31): a, c, t, g, unknown or other
aannnnnnnnnnnnnnnnnnnnnnnnnnnnntt